SEQUENCE LISTING (169 sequences)

SEQ ID NO: 1 - protein, 234 aa, Artificial Sequence
  Synthetic Construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 2 - protein, 215 aa, Artificial Sequence
  Synthetic Construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADSS

SEQ ID NO: 3 - protein, 215 aa, Artificial Sequence
  Synthetic Construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAESS

SEQ ID NO: 4 - protein, 229 aa, Artificial Sequence
  Synthetic Construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 5 - protein, 235 aa, Artificial Sequence
  Synthetic Construct
MDMRVPAQLLGLLLLWLPGAKCDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 6 - protein, 230 aa, Artificial Sequence
  Synthetic Construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 7 - protein, 213 aa, Artificial Sequence
  Synthetic Construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 8 - protein, 227 aa, Artificial Sequence
  Synthetic Construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 9 - protein, 214 aa, Artificial Sequence
  Synthetic Construct
DIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 10 - protein, 231 aa, Artificial Sequence
  Synthetic Construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 11 - protein, 219 aa, Artificial Sequence
  Synthetic Construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 12 - protein, 227 aa, Artificial Sequence
  Synthetic Construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 13 - protein, 214 aa, Artificial Sequence
  Synthetic Construct
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 14 - protein, 226 aa, Artificial Sequence
  Synthetic Construct
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 15 - protein, 214 aa, Artificial Sequence
  Synthetic Construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 226 aa, Artificial Sequence
  Synthetic Construct
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 17 - protein, 214 aa, Artificial Sequence
  Synthetic Construct
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 18 - protein, 227 aa, Artificial Sequence
  Synthetic Construct
QVQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 19 - protein, 219 aa, Artificial Sequence
  Synthetic Construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 20 - protein, 229 aa, Artificial Sequence
  Synthetic Construct
QVQLQQPGTELVRPGSSVKLSCKASGYTFTSYWVNWVKQRPDQGLEWIGRIDPYDSETHYNQKFTDKAISTIDTSSNTAYMQLSTLTSDASAVYYCSRSPRDSSTNLADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 21 - protein, 214 aa, Artificial Sequence
  Synthetic Construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 22 - protein, 227 aa, Artificial Sequence
  Synthetic Construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGAGAYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 23 - protein, 214 aa, Artificial Sequence
  Synthetic Construct
DIVMTQSPSSLSASVGDRVTITCRTSRSISAALAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 24 - protein, 494 aa, Artificial Sequence
  Synthetic Construct
METDTLLLWVLLLWVPGSTGDYKAKEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 1491 bp, Artificial Sequence
  Synthetic Construct
atggagaccgacaccctgttgctttgggtactgttactttgggtgcccggatctaccggtgattacaaggccaaggaggtgcagctgcagcagagcggagccgagctggtgaagccaggcgcttccgtgaagatgtcttgtaaggcctccggctacacattcaccagctacaatatgcactgggtaaagcagactccggggcagggcctggagtggataggtgccatctaccctggcaacggcgacaccagctacaaccagaagtttaaggggaaggctactctaacagcggacaagtcgtcctctaccgcctacatgcaactcagctccctgacgagcgaggactccgcggactactactgtgcccgctccaactactacggctctagctattggttcttcgacgtgtggggcgctggaacgaccgtgaccgtgtcttccggtggaggttccgggggcggaagcggcggtggcggcagttcggacatcgtgctgacccagagccctgccatcctgtccgcttccccgggggagaaagttacgatgacctgccgagcgagctccagtgtcaactacatggattggtaccagaagaagcccggcagcagtcccaagccgtggatttacgctactagcaacctggcgtccggtgtcccggctcgcttctcaggttctggctcgggtactagttattcattaaccatttctcgcgtggaggctgaggacgctgccacctactactgccaacagtggtctttcaaccctcccactttcggaggcggcaccaagctcgagatcaagggcgggggtggctccgcagcagccattgaggtgatgtatcctcctccctatttggacaacgagaagtcaaatggcaccatcatccacgttaagggcaagcacctgtgcccatctcccctgttcccaggcccctctaagcccttctgggtcctggtggtggtcggcggcgtcctggcatgttactctctgctggtgaccgtcgcgttcatcatcttttgggtccggtccaagcgcagccgcctgctccactccgactacatgaatatgactcctcgtaggcccggtccaacccgcaagcactaccagccgtacgcgccgcccagagactttgctgcttaccgatccagagtgaaattttctaggtcggccgaacctcccgcatatcagcagggccagaaccagctgtacaacgaactcaacttgggacggcgcgaggaatacgatgtgctggataaacgccgtggccgcgatcccgagatgggcgggaagccacgtcgcaaaaaccctcaggagggcctttacaacgagttgcagaaggacaaaatggcggaggcctactccgagatcggaatgaagggggagcgccggcgcggcaaagggcatgacggcctctaccagggcctgtccacagccacgaaagacacctatgacgccctgcatatgcaggccctgcccccgcgctgataatga

SEQ ID NO: 26 - protein, 248 aa, Artificial Sequence
  Synthetic Construct
QVQLQQSGPGLVKPSQTLSLTCAISGDSVSSNSAAWNWIRQSPSNWIRQSPSGLEWLGRTYYRSKWYNDYAVSVKSRITINPDTSKNQFSLQLNSVTPEDTAVYYCARENIAAWTWAFDIWGQGTMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 27 - protein, 214 aa, Artificial Sequence
  Synthetic Construct
EIVMTQSPGTLSLSPGERATLSCRASQSVSSSYLAWYQQKPGLAPRLLIYDTSLRATDIPDRFSGSGSGTAFTLTISRLEPEDFAVYYCQQYGSSPTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 28 - protein, 238 aa, Artificial Sequence
  Synthetic Construct
EVQLVQSGAEVKKPGSSVKVSCKASGGTFTGYYMHWVRQAPGQGLEWMGWINPNSGGTNYAQKFQGRVTMTRDTSISTAYMELSRLRSDDTAVYYCARDLSSGYSGYFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 29 - protein, 219 aa, Artificial Sequence
  Synthetic Construct
DVVMTQSPLSLPVTPGEPASISCRSSQSLLHSNGYNYLNWYLQKPGQSPQLLIYLGSNRASGVPDRFSGSGSGTDFTLKISRVEGEDVGDYYCMQALQSPFTFGQGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 30 - protein, 238 aa, Artificial Sequence; identical to SEQ ID NO: 28
  Synthetic Construct

SEQ ID NO: 31 - protein, 219 aa, Artificial Sequence
  Synthetic Construct
DVVMTQSPLSLPVTPGEPASISCRSSQSLLHSNGYNFLDWYLQKPGQSPQLLIYLGSNRASGVPDRFSGSGSGTDFTLKISRVEADDVGVYYCMQSLQTPWTFGHGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 32 - protein, 233 aa, Artificial Sequence
  Synthetic Construct
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 33 - protein, 219 aa, Artificial Sequence
  Synthetic Construct
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 34 - protein, 232 aa, Artificial Sequence
  Synthetic Construct
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGEIMDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 35 - protein, 214 aa, Artificial Sequence
  Synthetic Construct
DILLTQSPAILSVSPGERVSFSCRASQRIGTSIHWYQQRTTGSPRLLIKYASESISGIPSRFSGSGSGTDFTLSINSVESEDVADYYCQQSHGWPFTFGGGTKLEIERTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 36 - protein, 233 aa, Artificial Sequence
  Synthetic Construct
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 37 - protein, 214 aa, Artificial Sequence
  Synthetic Construct
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 38 - protein, 234 aa, Artificial Sequence; identical to SEQ ID NO: 1
  Synthetic Construct

SEQ ID NO: 39 - protein, 215 aa, Artificial Sequence; identical to SEQ ID NO: 3
  Synthetic Construct

SEQ ID NO: 40 - protein, 234 aa, Artificial Sequence
  Synthetic Construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 41 - protein, 215 aa, Artificial Sequence
  Synthetic Construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 42 - protein, 226 aa, Artificial Sequence
  Synthetic Construct
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 43 - protein, 214 aa, Artificial Sequence
  Synthetic Construct
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 227 aa, Artificial Sequence
  Synthetic Construct
QVQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 45 - protein, 219 aa, Artificial Sequence
  Synthetic Construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 240 aa, Artificial Sequence
  Synthetic Construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 47 - protein, 215 aa, Artificial Sequence
  Synthetic Construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 48 - protein, 234 aa, Artificial Sequence
  Synthetic Construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDNYSLDYWGQGTTLTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 49 - protein, 213 aa, Artificial Sequence
  Synthetic Construct
QIVLTQSPSSLSASLGEKVTMTCRASSSVSYMNWYQQKPGTSPKRWIYDTSKVASGVPDRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 50 - protein, 234 aa, Artificial Sequence
  Synthetic Construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 51 - protein, 216 aa, Artificial Sequence
  Synthetic Construct
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLGQPKANPTVTLFPPSSEELQANKATLVCLISDFYPGAVTVAWKADGSPVKAGVETTKPSKQSNNKYAACSYLSLTPEQWKSHRSYSCQVTHEGSTVEKTVAPTESS

SEQ ID NO: 52 - protein, 237 aa, Artificial Sequence
  Synthetic Construct
EVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 53 - protein, 214 aa, Artificial Sequence
  Synthetic Construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 54 - protein, 234 aa, Artificial Sequence
  Synthetic Construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 55 - protein, 213 aa, Artificial Sequence
  Synthetic Construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 56 - protein, 230 aa, Artificial Sequence
  Synthetic Construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 57 - protein, 213 aa, Artificial Sequence
  Synthetic Construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 58 - protein, 226 aa, Artificial Sequence
  Synthetic Construct
EVQLVESGGGLVQPGASLKLSCVASGFTFSDYWMSWVRQTPGKPMEWIGHIKYDGSYTNYAPSLKNRFTISRDNAKTTLYLQMSNVRSEDSATYYCAREAPGAASYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 59 - protein, 219 aa, Artificial Sequence
  Synthetic Construct
DVVLTQTPVAQPVTLGDQASISCRSSQSLVHSNGNTYLEWFLQKPGQSPQLLIYKVSNRFSGVPDRFIGSGSGSDFTLKISRVEPEDWGVYYCFQGTHDPYTFGAGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 60 - protein, 229 aa, Artificial Sequence
  Synthetic Construct
EVQLQQSGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 61 - protein, 214 aa, Artificial Sequence
  Synthetic Construct
DIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 62 - protein, 230 aa, Artificial Sequence
  Synthetic Construct
QVQLQQPGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 63 - protein, 219 aa, Artificial Sequence
  Synthetic Construct
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 64 - protein, 236 aa, Artificial Sequence
  Synthetic Construct
EVQLVESGGGLVQPGGSLRLSCAASGYSFTGHWMNWVRQAPGKGLEWVGMIHPSDSETRYNQKFKDRFTISVDKSKNTLYLQMNSLRAEDTAVYYCARGIYFYGTTYFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 65 - protein, 214 aa, Artificial Sequence
  Synthetic Construct
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 66 - protein, 235 aa, Artificial Sequence
  Synthetic Construct
EVQLVESGGDLVQPGRSLRLSCAASGFTFSDYYMSWVRQAPGKGLEWVAAIDNDGGSISYPDTVKGRFTISRDNAKNSLYLQMNSLRVEDTALYYCARQGRLRRDYFDYWGQGTLVTVSTASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 67 - protein, 218 aa, Artificial Sequence
  Synthetic Construct
DIQMTQSPSSLSASVGDRVTITCRASESVDSYGNSFMHWYQQKPGKAPKLLIYRASNLESGVPSRFSGSGSGTDFTFTISSLQPEDIATYYCQQSNEDPLTFGQGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 68 - protein, 239 aa, Artificial Sequence
  Synthetic Construct
EVQLVESGGGLVQPGGSLRLSCATSGYTFTEYTMHWMRQAPGKGLEWVAGINPKNGGTSHNQRFMDRFTISVDKSTSTAYMQMNSLRAEDTAVYYCARWRGLNYGFDVRYFDVWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 69 - protein, 214 aa, Artificial Sequence
  Synthetic Construct
DIQMTQSPSSLSASVGDRVTITCRASQDINNYLNWYQQKPGKAPKLLIYYTSTLHSGVPSRFSGSGSGTDYTLTISSLQPEDFATYYCQQGNTLPPTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 70 - protein, 237 aa, Artificial Sequence
  Synthetic Construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 71 - protein, 481 aa, Artificial Sequence
  Synthetic Construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGESGGGGSGGGGSGGGGSQVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSGGGGSGGGGSGGGGSGGGGSDIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 72 - protein, 231 aa, Artificial Sequence; identical to SEQ ID NO: 10
  Synthetic Construct

SEQ ID NO: 73 - protein, 219 aa, Artificial Sequence; identical to SEQ ID NO: 11
  Synthetic Construct

SEQ ID NO: 74 - protein, 235 aa, Artificial Sequence
  Synthetic Construct
EVQLVESGGGLVQPGGSLRLSCAASGFTFSNYAMSWVRQAPGKRLEWVSAISDHSTNTYYPDSVKGRFTISRDNAKNTLYLQMNSLRAEDTAVYYCARKYGGDYDPFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 75 - protein, 214 aa, Artificial Sequence
  Synthetic Construct
DIQMTQSPSSLSASVGDRVTITCQASQDINNYIAWYQHKPGKGPKLLIHYTSTLQPGIPSRFSGSGSGRDYTLTISSLQPEDFATYYCLQYDNLLFTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 76 - protein, 136 aa, Artificial Sequence
  Synthetic Construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTLVTVSSGGCGGHHHHHH

SEQ ID NO: 77 - protein, 132 aa, Artificial Sequence
  Synthetic Construct
EVQLVESGGGLVQAGGSLRLSCAASGSTFSDYGVGWFRQAPGKGREFVADIDWNGEHTSYADSVKGRFATSRDNAKNTAYLQMNSLKPEDTAVYYCAADALPYTVRKYNYWGQGTQVTVSSGGCGGHHHHHH

SEQ ID NO: 78 - protein, 138 aa, Artificial Sequence
  Synthetic Construct
EVQLVESGGGPVQAGGSLRLSCAASGRTYRGYSMGWFRQAPGKEREFVAAIVWSGGNTYYEDSVKGRFTISRDNAKNIMYLQMTSLKPEDSATYYCAAKIRPYIFKIAGQYDYWGQGTLVTVSSAGGGSGGHHHHHHC

SEQ ID NO: 79 - protein, 133 aa, Artificial Sequence
  Synthetic Construct
EVQLVESGGGLVQPGGSLRLSCAASGDIYKSFDMGWYRQAPGKQRDLVAVIGSRGNNRGRTNYADSVKGRFTISRDGTGNTVYLLMNKLRPEDTAIYYCNTAPLVAGRPWGRGTLVTVSSGGGSGGHHHHHHC

SEQ ID NO: 80 - protein, 138 aa, Artificial Sequence
  Synthetic Construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGCHHHHHHHH

SEQ ID NO: 81 - protein, 128 aa, Artificial Sequence
  Synthetic Construct
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGCGGHHHHHH

SEQ ID NO: 82 - protein, 132 aa, Artificial Sequence
  Synthetic Construct
EVQLVESGGGLVQPGGSLRLSCAASGSIFRLHTMEWYRRTPETQREWVATITSGGTTNYPDSVKGRFTISRDDTKKTVYLQMNSLKPEDTAVYYCHAVATEDAGFPPSNYWGQGTLVTVSSGGCGGHHHHHH

SEQ ID NO: 83 - protein, 135 aa, Artificial Sequence
  Synthetic Construct
EVQLVESGGGPVQAGGSLRLSCAASGRTYRGYSMGWFRQAPGREREFVAAIVWSDGNTYYEDSVKGRFTISRDNAKNTMYLQMTSLKPEDSATYYCAAKIRPYIFKIAGQYDYWGQGTLVTVSSGGCGGHHHHHH

SEQ ID NO: 84 - protein, 235 aa, Artificial Sequence
  Synthetic Construct
EVKLVESGGGLVQPGRSLRLSCAASGFNFYAYWMGWVRQAPGKGLEWIGEIKKDGTTINYTPSLKDRFTISRDNAQNTLYLQMTKLGSEDTALYYCAREERDGYFDYWGQGVMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 85 - protein, 215 aa, Artificial Sequence
  Synthetic Construct
QFVLTQPNSVSTNLGSTVKLSCKRSTGNIGSNYVNWYQQHEGRSPTTMIYRDDKRPDGVPDRFSGSIDRSSNSALLTINNVQTEDEADYFCQSYSSGIVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 86 - protein, 229 aa, Artificial Sequence
  Synthetic Construct
EVQLQQSGPELVKPGASVKMSCKASGYTFTSYVIQWVKQKPGQGLEWIGSINPYNDYTKYNEKFKGKATLTSDKSSITAYMEFSLTSEDSALYCARWGDGNYWGRGTLTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 87 - protein, 211 aa, Artificial Sequence
  Synthetic Construct
DIEMTQSPAIMSASLGERVTMTCTASSSVSSSYFHWYQKPGSSPKLCIYSTSNLASGVPPRFSGSGSTSYSLTISMEAEDAATYFCHQYHRSPTFGGGTKLETKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 88 - protein, 238 aa, Artificial Sequence
  Synthetic Construct
QVQLKESGPGLVAPSQSLSITCTVSGFSLTGYGVNWVRQPPGKGLEWLGMIWGDGSTDYNSALKSRLSITKDNSKSQVFLKMNSLQTDDTARYYCARDGYSNFHYYVMDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 89 - protein, 219 aa, Artificial Sequence
  Synthetic Construct
DIVLTQSPASLAVSLGQRATISCRASESVEYYVTSLMQWYQQKPGQPPKLLISAASNVESGVPARFSGSGSGTDFSLNIHPVEEDDIAMYFCQQSRKVPWTFGGGTKLEIKRRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 90 - protein, 237 aa, Artificial Sequence
  Synthetic Construct
QVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 91 - protein, 218 aa, Artificial Sequence
  Synthetic Construct
DIVLTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 92 - protein, 234 aa, Artificial Sequence
  Synthetic Construct
QVQLQESGPGLVKPSETLSLTCAVSGFSLTSYGVHWIRQPGKGLEWLGVIWPGTNFNSALMSRLTISEDTSKNQVSLKLSSVTAADTAVYCARDRAYGNYLYAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 93 - protein, 210 aa, Artificial Sequence
  Synthetic Construct
DIQMTQSPSLSASVGDRVTITCRASESVEYVTSLMQWYQKPGKAPKLLIYAASNVDSGVPSRFSGSGTDFTLTISLQPEDIATYCQSRKVPFTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 94 - protein, 233 aa, Artificial Sequence
  Synthetic Construct
QVQLVQSGAEVKKPGASVKVSCKASGYTFTDYKIHWVRQAPGQGLEWIGYIYPYSGSSDYNQKFKSRATLTVDNSISTAYMELSRLRSDDTAVYYCARGGDAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 95 - protein, 213 aa, Artificial Sequence
  Synthetic Construct
DIQMTQSPSSLSASVGDRVTITCGASENIYGALNWYQRKPGKAPKLLIYGATNLADGVPSRFSGSGSGRDYTLTISSLQPEDFATYFCQNILGTWTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 96 - protein, 234 aa, Artificial Sequence
  Synthetic Construct
EVQLVESGGGLVQPGGSLRLSCAASGFTFNIYYMSWVRQAPGKGLELVAAINPDGGNTYYPDTVKGRFTISRDNAKNSLYLQMNSLRAEDTAVYYCARYGGPGFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 97 - protein, 215 aa, Artificial Sequence
  Synthetic Construct
ENVLTQSPATLSLSPGERATLSCSASSSVSYMHWYQQKPGQAPRLWIYDTSKLASGIPARFSGSGSRNDYTLTISSLEPEDFAVYYCFPGSGFPFMYTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 98 - protein, 262 aa, Artificial Sequence
  Synthetic Construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSGGGGSGGGGSGGGGSGGGGSDIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKGGGSGGCGGHHHHHH

SEQ ID NO: 99 - protein, 243 aa, Artificial Sequence
  Synthetic Construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 100 - protein, 8 aa, Artificial Sequence
  Synthetic Construct
GSTFSDYG

SEQ ID NO: 101 - protein, 8 aa, Artificial Sequence
  Synthetic Construct
IDWNGEHT

SEQ ID NO: 102 - protein, 14 aa, Artificial Sequence
  Synthetic Construct
AADALPYTVRKYNY

SEQ ID NO: 103 - protein, 4 aa, Artificial Sequence
  Synthetic Construct
KERE

SEQ ID NO: 104 - protein, 4 aa, Artificial Sequence
  Synthetic Construct
KQRE

SEQ ID NO: 105 - protein, 4 aa, Artificial Sequence
  Synthetic Construct
GLEW

SEQ ID NO: 106 - protein, 5 aa, Artificial Sequence
  Synthetic Construct
KEREL

SEQ ID NO: 107 - protein, 5 aa, Artificial Sequence
  Synthetic Construct
KEREF

SEQ ID NO: 108 - protein, 5 aa, Artificial Sequence
  Synthetic Construct
KQREL

SEQ ID NO: 109 - protein, 5 aa, Artificial Sequence
  Synthetic Construct
KQREF

SEQ ID NO: 110 - protein, 5 aa, Artificial Sequence
  Synthetic Construct
KEREG

SEQ ID NO: 111 - protein, 5 aa, Artificial Sequence
  Synthetic Construct
KQREW

SEQ ID NO: 112 - protein, 5 aa, Artificial Sequence
  Synthetic Construct
KQREG

SEQ ID NO: 113 - protein, 4 aa, Artificial Sequence
  Synthetic Construct
TERE

SEQ ID NO: 114 - protein, 5 aa, Artificial Sequence
  Synthetic Construct
TEREL

SEQ ID NO: 115 - protein, 4 aa, Artificial Sequence
  Synthetic Construct
TQRE

SEQ ID NO: 116 - protein, 5 aa, Artificial Sequence
  Synthetic Construct
TQREL

SEQ ID NO: 117 - protein, 4 aa, Artificial Sequence
  Synthetic Construct
KECE

SEQ ID NO: 118 - protein, 5 aa, Artificial Sequence
  Synthetic Construct
KECEL

SEQ ID NO: 119 - protein, 5 aa, Artificial Sequence
  Synthetic Construct
KECER

SEQ ID NO: 120 - protein, 4 aa, Artificial Sequence
  Synthetic Construct
KQCE

SEQ ID NO: 121 - protein, 5 aa, Artificial Sequence
  Synthetic Construct
KQCEL

SEQ ID NO: 122 - protein, 4 aa, Artificial Sequence
  Synthetic Construct
RERE

SEQ ID NO: 123 - protein, 5 aa, Artificial Sequence
  Synthetic Construct
REREG

SEQ ID NO: 124 - protein, 4 aa, Artificial Sequence
  Synthetic Construct
RQRE

SEQ ID NO: 125 - protein, 5 aa, Artificial Sequence
  Synthetic Construct
RQREL

SEQ ID NO: 126 - protein, 5 aa, Artificial Sequence
  Synthetic Construct
RQREF

SEQ ID NO: 127 - protein, 5 aa, Artificial Sequence
  Synthetic Construct
RQREW

SEQ ID NO: 128 - protein, 4 aa, Artificial Sequence
  Synthetic Construct
QERE

SEQ ID NO: 129 - protein, 5 aa, Artificial Sequence
  Synthetic Construct
QEREG

SEQ ID NO: 130 - protein, 4 aa, Artificial Sequence
  Synthetic Construct
QQRE

SEQ ID NO: 131 - protein, 5 aa, Artificial Sequence
  Synthetic Construct
QQREW

SEQ ID NO: 132 - protein, 5 aa, Artificial Sequence
  Synthetic Construct
QQREL

SEQ ID NO: 133 - protein, 5 aa, Artificial Sequence
  Synthetic Construct
QQREF

SEQ ID NO: 134 - protein, 4 aa, Artificial Sequence
  Synthetic Construct
KGRE

SEQ ID NO: 135 - protein, 5 aa, Artificial Sequence
  Synthetic Construct
KGREG

SEQ ID NO: 136 - protein, 4 aa, Artificial Sequence
  Synthetic Construct
KDRE

SEQ ID NO: 137 - protein, 5 aa, Artificial Sequence
  Synthetic Construct
KDREV

SEQ ID NO: 138 - protein, 5 aa, Artificial Sequence
  Synthetic Construct
DECKL

SEQ ID NO: 139 - protein, 5 aa, Artificial Sequence
  Synthetic Construct
NVCEL

SEQ ID NO: 140 - protein, 4 aa, Artificial Sequence
  Synthetic Construct
GVEW

SEQ ID NO: 141 - protein, 4 aa, Artificial Sequence
  Synthetic Construct
EPEW

SEQ ID NO: 142 - protein, 4 aa, Artificial Sequence
  Synthetic Construct
GLER

SEQ ID NO: 143 - protein, 4 aa, Artificial Sequence
  Synthetic Construct
DQEW

SEQ ID NO: 144 - protein, 4 aa, Artificial Sequence
  Synthetic Construct
DLEW

SEQ ID NO: 145 - protein, 4 aa, Artificial Sequence
  Synthetic Construct
GIEW

SEQ ID NO: 146 - protein, 4 aa, Artificial Sequence
  Synthetic Construct
ELEW

SEQ ID NO: 147 - protein, 4 aa, Artificial Sequence
  Synthetic Construct
GPEW

SEQ ID NO: 148 - protein, 4 aa, Artificial Sequence
  Synthetic Construct
EWLP

SEQ ID NO: 149 - protein, 4 aa, Artificial Sequence
  Synthetic Construct
GPER

SEQ ID NO: 150 - protein, 10 aa, Artificial Sequence
  Synthetic Construct
  VARIANT 1, 2, 3, 4, 5, 6, 7, 8, 9: Can be present or absent
  VARIANT 1, 2, 3, 4, 5, 6, 7, 8, 9, 10: Xaa = A preferably naturally occurring amino acid residue that is independently chosen
XXXXXXXXXX

SEQ ID NO: 151 - protein, 5 aa, Artificial Sequence
  Synthetic Construct
  VARIANT 1, 2, 3, 4: Can be present or absent
  VARIANT 1, 2, 3, 4, 5: Xaa = A naturally occurring amino acid, preferably not cysteine
XXXXX

SEQ ID NO: 152 - protein, 5 aa, Artificial Sequence
  Synthetic Construct
  VARIANT (1)..(5): Can be present in repeats of 1, 2, 3, 4, 5, 6, 7 or more
GGGGS

SEQ ID NO: 153 - protein, 3 aa, Artificial Sequence
  Synthetic Construct
AAA

SEQ ID NO: 154 - protein, 5 aa, Artificial Sequence
  Synthetic Construct
GGGGS

SEQ ID NO: 155 - protein, 7 aa, Artificial Sequence
  Synthetic Construct
SGGSGGS

SEQ ID NO: 156 - protein, 8 aa, Artificial Sequence
  Synthetic Construct
GGGGSGGS

SEQ ID NO: 157 - protein, 9 aa, Artificial Sequence
  Synthetic Construct
GGGGSGGGS

SEQ ID NO: 158 - protein, 10 aa, Artificial Sequence
  Synthetic Construct
GGGGSGGGGS

SEQ ID NO: 159 - protein, 15 aa, Artificial Sequence
  Synthetic Construct
GGGGSGGGGSGGGGS

SEQ ID NO: 160 - protein, 18 aa, Artificial Sequence
  Synthetic Construct
GGGGSGGGGSGGGGSGGS

SEQ ID NO: 161 - protein, 20 aa, Artificial Sequence
  Synthetic Construct
GGGGSGGGGSGGGGSGGGGS

SEQ ID NO: 162 - protein, 25 aa, Artificial Sequence
  Synthetic Construct
GGGGSGGGGSGGGGSGGGGSGGGGS

SEQ ID NO: 163 - protein, 30 aa, Artificial Sequence
  Synthetic Construct
GGGGSGGGGSGGGGSGGGGSGGGGSGGGGS

SEQ ID NO: 164 - protein, 35 aa, Artificial Sequence
  Synthetic Construct
GGGGSGGGGSGGGGSGGGGSGGGGSGGGGSGGGGS

SEQ ID NO: 165 - protein, 40 aa, Artificial Sequence
  Synthetic Construct
GGGGSGGGGSGGGGSGGGGSGGGGSGGGGSGGGGSGGGGS

SEQ ID NO: 166 - protein, 15 aa, Artificial Sequence
  Synthetic Construct
EPKSCDKTHTCPPCP

SEQ ID NO: 167 - protein, 24 aa, Artificial Sequence
  Synthetic Construct
GGGGSGGGSEPKSCDKTHTCPPCP

SEQ ID NO: 168 - protein, 12 aa, Artificial Sequence
  Synthetic Construct
EPKTPKPQPAAA

SEQ ID NO: 169 - protein, 62 aa, Artificial Sequence
  Synthetic Construct
ELKTPLGDTTHTCPRCPEPKSCDTPPPCPRCPEPKSCDTPPPCPRCPEPKSCDTPPPCPRCP

(19) United States
(12) Patent Application Publication
(10) Pub. No.: US 2022/0218614 A1
(43) Pub. Date: Jul. 14, 2022

(54) IONIZABLE CATIONIC LIPIDS AND LIPID NANOPARTICLES, AND METHODS OF SYNTHESIS AND USE THEREOF

(71) Applicant: Tidal Therapeutics, Inc., Cambridge, MA (US)

(72) Inventors: Mir ALI, Woburn, MA (US); Austin Wayne BOESCH, Somerville, MA (US); Daryl Clark DRUMMOND, Lincoln, MA (US); William KUHLMAN, Somerville, MA (US); Ulrik NIELSEN, Quincy, MA (US)

(73) Assignee: Tidal Therapeutics, Inc., Cambridge, MA (US)

(21) Appl. No.: 17/542,348

(22) Filed: Dec. 3, 2021

Related U.S. Application Data
(60) Provisional application No. 63/172,024, filed on Apr. 7, 2021.
(60) Provisional application No. 63/169,395, filed on Apr. 1, 2021.
(60) Provisional application No. 63/169,296, filed on Apr. 1, 2021.
(60) Provisional application No. 63/166,205, filed on Mar. 25, 2021.
(60) Provisional application No. 63/121,801, filed on Dec. 4, 2020.

(51) Int. Cl. A61K 9/127; C12N 15/113; A61K 47/20; A61K 47/18; A61K 47/60

(52) U.S. Cl. CPC A61K 9/1272; C12N 15/113; B82Y 5/00; A61K 47/18; A61K 47/60; A61K 47/20

(57) ABSTRACT

Provided are ionizable cationic lipids and lipid nanoparticles for the delivery of nucleic acids to cells (e.g., immune cells), and methods of making and using such lipids and targeted lipid nanoparticles.

### CROSS-REFERENCE TO RELATED APPLICATIONS

This application claims priority to and the benefit of U.S. Provisional Application No. 63/121,801, filed on Dec. 4, 2020; U.S. Provisional Application No. 63/166,205, filed on Mar. 25, 2021; U.S. Provisional Application No. 63/169,296, filed on Apr. 1, 2021; U.S. Provisional Application No. 63/169,395, filed on Apr. 1, 2021; and U.S. Provisional Application No. 63/172,024, filed on Apr. 7, 2021, the entire disclosures of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 183952034000SEQLIST.TXT, date recorded: Dec. 3, 2021, size: 205,763 bytes).

### FIELD OF THE INVENTION

The invention provides ionizable cationic lipids and lipid nanoparticles for the delivery of nucleic acids to immune cells, and methods of making and using, such lipids and targeted lipid nanoparticles.

### BACKGROUND

In recent years, a number of therapeutic modalities have been developed that involve the delivery of one or more nucleic acids to a subject. Treatment modalities include, for example, gene therapies where a gene of interest in the form of deoxyribose nucleic acid (DNA) is introduced into a cell, which is then expressed to produce a gene product, for example, protein, for treating a disorder caused by or associated with a deficiency or absence of the gene product. In this approach, the gene is transcribed into a messenger ribonucleic acid (mRNA), whereupon the mRNA is translated to produce the gene product. In another approach, mRNA rather than a gene of interest can be delivered to the cell. The resulting expression product can ameliorate the deficiency or absence of a particular protein in a subject (for example, a protein deficiency occurring in certain forms of cystic fibrosis or lysosomal storage disorders), or can be used to modulate a cellular function, for example, reprogramming immune cells to initiate or otherwise modulate an immune response in the subject (for example, as a therapeutic agent for treating cancer or as a prophylactic vaccine for preventing or minimizing the risk or severity of a microbial or viral infection).

However, the delivery of mRNA to a cell for translation within the cell has been challenging for a variety of factors, such as nuclease degradation of the mRNA prior to entry into the cell and then after introduction into the cell but prior to translation.

RNA may be delivered to a subject using different delivery vehicles, for example, based on cationic polymers or lipids which, together with the RNA, form nanoparticles. The nanoparticles are intended to protect the RNA from degradation, enable delivery of the RNA to the target site and facilitate cellular uptake and processing by the target cells. For delivery efficacy, in addition to the molecular composition, parameters like particle size, charge, or grafting with molecular moieties, such as polyethylene glycol (PEG) or ligands, play a role. Grafting with PEG is believed to reduce serum interactions, increase serum stability and increase time in circulation, which can be helpful for certain targeting approaches.

Compared with DNA delivery technologies used in certain gene therapies, mRNA-based gene treatment has a number of superior features, for example, ease in manipulation, rapid and transient expression, and adaptive convertibility without mutagenesis.

However, the delivery of therapeutic RNAs to cells is difficult in view of the relative instability and low cell permeability of RNAs. Thus, there exists a need to develop methods and compositions to facilitate the delivery of RNAs such as mRNA to cells.

### SUMMARY

The invention provides ionizable cationic lipids, lipid-immune cell targeting group conjugates, and lipid nanoparticle compositions comprising such ionizable cationic lipids and/or lipid-immune cell (e.g., T-cell) targeting group conjugates, medical kits containing such lipids and/or conjugates, and methods of making and using, such lipids and conjugates.

The lipid nanoparticle compositions provided herein may further comprise a nucleic acid, such as an RNA, e.g., a messenger RNA or mRNA. The lipid nanoparticle compositions may be used for mRNA delivery to a cell (e.g., an immune cell, such as T-cell) in a subject. Messenger RNA based gene therapy requires efficient delivery of mRNA to circulating cells (e.g., immune cells, such as T-cells or NK cells) in plasma or to cells in a given tissue. The main challenges associated with efficient mRNA delivery to attain robust levels of protein expression include: (a) ability to protect the mRNA payload against prevalent serum nucleases upon administration to a subject; (b) the ability to specifically target mRNA delivery to and thereby maximize protein expression in the target cell (e.g., T-cell) population; and (c) the ability to maximally deliver the mRNA payload to the cytosolic compartment of cells (e.g., T-cells) for translation into proteins within the cytoplasm.

The invention provides ionizable cationic lipids for producing lipid nanoparticle compositions that facilitate the delivery of a payload (e.g., a nucleic acid, such as a DNA or RNA, such as an mRNA) disposed therein to cells, for example, mammalian cells, for example, immune cells. The lipids are designed to enable intracellular delivery of a nucleic acid, e.g., mRNA, to the cytosolic compartment of a target cell type and rapidly degrade into non-toxic components. These complex functionalities are achieved by the interplay between chemistry and geometry of the ionizable lipid head group, the hydrophobic “acyl-tail” groups and the linker connecting the head group and the acyl tail groups in the ionizable cationic lipids.

In one aspect, the present invention provides a compound represented by Formula I:

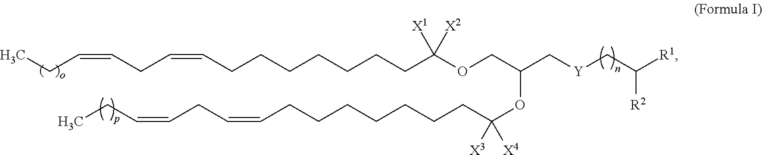

or a salt thereof, wherein the variables are as defined herein.

In another aspect, the present invention provides a compound represented by Formula II:

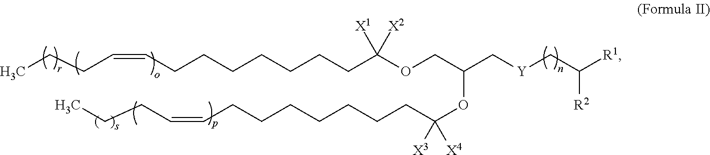

or a salt thereof, wherein the variables are as defined herein.

Provided herein, in part, is a compound selected from the group consisting of:

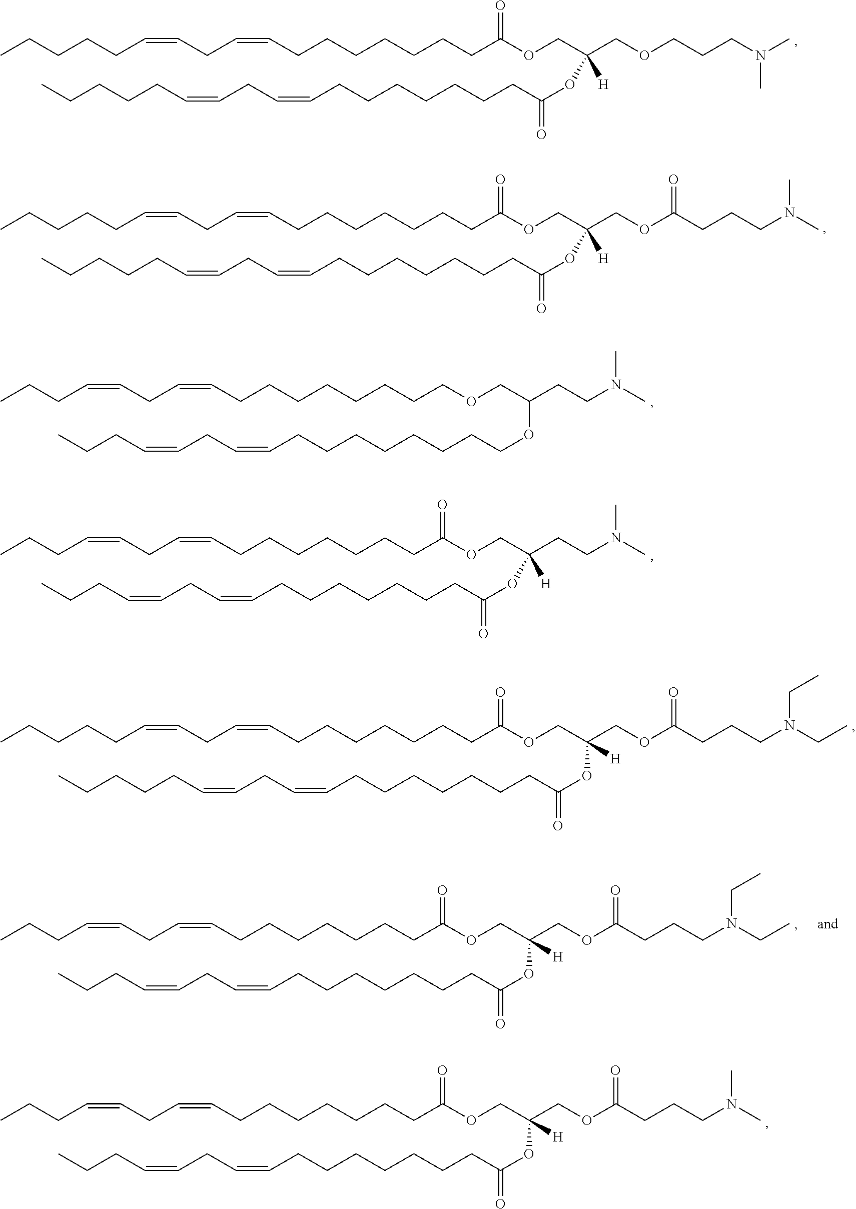

or a salt thereof.

In certain embodiments, the compound is a compound of Formula III:

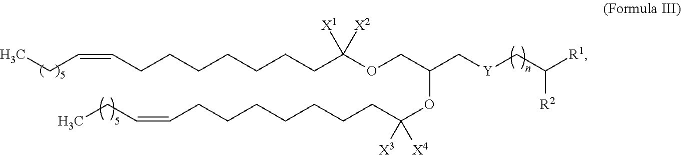

or a salt thereof, wherein the variables are as defined herein.

Also provided herein is a compound of the formula:

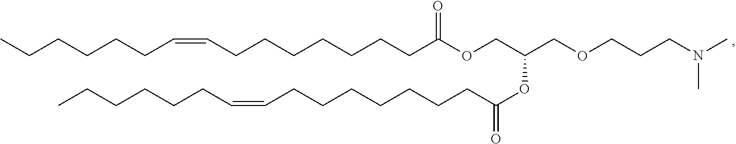 [SMILES: CCCCCC/C=C\CCCCCCCC(=O)OC[C@H](COCCCN(C)C)OC(=O)CCCCCCC/C=C\CCCCCC]

or a salt thereof.

Also provided herein is a compound of the formula:

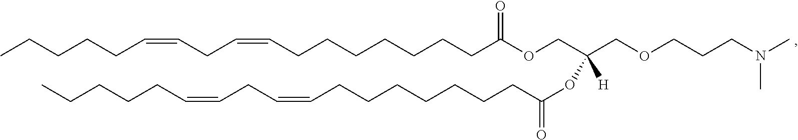 [SMILES: CCCCC/C=C\C/C=C\CCCCCCCC(=O)OC[C@H](COCCCN(C)C)OC(=O)CCCCCCC/C=C\C/C=C\CCCCC]

or a salt thereof.

Also provided herein is a compound of the formula:

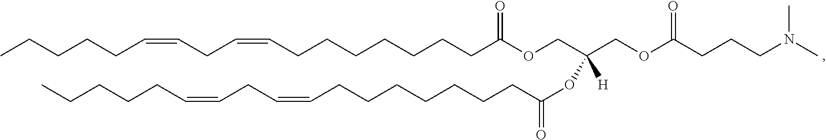 [SMILES: CCCCC/C=C\C/C=C\CCCCCCCC(=O)OC[C@H](COC(=O)CCCN(C)C)OC(=O)CCCCCCC/C=C\C/C=C\CCCCC]

or a salt thereof.

Also provided herein is a compound of the formula:

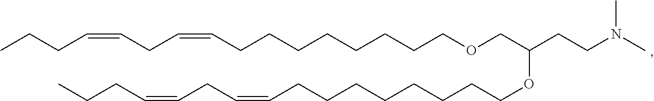 [SMILES: CCC/C=C\C/C=C\CCCCCCCCOCC(CCN(C)C)OCCCCCCCC/C=C\C/C=C\CCC]

or a salt thereof.

Also provided herein is a compound of the formula:

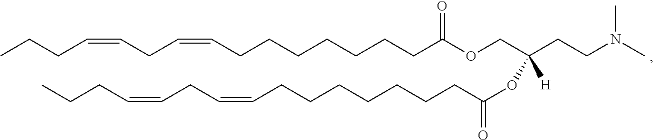 [SMILES: CCC/C=C\C/C=C\CCCCCCCC(=O)OC[C@H](CCN(C)C)OC(=O)CCCCCCC/C=C\C/C=C\CCC]

or a salt thereof.

Also provided herein is a compound of the formula:

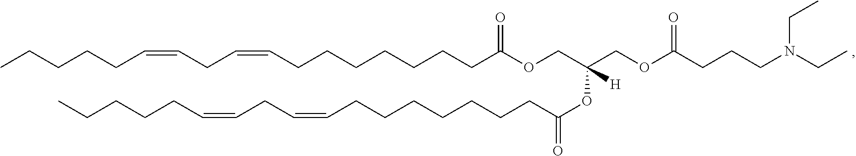 [SMILES: CCCCC/C=C\C/C=C\CCCCCCCC(=O)OC[C@H](COC(=O)CCCN(CC)CC)OC(=O)CCCCCCC/C=C\C/C=C\CCCCC]

or a salt thereof.

Also provided herein is a compound of the formula:

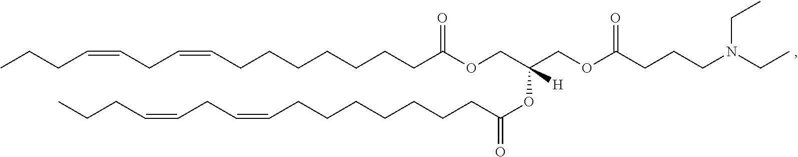 [SMILES: CCC/C=C\C/C=C\CCCCCCCC(=O)OC[C@H](COC(=O)CCCN(CC)CC)OC(=O)CCCCCCC/C=C\C/C=C\CCC]

or a salt thereof.

Also provided herein is a compound of the formula:

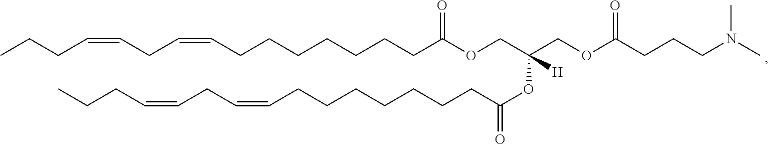 [SMILES: CCC/C=C\C/C=C\CCCCCCCC(=O)OC[C@H](COC(=O)CCCN(C)C)OC(=O)CCCCCCC/C=C\C/C=C\CCC]

or a salt thereof.

Also provided herein is a lipid nanoparticle (LNP) comprising a lipid blend comprising an ionizable cationic lipid and/or lipid-immune cell targeting group conjugate (e.g., a lipid-T-cell targeting group conjugate) provided herein.

In another aspect, provided herein is a method of delivering a nucleic acid to an immune cell (e.g., a T-cell), the method comprising exposing the immune cell to an LNP described herein containing the nucleic acid under conditions that permit the nucleic acid to enter the immune cell.

In another aspect, provided herein is a method of delivering a nucleic acid to an immune cell (e.g., a T-cell) in a subject in need thereof, the method comprising administering to the subject a composition comprising an LNP described herein containing a nucleic acid thereby to deliver the nucleic acid to the immune cell.

In another aspect, provided herein is a method of targeting the delivering of a nucleic acid (e.g., mRNA) to an immune cell (e.g., a T-cell) in a subject, the method comprising administering to the subject an LNP described herein containing the nucleic acid so as to facilitate targeted delivery of the nucleic acid to the immune cell.

In one aspect, provided herein are lipid nanoparticles (LNPs) comprising a lipid blend for targeted delivery of a nucleic acid into an immune cell. In some embodiments, the lipid blend comprises a lipid-immune cell targeting group conjugate comprising the compound of Formula IV: [Lipid]-[optional linker]-[immune cell targeting group]. In some embodiments, the lipid blend comprises an ionizable cationic lipid. In some embodiments, the ionizable cationic lipid comprises

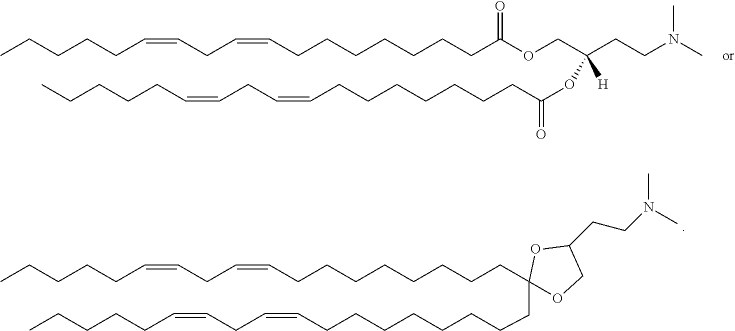

In some embodiments, the LNP comprises a nucleic acid disposed therein.

In some embodiments, the immune cell targeting group comprises an antibody that binds a T cell antigen. In some embodiments, the T cell antigen is CD3, CD4, CD7, or CD8, or a combination thereof (e.g., both CD3 and CD8, both CD4 and CD8, or both CD7 and CD8). In some embodiments, the immune cell targeting group comprises an antibody that binds a Natural Killer (NK) cell antigen. In some embodiments, the NK cell antigen is CD7, CD8, or CD56, or a combination thereof (e.g., both CD7 and CD8). In some embodiments, the antibody is a human or humanized antibody.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the PEG is PEG 2000.

In some embodiments, the lipid-immune cell targeting group conjugate is present in the lipid blend in a range of 0.002-0.2 mole percent. In some embodiments, the lipid blend comprises one or more of a structural lipid (e.g., a sterol), a neutral phospholipid, and a free PEG-lipid. In some embodiments, the ionizable cationic lipid is present in the lipid blend in a range of 40-60 mole percent. In some embodiments, the sterol is present in the lipid blend in a range of 30-50 mole percent. In some embodiments, the sterol is present in the lipid blend in a range of 20-70 mole percent. In some embodiments, the sterol is cholesterol.

In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), sphingomyelin (SM). In some embodiments, the neutral phospholipid is present in the lipid blend in a range of 1-10 mole percent.

In some embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl[methoxy(polyethylene glycol)] (PEG-ceramide), and DSPE-PEG-cysteine, or a derivative thereof. In some embodiments, the free PEG-lipid comprises a diacylphosphatidylethanolamines comprising Dipalmitoyl (C16) chain or Distearoyl (C18) chain. In some embodiments the free PEG-lipid is a mixture of two or more unique free PEG-lipids. In some embodiments, the free PEG-lipid is present in the lipid blend in a range of 1-4 mole percent, such as about 1-2 mole percent, or about 2-4 mole percent, or about 1.5 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about −10 mV to about +30 mV at pH 5.

In some embodiments, the nucleic acid is DNA or RNA. In some embodiments, the RNA is an mRNA, tRNA, siRNA, or microRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR). In some embodiments, the CAR comprises the amino acid sequence of SEQ ID NO: 24. In some embodiments, the mRNA encoding the CAR comprises the polynucleotide sequence of SEQ ID NO: 25.

In some embodiments, the immune cell targeting group comprises an antibody, and the antibody is a Fab or an immunoglobulin single variable domain, such as a Nanobody. In some embodiments, the immune cell targeting group comprises a Fab, F(ab′)2, Fab′-SH, Fv, or scFv fragment. In some embodiments, the immune cell targeting group comprises a Fab that is engineered to knock out one or more natural interchain disulfide bonds. For example, in some embodiments, the Fab comprises a heavy chain fragment that comprises C233S substitution, numbering according to Kabat, and/or a light chain fragment that comprises C214S substitution, numbering according to Kabat. In some embodiments, the immune cell targeting group comprises a Fab that is engineered to introduce one or more buried interchain disulfide bonds. For example, in some embodiments, the Fab antibody comprises a heavy chain fragment that comprises F174C substitution, numbering according to Kabat, and/or a light chain fragment that comprises S176C substitution, numbering according to Kabat. In some embodiments, the immune cell targeting group comprises a Fab that is engineered to knock out one or more natural interchain disulfide bonds, and to introduce one or more buried interchain disulfide bonds. In some embodiments, the immune cell targeting group comprises a Fab that comprises a cysteine at the C-terminus of the heavy or light chain fragment. In some embodiments, the Fab further comprises one or more amino acids between the heavy chain fragment of the Fab and the C-terminal cysteine. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker. In some embodiments, the Fab antibody is a DS Fab (Fab with wild type (natural) interchain disulfide bond), a NoDS Fab (Fab with natural disulfide bond knocked out, such as a Fab with C233S substitution on the heavy chain, and/or C214S substitution on the light chain, numbering according to Kabat), a bDS Fab (Fab without natural disulfide bond, and with introduced non-natural interchain buried disulfide bond, such as a Fab with F174C and C233S on the heavy chain, and/or S176C and C214S substitution on the light chain, numbering according to Kabat), or a bDS Fab-ScFv (a bDS Fab linked to a ScFv through a linker, such as (G4S)x), as demonstrated in FIG. 47.

In some embodiments, the immune cell targeting group comprises an immunoglobulin single variable domain, such as a Nanobody. In some embodiments, the immunoglobulin single variable domain comprises a cysteine at the C-terminus. In some embodiments, the Nanobody further comprises a spacer comprising one or more amino acids between the V_{HH} domain and the C-terminal cysteine. In some embodiments, the immune cell targeting group comprises two or more V_{HH} domains. In some embodiments, the two or more V_{HH} domains are linked by an amino acid linker. In some embodiments, the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds. In some embodiments, the immune cell targeting group comprises a V_{HH} domain linked to an antibody CH1 domain, and wherein the antibody CH1 domain is linked to an antibody light chain constant domain by one or more disulfide bonds. In some embodiments, the CH1 domain comprises F174C and C233S substitutions, and/or the light chain constant domain comprises S176C and C214S substitutions, numbering according to Kabat. In some embodiments, the antibody is a ScFv, a V_{HH} (Nb), a 2×V_{HH}, a V_{HH}-CH1/empty Vk, or a V_{HH}1-CH1/V_{HH}-2-Nb bDS, as demonstrated in FIG. 47.

In some embodiments, the immune cell targeting group comprises a Fab that comprises a heavy chain fragment comprising the amino acid sequence of SEQ ID NO: 1 and a light chain fragment comprising the amino acid sequence of SEQ ID NO:2 or 3. In some embodiments, the immune cell targeting group comprises a Fab that comprises a heavy chain fragment comprising the amino acid sequence of SEQ ID NO: 6 and a light chain fragment comprising the amino acid sequence of SEQ ID NO: 7. In some embodiments, the antibody is an antibody described in the examples.

In some embodiments, the immune cell targeting group comprises a Fab that comprises:

(a) a heavy chain fragment comprising the amino acid sequence of SEQ ID NO: 1 and a light chain fragment comprising the amino acid sequence of SEQ ID NO:2 or 3;

(b) a heavy chain fragment comprising the amino acid sequence of SEQ ID NO: 4 and a light chain fragment comprising the amino acid sequence of SEQ ID NO: 5;

(c) a heavy chain fragment comprising the amino acid sequence of SEQ ID NO: 6 and a light chain fragment comprising the amino acid sequence of SEQ ID NO: 7;

(d) a heavy chain fragment comprising the amino acid sequence of SEQ ID NO: 8 and a light chain fragment comprising the amino acid sequence of SEQ ID NO: 9;

(e) a heavy chain fragment comprising the amino acid sequence of SEQ ID NO: 10 and a light chain fragment comprising the amino acid sequence of SEQ ID NO: 11;

(f) a heavy chain fragment comprising the amino acid sequence of SEQ ID NO: 12 and a light chain fragment comprising the amino acid sequence of SEQ ID NO: 13;

(g) a heavy chain fragment comprising the amino acid sequence of SEQ ID NO: 14 and a light chain fragment comprising the amino acid sequence of SEQ ID NO: 15;

(h) a heavy chain fragment comprising the amino acid sequence of SEQ ID NO: 16 and a light chain fragment comprising the amino acid sequence of SEQ ID NO: 17;

(i) a heavy chain fragment comprising the amino acid sequence of SEQ ID NO: 18 and a light chain fragment comprising the amino acid sequence of SEQ ID NO: 19;

(j) a heavy chain fragment comprising the amino acid sequence of SEQ ID NO: 20 and a light chain fragment comprising the amino acid sequence of SEQ ID NO: 21; or
(k) a heavy chain fragment comprising the amino acid sequence of SEQ ID NO: 22 and a light chain fragment comprising the amino acid sequence of SEQ ID NO: 23.

In some embodiments, the immune cell targeting group comprises a Fab, F(ab′)2, Fab′-SH, Fv, or scFv fragment. In some embodiments, the immune cell targeting group comprises a Fab that is engineered to knock out the natural interchain disulfide bond at the C-terminus. In some embodiments, the Fab comprises a heavy chain fragment that comprises C233S substitution, and a light chain fragment that comprises C214S substitution, numbering according to Kabat. In some embodiments, the immune cell targeting group comprises a Fab that has a non-natural interchain disulfide bond (e.g., a engineered, buried interchain disulfide bond). In some embodiments, the Fab comprises F174C substitution in the heavy chain fragment, and S176C substitution in the light chain fragment, numbering according to Kabat. In some embodiments, the immune cell targeting group comprises a Fab that comprises a cysteine at the C-terminus of the heavy or light chain fragment. In some embodiments, the Fab further comprises one or more amino acids between the heavy chain fragment of the Fab and the C-terminal cysteine.

In some embodiments, the immune cell targeting group comprises an immunoglobulin single variable domain. In some embodiments, the immunoglobulin single variable domain comprises a cysteine at the C-terminus. In some embodiments, the immunoglobulin single variable domain comprises a VHH domain and further comprises a spacer comprising one or more amino acids between the VHH domain and the C-terminal cysteine. In some embodiments, the immune cell targeting group comprises two or more VHH domains. In some embodiments, the two or more V_{HH} domains are linked by an amino acid linker. In some embodiments, the immune cell targeting group comprises a first V_{HH} domain linked to an antibody CH1 domain and a second V_{HH} domain linked to an antibody light chain constant domain. In some embodiments, the antibody CH1 domain and the antibody light chain constant domain are linked by one or more disulfide bonds. In some embodiments, the immune cell targeting group comprises a VHH domain linked to an antibody CH1 domain. In some embodiments, the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat.

In some embodiments, the immune cell targeting group comprises a Fab that comprises: (a) a heavy chain fragment comprising the amino acid sequence of SEQ ID NO: 1 and a light chain fragment comprising the amino acid sequence of SEQ ID NO:2 or 3; or (b) a heavy chain fragment comprising the amino acid sequence of SEQ ID NO: 6 and a light chain fragment comprising the amino acid sequence of SEQ ID NO: 7.

In another aspect, provided herein are methods of targeting the delivery of a nucleic acid to an immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the compound of the following formula: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, an aspect of the disclosure relates to an LNP or a pharmaceutical composition containing thereof, as disclosed herein, for use in a method of targeting the delivery of a nucleic acid to an immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP). In some embodiments, the LNP is an LNP as described herein in the present disclosure.

In some embodiments, the LNP provides at least one of the following benefits:

(i) increased specificity of targeted delivery to the immune cell compared to a reference LNP;
(ii) increased half-life of the nucleic acid or a polypeptide encoded by the nucleic acid in the immune cell compared to a reference LNP;
(iii) increased transfection rate compared to a reference LNP; and
(iv) a low level of dye accessible mRNA (<15%) and high RNA encapsulation efficiencies, wherein at least 80% mRNA was recovered in final formulation relative to the total RNA used in LNP batch preparation.

In some aspect, provided are methods of expressing a polypeptide of interest in a targeted immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition containing thereof, as disclosed herein, for use in a method of expressing a polypeptide of interest in a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:

(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP; and
(v) a low level of dye accessible mRNA (<15%) and high RNA encapsulation efficiencies, wherein at least 80% mRNA was recovered in final formulation relative to the total RNA used in LNP batch preparation.
In some aspects, provided are methods of modulating cellular function of a target immune cell of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the immune cell. In some embodiments, an aspect of the disclosure relates to an LNP or a pharmaceutical composition containing thereof, as disclosed herein, for use in a method of modulating cellular function of a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:

(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP;
(v) the LNP can be administered at a lower dose compared to a reference LNP to reach the same biologic effect in the immune cell; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the modulation of cell function comprises reprogramming the immune cells to initiate an immune response. In some embodiments, the modulation of cell function comprises modulating antigen specificity of the immune cell.

In some aspect, provided are methods of treating, ameliorating, or preventing a symptom of a disorder or disease in a subject in need thereof. In some embodiments, the method comprises administering to the subject a lipid nanoparticle (LNP) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the nucleic acid modulates the immune response of the immune cell, therefore to treat or ameliorate the symptom. In some embodiments, an aspect of the disclosure relates to an LNP or a pharmaceutical composition containing thereof, as disclosed herein, for use in a method of treating, ameliorating, or preventing a symptom of a disorder or disease in a subject in need thereof. A disease or disorder may be as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:

(i) increased specificity of delivery of the nucleic acid into the immune cell compared to a reference LNP;
(ii) increased half-life of the nucleic acid or a polypeptide encoded by the nucleic acid in the immune cell compared to a reference LNP;
(iii) increased transfection rate compared to a reference LNP;
(iv) the LNP can be administered at a lower dose compared to a reference LNP to reach the same treatment efficacy;
(v) increased level of gain of function by an immune cell compared to a reference LNP; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the disorder is an immune disorder, an inflammatory disorder, or cancer. In some embodiments, the nucleic acid encodes an antigen for use in a therapeutic or prophylactic vaccine for treating or preventing an infection by a pathogen. In some embodiments, the Fab antibody comprises a heavy chain fragment that comprises F174C substitution, numbering according to Kabat, and/or a light chain fragment that comprises S176C substitution, numbering according to Kabat

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In one aspect, provided are lipid nanoparticles (LNPs) for delivering a nucleic acid into NK cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (0 the nucleic acid. In some embodiments, the immune cell targeting group comprises an antibody that binds CD56.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (0 the nucleic acid. In some embodiments, the immune cell targeting group comprises an antibody that binds CD7 or CD8, and the free PEG lipid is DMG-PEG.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody, and the antibody is a Fab or an immunoglobulin single variable domain. In some embodiments, the Fab is engineered to knock out the natural interchain disulfide at the C-terminus. In some embodiments, the Fab has a buried interchain disulfide. In some embodiments, the antibody is an immunoglobulin single variable (ISV) domain, and the ISV domain an Nanobody® ISV. In some embodiments, the free PEG lipid comprise a PEG having a molecular weight of at least 2000 daltons. In some embodiments, the PEG has a molecular weight of about 3000 to 5000 daltons. In some embodiments, the Fab is an anti-CD3 antibody, and the free PEG lipid in the LNP comprises a PEG having a molecular weight of about 2000 daltons. In some embodiments, the Fab is an anti-CD4 antibody, and the free PEG lipid in the LNP comprises a PEG having a molecular weight of about 3000 to 3500 daltons.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (0 the nucleic acid. In some embodiments, the immune cell targeting group comprises an antibody that binds CD3, and an antibody that binds CD11a or CD18.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (0 the nucleic acid. In some embodiments, the immune cell targeting group comprises an antibody that binds CD7, and an antibody that binds CD8.

In one aspect, provided are lipid nanoparticles (LNPs) for delivering a nucleic acid into two different types of immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid.

In some embodiments, the immune cell targeting group comprise a bispecific targeting moiety. In some embodiments, the bispecific targeting moiety binds to the two different types of immune cells. In some embodiments, the two different types of immune cells are CD4+ T cells and CD8+ T cell. In some embodiments, the bispecific targeting moiety is a bispecific antibody. In some embodiments, the bispecific antibody is a Fab-ScFv.

In one aspect, provided are lipid nanoparticles (LNPs) for delivering a nucleic acid into both CD4+ and CD8+ T cells of a subject. The LNP comprises (a) An ionizable cationic lipid, (b) A conjugate comprising the following structure: [Lipid]-[optional linker]
[immune cell targeting group]; (c) A sterol or other structural lipid; (d) A neutral phospholipid; (e) A free Polyethylene glycol (PEG) lipid, and (0 the nucleic acid. In some embodiments, the immune cell targeting group comprise a single antibody that binds to CD3 or CD7.

Further provided is a lipid nanoparticle (LNP) for delivering a nucleic acid into an immune cell of a subject, wherein the LNP comprises: (a) an ionizable cationic lipid, (b) a conjugate comprising the following structure: [Lipid]-[optional linker]-[immune cell targeting group]; (c) a sterol or other structural lipid; (d) a neutral phospholipid; (e) a free Polyethylene glycol (PEG) lipid, and (0 the nucleic acid, wherein the immune cell targeting group comprises a Fab lacking the native interchain disulfide bond. In some embodiments, the Fab is engineered to replace one or both cysteines on the native constant light chain and the native constant heavy chain that form the native interchain disulfide with a non-cysteine amino acid, therefor to remove the native interchain disulfide bond in the Fab.

Also provided is an immunoglobulin single variable domain (ISVD) that binds to human CD8. In some embodiments, the ISVD comprises three complementarity determining domains CDR1, CDR2, and CDR3, wherein

(a) the CDR1 comprises GSTFSDYG (SEQ ID NO: 100),
(b) the CDR2 comprises IDWNGEHT (SEQ ID NO: 101), and
(c) the CDR3 comprises AADALPYTVRKYNY (SEQ ID NO: 102).

In some embodiments, the ISVD is humanized.

In some embodiments, the ISVD comprises, consists of, or consists essentially of SEQ ID NO: 77.

Also provided is a polypeptide comprising GSTFSDYG (SEQ ID NO: 100), IDWNGEHT (SEQ ID NO: 101), and AADALPYTVRKYNY (SEQ ID NO: 102).

In some embodiments, the polypeptide comprises the ISVD as described herein.

In some embodiments, the polypeptide further comprises a second binding moiety, wherein the second binding moiety binds to CD8 or another different target. In some embodiments, the second binding moiety is also an ISVD.

In some embodiments, the polypeptide further comprises a detectable marker, or a therapeutic agent.

Also provided is a composition comprising the ISVD or the polypeptide as described herein.

Further provided is a pharmaceutical composition comprising the ISVD or the polypeptide as described herein, and a pharmaceutically acceptable carrier.

Further provided is a method of treating a disease or disorder related to CD8 in a subject, comprising administering the pharmaceutical composition as described herein to the subject.

In some embodiments, the disease is cancer. In some embodiments, the disorder is an immune disorder, an inflammatory disorder, or cancer.

In some embodiments, the nucleic acid encodes an antigen for use in a therapeutic or prophylactic vaccine for treating or preventing an infection by a pathogen. In some embodiments, the ionizable cationic lipid is

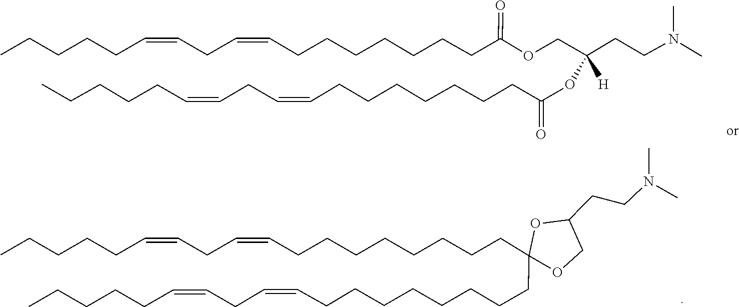

In some embodiments, the immune cell targeting group comprises an antibody that binds a T cell antigen. In some embodiments, the T cell antigen is CD3, CD8, or both CD3 and CD8.60. In some embodiments, the immune cell targeting group comprises an antibody that binds a Natural Killer (NK) cell antigen. In some embodiments, the NK cell antigen is CD56. In some embodiments, the antibody is a human or humanized antibody.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the PEG is PEG 2000.

In some embodiments, the lipid-immune cell targeting group conjugate is present in the lipid blend in a range of 0.002-0.2 mole percent. In some embodiments, the ionizable cationic lipid is present in the lipid blend in a range of 40-60 mole percent.

In some embodiments, the sterol is cholesterol. In some embodiments, the sterol is present in the lipid blend in a range of 30-50 mole percent. In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), sphingomyelin (SM).

In some embodiments, the neutral phospholipid is present in the lipid blend in a range of 1-10 mole percent.

In some embodiments, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

In some embodiments, the free PEG-lipid comprises a diacylphosphatidylethanolamines comprising Dipalmitoyl (C16) chain or Distearoyl (C18) chain. In some embodiments, the free PEG-lipid is present in the lipid blend in a range of 2-4 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about −10 mV to about +30 mV at pH 5.

In some embodiments, the nucleic acid is DNA or RNA. In some embodiments, the RNA is an mRNA, tRNA, siRNA, or microRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

In some embodiments, the immune cell targeting group comprises an antibody, and the antibody is a Fab or an immunoglobulin single variable domain. In some embodiments, the immune cell targeting group comprises an antibody fragment selected from the group consisting of a Fab, F(ab′)2, Fab′-SH, Fv, and scFv fragment. In some embodiments, the immune cell targeting group comprises a Fab that comprises one or more interchain disulfide bonds. In some embodiments, the Fab comprises a heavy chain fragment that comprises F174C and C233S substitutions, and a light chain fragment that comprises S176C and C214S substitutions, numbering according to Kabat. In some embodiments, the immune cell targeting group comprises a Fab that comprises a cysteine at the C-terminus of the heavy or light chain fragment.

In some embodiments, the Fab further comprises one or more amino acids between the heavy chain fragment of the Fab and the C-terminal cysteine. In some embodiments, the Fab comprises a heavy chain variable domain linked to an antibody CH1 domain and a light chain variable domain linked to an antibody light chain constant domain. In some embodiments, the CH1 domain and the light chain constant domain are linked by one or more interchain disulfide bonds. In some embodiments, the immune cell targeting group further comprises a single chain variable fragment (scFv) linked to the C-terminus of the light chain constant domain by an amino acid linker.

In some embodiments, the immune cell targeting group comprises an immunoglobulin single variable domain. In some embodiments, the immunoglobulin single variable domain comprises a cysteine at the C-terminus. In some embodiments, the immunoglobulin single variable domain comprises a VHH domain and further comprises a spacer comprising one or more amino acids between the VHH domain and the C-terminal cysteine. In some embodiments, the immune cell targeting group comprises two or more VHH domains. In some embodiments, the two or more VHH domains are linked by an amino acid linker. In some embodiments, the immune cell targeting group comprises a first VHH domain linked to an antibody CH1 domain and a second VHH domain linked to an antibody light chain constant domain. In some embodiments, the antibody CH1 domain and the antibody light chain constant domain are linked by one or more disulfide bonds. In some embodiments, the immune cell targeting group comprises a VHH domain linked to an antibody CH1 domain. In some embodiments, the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat.

In some embodiments, the immune cell targeting group comprises a Fab that comprises:

(a) a heavy chain fragment comprising the amino acid sequence of SEQ ID NO: 1 and a light chain fragment comprising the amino acid sequence of SEQ ID NO:2 or 3;

(b) a heavy chain fragment comprising the amino acid sequence of SEQ ID NO: 6 and a light chain fragment comprising the amino acid sequence of SEQ ID NO: 7.

In some embodiments, no more than 5% non-immune cells are transfected by the LNP. In some embodiments, half-life of the nucleic acid delivered by the LNP or a polypeptide encoded by the nucleic acid delivered by the LNP is at least 10% longer than half-life of nucleic acid delivered by a reference LNP or a polypeptide encoded by the nucleic acid delivered by the reference LNP. In some embodiments, at least 10% immune cells are transfected by the LNP. In some embodiments, expression level of the nucleic acid delivered by the LNP is at least 10% higher than expression level of nucleic acid delivered by a reference LNP.

In some aspects, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises free Polyethylene glycol (PEG) lipid. In some embodiments, the LNP comprises the nucleic acid. In some embodiments, the immune cell is an NK cell. In some embodiments, the immune cell targeting group comprises an antibody that binds CD56.

In some aspect, provided herein are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group comprises an antibody that binds CD7 or CD8. In some embodiments, the free PEG lipid is DMG-PEG.

In some aspect, provided are lipid nanoparticles (LNPs) for delivering a nucleic acid into an immune cell of the subject. In some embodiments, the LNP comprises an ionizable cationic lipid. In some embodiments, the LNP comprises an conjugate comprising the following structure: [Lipid]-[optional linker]-[immune cell targeting group]. In some embodiments, the LNP comprises a sterol or other structural lipid. In some embodiments, the LNP comprises neutral phospholipid. In some embodiments, the LNP comprises a free Polyethylene glycol (PEG) lipid. In some embodiments, the LNP comprises the nucleic acid. In some embodiments, the immune cell targeting group comprises an antibody. In some embodiments, the antibody is a Fab or an immunoglobulin single variable domain.

In some embodiments, the Fab is engineered to knock out the natural interchain disulfide at the C-terminus. In some embodiments, the Fab comprises a heavy chain fragment that comprises C233S substitutions, and a light chain fragment that comprises C214S substitutions. In some embodiments, the Fab comprises a non-natural interchain disulfide. In some embodiments, the Fab comprises F174C substitution in the heavy chain fragment, and S176C substitution in the light chain fragment. In some embodiments, the antibody is an immunoglobulin single variable (ISV) domain, and the ISV domain is an Nanobody® ISV. In some embodiments, the free PEG lipid comprise a PEG having a molecular weight of at least 2000 daltons. In some embodiments, the PEG has a molecular weight of about 3000 to 5000 daltons. In some embodiments, the antibody is a Fab. In some embodiments, the Fab binds CD3, and the free PEG lipid in the LNP comprises a PEG having a molecular weight of about 2000 daltons. In some embodiments, the Fab is an anti-CD4 antibody, and the free PEG lipid in the LNP comprises a PEG having a molecular weight of about 3000 to 3500 daltons.

In some embodiments, the immune cell targeting group comprises two or more VHH domains. In some embodiments, the two or more VHH domains are linked by an amino acid linker. In some embodiments, the immune cell targeting group comprises a first VHH domain linked to an antibody CH1 domain and a second VHH domain linked to an antibody light chain constant domain.

In some aspect, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the LNP binds CD3, and also binds CD11a or CD18. In some embodiments, the LNP comprises two conjugates. In some embodiments, the first conjugate comprises an antibody that binds CD3. In some embodiments, the second conjugate comprises an antibody that binds CD11a or CD18. In some embodiments, the LNP comprises one conjugate. In some embodiments, the conjugate comprises a bispecific antibody that binds both CD3 and CD11a. In some embodiments, the conjugate comprises a bispecific antibody that binds both CD3 and CD18. In some embodiments, the bispecific antibody is an immunoglobulin single variable domain or Fab-ScFv.

In some aspect, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the LNP binds CD7 and CD8 of the immune cell.

In some embodiments, the LNP comprises two conjugates. In some embodiments, the first conjugate comprises an antibody that binds CD7, and a second conjugate that binds CD8. In some embodiments, the LNP comprises one conjugate. In some embodiments, the conjugate comprises a bispecific antibody that binds CD7 and CD8. In some embodiments, the bispecific antibody is an immunoglobulin single variable domain or Fab-ScFv.

In some aspect, provided are lipid nanoparticles (LNPs) for delivering a nucleic acid into two different types of immune cells of the subject. In some embodiments, the LNP comprises: an ionizable cationic lipid. In some embodiments, the LNP comprises a conjugate comprising the following structure: [Lipid]-[optional linker]-[immune cell targeting group]. In some embodiments, the LNP comprises sterol or other structural lipid. In some embodiments, the LNP comprises neutral phospholipid. In some embodiments, the LNP comprises a free Polyethylene glycol (PEG) lipid. In some embodiments, the LNP comprises the nucleic acid. In some embodiments, the LNP binds to a first antigen on the surface of the first type of immune cell, and also binds to a second antigen on the surface of the second type of immune cell. In some embodiments, the two different types of immune cells are CD4+ T cells and CD8+ T cell. In some embodiments, the LNP comprises two conjugates. In some embodiments, the first conjugate comprises a first antibody that binds to the first antigen of the first type of immune cell, and the second conjugate comprises a second antibody that binds to the second antigen of the second type of immune cell. In some embodiments, the LNP comprises one conjugate. In some embodiments, the conjugate comprises a bispecific antibody. In some embodiments, the bispecific antibody binds to both the first antigen on the first type of immune cell, the second antigen on the second type of immune cells. In some embodiments, the bispecific antibody is an immunoglobulin single variable domain or a Fab-ScFv.

In some aspect, provided are lipid nanoparticles (LNPs) for delivering a nucleic acid into both CD4+ and CD8+ T cells of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group comprises a single antibody that binds to CD3 or CD7.

In some aspect, provided are lipid nanoparticles (LNPs) for delivering a nucleic acid into both T cells and NK cells of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group binds to CD7, CD8, or both CD7 and CD8.

In some aspect, provided are lipid nanoparticles (LNPs) for delivering a nucleic acid into both T cells and NK cells of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group binds to (i) both CD3 and CD56; (ii) both CD8 and CD56; or (iii) both CD7 and CD56.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the lipid-immune cell targeting group conjugate is present in the lipid blend in a range of 0.002-0.2 mole percent. In some embodiments, the lipid blend further comprises one or more of a structural lipid (e.g., a sterol), a neutral phospholipid, and a free PEG-lipid.

In some embodiments, the ionizable cationic lipid is present in the lipid blend in a range of 40-60 mole percent.

In some embodiments, the sterol is present in the lipid blend in a range of 30-50 mole percent. In some embodiments, the sterol is cholesterol.

In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC). In some embodiments, the neutral phospholipid is present in the lipid blend in a range of 1-10 mole percent.

In some embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl[methoxy(polyethylene glycol)] (PEG-ceramide), and DSPE-PEG-cysteine, or a derivative thereof. In some embodiments, the free PEG-lipid comprises a diacylphosphatidylethanolamines comprising Dipalmitoyl (C16) chain or Distearoyl (C18) chain. In some embodiments, the free PEG-lipid is present in the lipid blend in a range of 1-2 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about −10 mV to about +30 mV at pH 5.

In some embodiments, the nucleic acid is DNA or RNA. In some embodiments, the RNA is an mRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

In some aspect, provided are lipid nanoparticles (LNPs) for delivering a nucleic acid into an immune cell of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the immune cell targeting group comprises a Fab lacking the native interchain disulfide bond. In some embodiments, the Fab is engineered to replace one or both cysteines on the native constant light chain and the native constant heavy chain that form the native interchain disulfide with a non-cysteine amino acid, therefor to remove the native interchain disulfide bond in the Fab.

In some aspect, provided are methods of targeting the delivery of a nucleic acid to an immune cell of a subject. In some embodiments, the method comprises contacting the immune cell with a lipid nanoparticle (LNP) provided herein. In some embodiments, the method is for targeting NK cells. In some embodiments, the immune cell targeting group binds to CD56. In some embodiments, the method is for targeting both T cells and NK cells simultaneously. In some embodiments, the immune cell targeting group binds to CD7, CD8, or both CD7 and CD8. In some embodiments, the method is for targeting both CD4+ and CD8+ T cells simultaneously. In some embodiments, the immune cell targeting group comprises a polypeptide that binds to CD3 or CD7.

In some aspect, provided are methods of expressing a polypeptide of interest in a targeted immune cell of a subject. In some embodiments, the method comprises contacting the immune cell with a lipid nanoparticle (LNP) provided herein.

In some aspect, provided are method of modulating cellular function of a target immune cell of a subject. In some embodiments, the method comprises administering to the subject a lipid nanoparticle (LNP) provided herein.

In some aspect, provided are method of treating, ameliorating, or preventing a symptom of a disorder or disease in a subject in need thereof. In some embodiments, the method comprises administering to the subject a lipid nanoparticle (LNP) provided herein.

In some aspect, provided are immunoglobulin single variable domains (ISVDs) that bind to human CD8. In some embodiments, the ISVD comprises three complementarity determining domains CDR1, CDR2, and CDR3. In some embodiments, the CDR1 comprises GSTFSDYG (SEQ ID NO: 100). In some embodiments, the CDR2 comprises IDWNGEHT (SEQ ID NO: 101). In some embodiments, the CDR3 comprises AADALPYTVRKYNY (SEQ ID NO: 102). In some embodiments, the ISVD is humanized. In some embodiments, the ISVD comprises SEQ ID NO: 77.

In some aspect, provided are polypeptides comprising GSTFSDYG (SEQ ID NO: 100), IDWNGEHT (SEQ ID NO: 101), and AADALPYTVRKYNY (SEQ ID NO: 102). In another aspect, provided are polypeptides comprising the ISVD provided herein. In some embodiments, the polypeptide comprises a second binding moiety. In some embodiments, the second binding moiety binds to CD8 or another different target. In some embodiments, the second binding moiety is also an ISVD. In some embodiments, the polypeptide comprises a detectable marker. In some embodiments, the polypeptide comprises a therapeutic agent.

In some aspect, provided are compositions comprising the ISVD provided herein or the polypeptide provided herein.

In some aspect, provided are pharmaceutical compositions comprising the ISVD provided herein or the polypeptide provided herein, and a pharmaceutically acceptable carrier.

In some aspect, provided are methods of treating a disease or disorder related to CD8 in a subject. In some embodiments, the method comprises administering a pharmaceutical composition described herein to the subject. In some embodiments, the disease or disorder is cancer.

Various aspects and embodiments of the invention are described in further detail below.

### BRIEF DESCRIPTION OF THE DRAWINGS

FIG. 1 depicts an NMR spectrum of Lipid 1.

FIGS. 2A and 2B depict LC-MS spectra of Lipid 1.

FIG. 3 depicts an NMR spectrum of Lipid 2.

FIGS. 4A and 4B depict LC-MS spectra of Lipid 2.

FIG. 5 depicts an NMR spectrum of Lipid 3.

FIG. 6 depicts an NMR spectrum of Lipid 4.

FIGS. 7A and 7B depict LC-MS spectra of Lipid 4.

FIG. 8A depicts Lipid 2 and Lipid 6 LNP pKa (TNS). FIG. 8B depicts Lipid 5 and Lipid 7 LNP pKa (TNS).

FIG. 9A depicts hydrodynamic diameter of Lipid 2, and Lipid 6 derived LNPs.

FIG. 9B depicts polydispersity (Dynamic Light Scattering) of Lipid 2 and Lipid 6 derived LNPs.

FIG. 10A depicts hydrodynamic diameter of Lipid 5, and Lipid 7 derived LNPs.

FIG. 10B depicts polydispersity (Dynamic Light Scattering) of Lipid 5 and Lipid 7 derived LNPs.

FIGS. 11A-E depict in vitro T-cell transfection of GFP mRNA using Lipid 2 and Lipid 6 derived LNPs, % GFP+ cells (FIG. 11A), GFP mean fluorescence intensity (MFI) (FIG. 11B), % Cy5-GFP+ cells (FIG. 11C), Cy5-GFP MFI (FIG. 11D), and T-cell viability (FIG. 11E).

FIGS. 12A-E depict in vitro T-cell transfection of GFP mRNA using Lipid 5 and Lipid 7 derived LNPs, % GFP+ cells (FIG. 12A), GFP mean fluorescence intensity (MFI) (FIG. 12B), % Cy5-GFP+ cells (FIG. 12C), Cy5-GFP MFI (FIG. 12D), T-cell viability (FIG. 12E).

FIG. 13A depicts % GFP+(translation) human CD8 T cells post 24 hr transfection. FIG. 13B depicts % Cy5+(binding) human CD8 T cells post 24 hr transfection.

FIG. 14A depicts % Viable human CD8 T cells post 24 hr transfection. FIG. 14B depicts Human IFNγ measured from cell culture supernatant post 24 hr transfection.

FIG. 15A depicts % TTR-023+(anti-CD20 CAR) CD8 T cells post 24 hr transfection with mRNA LNPs. FIG. 15B depicts % TTR-023+(anti-CD20 CAR) CD4 T cells post 24 hr transfection with mRNA LNPs.

FIG. 16A depicts % CD69+CD8 cells post 24 hr transfection with anti-CD20 CAR mRNA LNPs. FIG. 16B depicts % CD69+CD4 T cells post 24 hr transfection with anti-CD20 CAR mRNA LNPs.

FIG. 17 depicts Human IFNγ secretion by T cells post 24 hr transfection with anti-CD20 CAR mRNA LNPs.

FIG. 18A depicts % GFP+(transfection/translation) of CD8 T cells post 24 hr transfection with Cy5/GFP mRNA at 2.5 ug/mL for 24 hr. FIG. 18B depicts % GFP+(transfection/translation) Mean Fluorescence Intensity (MFI) of CD8 T cells post 24 hr transfection with Cy5/GFP mRNA at 2.5 ug/mL for 24 hr.

FIG. 19A depicts % Cy5+(binding) of CD8 T cells post 24 hr transfection with Cy5/GFP mRNA at 2.5 ug/mL for 24 hr. FIG. 19B depicts Cy5 (binding) Mean Fluorescence Intensity (MFI) of CD8 T cells post 24 hr transfection with Cy5/GFP mRNA at 2.5 ug/mL for 24 hr.

FIG. 20A depicts % GFP+(transfection/translation) CD8 cells of human CD3 cells transfected with 2.5 ug/mL Cy5/GFP mRNA LNPs for 24 hr. FIG. 20B depicts % GFP+(transfection/translation) CD4 cells of human CD3 cells transfected with 2.5 ug/mL Cy5/GFP mRNA LNPs for 24 hr.

FIG. 21A depicts % Cy5+(binding) CD8 cells of human CD3 cells transfected with 2.5 ug/mL Cy5/GFP mRNA LNPs for 24 hr. FIG. 21B depicts % Cy5+(binding) CD4 cells of human CD3 cells transfected with 2.5 ug/mL Cy5/GFP mRNA LNPs for 24 hr.

FIG. 22A depicts % CD69+CD8 cells of human CD3 cells transfected with 2.5 ug/mL Cy5/GFP mRNA LNPs for 24 hr. FIG. 22B depicts % CD69+CD4 cells of human CD3 cells transfected with 2.5 ug/mL Cy5/GFP mRNA LNPs for 24 hr.

FIG. 23 depicts Human IFNγ secretion from human CD3 cells transfected with 2.5 μg/mL Cy5/GFP mRNA LNPs for 24 hr.

FIG. 24A depicts % m Cherry+CD8 T cells transfected in whole blood at 2.5 μg/mL mCherry mRNA LNPs for 24 hr. FIG. 24B depicts % m Cherry+CD4 T cells transfected in whole blood at 2.5 μg/mL mCherry mRNA LNPs for 24 hr.

FIG. 25A depicts % m Cherry+ B cells transfected in whole blood at 2.5 μg/mL mCherry mRNA LNPs for 24 hr. FIG. 25B depicts % m Cherry+NK cells transfected in whole blood at 2.5 μg/mL mCherry mRNA LNPs for 24 hr.

FIG. 26A depicts % m Cherry+Granulocytes transfected in whole blood at 2.5 μg/mL mCherry mRNA LNPs for 24 hr. FIG. 26B depicts % CD69+CD8 T cells transfected in whole blood at 2.5 ug/mL mCherry mRNA LNPs for 24 hr. FIG. 26C depicts % CD69+CD4 T cells transfected in whole blood at 2.5 ug/mL mCherry mRNA LNPs for 24 hr.

FIGS. 27A and 27B depict time course for in vivo reprogramming of CD8+ T cells and CD4+ T cells respectively with CD3 targeted mCherry LNPs in blood. Each symbol represents one mouse. Open symbol represents mice that were buffer control treated and closed symbol represents mcherry LNP treated. Circles represent 24 hr, triangles represent 48 hr and diamonds represents 96 hr. FIGS. 27C and 27D depict time course for in vivo reprogramming of CD8+ T cells and CD4+ T cells respectively in liver. Each symbol represents one mouse. Open symbol represents mice that were buffer control treated and closed symbol represent mCherry LNP treated. Circles represent 24 hr, triangles represent 48 hr and diamonds represents 96 hr. FIGS. 27E and 27F depict time course for in vivo reprogramming of CD8+ T cells and CD4+ T cells respectively with CD3 targeted mCherry LNPs in spleen. Each symbol represents one mouse. Open symbol represents mice that were buffer control treated and closed symbol represent mCherry LNP treated. Circles represent 24 hr, triangles represent 48 hr and diamonds represents 96 hr.

FIG. 28 depicts minimal expression of mCherry in liver myeloid and Kupffer cells after 24 hr treated with CD3 targeted mcherry LNP. Each symbol represents one mouse. Open symbols represent mice that were buffer control treated and closed symbol represent mCherry LNP treated.

FIG. 29A depicts in vivo reprogramming after 24 hr of Pt dose of mCherry expressing LNPs in blood. Each symbol represents one mouse. Open circles are CD4+ T cells and open square are CD8+ T cells expressing mCherry. FIG. 29B depicts In vivo reprogramming after 24 hr of Pt dose of TTR-023 expressing LNPs in blood. Each symbol represents one mouse. Open circles are CD4+ T cells and open square are CD8+ T cells expressing anti-CD20 CAR.

FIGS. 30A-E depict in vivo reprogramming after 40 hr of 2nd dose of with TTR-023 expressing LNP in blood (FIG. 30A), Spleen (FIG. 30B), Liver (FIG. 30C), Bone Marrow (FIG. 30D), and Thymus (FIG. 30E). Each symbol represents one mouse. Open circle is CD4+ T cells and open square is CD8+ T cells expressing ant-CD20 CAR.

FIGS. 31A-E depict in vivo reprogramming after 40 h of 2nd dose of with mCherry expressing LNP in blood in blood (FIG. 31A), Spleen (FIG. 31B), Liver (FIG. 31C), Bone Marrow (FIG. 31D), and Thymus (FIG. 31E). Each symbol represents one mouse. Open circle isCD4+ T cells and open square is CD8+ T cells expressing mCherry.

FIG. 32 depicts dosing and bleeding schema for the PK study.

FIG. 33 depicts calculated mRNA concentration based on converted DiI-C18(3)-DS measurements from mouse serum samples.

FIG. 34A depicts % DiR+CD4 T-cells after 2 hr incubation with 2.5 μg/mL mRNA LNPs. FIG. 34B depicts DiR Mean Fluorescence Intensity (MFI) CD4 T-cells after 2 hr incubation with 2.5 μg/mL mRNA LNPs.

FIG. 35A depicts % DiR+CD8 T-cells after 2 hr incubation with 2.5 μg/mL mRNA LNPs. FIG. 35B depicts DiR Mean Fluorescence Intensity (MFI) CD8 T-cells after 2 hr incubation with 2.5 μg/mL mRNA LNPs.

FIG. 36A depicts % m Cherry+CD4 T-cells after 24 hr incubation with 2.5 μg/mL mRNA LNPs. FIG. 36B depicts % m Cherry+CD8 T-cells after 24 hr incubation with 2.5 μg/mL mRNA LNPs.

FIG. 37A depicts hydrodynamic diameter of Lipid 5, Lipid 8 and DLn-MC3-DMA derived LNPs. FIG. 37B depicts polydispersity (Dynamic Light Scattering) of Lipid 5, Lipid 8 and DLn-MC3-DMA derived LNPs.

FIGS. 38A-E depict in vitro T-cell transfection of GFP mRNA using Lipid 5, Lipid 8, and DLn-MC3-DMA derived LNPs, % GFP+ cells (FIG. 38A), GFP mean fluorescence intensity (MFI) (FIG. 38B), % Cy5-GFP+ cells (FIG. 38C), Cy5-GFP MFI (FIG. 38D), and T-cell viability (FIG. 38E).

FIG. 39 depicts an NMR spectrum of Lipid 5.

FIGS. 40A and 40B depict LC-MS spectra of Lipid 5.

FIG. 41 depicts an NMR spectrum of Lipid 6.

FIGS. 42A and 42B depict LC-MS spectra of Lipid 6.

FIG. 43 depicts an NMR spectrum of Lipid 7.

FIGS. 44A and 44B depict LC-MS spectra of Lipid 7.

FIG. 45A depicts hydrodynamic diameter of Lipid 8, and Lipid 5 derived LNPs.

FIG. 45B depicts polydispersity (Dynamic Light Scattering) of Lipid 8 and Lipid 5 derived LNPs.

FIGS. 46A-E depict in vitro T-cell transfection of GFP mRNA using Lipid 8 and Lipid 5 (0 and N) derived LNPs, % GFP+ cells (FIG. 46A), GFP mean fluorescence intensity (MFI) (FIG. 46B), % Cy5-GFP+ cells (FIG. 46C), Cy5-GFP MFI (FIG. 46D), T-cell viability (FIG. 46E).

FIG. 47 depicts structures of various Fab, VHH (Nb), ScFv, Fab-ScFv and Fab-VHH hybrids.

FIG. 48A depicts and NMR spectrum of Lipid 9. FIG. 48B and FIG. 48C depict the Mass spectrum and LC chromatogram of Lipid 9.

FIG. 49A depicts and NMR spectrum of Lipid 10. FIG. 49B and FIG. 49C depict the Mass spectrum and LC chromatogram of Lipid 10.

FIG. 50A depicts and NMR spectrum of Lipid 11. FIG. 50B and FIG. 50C depict the Mass spectrum and LC chromatogram of Lipid 11.

FIG. 51A depicts and NMR spectrum of Lipid 12. FIG. 51B and FIG. 51C depict the Mass spectrum and LC chromatogram of Lipid 12.

FIG. 52A depicts and NMR spectrum of Lipid 13. FIG. 52B and FIG. 52C depict the Mass spectrum and LC chromatogram of Lipid 13.

FIG. 53A depicts hydrodynamic diameter (DLS) of Lipid 5 and Lipid 8 prior to and after antibody conjugate insertion. FIG. 53B depicts polydispersity (DLS) prior to and after antibody conjugate insertion. FIGS. 53C and 53D depict LNP surface charge (Zeta Potential, DLS) prior to and after antibody conjugate insertion in pH 5.5 MES and pH 7.4 HEPES buffer.

FIGS. 54A to 54E depict in vitro T-cell transfection of GFP mRNA using Lipid 5 and Lipid 8 derived LNPs: % GFP+ cells (FIG. 54A), GFP mean fluorescence intensity (MFI) (FIG. 54B), % DiI+ cells (FIG. 54C), and DiI MFI (FIG. 54D), and T-cell viability (FIG. 54E).

FIG. 55A depicts hydrodynamic diameter (DLS) of Lipid 5, Lipid 8 and DLn-MC3-DMA prior to and after antibody conjugate insertion. FIG. 55B depicts polydispersity (DLS) prior to and after antibody conjugate insertion. FIG. 55C depicts LNP surface charge (Zeta Potential, DLS) prior to antibody conjugate insertion in pH 5.5 MES and pH 7.4 HEPES buffer. FIG. 55D depicts the accessible RNA content and RNA encapsulation efficiency.

FIGS. 56A to 56E depict in vitro T-cell transfection of GFP mRNA using Lipid 5, Lipid 8 and DLn-MC3-DMA derived LNPs: % GFP+ cells (FIG. 56A), GFP mean fluorescence intensity (MFI) (FIG. 56B), % DiI+ cells (FIG. 56C), and DiI MFI (FIG. 56D), and T-cell viability (FIG. 56E).

FIG. 57A depicts hydrodynamic diameter (DLS) of Lipid 5 formulations stored at 4 C or after storage at −80 C; Formulations were frozen either by placing in a −80 C freezer or flash frozen in Liquid Nitrogen. FIG. 57B depicts formulation polydispersities (DLS) before and after frozen storage.

FIGS. 58A to 58E depict in vitro T-cell transfection of GFP mRNA and T-cell viability resulting from Lipid 5 LNP formulations that were stored at 4 C or after storage at −80 C; formulations were frozen either by placing in the −80 C freezer or flash frozen in liquid Nitrogen. % GFP+ cells (FIG. 58A), GFP mean fluorescence intensity (MFI) (FIG. 58B), % DiI+ cells (FIG. 58C), and DiI MFI (FIG. 58D), and T-cell viability (FIG. 58E).

FIGS. 59A to 59T depict results of in vivo reprogramming of immune cells with CD3-targeted DiI/GFP LNP at the dose of 0.3 mg/kg after 24 or 48 h with either DMG, DPG or DSG-PEG 2.5% or after 24 h with either DPPE or DSPE 1.5 or 2.5%. Each symbol represents one mouse. Open circle is CD4+ T cells and open square is CD8+ T cells expressing; % GFP (FIG. 59A) in blood, (FIG. 59B) in liver, (FIG. 59C) in lung, (FIG. 59D) in spleen, (FIG. 59E) in bone marrow; GFP MFI (FIG. 59F) in blood, (FIG. 59G) in liver, (FIG. 59H) in lung, (FIG. 59I) in spleen, (FIG. 59J) in bone marrow; % DiI in (FIG. 59K) in blood, (FIG. 59L) in liver, (FIG. 59M) in lung, (FIG. 59N) in spleen, (FIG. 59O) in bone marrow; DiI MFI (FIG. 59P) in blood, (FIG. 59Q) in liver, (FIG. 59R) in lung, (FIG. 59S) in spleen, and (FIG. 59T) in bone marrow.

FIGS. 60A to 60T depict results of in vivo reprogramming with CD3, CD8 antibody/Nanobody targeted DiI/GFP LNP at 0.3 mg/kg of Lipid 5 with either DMG, DPG, 1.5 or 2.5% after 24h. Each symbol represents one mouse. Open circle is CD4+ T cells and open square is CD8+ T cells expressing; % GFP (60A) in blood, (60B) in liver, (60C) in lung, (60D) in spleen, (60E) in bone marrow; GFP MFI (60F) in blood, (60G) in liver, (60H) in lung, (60I) in spleen, (60J) in bone marrow; % DiI in (60K) in blood, (60L) in liver, (60M) in lung, (60N) in spleen, (60O) in bone marrow; DiI MFI (60P) in blood, (60Q) in liver, (60R) in lung, (60S) in spleen, (60T) in bone marrow.

FIGS. 61A to 61T depict results of in vivo reprogramming with either CD8, CD11a, CD4 Nanobody or CD4 antibody targeted DiI/GFP LNP at 0.3 mg/kg of Lipid 5 with either DMG or DPG, 1.5% after 24h. Each symbol represents one mouse. Open circle is CD4+ T cells and open square is CD8+ T cells expressing; % GFP (61A) in blood, (61B) in liver, (61C) in lung, (61D) in spleen, (61E) in bone marrow; GFP MFI (61F) in blood, (61G) in liver, (61H) in lung, (61I) in spleen, (61J) in bone marrow; % DiI in (61K) in blood, (61L) in liver, (61M) in lung, (61N) in spleen, (61O) in bone marrow; DiI MFI (61P) in blood, (61Q) in liver, (61R) in lung, (61S) in spleen, (61T) in bone marrow.

FIGS. 62A to 62S depict in vivo reprogramming comparing ionizable lipids (DLn-MC3-DMA, Lipid 5 and Lipid 8) with CD3 (hsp34) antibody targeted DiI/GFP LNP at 0.1 mg/kg with DPG-PEG, 1.5% after 24h. Each symbol represents one mouse. Open circle is CD4+ T cells and open square is CD8+ T cells expressing; % GFP (62A) in blood, (62B) in liver, (62C) in lung, (62D) in spleen, (62E) in bone marrow; GFP MFI (62F) in blood, (62G) in liver, (62H) in lung, (62I) in spleen, (62J) in bone marrow; % DiI in (62K) in blood, (62L) in liver, (62M) in lung, (62N) in spleen, (62O) in bone marrow; DiI MFI (62P) in blood, (62Q) in liver, (62R) in lung, (62S) in spleen, (62T) in bone marrow.

FIGS. 63A to 63T depict in vivo reprogramming with CD7 VHH/Nanobody targeted DiI/GFP LNP at 0.3 mg/kg of Lipid 5 with either DMG, DPG, 1.5 or 2.5% after 24h. Each symbol represents one mouse. Open circle is CD4+ T cells and open square is CD8+ T cells expressing; % GFP (63A) in blood, (63B) in liver, (63C) in lung, (63D) in spleen, (63E) in bone marrow; GFP MFI (63F) in blood, (63G) in liver, (63H) in lung, (63I) in spleen, (63J) in bone marrow; % DiI in (63K) in blood, (63L) in liver, (63M) in lung, (63N) in spleen, (63O) in bone marrow; DiI MFI (63P) in blood, (63Q) in liver, (63R) in lung, (63S) in spleen, (63T) in bone marrow.

FIG. 64A depicts % GFP Transfection of co-cultured T cells and NK cells after incubation with targeted LNPs at 2.5 ug/mL mRNA for approximately 24 hrs with Fabs or Nbs post inserted at densities that gave the highest levels of transfection evaluated. FIG. 64B depicts GFP Expression levels by mean fluorescence intensity (MFI) co-cultured T cells and NK after incubation with targeted LNPs at 2.5 ug/mL mRNA for approximately 24 hrs with Fabs or Nbs post inserted at densities that gave the highest levels of transfection evaluated.

FIG. 64C depicts % DiI uptake of co-cultured T cells and NK cells after incubation with targeted LNPs at 2.5 ug/mL mRNA for approximately 24 hrs with Fabs or Nbs post inserted at densities that gave the highest levels of transfection evaluated. FIG. 64D depicts % DiI uptake levels by mean fluorescence intensity (MFI) co-cultured T cells and NK after incubation with targeted LNPs at 2.5 ug/mL mRNA for approximately 24 hrs with Fabs or Nbs post inserted at densities that gave the highest levels of transfection evaluated.

FIG. 65A depicts SDS-PAGE of SP34-hlam DS (contains WT inter-chain disulfide) Fab conjugates produced by reduction at varying TCEP concentrations prior to conjugation. FIG. 65B depicts SDS-PAGE of SP34-hlam NoDS (No inter-chain disulfide, e.g., C to S mutation in HC and LC) Fab conjugates produced by reduction at varying TCEP concentrations prior to conjugation. FIG. 65C depicts R8 RP-HPLC chromatograms of hSP34-hlam DS Fab and Fab conjugate produced with a 0.025 mM TCEP reduction condition prior to conjugation. FIG. 65D depicts R8 RP-HPLC chromatograms of hSP34-hlam NoDS Fab and Fab conjugates produced with various TCEP reduction conditions prior to conjugation. FIG. 65E depicts % GFP Transfection of T cells after incubation with targeted LNPs at 2.5 ug/mL mRNA for approximately 24 hrs with Fabs post inserted at various densities. FIG. 65F depicts GFP Expression levels by mean fluorescence intensity (MFI) T cells after incubation with targeted LNPs at 2.5 ug/mL mRNA for approximately 24 hrs with Fabs post inserted at various densities.

FIG. 66A depicts SDS-PAGE of TS2/18.1 and 9.6 (contain WT inter-chain disulfide) Fab conjugates produced by reduction at varying TCEP concentrations prior to conjugation. Left: TS2/18.1; Right: 9.6 FIG. 66B depicts SDS-PAGE of TS2/18.1, 9.6 and TRX2 NoDS Fab and Fab conjugates produced by reduction at varying TCEP concentrations prior to conjugation. FIG. 66C depicts R8 RP-HPLC chromatograms of TS2/18.1 DS and NoDS Fab and Fab conjugate produced with various TCEP reduction conditions prior to conjugation. FIG. 66D depicts R8 RP-HPLC chromatograms of 9.6 and TRX2 NoDS Fab and Fab conjugate produced with various TCEP reduction conditions prior to conjugation.

FIG. 67A depicts % GFP Transfection of T cells after incubation with targeted LNPs at 2.5 ug/mL mRNA for approximately 24 hrs with Fabs post inserted at densities that gave the highest levels of transfection evaluated. FIG. 67B depicts GFP Expression levels by mean fluorescence intensity (MFI) T cells after incubation with targeted LNPs at 2.5 ug/mL mRNA for approximately 24 hrs with Fabs post inserted at densities that gave the highest levels of transfection evaluated. FIG. 67C depicts IFNγ secretion into supernatant from T cells after incubation with targeted LNPs at 2.5 ug/mL mRNA for approximately 24 hrs with Fabs post inserted at densities that gave the highest levels of transfection evaluated.

FIG. 68A depicts % GFP Transfection of T cells after incubation with targeted LNPs at 2.5 ug/mL mRNA for approximately 24 hrs with Fabs post inserted at densities that gave the highest levels of transfection evaluated. FIG. 68B depicts GFP Expression levels by mean fluorescence intensity (MFI) of CD8 T cells after incubation with targeted LNPs at 2.5 ug/mL mRNA for approximately 24 hrs with Fabs post inserted at densities that gave the highest levels of transfection evaluated.

FIG. 69A depicts % GFP Transfection of T cells after incubation with targeted LNPs at 2.5 ug/mL mRNA for approximately 24 hrs with Fabs post inserted individually or together at the same densities as the single targeted conditions. FIG. 69B depicts GFP Expression levels by mean fluorescence intensity (MFI) T cells after incubation with targeted LNPs at 2.5 ug/mL mRNA for approximately 24 hrs with Fabs post inserted individually or together at the same densities as the single targeted conditions. FIG. 69C depicts IFNγ secretion into supernatant from T cells after incubation with targeted LNPs at 2.5 ug/mL mRNA for approximately 24 hrs with Fabs post inserted individually or together at the same densities as the single targeted conditions.

FIG. 70A depicts % GFP Transfection of T cells after incubation with targeted LNPs at 2.5 ug/mL mRNA for approximately 24 hrs with Fabs or Fab-ScFv post inserted at densities that gave the highest levels of transfection evaluated. FIG. 70B depicts GFP Expression levels by mean fluorescence intensity (MFI) T cells after incubation with targeted LNPs at 2.5 ug/mL mRNA for approximately 24 hrs with Fabs or Fab-ScFv post inserted at densities that gave the highest levels of transfection evaluated. FIG. 70C depicts IFNγ secretion into supernatant T cells after incubation with targeted LNPs at 2.5 ug/mL mRNA for approximately 24 hrs with Fabs or Fab-ScFv post inserted at densities that gave the highest levels of transfection evaluated.

FIG. 71A depicts % GFP Transfection of T cells after incubation with targeted LNPs at 2.5 ug/mL mRNA for approximately 24 hrs with Fabs post inserted at densities that gave the highest levels of transfection evaluated. FIG. 71B depicts GFP Expression levels by mean fluorescence intensity (MFI) T cells after incubation with targeted LNPs at 2.5 ug/mL mRNA for approximately 24 hrs with Fabs post inserted at densities that gave the highest levels of transfection evaluated. FIG. 71C depicts IFNγ secretion into supernatant T cells after incubation with targeted LNPs at 2.5 ug/mL mRNA for approximately 24 hrs with Fabs post inserted at densities that gave the highest levels of transfection evaluated.

FIG. 72A depicts % GFP Transfection of T cells after incubation with targeted LNPs at 2.5 ug/mL mRNA for approximately 24 hrs with Fabs and Nb post inserted at densities that gave the highest levels of transfection evaluated. FIG. 72B depicts GFP Expression levels by mean fluorescence intensity (MFI) T cells after incubation with targeted LNPs at 2.5 ug/mL mRNA for approximately 24 hrs with Fabs and Nb post inserted at densities that gave the highest levels of transfection evaluated. FIG. 72C depicts IFNγ secretion into supernatant from T cells after incubation with targeted LNPs at 2.5 ug/mL mRNA for approximately 24 hrs with Fabs and Nb post inserted at densities that gave the highest levels of transfection evaluated.

FIG. 73A depicts % GFP Transfection of T cells after incubation with targeted LNPs at 2.5 ug/mL mRNA for approximately 24 hrs with Fabs and Nb post inserted at densities that gave the highest levels of transfection evaluated. FIG. 73B depicts GFP Expression levels by mean fluorescence intensity (MFI) T cells after incubation with targeted LNPs at 2.5 ug/mL mRNA for approximately 24 hrs with Fabs and Nb post inserted at densities that gave the highest levels of transfection evaluated. FIG. 73C depicts IFNγ secretion into supernatant from T cells after incubation with targeted LNPs at 2.5 ug/mL mRNA for approximately 24 hrs with Fabs and Nb post inserted at densities that gave the highest levels of transfection evaluated.

FIG. 74A depicts % GFP Transfection of T cells after incubation with targeted LNPs at 2.5 ug/mL mRNA for approximately 24 hrs with Fabs or Nbs post inserted at densities that gave the highest levels of transfection evaluated. FIG. 74B depicts GFP Expression levels by mean fluorescence intensity (MFI) T cells after incubation with targeted LNPs at 2.5 ug/mL mRNA for approximately 24 hrs with Fabs or Nbs post inserted at densities that gave the highest levels of transfection evaluated.

FIG. 75A depicts % GFP Transfection of CD8 T cells after incubation with targeted LNPs at 2.5 ug/mL mRNA for approximately 24 hrs with Fabs or Nbs post inserted at densities that gave the highest levels of transfection evaluated. FIG. 75B depicts % GFP Transfection of CD4 T cells after incubation with targeted LNPs at 2.5 ug/mL mRNA for approximately 24 hrs with Fabs or Nbs post inserted at densities that gave the highest levels of transfection evaluated. FIG. 75C depicts GFP Expression levels by mean fluorescence intensity (MFI) CD8 T cells after incubation with targeted LNPs at 2.5 ug/mL mRNA for approximately 24 hrs with Fabs or Nbs post inserted at densities that gave the highest levels of transfection evaluated. FIG. 75D depicts GFP Expression levels by mean fluorescence intensity (MFI) CD4 T cells after incubation with targeted LNPs at 2.5 ug/mL mRNA for approximately 24 hrs with Fabs or Nbs post inserted at densities that gave the highest levels of transfection evaluated.

FIG. 76A depicts % GFP Transfection of T cells after incubation with targeted LNPs at 2.5 ug/mL mRNA for approximately 24 hrs with Fabs and Nb post inserted at densities that gave the highest levels of transfection evaluated. FIG. 76B depicts GFP Expression levels by mean fluorescence intensity (MFI) T cells after incubation with targeted LNPs at 2.5 ug/mL mRNA for approximately 24 hrs with Fabs and Nb post inserted at densities that gave the highest levels of transfection evaluated.

FIG. 77A depicts % GFP Transfection of T cells after incubation with targeted LNPs at 2.5 ug/mL mRNA for approximately 24 hrs with Fabs and Nb post inserted at densities that gave the highest levels of transfection evaluated. FIG. 77B depicts GFP Expression levels by mean fluorescence intensity (MFI) T cells after incubation with targeted LNPs at 2.5 ug/mL mRNA for approximately 24 hrs with Fabs and Nb post inserted at densities that gave the highest levels of transfection evaluated.

FIG. 78A depicts % GFP Transfection of T cells after incubation with targeted LNPs at 2.5 ug/mL mRNA for approximately 24 hrs with Fabs and Nb post inserted at densities that gave the highest levels of transfection evaluated. FIG. 78B depicts GFP Expression levels by mean fluorescence intensity (MFI) T cells after incubation with targeted LNPs at 2.5 ug/mL mRNA for approximately 24 hrs with Fabs and Nb post inserted at densities that gave the highest levels of transfection evaluated.

FIG. 79A depicts % GFP Transfection of T cells after incubation with targeted LNPs at 2.5 ug/mL mRNA for approximately 24 hrs with Fabs post inserted at densities that gave the highest levels of transfection evaluated. FIG. 79B depicts GFP Expression levels by mean fluorescence intensity (MFI) T cells after incubation with targeted LNPs at 2.5 ug/mL mRNA for approximately 24 hrs with Fabs post inserted at densities that gave the highest levels of transfection evaluated. FIG. 79C depicts IFNγ secretion into supernatant T cells after incubation with targeted LNPs at 2.5 ug/mL mRNA for approximately 24 hrs with Fabs post inserted at densities that gave the highest levels of transfection evaluated.

FIG. 80A depicts % GFP Transfection of CD8 T cells after incubation with targeted LNPs at 2.5 ug/mL mRNA for approximately 24 hrs with Fabs or Nbs post inserted at densities that gave the highest levels of transfection evaluated. FIG. 80B depicts % GFP Transfection of CD4 T cells after incubation with targeted LNPs at 2.5 ug/mL mRNA for approximately 24 hrs with Fabs or Nbs post inserted at densities that gave the highest levels of transfection evaluated. FIG. 80C depicts GFP Expression levels by mean fluorescence intensity (MFI) CD8 T cells after incubation with targeted LNPs at 2.5 ug/mL mRNA for approximately 24 hrs with Fabs or Nbs post inserted at densities that gave the highest levels of transfection evaluated. FIG. 80D depicts GFP Expression levels by mean fluorescence intensity (MFI) CD4 T cells after incubation with targeted LNPs at 2.5 ug/mL mRNA for approximately 24 hrs with Fabs or Nbs post inserted at densities that gave the highest levels of transfection evaluated. FIG. 80E depicts IFNγ secretion into supernatant T cells after incubation with targeted LNPs at 2.5 ug/mL mRNA for approximately 24 hrs with Fabs post inserted at densities that gave the highest levels of transfection evaluated.

FIG. 81A depicts % GFP Transfection of T cells after incubation with targeted LNPs at 2.5 ug/mL mRNA for approximately 24 hrs with Fabs or Nbs post inserted at densities that gave the highest levels of transfection evaluated. FIG. 81B depicts GFP Expression levels by mean fluorescence intensity (MFI) T cells after incubation with targeted LNPs at 2.5 ug/mL mRNA for approximately 24 hrs with Fabs or Nbs post inserted at densities that gave the highest levels of transfection evaluated. FIG. 81C depicts IFNγ secretion from T cells after incubation with targeted LNPs at 2.5 ug/mL mRNA for approximately 24 hrs with Fabs or Nbs post inserted at densities that gave the highest levels of transfection evaluated.

FIG. 82A depicts % GFP Transfection of T cells after incubation with targeted LNPs at 2.5 ug/mL mRNA for approximately 24 hrs with Fabs or Nbs post inserted at densities that gave the highest levels of transfection evaluated. FIG. 82B depicts GFP Expression levels by mean fluorescence intensity (MFI) T cells after incubation with targeted LNPs at 2.5 ug/mL mRNA for approximately 24 hrs with Fabs or Nbs post inserted at densities that gave the highest levels of transfection evaluated. FIG. 82C depicts IFNγ secretion from T cells after incubation with targeted LNPs at 2.5 ug/mL mRNA for approximately 24 hrs with Fabs or Nbs post inserted at densities that gave the highest levels of transfection evaluated.

FIG. 83A depicts hydrodynamic diameter (DLS) of Lipid 2, Lipid 6, Lipid 12 and Lipid 13 prior to and after antibody conjugate insertion. FIG. 83B depicts polydispersity (DLS) prior to and after antibody conjugate insertion. FIG. 83C depicts LNP surface charge (Zeta Potential, DLS) prior to antibody conjugate insertion in pH 5.5 MES and pH 7.4 HEPES buffer. FIG. 83D depict the percent accessible RNA and total RNA content (ug/mL).

FIGS. 84A to 84E depict in vitro T-cell transfection of GFP mRNA using Lipid 2, Lipid 6, Lipid 12 and Lipid 13 derived LNPs, % GFP+ cells (FIG. 84A), GFP mean fluorescence intensity (MFI) (FIG. 84B), % DiI+ cells (FIG. 84C), and DiI MFI (FIG. 84D), and T-cell viability (FIG. 84E).

FIGS. 85A to 85E depict in vitro T-cell transfection of GFP mRNA using Lipid 2, Lipid 6, Lipid 12 and Lipid 13 derived LNPs, % GFP+ cells (FIG. 85A), GFP mean fluorescence intensity (MFI) (FIG. 85B), % DiI+ cells (FIG. 85C), and DiI MFI (FIG. 85D), and T-cell viability (FIG. 85E).

### DETAILED DESCRIPTION

The invention provides ionizable cationic lipids, lipid-immune cell targeting group conjugates, and lipid nanoparticle compositions comprising such ionizable cationic lipids and/or lipid-immune cell (e.g., T-cell) targeting group conjugates, medical kits containing such lipids and/or conjugates, and methods of making and using, such lipids and conjugates.

The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts. In addition, when a chemical group is a diradical, for example, it is understood a that the chemical groups can be bonded to their adjacent atoms in the remainder of the structure in one or both orientations, for example, —OC(O)— is interchangeable with —C(O)O— or —OC(S)— is interchangeable with —C(S)O—.

The terms “a” and “an” as used herein mean “one or more” and include the plural unless the context is inappropriate.

The term “alkyl” as used herein refers to a saturated straight or branched hydrocarbon, such as a straight or branched group of 112, 110, or 1-6 carbon atoms, referred to herein as C1-C12alkyl, C1-C10alkyl, and C1-C6alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. An exemplary alkylene group is —CH2CH2-.

The term “haloalkyl” refers to an alkyl group that is substituted with at least one halogen. For example, —CH2F, —CHF2, —CF3, —CH2CF3, —CF2CF3, and the like.

The term “oxo” is art-recognized and refers to a “═O” substituent. For example, a cyclopentane substituted with an oxo group is cyclopentanone.

The term “morpholinyl” refers to a substituent having the structure of:

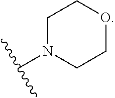 [SMILES: *N1CCOCC1]

The term “piperidinyl” refers to a substituent having a structure of:

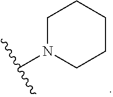 [SMILES: *N1CCCCC1]

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, an optional substituent may be selected from the group consisting of: C₁₋₆alkyl, cyano, halogen, —O—C₁₋₆alkyl, C₁₋₆haloalkyl, C₃₋₇cycloalkyl, 3-7 membered heterocyclyl, 5-6 membered heteroaryl, and phenyl, wherein R^{a} is hydrogen or C₁₋₆alkyl. In some embodiments, an optional substituent may be selected from the group consisting of: C₁₋₆alkyl, halogen, —O—C₁₋₆alkyl, and —CH₂N(Rₐ)₂, wherein R^{a} is hydrogen or C₁₋₆alkyl.

The term “haloalkyl” refers to an alkyl group that is substituted with at least one halogen. For example, —CH₂F, —CHF₂, —CF₃, —CH₂CF₃, —CF₂CF₃, and the like.

The term “cycloalkyl” refers to a monovalent saturated cyclic, bicyclic, bridged cyclic (e.g., adamantyl), or spirocyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems (e.g., fused, spiro, bridged bicyclic). A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, C₀2alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula —N(R¹⁰)(R¹¹), wherein R¹⁰ and R¹¹ each independently represent hydrogen, alkyl, cycloalkyl, heterocyclyl, alkenyl, aryl, aralkyl, or (CH₂)ₘ—R¹²; or R¹⁰ and R¹¹, taken together with the N atom to which they are attached complete a heterocycle having from 4 to 8 atoms in the ring structure; R¹² represents an aryl, a cycloalkyl, a cycloalkenyl, a heterocycle or a polycycle; and m is zero or an integer in the range of 1 to 8. In certain embodiments, R¹⁰ and R¹¹ each independently represent hydrogen, alkyl, alkenyl, or —(CH₂)ₘ—R¹².

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, O-alkynyl, —O—(CH₂)ₘ—R¹², where m and R¹² are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol “ [symbol] ” denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³⁵S, ¹⁸F, and ³⁶Cl, respectively.

Certain isotopically-labeled disclosed compounds (e.g., those labeled with 3H and 14C) are useful in compound and/or substrate tissue distribution assays. Tritiated (i.e., 3H) and carbon-14 (i.e., 14C) isotopes are particularly preferred for their ease of preparation and detectability. Further, substitution with heavier isotopes such as deuterium (i.e., 2H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable excipient” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Remington's The Science and Practice of Pharmacy, 21st Edition, A. R. Gennaro; Lippincott, Williams & Wilkins, Baltimore, Md., 2006.

As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

Abbreviations as used herein include diisopropylethylamine (DIPEA); 4-dimethylaminopyridine (DMAP); tetrabutylammonium iodide (TBAI); 1-ethyl-3-(3-dimethylaminopropyl)carbodiimide (EDC); benzotriazol-1-yl-oxytripyrrolidinophosphonium hexafluorophosphate (PyBOP), 9-Fluorenylmethoxycarbonyl (Fmoc), tetrabutyldimethylsilyl chloride (TBDMSC1), hydrogen fluoride (HF), phenyl (Ph), bis(trimethylsilyl)amine (HMDS), dimethylformamide (DMF); methylene chloride (DCM); tetrahydrofuran (THF); high-performance liquid chromatography (HPLC); mass spectrometry (MS), evaporative light scattering detector (ELSD), electrospray (ES)); nuclear magnetic resonance spectroscopy (NMR).

As used herein, the term “effective amount” refers to the amount of a compound (e.g., a nucleic acid, e.g., an mRNA) sufficient to effect beneficial or desired results. An effective amount can be administered in one or more administrations, applications or dosages and is not intended to be limited to a particular formulation or administration route. The term effective amount can be considered to include therapeutically and/or prophylactically effective amounts of a compound.

The phrase “therapeutically effective amount” as used herein means that amount of a compound (e.g., a nucleic acid, e.g., an mRNA), material, or composition comprising a compound (e.g., a nucleic acid, e.g., an mRNA) which is effective for producing some desired therapeutic effect in at least a sub-population of cells in a mammal, for example, a human, or a subject (e.g., a human subject) at a reasonable benefit/risk ratio applicable to any medical treatment.

The phrase “prophylactically effective amount” as used herein means that amount of a compound (e.g., a nucleic acid, e.g., an mRNA), material, or composition comprising a compound (e.g., a nucleic acid, e.g., an mRNA) which is effective for producing some desired prophylactic effect in at least a sub-population of cells in a mammal, for example, a human, or a subject (e.g., a human subject) by reducing, minimizing or eliminating the risk of developing a condition or the reducing or minimizing severity of a condition at a reasonable benefit/risk ratio applicable to any medical treatment.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also include the specific quantitative value itself, unless specifically stated otherwise. As used herein, the term “about” refers to a ±10% variation from the nominal value unless otherwise indicated or inferred.

As used herein, unless otherwise indicated, the term “antibody” means any antigen-binding molecule or molecular complex comprising at least one complementarity determining region (CDR) that specifically binds to or interacts with a particular antigen. It is understood the term encompasses an intact antibody (e.g., an intact monoclonal antibody), or a fragment thereof, such as an Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified or engineered. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). The term also encompasses an immunoglobulin single variable domain, such as a Nanobody (e.g., a V_{HH}).

As used here, an “antibody that binds to X” (i.e., X being a particular antigen), or “an anti-X antibody”, is an antibody that specifically recognizes the antigen X.

As used herein, a “buried interchain disulfide bond” or an “interchain buried disulfide bond” refers to a disulfide bond on a polypeptide which is not readily accessible to water soluble reducing agents, or effectively “buried” in the hydrophobic regions of the polypeptide, such that it is unavailable to both reducing agents and for conjugation to other hydrophilic PEGs. Buried interchain disulfide bonds are further described in WO2017096361A1, which is incorporated by reference in its entirety.

As used herein, specificity of the targeted delivery by an LNP is defined by the ratio between % of a desired immune cell type that receives the delivered nucleic acid (e.g., on-target delivery), and % of an undesired immune cell type that is not meant to be the destination of the delivery, but receives the delivered nucleic acid (e.g., off-target delivery). For example, the specificity is higher when more desired immune cells receive the delivered nucleic acid, while less undesired immune cells receive the delivered nucleic acid. Specificity of the targeted delivery by an LNP can also be defined the ratio of amount of nucleic acid being delivered to the desired immune cells (e.g., on-target delivery) and amount of nucleic acid being delivered to the undesired immune cells (e.g., off-target delivery). Specificity of the delivery can be determined using any suitable method. As a non-limiting example, expression level of the nucleic acid in the desired immune cell type can be measured and compared to that of a different immune cell type that is not meant to be the destination of the delivery.

As used herein, in some embodiments, a reference LNP is an LNP that does not have the immune cell targeting group but is otherwise the same as the tested LNP. In some other embodiments, a reference LNP is an LNP that has a different ionizable cationic lipid but is otherwise the same as the tested LNP. In some embodiments, a reference LNP comprises D-Lin-MC3-DMA as the ionizable cationic lipid which is different from the ionizable cationic lipid in a tested LNP, but is otherwise the same as the tested LNP.

As used herein, a humanized antibody is an antibody which is wholly or partially of non-human origin and whose protein sequence has been modified to replace certain amino acids, for instance that occur at the corresponding position(s) in the framework regions of the VH and VL domains in a sequence of antibody from a human being, to increase its similarity to antibodies produced naturally in humans, in order to avoid or minimize an immune response in humans. For example, using techniques of genetic engineering, the variable domains of a non-human antibodies of interest may be combined with the constant domains of human antibodies. The constant domains of a humanized antibody are most of the time human CH and CL domains.

As used herein, the term “structural lipid” refers to sterols and also to lipids containing sterol moieties.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C1-C₆, C₁-C₅, C₁-C₄, C₁C₃, C₁-C₂, C₂-C₆, C₂C₅, C₂C₄, C₂C₃, C₃C₆, C₃C₅, C₃C₄, C₄C₆, C₄C₅, and C₅C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### Immunoglobulin Single Variable Domain

In some embodiments, the immune cell targeting group of the LNPs as described herein comprise an immunoglobulin single variable domain, such as an Nanobody.

The term “immunoglobulin single variable domain” (ISV), interchangeably used with “single variable domain,” defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)2, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain. Hence, the antigen binding site of an immunoglobulin single variable domain is formed by no more than three CDRs.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISV) can for example be a heavy chain ISV, such as a V_{H}, V_{HH}, including a camelized V_{H} or humanized Vim. In one embodiment, it is a Vim, including a camelized V_{H} or humanized Vim. Heavy chain ISVs can be derived from a conventional four-chain antibody or from a heavy chain antibody.

For example, the immunoglobulin single variable domain may be a (single) domain antibody (or an amino acid sequence that is suitable for use as a single domain antibody), a “dAb” or dAb (or an amino acid sequence that is suitable for use as a dAb) or a Nanobody® ISV (as defined herein and including but not limited to a Vim); other single variable domains, or any suitable fragment of any one thereof.

In particular, the immunoglobulin single variable domain may be a Nanobody® ISV (such as a V_{HH}, including a humanized V_{HH} or camelized V_{H}) or a suitable fragment thereof. [Note: Nanobody® is a registered trademark of Ablynx N.V.].

“V_{HH} domains”, also known as V_{HHS}, V_{HH} antibody fragments, and Vim antibodies, have originally been described as the antigen binding immunoglobulin variable domain of “heavy chain antibodies” (i.e., of “antibodies devoid of light chains”; Hamers-Casterman et al. 1993 (Nature 363: 446-448). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans 2001 (Reviews in Molecular Biotechnology 74: 277-302).

For the term “dAb's” and “domain antibody”, reference is for example made to Ward et al. 1989 (Nature 341: 544), to Holt et al. 2003 (Trends Biotechnol. 21: 484); as well as to for example WO 2004/068820, WO 2006/030220, WO 2006/003388 and other published patent applications of Domantis Ltd. It should also be noted that, although less preferred in the context of the present invention because they are not of mammalian origin, single variable domains can be derived from certain species of shark (for example, the so-called “IgNAR domains”, see for example WO 2005/18629).

Typically, the generation of immunoglobulins involves the immunization of experimental animals, fusion of immunoglobulin producing cells to create hybridomas and screening for the desired specificities. Alternatively, immunoglobulins can be generated by screening of naïve, immune or synthetic libraries e.g. by phage display.

The generation of immunoglobulin sequences, such as VHHs, has been described extensively in various publications, among which WO 1994/04678, Hamers-Casterman et al. 1993 (Nature 363: 446-448) and Muyldermans et al. 2001 (Reviews in Molecular Biotechnology 74: 277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be used herein. Also, fully human, humanized or chimeric sequences can be used in the method described herein. For example, camelid immunoglobulin sequences and humanized camelid immunoglobulin sequences, or camelized domain antibodies, e.g. camelized dAb as described by Ward et al. 1989 (Nature 341: 544), WO 1994/04678, and Davis and Riechmann (1994, Febs Lett., 339:285-290; and 1996, Prot. Eng., 9:531-537) can be used herein. Moreover, the ISVs are fused forming a multivalent and/or multispecific construct (for multivalent and multispecific polypeptides containing one or more V_{HH} domains and their preparation, reference is also made to Conrath et al. 2001 (J. Biol. Chem., Vol. 276, 10. 7346-7350) as well as to for example WO 1996/34103 and WO 1999/23221).

A “humanized Vim” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the prior art (e.g. WO 2008/020079). Again, it should be noted that such humanized Vis can be obtained in any suitable manner known per se and thus are not strictly limited to polypeptides that have been obtained using a polypeptide that comprises a naturally occurring V_{HH} domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the description in the prior art (e.g. Davies and Riechman 1994, FEBS 339: 285; 1995, Biotechnol. 13: 475; 1996, Prot. Eng. 9: 531; and Riechman 1999, J. Immunol. Methods 231: 25). Such “camelizing” substitutions are inserted at amino acid positions that form and/or are present at the V_{H}-V_{L} interface, and/or at the so-called Camelidae hallmark residues, as defined herein (see for example WO 1994/04678 and Davies and Riechmann (1994 and 1996, supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence or V_{HH} sequence). In one particular aspect, the framework sequences are either framework sequences that have been derived from a Vim-sequence (in which said framework sequences may optionally have been partially or fully humanized) or are conventional V_{H} sequences that have been camelized (as defined herein).

In particular, the framework sequences present in the ISV sequence described herein may contain one or more of hallmark residues (as defined herein), such that the ISV sequence is a Nanobody® ISV, such as e.g. a V_{HH}, including a humanized V_{HH} or camelized V_{H} Non-limiting examples of (suitable combinations of) such framework sequences will become clear from the further disclosure herein.

The total number of amino acid residues in a V_{H} domain and a V_{HH} domain will usually be in the range of from 110 to 120, often between 112 and 115. It should however be noted that smaller and longer sequences may also be suitable for the purposes described herein.

However, it should be noted that the ISVs described herein is not limited as to the origin of the ISV sequence (or of the nucleotide sequence used to express it), nor as to the way that the ISV sequence or nucleotide sequence is (or has been) generated or obtained. Thus, the ISV sequences may be naturally occurring sequences (from any suitable species) or synthetic or semi-synthetic sequences. In a specific but non-limiting aspect, the ISV sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, Nanobody® ISVs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a Nanobody® ISV can be defined as an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISV can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody® ISV can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table 2A below.

TABLE 2A
Hallmark Residues in Nanobody ® ISVs

| Position | Human VH3 | Hallmark Residues |
|---|---|---|
| 11 | L, V; predominantly L | L, S, V, M, W, F, T, Q, E, A, R, G, K, Y, N, P, I; |
| | | preferably L |
| 37 | V, I, F; usually V | F⁽¹⁾ Y, V, L, A, H, S, I, W, C, N, G, D, T, P, |
| | | preferably F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; |
| | | most preferably G⁽²⁾ or Q⁽³⁾ |
| 45⁽⁸⁾ | L | L⁽²⁾ R⁽³⁾ P, H, F, G, Q, S, E, T, Y, C, I, D, V; |
| | | preferably L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, |
| | | H, K, Q, N, D; preferably W^{(2),} L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | P⁽⁵⁾, S, H, L, A, V, I, T, F, D, R, Y, N, Q, G, E; |
| | | preferably P |
| 103 | W | W⁽⁴⁾, R⁽⁶⁾, G, S, K, A, M, Y, L, F, T, N, V, Q, P⁽⁶⁾, |
| | | E, C; preferably W |
| 104 | G | G, A, S, T, D, P, N, E, C, L; preferably G |
| 108 | L, M or T; | Q, L⁽⁷⁾, R, P, E, K, S, T, M, A, H; preferably Q or |
| | predominantly L | L⁽⁷⁾ |

Notes:
In particular, but not exclusively, in combination with KERE (SEQ ID NO: 103) or KQRE (SEQ ID NO: 104) at positions 43-46.Usually as GLEW (SEQ ID NO: 105) at positions 44-47. Usually as KERE (SEQ ID NO: 103) or KQRE (SEQ ID NO: 104) at positions 43-46, e.g. as KEREL (SEQ ID NO: 106), KEREF (SEQ ID NO: 107), KQREL (SEQ ID NO: 108), KQREF (SEQ ID NO: 109), KEREG (SEQ ID NO: 110), KQREW (SEQ ID NO: 111) or KQREG (SEQ ID NO: 112) at positions 43-47. Alternatively, also sequences such as TERE (SEQ ID NO: 113) (for example TEREL (SEQ ID NO: 114)), TQRE (SEQ ID NO: 115) (for example TQREL (SEQ ID NO: 116)), KECE (SEQ ID NO: 117) (for example KECEL (SEQ ID NO: 118) or KECER(SEQ ID NO: 119)), KQCE (SEQ ID NO: 120) (for example KQCEL (SEQ ID NO: 121)), RERE (SEQ ID NO: 122) (for example REREG (SEQ ID NO: 123)), RQRE (SEQ ID NO: 124) (for example RQREL (SEQ ID NO: 125), RQREF (SEQ ID NO: 126) or RQREW (SEQ ID NO:127)), QERE (SEQ ID NO: 128) (for example QEREG (SEQ ID NO: 129)), QQRE (SEQ ID NO: 130), (for example QQREW (SEQ ID NO: 131), QQREL (SEQ ID NO: 132) or QQREF (SEQ ID NO: 133)), KGRE (SEQ ID NO: 134) (for example KGREG (SEQ ID NO: 135)), KDRE (SEQ ID NO: 136) (for example KDREV (SEQ ID NO: 137)) are possible. Some other possible, but less preferred sequences include for example DECKL (SEQ ID NO: 138) and NVCEL (SEQ ID NO: 139). With both GLEW (SEQ ID NO: 105) at positions 44-47 and KERE (SEQ ID NO: 103) or KQRE (SEQ ID NO: 104) at positions 43-46. Often as KP or EP at positions 83-84 of naturally occurring VHH domains. In particular, but not exclusively, in combination with GLEW (SEQ ID NO: 105) at positions 44-47. With the proviso that when positions 44-47 are GLEW (SEQ ID NO: 105), position 108 is always Q in (non-humanized) VHH sequences that also contain a W at 103. The GLEW group also contains GLEW-like sequences at positions 44-47, such as for example GVEW (SEQ ID NO: 140), EPEW (SEQ ID NO: 141), GLER (SEQ ID NO: 142), DQEW (SEQ ID NO: 143), DLEW (SEQ ID NO: 144), GIEW (SEQ ID NO: 145), ELEW (SEQ ID NO: 146), GPEW (SEQ ID NO: 147), EWLP (SEQ ID NO: 148), GPER (SEQ ID NO: 149), GLER (SEQ ID NO: 142) and ELEW (SEQ ID NO: 146).

In one embodiment, the immunoglobulin single variable domain has certain amino acid substitutions in the framework regions effective in preventing or reducing binding of so-called “pre-existing antibodies” to the polypeptides. ISVs in which (i) the amino acid residue at position 112 is one of K or Q; and/or (ii) the amino acid residue at position 89 is T; and/or (iii) the amino acid residue at position 89 is L and the amino acid residue at position 110 is one of K or Q; and (iv) in each of cases (i) to (iii), the amino acid at position 11 is preferably V have been described in WO2015/173325.

### Polypeptides

The immunoglobulin single variable domains may form part of a protein or polypeptide, which may comprise or essentially consist of one or more (at least one) immunoglobulin single variable domains and which may optionally further comprise one or more further amino acid sequences (all optionally linked via one or more suitable linkers). The term “immunoglobulin single variable domain” may also encompass such polypeptides. The one or more immunoglobulin single variable domains may be used as a binding unit in such a protein or polypeptide, which may optionally contain one or more further amino acids that can serve as a binding unit, so as to provide a monovalent, multivalent or multispecific polypeptide of the invention, respectively (for multivalent and multispecific polypeptides containing one or more V_{HH} domains and their preparation, reference is also made to Conrath et al. 2001 (J. Biol. Chem. 276: 7346), as well as to for example WO 1996/34103, WO 1999/23221 and WO 2010/115998).

The polypeptides may comprise or essentially consist of one immunoglobulin single variable domain, as outlined above. Such polypeptides are also referred to herein as monovalent polypeptides.

The term “multivalent” indicates the presence of multiple ISVs in a polypeptide. In one embodiment, the polypeptide is “bivalent”, i.e., comprises or consists of two ISVs. In one embodiment, the polypeptide is “trivalent”, i.e., comprises or consists of three ISVs. In another embodiment, the polypeptide is “tetravalent”, i.e. comprises or consists of four ISVDs. The polypeptide can thus be “bivalent”, “trivalent”, “tetravalent”, “pentavalent”, “hexavalent”, “heptavalent”, “octavalent”, “nonavalent”, etc., i.e., the polypeptide comprises or consists of two, three, four, five, six, seven, eight, nine, etc., ISVs, respectively. In one embodiment the multivalent ISV polypeptide is trivalent. In another embodiment the multivalent ISV polypeptide is tetravalent. In still another embodiment, the multivalent ISV polypeptide is pentavalent.

In one embodiment, the multivalent ISV polypeptide can also be multispecific. The term “multispecific” refers to binding to multiple different target molecules (also referred to as antigens). The multivalent ISV polypeptide can thus be “bispecific”, “trispecific”, “tetraspecific”, etc., i.e., can bind to two, three, four, etc., different target molecules, respectively.

For example, the polypeptide may be bispecific-trivalent, such as a polypeptide comprising or consisting of three ISVs, wherein two ISVs bind to a first target and one ISV binds to a second target different from the first target. In another example, the polypeptide may be trispecific-tetravalent, such as a polypeptide comprising or consisting of four ISVs, wherein one ISV binds to a first target, two ISVs bind to a second target different from the first target and one ISV binds to a third target different from the first and the second target. In still another example, the polypeptide may be trispecific-pentavalent, such as a polypeptide comprising or consisting of five ISVs, wherein two ISVs bind to a first target, two ISVs bind to a second target different from the first target and one ISV binds to a third target different from the first and the second target.

In one embodiment, the multivalent ISV polypeptide can also be multiparatopic. The term “multiparatopic” refers to binding to multiple different epitopes on the same target molecules (also referred to as antigens). The multivalent ISV polypeptide can thus be “biparatopic”, “triparatopic”, etc., i.e., can bind to two, three, etc., different epitopes on the same target molecules, respectively.

In another aspect, the polypeptide of the invention that comprises or essentially consists of one or more immunoglobulin single variable domains (or suitable fragments thereof), may further comprise one or more other groups, residues, moieties or binding units. Such further groups, residues, moieties, binding units or amino acid sequences may or may not provide further functionality to the immunoglobulin single variable domain (and/or to the polypeptide in which it is present) and may or may not modify the properties of the immunoglobulin single variable domain.

For example, such further groups, residues, moieties or binding units may be one or more additional amino acids, such that the compound, construct or polypeptide is a (fusion) protein or (fusion) polypeptide. In a preferred but non-limiting aspect, said one or more other groups, residues, moieties or binding units are immunoglobulins. Even more preferably, said one or more other groups, residues, moieties or binding units are chosen from the group consisting of domain antibodies, amino acids that are suitable for use as a domain antibody, single domain antibodies, amino acids that are suitable for use as a single domain antibody, “dAb”s, amino acids that are suitable for use as a dAb, or Nanobodies.

Alternatively, such groups, residues, moieties or binding units may for example be chemical groups, residues, moieties, which may or may not by themselves be biologically and/or pharmacologically active. For example, and without limitation, such groups may be linked to the one or more immunoglobulin single variable domain so as to provide a “derivative” of the immunoglobulin single variable domain.

In another embodiment, said further residues may be effective in preventing or reducing binding of so-called “pre-existing antibodies” to the polypeptides. For this purpose, the polypeptides and constructs may contain a C-terminal extension (X)n (SEQ ID NO: 150) (in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I), for which reference is made to WO 2012/175741. Accordingly, the polypeptide may further comprise a C-terminal extension (X)n (SEQ ID NO: 151), in which n is 1 to 5, such as 1, 2, 3, 4 or 5, and in which X is a naturally occurring amino acid, preferably no cysteine.

In the polypeptides described above, the one or more immunoglobulin single variable domains and the one or more groups, residues, moieties or binding units may be linked directly to each other and/or via one or more suitable linkers or spacers. For example, when the one or more groups, residues, moieties or binding units are amino acids, the linkers may also be an amino acid, so that the resulting polypeptide is a fusion protein or fusion polypeptide.

As used herein, the term “linker” denotes a peptide that fuses together two or more ISVs into a single molecule. The use of linkers to connect two or more (poly)peptides is well known in the art. Further exemplary peptidic linkers are shown in Table 2B. One often used class of peptidic linker are known as the “Gly-Ser” or “GS” linkers. These are linkers that essentially consist of glycine (G) and serine (S) residues, and usually comprise one or more repeats of a peptide motif such as the GGGGS (SEQ ID NO:154) motif (for example, having the formula (Gly-Gly-Gly-Gly-Ser)n (SEQ ID NO: 152) in which n may be 1, 2, 3, 4, 5, 6, 7 or more). Some often-used examples of such GS linkers are 9GS linkers (GGGGSGGGS, SEQ ID NO: 157), 15GS linkers (n=3) and 35GS linkers (n=7). Reference is for example made to Chen et al. 2013 (Adv. Drug Deliv. Rev. 65(10): 1357-1369) and Klein et al. 2014 (Protein Eng. Des. Sel. 27 (10): 325-330).

TABLE 2B
Linker sequences (“ID” refers to the SEQ ID NO as used herein)

| Name | ID | Amino acid sequence |
|---|---|---|
| 3A linker | 153 | AAA |
| 5GS linker | 154 | GGGGS |
| 7GS linker | 155 | SGGSGGS |
| 8GS linker | 156 | GGGGSGGS |
| 9GS linker | 157 | GGGGSGGGS |
| 10GS linker | 158 | GGGGSGGGGS |
| 15GS linker | 159 | GGGGSGGGGSGGGGS |
| 18GS linker | 160 | GGGGSGGGGSGGGGSGGS |
| 20GS linker | 161 | GGGGSGGGGSGGGGSGGGGS |
| 25GS linker | 162 | GGGGSGGGGSGGGGSGGGGSGGGGS |
| 30GS linker | 163 | GGGGSGGGGSGGGGSGGGGSGGGGSGGGGS |
| 35GS linker | 164 | GGGGSGGGGSGGGGSGGGGSGGGGSGGGGSGGGGS |
| 40GS linker | 165 | GGGGSGGGGSGGGGSGGGGSGGGGSGGGGSGGGGSGGGGS |
| G1 hinge | 166 | EPKSCDKTHTCPPCP |
| 9GS-G1 hinge | 167 | GGGGSGGGSEPKSCDKTHTCPPCP |
| Llama upper long | 168 | EPKTPKPQPAAA |
| hinge region | | |
| G3 hinge | 169 | ELKTPLGDTTHTCPRCPEPKSCDTPPPCPRCPEPKSCDTPP |
| | | PCPRCPEPKSCDTPPPCPRCP |

In one aspect, the disclosure also relates to such amino acid sequences and/or Nanobodies that can bind to and/or are directed against CD8 and that comprise CDR sequences that are generally as further defined herein, to suitable fragments thereof, as well as to polypeptides that comprise or essentially consist of one or more of such Nanobodies and/or suitable fragments. In some aspect, the disclosure relates to Nanobodies with SEQ ID NO: 77. In particular, the disclosure in some specific aspects provides:

I) amino acid sequences that are directed against CD8 and that have at least 80%, preferably at least 85%, such as 90% or 95% or more sequence identity with at least one of the amino acid sequences of SEQ ID NO: 77;

II) amino acid sequences that cross-block the binding of the amino acid sequence of SEQ ID NO: 77 to CD8 and/or that compete with at least the amino acid sequence of SEQ ID NO: 77 for binding to CD8;

Such amino acid sequences may be as further described herein (and may for example be Nanobodies); as well as polypeptides of the disclosure that comprise one or more of such amino acid sequences (which may be as further described herein), and particularly bispecific (or multispecific) polypeptides as described herein, and nucleic acid sequences that encode such amino acid sequences and polypeptides. Such amino acid sequences and polypeptides do not include any naturally occurring ligands.

In some embodiments, the CD8 is derived from a mammalian animal, such as a human being. In one specific, but non-limiting aspect, the disclosure relates to an amino acid sequence directed against CD8, that comprises:

a) the amino acid sequence of SEQ ID NO: 77;

b) amino acid sequences that have at least 80% amino acid identity with at least one of the amino acid sequences of SEQ ID NO: 77, or

c) amino acid sequences that have 3, 2, or 1 amino acid difference with at least one of the amino acid sequences of SEQ ID NO: 77;

or any suitable combination thereof.

In some embodiments, disclosed is a Nanobody against CD8, which consist of 4 framework regions (FR1 to FR4 respectively) and 3 complementarity determining regions (CDR1 to CDR3 respectively). In some embodiments, in such a Nanobody:

(I) CDR1 comprises or essentially consists of an amino acid sequence of GSTFSDYG (SEQ ID NO: 100),

or amino acid sequences that have at least 80%, at least 90%, at least 95%, at least 99% or more sequence identity with GSTFSDYG (SEQ ID NO: 100), in which (1) any amino acid substitution is a conservative amino acid substitution; and/or (2) said amino acid sequence only contains amino acids substitutions, and no amino acid deletions or insertions, compared to GSTFSDYG (SEQ ID NO: 100);

and/or from the group consisting of amino acids sequences that have 2 or only 1 amino acid difference(s) with GSTFSDYG (SEQ ID NO: 100), in which

any amino acid substitution is a conservative amino acid substitution; and/or

said amino acid sequence only contains amino acid substitutions, and no amino acid deletions or insertions, compared to GSTFSDYG (SEQ ID NO: 100).

(II) CDR2 comprises or essentially consists of an amino acid sequence of IDWNGEHT (SEQ ID NO: 101),

or amino acid sequences that have at least 80%, at least 90%, at least 95%, at least 99% or more sequence identity with IDWNGEHT (SEQ ID NO: 101), in which (1) any amino acid substitution is a conservative amino acid substitution; and/or (2) said amino acid sequence only contains amino acids substitutions, and no amino acid deletions or insertions, compared to IDWNGEHT (SEQ ID NO: 101);

and/or from the group consisting of amino acids sequences that have 2 or only 1 amino acid difference(s) with IDWNGEHT (SEQ ID NO: 101), in which

any amino acid substitution is a conservative amino acid substitution; and/or

said amino acid sequence only contains amino acid substitutions, and no amino acid deletions or insertions, compared to IDWNGEHT (SEQ ID NO: 101).

(III) CDR3 comprises or essentially consists of an amino acid sequence of AADALPYTVRKYNY (SEQ ID NO: 102),

or amino acid sequences that have at least 80%, at least 90%, at least 95%, at least 99% or more sequence identity with AADALPYTVRKYNY (SEQ ID NO: 102), in which (1) any amino acid substitution is a conservative amino acid substitution; and/or (2) said amino acid sequence only contains amino acids substitutions, and no amino acid deletions or insertions, compared to AADALPYTVRKYNY (SEQ ID NO: 102);

and/or from the group consisting of amino acids sequences that have 2 or only 1 amino acid difference(s) with AADALPYTVRKYNY (SEQ ID NO: 102), in which

any amino acid substitution is a conservative amino acid substitution; and/or

said amino acid sequence only contains amino acid substitutions, and no amino acid deletions or insertions, compared to AADALPYTVRKYNY (SEQ ID NO: 102).

CD8 Nanobodies as disclosed herein may comprise one, two or all three of the CDRs explicitly listed above. In some embodiments, the CD8 Nanobody comprises:

CDR1: GSTFSDYG (SEQ ID NO: 100), based on IMGT designation;

CDR2: IDWNGEHT (SEQ ID NO: 101), based on IMGT designation; and

CDR3: AADALPYTVRKYNY (SEQ ID NO: 102), based on IMGT designation.

In the Nanobodies of the disclosure that comprise the combinations of CDR's mentioned above, each CDR can be replaced by a CDR chosen from the group consisting of amino acid sequences that have at least 80%, preferably at least 90%, more preferably at least 95%, even more preferably at least 99% sequence identity with the mentioned CDR's; in which:

(1) any amino acid substitution is preferably a conservative amino acid substitution; and/or

(2) said amino acid sequence preferably only contains amino acid substitutions, and no amino acid deletions or insertions, compared to the above amino acid sequence(s);

and/or chosen from the group consisting of amino acid sequences that have 3, 2 or only 1 (as indicated in the preceding paragraph) “amino acid difference(s)” with the mentioned CDR(s) one of the above amino acid sequences, in which:

(1) any amino acid substitution is preferably a conservative amino acid substitution; and/or

(2) said amino acid sequence preferably only contains amino acid substitutions, and no amino acid deletions or insertions, compared to the above amino acid sequence(s).

In one embodiment, the CD8 Nanobody is DbSNP:

Anti-CD8 BDSn Nb sequence (CDR1, CDR2, CDR3
underlined based on IMGT designation):
(SEQ ID NO: 77)
EVQLVESGGGLVQAGGSLRLSCAASGSTFSDYGVGWFRQAPGKGREFVADI
DWNGEHTSYADSVKGRFATSRDNAKNTAYLQMNSLKPEDTAVYYCAADALP
YTVRKYNYWGQGTQVTVSSGGCGGHHHHHH

In some embodiments, a CD8 Nanobody of the present disclosure binds to CD8 with an dissociation constant (KD) of 10⁻⁵ to 10⁻¹² moles/liter (M) or less, and preferably 10⁻⁷ to 10-12 moles/liter (M) or less and more preferably 10⁻⁸ to 10⁻¹² moles/liter (M), and/or with an association constant (KA) of at least 107 M−1, preferably at least 10⁸ M⁻¹, more preferably at least 10⁹ M⁻¹, such as at least 10¹² M⁻¹; and in particular with a KD less than 500 nM, preferably less than 200 nM, more preferably less than 10 nM, such as less than 500 μM. The KD and KA values of the Nanobody of the disclosure against vWF can be determined in a manner known per se, for example using the assay described herein. More generally, the Nanobodies described herein preferably have a dissociation constant with respect to vWF that is as described in this paragraph.

Generally, it should be noted that the term Nanobody as used herein in its broadest sense is not limited to a specific biological source or to a specific method of preparation. For example, as will be discussed in more detail below, the Nanobodies can be obtained (1) by isolating the V_{HH} domain of a naturally occurring heavy chain antibody; (2) by expression of a nucleotide sequence encoding a naturally occurring V_{HH} domain; (3) by “humanization” (as described below) of a naturally occurring V_{HH} domain or by expression of a nucleic acid encoding a such humanized V_{HH} domain; (4) by “camelization” (as described below) of a naturally occurring V_{H} domain from any animal species, in particular a species of mammal, such as from a human being, or by expression of a nucleic acid encoding such a camelized V_{H} domain; (5) by “camelisation” of a “domain antibody” or “Dab” as described by Ward et al (supra), or by expression of a nucleic acid encoding such a camelized V_{H} domain; (6) using synthetic or semi-synthetic techniques for preparing proteins, polypeptides or other amino acid sequences; (7) by preparing a nucleic acid encoding a Nanobody using techniques for nucleic acid synthesis, followed by expression of the nucleic acid thus obtained; and/or (8) by any combination of the foregoing. Suitable methods and techniques for performing the foregoing will be clear to the skilled person based on the disclosure herein and for example include the methods and techniques described in more detail hereinbelow.

In some embodiments, the CD8 Nanobodies of the present disclosure do not have an amino acid sequence that is exactly the same as (i.e. as a degree of sequence identity of 100% with) the amino acid sequence of a naturally occurring V_{H} domain, such as the amino acid sequence of a naturally occurring V_{H} domain from a mammal, and in particular from a human being.

One class of CD8 Nanobodies of the disclosure comprises Nanobodies with an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence by one or more of the amino acid residues that occur at the corresponding position(s) in a V_{H} domain from a conventional 4-chain antibody from a human being (e.g. indicated above). It should be noted that such humanized CD8 Nanobodies of the present disclosure can be obtained in any suitable manner known per se (i.e. as indicated under points (1)-(8) above) and thus are not strictly limited to polypeptides that have been obtained using a polypeptide that comprises a naturally occurring V_{HH} domain as a starting material.

Another class of CD8 Nanobodies of the present disclosure comprises Nanobodies with an amino acid sequence that corresponds to the amino acid sequence of a naturally occurring V_{H} domain that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description below. Reference is also made to WO 94/04678. Such camelization may preferentially occur at amino acid positions which are present at the V_{H}-V_{L} interface and at the so-called Camelidae hallmark residues (see for example also WO 94/04678), as also mentioned below. In some embodiments, the V_{H} domain or sequence that is used as a starting material or starting point for generating or designing the camelized Nanobody is a V_{H} sequence from a mammal, e.g., V_{H} sequence of a human being. It should be noted that such camelized Nanobodies of the present disclosure can be obtained in any suitable manner known per se and thus are not strictly limited to polypeptides that have been obtained using a polypeptide that comprises a naturally occurring V_{H} domain as a starting material.

For example, both “humanization” and “camelization” can be performed by providing a nucleotide sequence that encodes such a naturally occurring V_{HH} domain or V_{H} domain, respectively, and then changing, in a manner known per se, one or more codons in said nucleotide sequence such that the new nucleotide sequence encodes a humanized or camelized Nanobody of the present disclosure, respectively, and then expressing the nucleotide sequence thus obtained in a manner known per se so as to provide the desired Nanobody. Alternatively, based on the amino acid sequence of a naturally occurring V_{HH} domain or V_{H} domain, respectively, the amino acid sequence of the desired humanized or camelized Nanobody of the present disclosure, respectively, can be designed and then synthesized de novo using techniques for peptide synthesis known per se. Also, based on the amino acid sequence or nucleotide sequence of a naturally occurring V_{HH} domain or V_{H} domain, respectively, a nucleotide sequence encoding the desired humanized or camelized Nanobody can be designed and then synthesized de novo using techniques for nucleic acid synthesis known per se, after which the nucleotide sequence thus obtained can be expressed in a manner known per se so as to provide the desired Nanobody.

Other suitable ways and techniques for obtaining Nanobodies and/or nucleotide sequences and/or nucleic acids encoding the same, starting from (the amino acid sequence of) naturally occurring V_{H} domains or preferably V_{HH} domains and/or from nucleotide sequences and/or nucleic acid sequences encoding the same will be clear from the skilled person, and may for example comprising combining one or more amino acid sequences and/or nucleotide sequences from naturally occurring V_{H} domains (such as one or more FR's and/or CDR's) with one or more one or more amino acid sequences and/or nucleotide sequences from naturally occurring V_{HH} domains (such an one or more FR's or CDR's), in a suitable manner so as to provide (a nucleotide sequence or nucleic acid encoding) a Nanobody. Also provided are compounds and constructs, and in particular proteins and polypeptides that comprise or essentially consists of at least one such amino acid sequence and/or Nanobody of the disclosure (or suitable fragments thereof), and optionally further comprises one or more other groups, residues, moieties or binding units. In some embodiments, such further groups, residues, moieties, binding units or amino acid sequences may or may not provide further functionality to the amino acid sequence and/or Nanobody (and/or to the compound or construct in which it is present) and may or may not modify the properties of the amino acid sequence and/or Nanobody.

The disclosure also encompasses any polypeptide of the present disclosure that has been glycosylated at one or more amino acid positions, usually depending on the hot used to express the polypeptide. a polypeptide can comprise an amino acid sequence of a CD8 Nanobody of the present disclosure, which is fused at its amino terminal end, at its carboxy terminal end, or both at its amino terminal end and at its carboxy terminal end with at least one further amino acid sequence. Such further amino acid sequence may comprise at least one further Nanobody, so as to provide a polypeptide that comprises at least two, such as three, four or five, Nanobodies, in which said Nanobodies may optionally be linked via one or more linker sequences (as defined herein). Polypeptides of comprising CD8 Nanobody of the present disclosure and one or more another Nanobodies are multivalent polypeptides. In a multivalent polypeptide, the two or more Nanobodies may be the same or different. For example, the two or more Nanobodies in a multivalent polypeptide:

may be directed against the same antigen, i.e. against the same parts or epitopes of said antigen or against two or more different parts or epitopes of said antigen; and/or:

may be directed against the different antigens;

or a combination thereof.

Thus, a bivalent polypeptide, for example:

may comprise two identical Nanobodies;

may comprise a first Nanobody directed against a first part or epitope of an antigen and a second Nanobody directed against the same part or epitope of said antigen or against another part or epitope of said antigen;

or may comprise a first Nanobody directed against a first antigen and a second Nanobody directed against a second antigen different from said first antigen;
whereas a trivalent Polypeptide of the Invention for example:

may comprises three identical or different Nanobodies directed against the same or different parts or epitopes of the same antigen;

may comprise two identical or different Nanobodies directed against the same or different parts or epitopes on a first antigen and a third Nanobody directed against a second antigen different from said first antigen; or

may comprise a first Nanobody directed against a first antigen, a second Nanobody directed against a second antigen different from said first antigen, and a third Nanobody directed against a third antigen different from said first and second antigen,

The CD8 Nanobodies and polypeptides as disclosed herein can also be introduced and expressed in one or more cells, tissues or organs of a multicellular organism, for example for prophylactic and/or therapeutic purposes (e.g. as a gene therapy). For this purpose, the nucleotide sequences encoding the CD8 Nanobodies or polypeptides as disclosed herein can be introduced into the cells or tissues in any suitable way, for example as such (e.g. using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patent by administering a nucleic acid of the invention or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the invention and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y.). Giordano, Nature F Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. Nos. 5,580,859; 1 5,589,5466; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

Accordingly, nucleic acid sequences encoding the CD8 Nanobodies as described herein, and expression construct and host cells comprising the nucleic acid sequence are also provided.

Also disclosed are methods of using CD8 Nanobodies and polypeptides of the present disclosure.

In some embodiments, a polypeptide comprising a CD8 Nanobody can be used in the lipid nanoparticles of the present disclosure for delivering a nucleic acid into an immune cell, as described herein. In some embodiments, CD8 Nanobodies and polypeptides of the present disclosure can be used to treat a condition or a disease in a subject in need thereof. In some embodiments, such conditions or diseases include, but are not limited to, cancer, infections, immune disorders, autoimmune diseases.

In some embodiments, a polypeptide comprising a CD8 Nanobody can be used in an imaging agent. In some embodiments, the imaging agent allows for the detection of human CD8 which is a specific biomarker found on the surface of a subset of T-cell for diagnostic imaging of the immune system. Imaging of CD8 allows for the in vivo detection of T-cell localization. Changes in T-cell localization can reflect the progression of an immune response and can occur over time as a result of various therapeutic treatments or even disease states. In some embodiments, it is used for imaging T-cell localization for immunotherapy.

In addition, CD8 plays a role in activating downstream signaling pathways that are important for the activation of cytolytic T cells that function to clear viral pathogens and provide immunity to tumors. CD8 positive T cells can recognize short peptides presented within the MHCI protein of antigen presenting cells. In some embodiments, a polypeptide comprising a CD8 Nanobody can potentiate signaling through the T cell receptor and enhance the ability of a subject to clear viral pathogens and respond to tumor antigens. Thus, in some embodiments, the antigen binding constructs provided herein can be agonists and can activate the CD8 target.

### II. Ionizable Cationic Lipids

Provided herein are ionizable cationic lipids that can be used to produce lipid nanoparticle compositions to facilitate the delivery of a payload (e.g., a nucleic acid, such as a DNA or RNA, such as an mRNA) disposed therein to cells, e.g., mammalian cells, e.g., immune cells. The ionizable cationic lipids have been designed to enable intracellular delivery of a nucleic acid, e.g., mRNA, to the cytosolic compartment of a target cell type and rapidly degrade into non-toxic components. The complex functionalities of the ionizable cationic lipids are facilitated by the interplay between the chemistry and geometry of the ionizable lipid head group, the hydrophobic “acyl-tail” groups and the linkers connecting the head group and the acyl tail groups. Typically, the pKₐ of the ionizable amine head group is designed to be in the range of 6-8, such as between 6.2-7.4, or between 6.5-7.1, such that it remains strongly cationic under acidic formulation conditions (e.g., pH 4-pH 5.5), neutral in physiological pH (7.4) and cationic in the early and late endosomal compartments (e.g., pH 5.5-pH 7). The acyl-tail groups play a key role in fusion of the lipid nanoparticle with endosomal membranes and membrane destabilization through structural perturbation. The three-dimensional structure of the acyl-tail (determined by its length, and degree and site of unsaturation) along with the relative sizes of the head group and tail group are thought to play a role in promoting membrane fusion, and hence lipid nanoparticle endosomal escape (a key requirement for cytosolic delivery of a nucleic acid payload). The linker connecting the head group and acyl tail groups is designed to degrade by physiologically prevalent enzymes (e.g., esterases, or proteases) or by acid catalyzed hydrolysis.

In one aspect, the present invention provides a compound represented by Formula

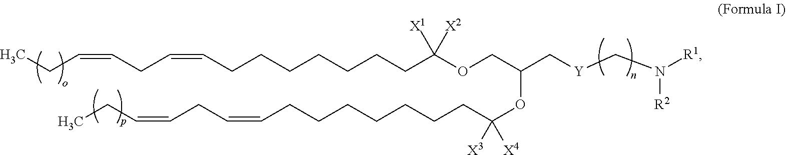

or a salt thereof, wherein:
R¹ and R² are independently C₁₋₃alkyl, or R¹ and R² are taken together with the nitrogen atom to form an optionally substituted piperidinyl or morpholinyl;
Y is selected from the group consisting of —O—, —OC(O)—, —OC(S)—, and —CH₂—;
X¹, X², X³, and X⁴ are hydrogen or X¹ and X² or X³ and X⁴ independently are taken together to form an oxo;
n is 0 or 3;
o and p are independently an integer selected from 2-6;
wherein the compound is not a compound selected from the group consisting of

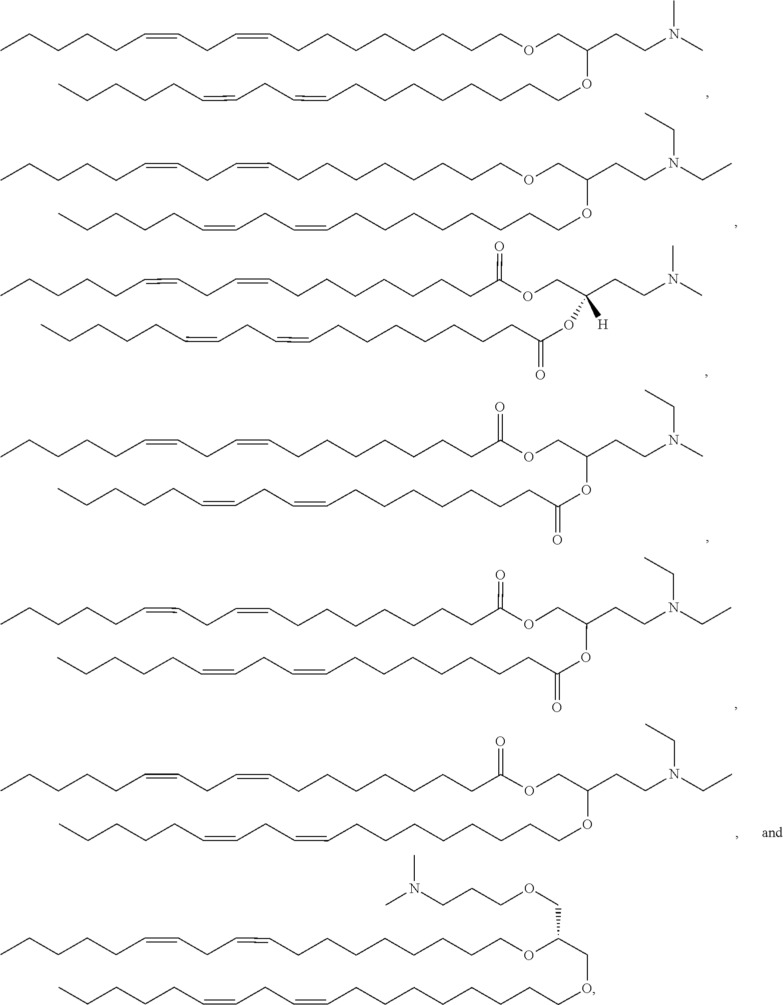

or a salt thereof.

In certain embodiments, o and p may be 2. In certain embodiments, o and p may be 3. In other embodiments, o and p may be 4. In some embodiments, o and p may be 5. In other embodiments, o and p may be 6.

In certain embodiments, X¹ and X² may be taken together to form an oxo and X³ and X⁴ are taken together to form an oxo. In other embodiments, X¹, X², X³, and X⁴ may be hydrogen.

In certain embodiments, Y may be selected from the group consisting of —O—, —OC(O)—, OC(S)— and —CH₂—. For example, in certain embodiments, Y may be —O—. In certain embodiments, Y may be —OC(O)—. In certain embodiments, Y may be —CH2-. In certain embodiments, Y may be —OC(S)—.

In certain embodiments, R¹ and R² may be independently C₁₋₃alkyl. In other embodiments, R¹ and R² may be —CH₃. In certain embodiments, R¹ and R² are —CH₂CH₃. In certain embodiments, R¹ and R² are C₃ alkyl.

In certain embodiments, n may be 0. In other embodiments, n may be 3.

Also provided herein, in part, is a compound represented by Formula II:

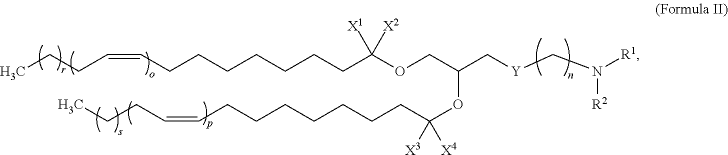

or a salt thereof, wherein:
R¹ and R² are independently C₁₋₃alkyl, or R¹ and R² are taken together with the nitrogen atom to form an optionally substituted piperidinyl or morpholinyl;
Y is selected from the group consisting of —O—, —OC(O)—, —OC(S)—, and —CH₂—;
X¹, X², X³, and X⁴ are hydrogen or X¹ and X² or X³ and X⁴ are taken together to form an oxo;
n is 0-4;
o is 1 and r is an integer selected from 3-8 or o is 2 and r is an integer selected from 1-8,
p is 1 and s is an integer selected from 3-8 or p is 2 and s is an integer selected from 1-8,
wherein,
when o and p are both 1, r and s are independently 4, 5, 7, or 8, and
when o and p are both 2, r and s are independently 1, 2, 4, or 5.

In certain embodiments, X¹ and X² may be taken together to form an oxo and X³ and X⁴ may be taken together to form an oxo. In other embodiments, X¹, X², X³, and X⁴ may be hydrogen.

In certain embodiments, Y may be selected from the group consisting of —O—, —OC(O)—, and —CH₂—. For example, in certain embodiments, Y may be —O—. In certain embodiments, Y may be —OC(O)—. In certain embodiments, Y may be —CH2-. In certain embodiments, Y may be —OC(S)—.

In certain embodiments, R¹ and R² may be independently C₁₋₃alkyl. In other embodiments, R¹ and R² may be —CH3. In certain embodiments, R¹ and R² may be —CH2CH3. In some embodiments, R¹ and R² may be C3 alkyl. In certain embodiments, R¹ and R² are taken together with the nitrogen atom to form an optionally substituted piperidinyl.

In certain embodiments, n may be 0. In other embodiments, n may be 3.

Provided herein, in part, is a compound selected from the group consisting of:

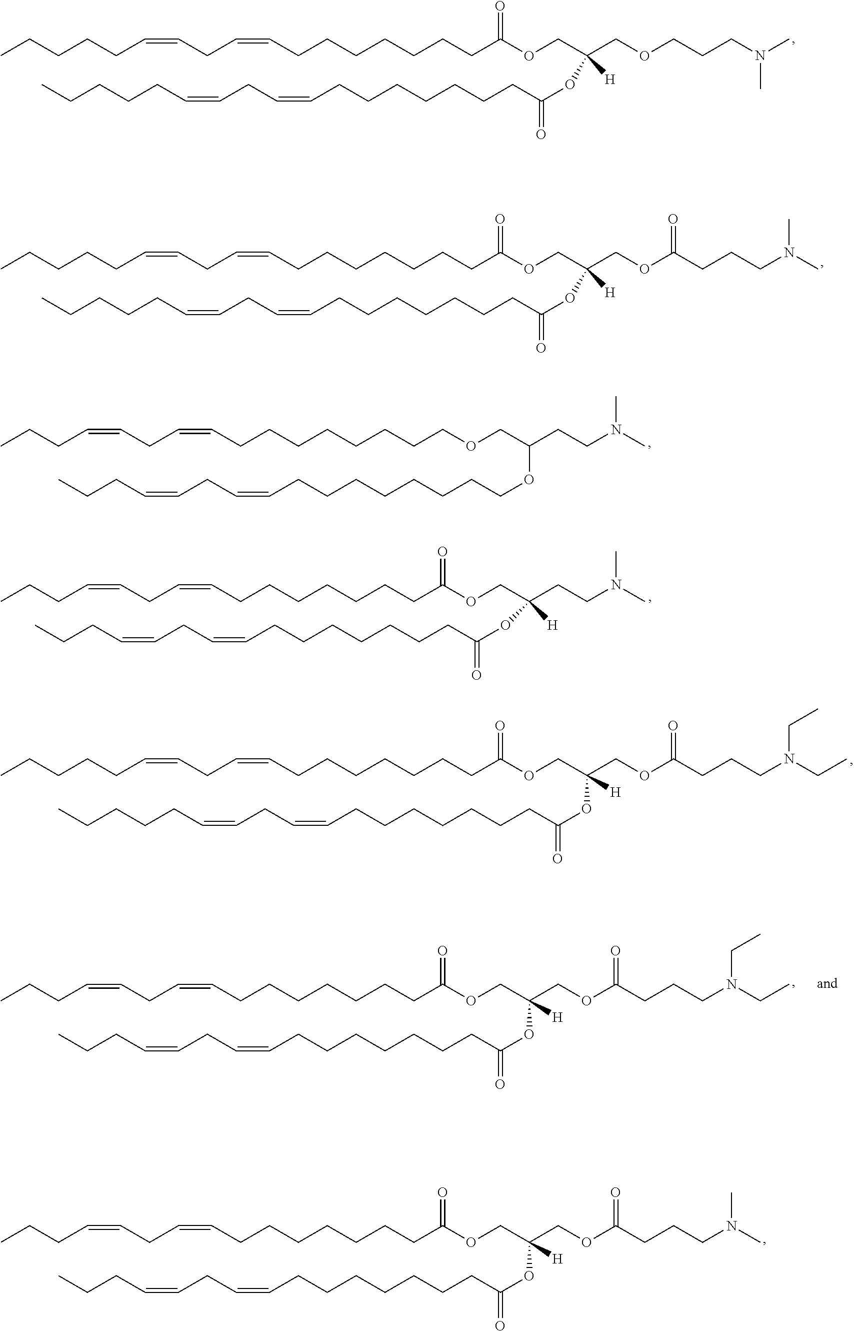

or a salt thereof.

Provided herein, in part, is a compound of formula:

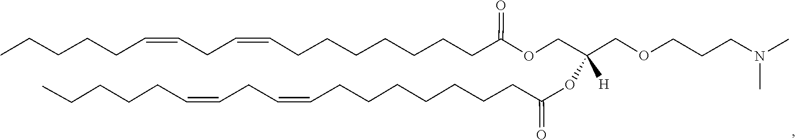 [SMILES: CCCCC/C=C\C/C=C\CCCCCCCC(=O)OC[C@H](COCCCN(C)C)OC(=O)CCCCCCC/C=C\C/C=C\CCCCC]

or a salt thereof.

Provided herein, in part, is a compound of formula:

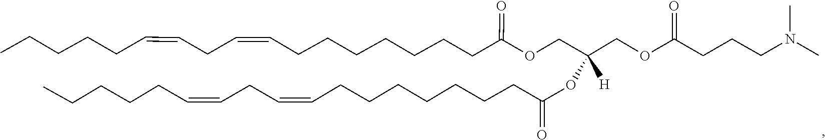 [SMILES: CCCCC/C=C\C/C=C\CCCCCCCC(=O)OC[C@H](COC(=O)CCCN(C)C)OC(=O)CCCCCCC/C=C\C/C=C\CCCCC]

or a salt thereof.

Provided herein, in part, is a compound of formula:

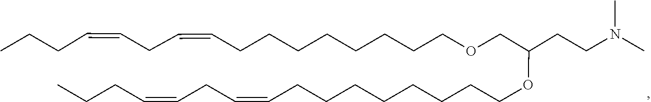 [SMILES: CCC/C=C\C/C=C\CCCCCCCCOCC(CCN(C)C)OCCCCCCCC/C=C\C/C=C\CCC]

or a salt thereof.

Provided herein, in part, is a compound of formula:

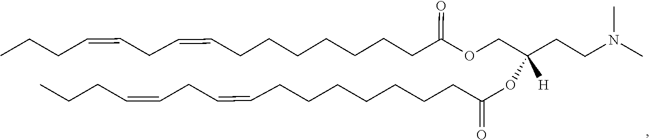 [SMILES: CCC/C=C\C/C=C\CCCCCCCC(=O)OC[C@H](CCN(C)C)OC(=O)CCCCCCC/C=C\C/C=C\CCC]

or a salt thereof.

Provided herein, in part, is a compound of formula:

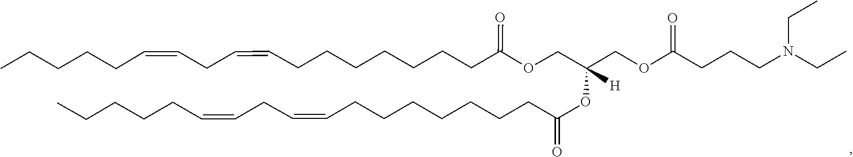 [SMILES: CCCCC/C=C\C/C=C\CCCCCCCC(=O)OC[C@H](COC(=O)CCCN(CC)CC)OC(=O)CCCCCCC/C=C\C/C=C\CCCCC]

or a salt thereof.

Provided herein, in part, is a compound of formula:

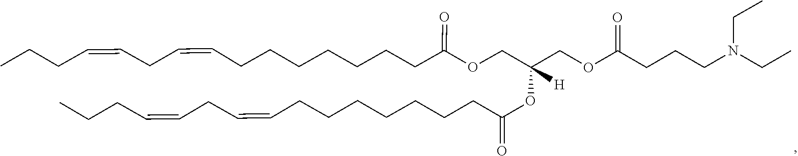 [SMILES: CCC/C=C\C/C=C\CCCCCCCC(=O)OC[C@H](COC(=O)CCCN(CC)CC)OC(=O)CCCCCCC/C=C\C/C=C\CCC]

or a salt thereof.

Provided herein, in part, is a compound of formula:

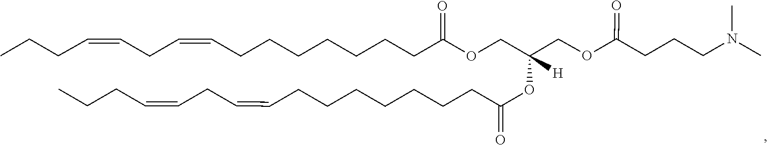 [SMILES: CCC/C=C\C/C=C\CCCCCCCC(=O)OC[C@H](COC(=O)CCCN(C)C)OC(=O)CCCCCCC/C=C\C/C=C\CCC]

or a salt thereof.

In certain embodiments, the compound is a compound of Formula III:

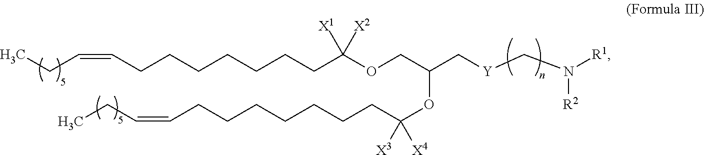

or a salt thereof, wherein:

R¹ and R² are independently C₁₋₃alkyl, or R¹ and R² are taken together with the nitrogen atom to form an optionally substituted piperidinyl or morpholinyl;

Y is selected from the group consisting of —O—, —OC(O)—, —OC(S)—, and —CH₂—;
X¹, X², X³, and X⁴ are hydrogen or X¹ and X² or X³ and X⁴ are taken together to form an oxo; and n is an integer selected from 0-4.

In certain embodiments, X¹ and X² may be taken together to form an oxo and X³ and X⁴ may be taken together to form an oxo. In other embodiments, X¹, X², X³, and X⁴ may be hydrogen.

In certain embodiments, Y may be selected from the group consisting of —O—, —OC(O)—, and —CH₂—. For example, in certain embodiments, Y may be —O—. In certain embodiments, Y may be —OC(O)—. In certain embodiments, Y may be —CH₂—. In certain embodiments, Y may be —OC(S)—.

In certain embodiments, R¹ and R² may be independently C₁₋₃alkyl. In other embodiments, R¹ and R² may be —CH₃. In certain embodiments, R¹ and R² may be —CH₂CH₃. In some embodiments, R¹ and R² may be C₃ alkyl. In certain embodiments, R¹ and R² are taken together with the nitrogen atom to form an optionally substituted piperidinyl.

In certain embodiments, n may be 0. In other embodiments, n may be 3.

Also provided herein is a compound of the formula:

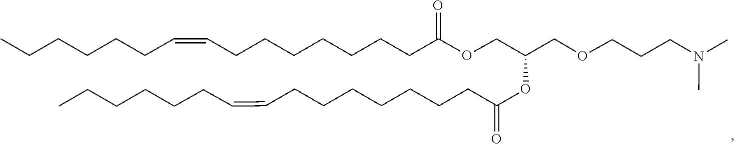 [SMILES: CCCCCC/C=C\CCCCCCCC(=O)OC[C@H](COCCCN(C)C)OC(=O)CCCCCCC/C=C\CCCCCC]

or a salt thereof

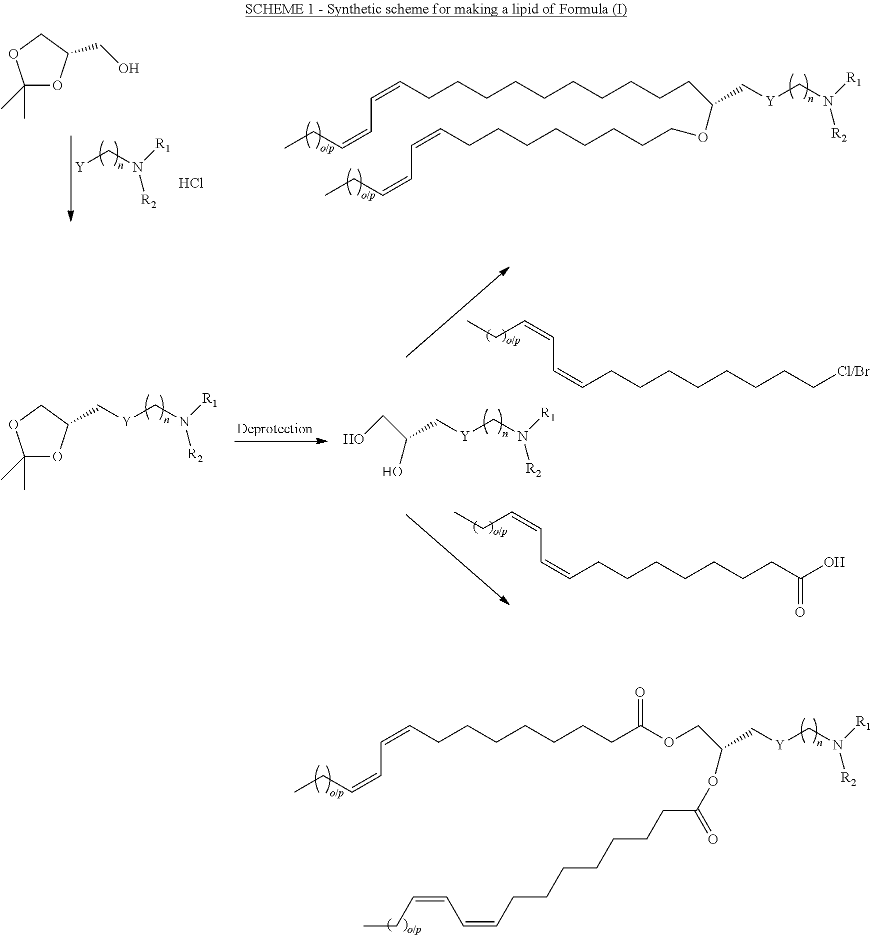

A compound of Formula I may be prepared, e.g., according to Scheme 1. A hydroxy-functional protected propane diol is converted to the corresponding dimethyl amino-function ether (Y=oxo) or ester (Y=O—C(0)). The ether bond formation results from a reaction of the alkyl halide with alcohol in the presence of tertiary butylammonium iodide/NaOH in THF at 80° C. The ester bond formation utilizes treatment of an acid functional dimethylamine with alcohol under carbodiimide activation (DCM, EDC, DIEPA, DMAP). The diol deprotection yields a vicinal diol intermediate that is subsequently converted to the corresponding ether linked or ester linked diacyl lipids by treatment with TBAI/NaOH and bromo-acyl or by carbodiimide mediated carboxylic acid activation for ester bond formation, respectively.

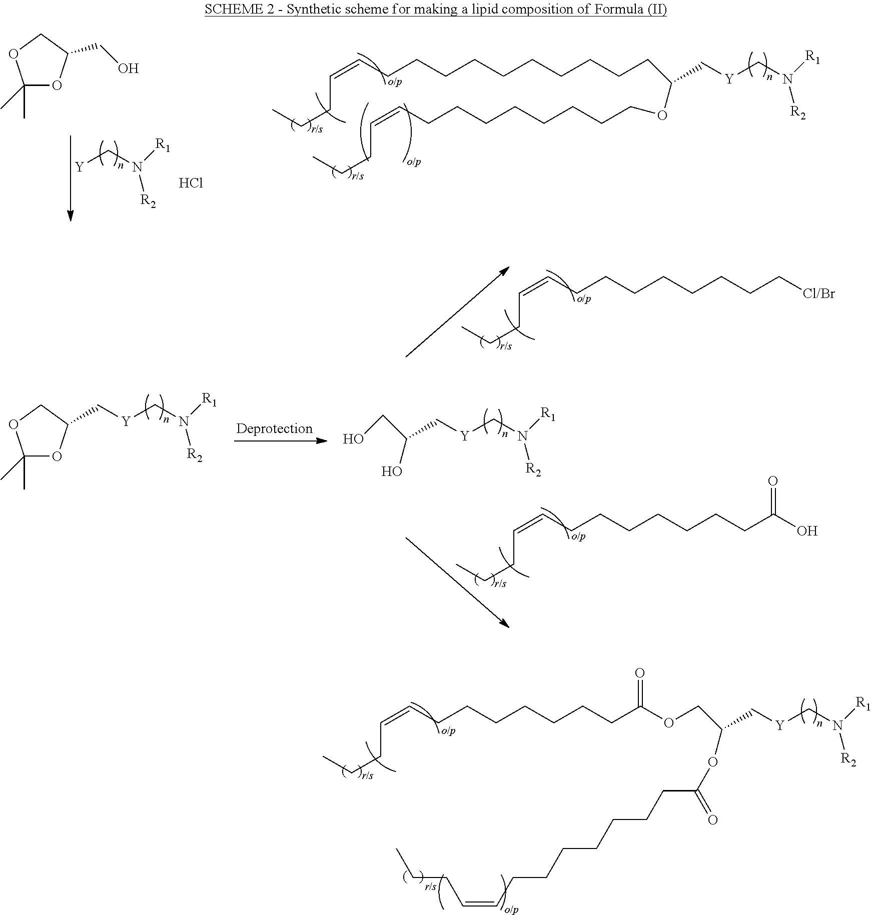

A compound of Formula II may be prepared, e.g., according to Scheme 2. The synthetic procedure is as outlined above for Scheme 1; however, in Scheme 2, either bis-unsaturated acyl groups or mono-unsaturated acyl groups may be employed to obtain a lipid of Formula II.

In some embodiments, ionizable cationic lipid used in the LNPs of the present disclosure is selected from the lipids in Table 1, or a combination thereof. In some embodiments, the ionizable cationic lipid is:

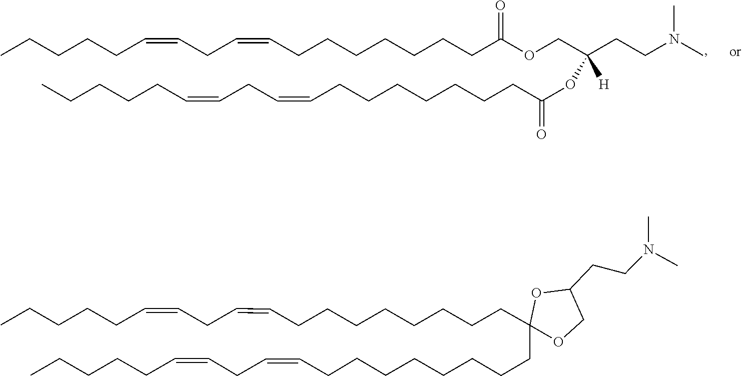

In some embodiments, the ionizable cationic lipid is not Dlin-MC3-DMA.

### III. Lipid-Immune Cell Targeting Group Conjugates

As discussed herein, the LNPs may be targeted to a particular cell type, e.g., an immune cell, e.g., a T cell, B cell, or natural killer (NK) cell. This can be accomplished by using one or more of the lipids described herein. Furthermore, targeting can be enhanced by including a targeting group at a solvent accessible surface of an LNP particle. For example, targeting groups may include a member of a specific binding pair, e.g., an antibody-antigen pair, a ligand-receptor pair, etc. In certain embodiments, the targeting group is an antibody. Targeting can be implemented, for example, by using lipid-immune cell targeting group conjugates described herein.

Optionally, the targeting moiety is an antibody fragment without an Fc component. Previous attempts to target circulating immune cells with LNPs have employed full antibodies (WO 2016/189532 A1). Liposomes or lipid based particles with conjugated full antibodies clear more quickly from the circulation due to engagement of the Fc, reducing their potential for reaching the target cell of interest (Harding et al. (1997) Biochim Biophys. Acta 1327, 181-192; Sapra et al. (2004) Clin Cancer Res 10, 1100-1111; Aragnol et al., (1986) Proc Natl Acad Sci USA 83, 2699-2703). Liposomes targeted with antibody fragments retain their long circulating properties, like those targeted to EGFR (Mamot et al., (2005) Cancer Res 65, 11631-11638), ErbB2 (Park et al. (2002) Clin Cancer Res 8, 1172-1181), or EphA2 (Kamoun et al., 2019 Nat. Biomed. Eng 3, 264-280). In addition, lipid based carriers can be prepared using a micellar insertion process that allows for the nearly quantitative incorporation of the antibody conjugation following it's separate manufacturing (Nellis et al. (2005) Biotechnol Prog 21, 221-232), compared to a highly inefficient insertion when conjugating full IgGs (Ishida et al. (1999) FEBS Lett. 460, 129-133) or the need to complete conjugation directly on an intact LNP (WO 2016/189532 A1). scFv, Fab, or V_{HH} fragments can also be directly conjugated to activated PEG-lipids to make insertable conjugates.

In certain embodiments, a targeting group may be a surface-bound antibody or surface bound antigen binding fragment thereof, which can permit tuning of cell targeting specificity. This is especially useful since highly specific antibodies can be raised against an epitope of interest for the desired targeting site. In one embodiment, multiple different antibodies can be incorporated into, and presented at the surface of an LNP, where each antibody binds to different epitopes on the same antigen or different epitopes on different antigens. Such approaches can increase the avidity and specificity of targeting interactions to a particular target cell.

A targeting group or combination of targeting groups can be selected based on the desired localization, function, or structural features of a given target cell. For example, in order to target a T-cell, T-cell population or T-cell subpopulation, one or more antibodies or antigen binding fragments or antigen binding derivatives thereof may be selected that target a T-cell, such as via a T-cell surface antigen. Exemplary T-cell surface antigens include, but are not limited to, for example, CD2, CD3, CD4, CD5, CD7, CD8, CD28, CD39, CD69, CD103, CD137, CD45, T-cell receptor (TCR) β, TCR-α, TCR-α/β, TCR-γ/δ, PD1, CTLA4, TIM3, LAG3, CD18, IL-2 receptor, CD11a, GL7, TLR2, TLR4, TLR5 and IL-15 receptor. In order to target an NK cell, or NK cell population, one or more antibodies, antigen binding fragments or antigen binding derivatives thereof maybe selected that target an NK cell such as via a NK cell surface antigen. Exemplary NK cell surface antigens include, but are not limited to, CD48, CD56, CD85a, CD85c, CD85d, CD85e, CD85f, CD85i, CD85j, CD158b2, CD161, CD244, CD16a, CD16b, IL-2 receptor, CD27, CD28, CD48, CD69, CD70, CD86, CD112, CD122, CD155, CD161, CD244, CD266, CD314/NKG2D, CD336/NKP44, CD337/NKP30. In order to target a B cell or B cell population, one or more antibodies, antigen binding fragments or antigen binding derivatives thereof maybe selected that target a B cell such as via a B cell antigen. Exemplary B cell antigens include, but are not limited to, CD19 for all B cells except plasma cells, CD19, CD25, and CD30 for activated B cells, CD27, CD38, CD78, CD138, and CD319 for plasma cells, CD20, CD27, CD40, CD80 and PDL-2 for memory cells, Notch2, CD1, CD21, and CD27 for marginal zone B cells, CD21, CD22, and CD23 for follicular B cells, and CD1, CD5, CD21, CD24, and TLR4 for regulatory B cells.

In certain embodiments, targeting can be implemented, for example, by using lipid-immune cell targeting group conjugates described herein. Exemplary lipid-immune cell targeting group conjugates can include compounds of Formula IV,

[Lipid]-[optional linker]-[immune cell targeting group, e.g., T-cell targeting molecule, e.g., an anti-CD2 antibody, anti-CD3 antibody, anti-CD7 antibody, or anti-CD8 antibody] (Formula IV).

In some embodiments, the immune cell targeting group is a polypeptide, and the lipid is conjugated to the N-terminus, C-terminus, or anywhere in the middle part of the polypeptide.

In certain embodiments, the targeting group or targeting molecule is a T-cell targeting agent, for example, an antibody, that binds to a T-cell antigen selected from the group consisting of CD2, CD3, CD4, CD5, CD7, CD8, CD28, CD137, CD45, T-cell receptor (TCR)β, TCR-α, TCR-α/β, TCR-y/8, PD1, CTLA4, TIM3, LAG3, CD18, IL-2 receptor, CD11a, TLR2, TLR4, TLR5, IL-7 receptor, or IL-15 receptor. In certain embodiments, the T cell antigen may be CD2, and the targeting group can be, for example, an anti-CD2 antibody. In certain embodiments, the T cell antigen may be CD3, and the targeting group can be, for example, an anti-CD3 antibody. In certain embodiments, the T cell antigen may be CD4, and the targeting group can be, for example, an anti-CD4 antibody. In certain embodiments, the T cell antigen may be CD5, and the targeting group can be, for example, an anti-CD5 antibody. In certain embodiments, the T cell antigen may be CD7, and the targeting group can be, for example, an anti-CD7 antibody. In certain embodiments, the T cell antigen may be CD8, and the targeting group can be, for example, an anti-CD8 antibody. In certain embodiments, the T cell antigen may be TCR (3, and the targeting group can be, for example, an anti-TCR (3 antibody. In some embodiments, the antibody is a human or humanized antibody.

An exemplary CD2 binding agent can be an antibody selected from the group consisting of 9.6 (https://academic.oup.com/intimm/article/10/12/1863/744536), 9-1 (https://academic.oup.com/intimm/article/10/12/1863/744536), TS2/18.1.1 (ATCC HB-195), Lo-CD2b (ATCC PTA-802), Lo-CD2a/BTI-322 (U.S. Pat. No. 6,849,258B1), Sipilzumab/MEDI-507 (U.S. Pat. No. 6,849,258B1/en), 35.1 (ATCC HB-222), OKT11 (ATCC CRL-8027), RPA-2.1 (PCT Publication WO2020023559A1), AF1856 (R&D Systems), MAB18562 (R&D Systems), MAB18561 (R&D Systems), MAB1856 (R&D Systems), PAB30359 (Abnova Corporation), 10299-1 (Abnova Corporation), and antigen binding fragments thereof. In certain embodiments, the binding agent comprises a heavy chain variable domain (V_{H}) and a light chain variable domain (V_{L}) of an antibody selected from the group consisting of AF1856 (R&D Systems), MAB18562 (R&D Systems), MAB18561 (R&D Systems), MAB1856 (R&D Systems), PAB30359 (Abnova Corporation), and 10299-1 (Abnova Corporation). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of AF1856 (R&D Systems), MAB18562 (R&D Systems), MAB18561 (R&D Systems), MAB1856 (R&D Systems), PAB30359 (Abnova Corporation), and 10299-1 (Abnova Corporation).

An exemplary CD2 binding agent can also be selected from antibodies or antibody fragments employing CDRs of clones 9.6, 9-1, TS2/18.1.1, Lo-CD2b, Lo-CD2a, BTI-322, sipilzumab, 35.1, OKT11, RPA-2.1, SQB-3.21, LT2, TS1/8, UT329, 4F22, OX-34, UQ2/42, MU3, U7.4, NFN-76, or MOM-181-4-F(E).

An exemplary CD3 binding agent (CD3γ/δ/ε, CD3γ, CD3δ, CD3γ/ε, CD3δ/ε, or CD3ε) can be an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), 16669-1-AP (CD3δ, Invitrogen) and antigen binding fragments thereof. In certain embodiments, the binding agent comprises a V_{H} domain and a V_{L} domain of an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3δ, Invitrogen). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3δ, Invitrogen).

An exemplary CD3 binding agent can also be selected from antibodies or antibody fragments employing CDRs of clones hsp34, OKT-3, UCHT1, 38.1, HIT3a, RFT8, SK7, BC3, SP34-2, HU291, TRX4, Catumaxomab, teplizumab, 3-106, 3-114, 3-148, 3-190, 3-271, 3-550, 4-10, 4-48, H2C, F12Q, I2C, SP7, 3F3A1, CD3-12, 301, RIV9, JB38-29, JE17-74, GT0013, 4E2, 7A4, 4D10A6, SPV-T3b, M2AB, ICO-90, 30A1 or Hu38E4.v1 (US Patent Application 20200299409A1), REGN5458 (US Patent Application 20200024356A1), Blinatumomab (https://go.drugbank.com/drugs/DB09052/polypeptide sequences.fasta). In some embodiments, the conjugate comprises a Fab, wherein the Fab comprises (a) a heavy chain fragment comprising the amino acid sequence of SEQ ID NO: 1 and a light chain fragment comprising the amino acid sequence of SEQ ID NO:2 or 3.

An exemplary CD4 binding agent can be an antibody selected from the group consisting of Ibalizumab (https://www.genome.jp/dbget-bin/www_bget?D09575), AF1856 (R&D Systems), MAB554 (R&D Systems), BF0174 (Affinity Biosciences), PAB31115 (Abnova Corporation), CAL4 (Abcam), and antigen binding fragments thereof. In certain embodiments, the binding agent comprises a V_{H} domain and a V_{L} domain of an antibody selected from the group consisting of AF1856 (R&D Systems), MAB554 (R&D Systems), BF0174 (Affinity Biosciences), PAB31115 (Abnova Corporation), and CAL4 (Abcam). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of AF1856 (R&D Systems), MAB554 (R&D Systems), BF0174 (Affinity Biosciences), PAB31115 (Abnova Corporation), and CAL4 (Abcam).

An exemplary CD4 binding agent can also be selected from antibodies or antibody fragments employing CDRs of clones Ibalizumab, OKT4, RPA-T4, S3.5, SK3, N1UGO, RIV6, OTI18E3, MEM-241, B486A1, RFT-4 g, 7E14, MDX.2, MEM-115, MEM-16, ICO-86, Edu-2, or ilbalizumab.

An exemplary CD5 binding agent can be an antibody selected from the group consisting of He3, MAB1636 (R&D Systems), AF1636 (R&D Systems), MAB115 (R&D Systems), C5/473+CD5/54/F6 (Abcam), CD5/54/F6 (Abcam), 65152 (Proteintech), and antigen binding fragments thereof. In some embodiments, the binding agent comprises a V_{H} domain and a V_{L} of an antibody selected from the group consisting of MAB1636 (R&D Systems), AF1636 (R&D Systems), MAB115 (R&D Systems), C5/473+CD5/54/F6 (Abcam), CD5/54/F6 (Abcam), and 65152 (Proteintech). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of MAB1636 (R&D Systems), AF1636 (R&D Systems), MAB115 (R&D Systems), C5/473+CD5/54/F6 (Abcam), CD5/54/F6 (Abcam), and 65152 (Proteintech).

An exemplary CD5 binding agent can also be selected from antibodies or antibody fragments employing CDRs of clones of zolimomab, 5D7, L17F12, and UCHT2, 1D8, 3121, 4H10, 8J23, 504, 4H2, 5G2, 8G8, 6M4, 2E3, 4E24, 4F10, 7J9, 7P9, 8E24, 6L18, 7H7, 1E7, 8J21, 7111, 8M9, 1P21, 2H11, 3M22, 5M6, 5H8, 7119, 1A2, 8E15, 8C10, 3P16, 4F3, 5M24, 5024, 7B16, 1E8, 2H16, BLa1, 1804, DK23, Cris1, MEM-32, H65, 4C7, OX-19, Leu-1, 53-7.3, 4H8E6, T101, EP2952, D-9, H-3, HK231, N-20, Y2/178, H-300, CD5/54/F6, Q-20, CC17, MOM-18539-S(P), or MOM-18885-S(P).

An exemplary CD7 binding agent can be an antibody selected from the group consisting of MAB7579 (R&D Systems), AF7579 (R&D Systems), EPR22065 (Abcam), 1G10D8 (Proteintech), NBP2-32097 (Novus Biologicals), NBP2-38440 (Novus Biologicals), and antigen binding fragments thereof. In certain embodiments, the binding agent comprises a V_{H} domain and a V_{L} of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals).

An exemplary CD7 binding agent can also be selected from antibodies or antibody fragments employing CDRs of clones TH-69, 3Afl1, T3-3A1, 124-1D1, 3A1f, CD7-6B7, or V_{HH}6.

An exemplary CD8 (CD8α, CD8α/α, CD8α/β or CD8β) binding agent can be an antibody selected from the group consisting of 2.43 (Invitrogen), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), YTS105.18 (CD8α, Novus Biologicals), TRX2 (https://patents.justia.com/patent/20170198045), and antigen binding fragments thereof. In certain embodiments, the binding agent comprises a V_{H} domain and a V_{L} domain of an antibody selected from the group consisting of 2.43 (Invitrogen), 51.1 (ATCC HB-230), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), and YTS105.18 (CD8α, Novus Biologicals). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of 2.43 (Invitrogen), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), and YTS105.18 (CD8α, Novus Biologicals).

An exemplary CD8 binding agent can also be selected from antibodies or antibody fragments employing CDRs of clones OKT-8, 51.1, S6F1, TRX2, and UCHT4, SP16, 3B5, C8-144B, HIT8a, RAVB3, LT8, 17D8, MEM-31, MEM-87, RIV11, DK-25, YTC141.1HL, or YTC182.20. In some embodiments, the conjugate comprises a Fab, wherein the Fab comprises a heavy chain fragment comprising the amino acid sequence of SEQ ID NO: 6 and a light chain fragment comprising the amino acid sequence of SEQ ID NO: 7.

An exemplary CD137 binding agent can be selected from antibodies or antibody fragments employing CDRs of clones 4B4-1, P566, or Urelumab. An exemplary CD28 binding agent can be selected from antibodies or antibody fragments employing CDRs of clone TAB08. An exemplary CD45 binding agent can be selected from antibodies or antibody fragments employing CDRs of clones BC8, 9.4, 4B2, Tu116, or GAP8.3. An exemplary CD18 binding agent can be selected from antibodies or antibody fragments employing CDRs of clones 1B4, TS1/18, MEM-48, YFC118-3, TA-4, MEM-148, or R3-3, 24. An exemplary CD11a binding agent can be selected from antibodies or antibody fragments employing CDRs of clone MHM24 or Efalizumab. An exemplary IL-2 receptor binding agent can be selected from of antibodies or antibody fragments employing CDRs of clones YTH 906.9HL, IL2R.1, BC96, B-B10, 216, MEM-181, ITYV, MEM-140, ICO—105, Daclizumab, or from the group consisting of IL2 or fragments of IL2. An exemplary IL-15R binding agent can be selected from antibodies or antibody fragments employing CDRs of clones JM7A4, or OTI3D5, or from the group consisting of IL15 or fragments of IL15. An exemplary TLR2 binding agent can be selected from antibodies or antibody fragments employing CDRs of clones JM22-41, TL2.1, 11G7, or TLR2.45. An exemplary TLR4 binding agent can be selected from antibodies or antibody fragments employing CDRs of clones HTA125, or 76B357-1. An exemplary TLR5 binding agent can be selected from antibodies or antibody fragments employing CDRs of clones 85B152-5, or 9D759-2. An exemplary GL7 binding agent can be selected from antibodies or antibody fragments employing CDRs of clone GL7.

An exemplary PD1 binding agent can be selected from antibodies or antibody fragments employing CDRs of clones MIH4, J116, J150, OTIB11, OTI17B10, OTI3A1, or OTI16D4. In addition, exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

An exemplary CTLA-4 binding agent can be selected from antibodies or antibody fragments employing CDRs of clones ER4.7G.11 [7G11], OTI9G4, OTI9F3, OTI3A5, A3.4H2.H12, 14D3, OTI3A12, OTI1A11, OTI1E8, OTI3B11, OTI3D2, OTI10C8, OTI2E9, OTI6F1, OTI7D3, OTI85B, OTI12C6. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

An exemplary TCR β binding agent can be an antibody selected from the group consisting of H57-597 (Invitrogen), 8A3 (Novus Biologicals), R73 (TCRα/β, Abcam), E6Z3S (TRBC1/TCRβ, Cell Signaling Technology), and antigen binding fragments thereof. In certain embodiments, the binding agent comprises a V_{H} domain and a V_{L} of an antibody selected from the group consisting of H57-597 (Invitrogen), 8A3 (Novus Biologicals), R73 (TCRα/β, Abcam), and E6Z3S (TRBC1/TCRβ, Cell Signaling Technology). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the Vx and V_{L} sequences of an antibody selected from the group consisting of H57-597 (Invitrogen), 8A3 (Novus Biologicals), R73 (TCRα/(3, Abcam), and E6Z3S (TRBC1/TCR(3, Cell Signaling Technology).

An exemplary CD137 binding agent can be selected from antibodies or antibody fragments employing CDRs of clones 4B4-1, P566, or Urelumab.

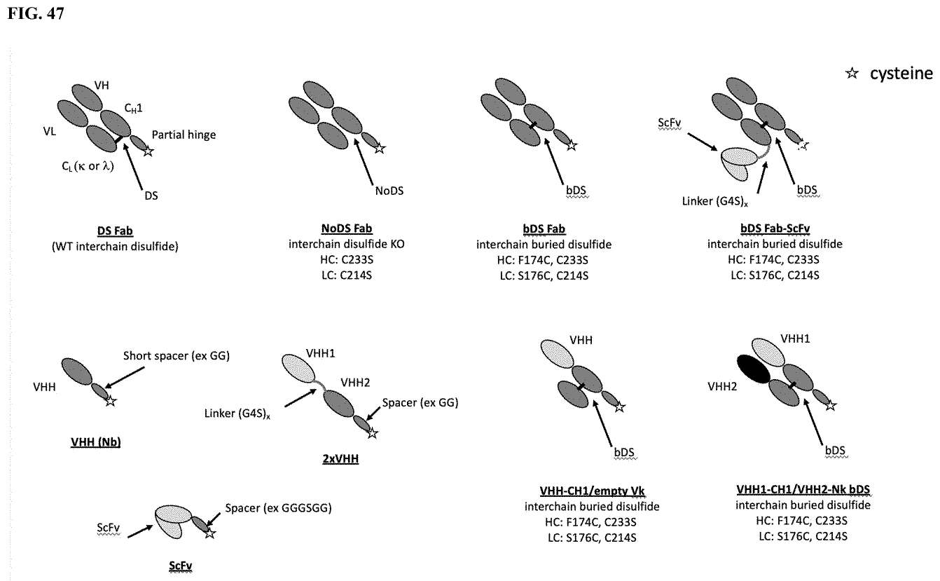

In some embodiments, the immune cell targeting group comprises an antibody selected from the group consisting of a Fab, F(ab′)2, Fab′-SH, Fv, and scFv fragment. In some embodiments, the antibody is a human or humanized antibody. In some embodiments, the immune cell targeting group comprises a Fab or an immunoglobulin single variable domain, such as a Nanobody. In some embodiments, the immune cell targeting group comprises a Fab that does not comprise a natural interchain disulfide bond. For example, in some embodiments, the Fab comprises a heavy chain fragment that comprises a C233S substitution, and/or a light chain fragment that comprises a C214S substitution, numbering according to Kabat. In some embodiments, the immune cell targeting group comprises a Fab that comprises one or more non-native interchain disulfide bonds. In some embodiments, the interchain disulfide bonds are between two non-native cysteine residues on the light chain fragment and heavy chain fragment, respectively. For example, in some embodiments, the Fab comprises a heavy chain fragment that comprises F174C substitution, and/or a light chain fragment that comprises S176C substitution, numbering according to Kabat. In some embodiments, the Fab comprises a heavy chain fragment that comprises F174C and C233S substitutions, and/or a light chain fragment that comprises S176C and C214S substitutions, numbering according to Kabat. In some embodiments, the immune cell targeting group comprises a C-terminal cysteine residue. In some embodiments, the immune cell targeting group comprises a Fab that comprises a cysteine at the C-terminus of the heavy or light chain fragment. In some embodiments, the Fab further comprises one or more amino acids between the heavy chain of the Fab and the C-terminal cysteine. For example, in some embodiments, the Fab comprises two or more amino acids derived from an antibody hinge region (e.g., a partial hinge sequence) between the C-terminus of the Fab and the C-terminal cysteine. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker. In some embodiments, the Fab antibody is a DS Fab, a NoDS Fab, a bDS Fab, a bDS Fab-ScFv, as demonstrated in FIG. 47.

In some embodiments, the immune cell targeting group comprises an immunoglobulin single variable domain, such as a Nanobody (e.g., a Vim). In some embodiments, the Nanobody comprises a cysteine at the C-terminus. In some embodiments, the Nanobody further comprises a spacer comprising one or more amino acids between the V_{HH} domain and the C-terminal cysteine. In some embodiments, the spacer comprises one or more glycine residues, e.g., two glycine residues. In some embodiments, the immune cell targeting group comprises two or more V_{HH} domains. In some embodiments, the two or more V_{HH} domains are linked by an amino acid linker. In some embodiments, the amino acid linker comprises one or more glycine and/or serine residues (e.g., one or more repeats of the sequence GGGGS). In some embodiments, the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds (e.g., interchain disulfide bonds). In some embodiments, the immune cell targeting group comprises a V_{HH} domain linked to an antibody CH1 domain, and wherein the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat. In some embodiments, the antibody is a ScFv, a V_{HH}, a 2×V_{HH}, a V_{HH}-CH1/empty Vk, or a V_{HH}1-CH1/V_{HH}-2-Nb bDS, as demonstrated in FIG. 47.

An exemplary targeting moiety may have an amino sequence as set forth below:

Anti-CD3 hSP34-Fab sequences:
hSP34 heavy chain (HC) sequence (SEQ ID NO: 1):
EVQLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRHG
NFGNSYISYWAYWGQGTLVTVSSASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSSDKTHTC
hSP34-mlam light chain (LC) sequence (mouse lambda)
(SEQ ID NO: 2):
QTVVTQEPSLTVSPGGTVTLTCGSSTGAVTSGNYPNWVQQKPGQAPRGLIG
GTKFLAPGTPARFSGSLLGGKAALTLSGVQPEDEAEYYCVLWYSNRWVFGG
GTKLTVLGQPKSSPSVTLFPPSSEELETNKATLVCTITDFYPGVVTVDWKV
DGTPVTQGMETTQPSKQSNNKYMASSYLTLTARAWERHSSYSCQVTHEGHT
VEKSLSRADSS
SP34-hlam LC (human lambda) (SEQ ID NO: 3):
QTVVTQEPSLTVSPGGTVTLTCGSSTGAVTSGNYPNWVQQKPGQAPRGLIG
GTKFLAPGTPARFSGSLLGGKAALTLSGVQPEDEAEYYCVLWYSNRWVFGG
GTKLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGST
VEKTVAPAESS
Anti-CD3 Hu291-Fab sequences:
Hu291 HC (SEQ ID NO: 4):
QVQLVQSGAEVKKPGASVKVSCKASGYTFISYTMHWVRQAPGQGLEWMGYI
NPRSGYTHYNQKLKDKATLTADKSASTAYMELSSLRSEDTAVYYCARSAYY
DYDGFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICN
VNHKPSNTKVDKKVEPKSSDKTHTC
Hu 291 LC (SEQ ID NO: 5):
MDMRVPAQLLGLLLLWLPGAKCDIQMTQSPSSLSASVGDRVTITCSASSSV
SYMNWYQQKPGKAPKRLIYDTSKLASGVPSRFSGSGSGTDFTLTISSLQPE
DFATYYCQQWSSNPPTFGGGTKVEIKRTVAAPSVFIFPPSDEQLKSGTASV
VCLLNNFYPREAKVQWKVDNALQSGNSQESVTEQDSKDSTYSLSSTLTLSK
ADYEKHKVYACEVTHQGLSSPVTKSFNRGES
Anti-CD8 TRX2-Fab sequences:
TRX2 HC (SEQ ID NO: 6):
QVQLVESGGGVVQPGRSLRLSCAASGFTFSDFGMNWVRQAPGKGLEWVALI
YYDGSNKFYADSVKGRFTISRDNSKNTLYLQMNSLRAEDTAVYYCAKPHYD
GYYHFFDSWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYIC
NVNHKPSNTKVDKKVEPKSSDKTHTC
TRX2 LC (SEQ ID NO: 7):
DIQMTQSPSSLSASVGDRVTITCKGSQDINNYLAWYQQKPGKAPKLLIYNT
DILHTGVPSRFSGSGSGTDFTFTISSLQPEDIATYYCYQYNNGYTFGQGTK
VEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPV
TKSFNRGES
Anti-CD8 OKT8-Fab sequences:
OKT8 HC (SEQ ID NO: 8):
QVQLVQSGAEDKKPGASVKVSCKASGFNIKDTYIHWVRQAPGQGLEWMGRI
DPANDNTLYASKFQGRVTITADTSSNTAYMELSSLRSEDTAVYYCGRGYGY
YVFDHWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSSDKTHTC
OKT8 LC (SEQ ID NO: 9):
DIVMTQSPSSLSASVGDRVTITCRTSRSISQYLAWYQEKPGKAPKLLIYSG
STLQSGVPSRFSGSGSGTDFTLTISSLQPEDFATYYCQQHNENPLTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES
Anti-CD4 Ibalizumab-Fab sequences:
Ibalizumab HC (SEQ ID NO: 10):
QVQLQQSGPEVVKPGASVKMSCKASGYTFTSYVIHWVRQKPGQGLDWIGYI
NPYNDGTDYDEKFKGKATLTSDTSTSTAYMELSSLRSEDTAVYYCAREKDN
YATGAWFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYI
CNVNHKPSNTKVDKKVEPKSSDKTHTC
Ibalizumab LC (SEQ ID NO: 11):
DIVMTQSPDSLAVSLGERVTMNCKSSQSLLYSTNQKNYLAWYQQKPGQSPK
LLIYWASTRESGVPDRFSGSGSGTDFTLTISSVQAEDVAVYYCQQYYSYRT
FGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGES
anti-CD5 He3-Fab sequences:
He3 HC (SEQ ID NO: 12):
EIQLVQSGGGLVKPGGSVRISCAASGYTFTNYGMNWVRQAPGKGLEWMGWI
NTHTGEPTYADSFKGRFTFSLDDSKNTAYLQINSLRAEDTAVYFCTRRGYD
WYFDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSSDKTHTC
He3 LC (SEQ ID NO: 13):
DIQMTQSPSSLSASVGDRVTITCRASQDINSYLSWFQQKPGKAPKTLIYRA
NRLESGVPSRFSGSGSGTDYTLTISSLQYEDFGIYYCQQYDESPWTFGGGT
KLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES
anti-CD7 TH-69-Fab sequences:
TH-69 HC (SEQ ID NO: 14):
EVQLVESGGGLVKPGGSLKLSCAASGLTFSSYAMSWVRQTPEKRLEWVASI
SSGGFTYYPDSVKGRFTISRDNARNILYLQMSSLRSEDTAMYYCARDEVRG
YLDVWGAGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNH
KPSNTKVDKKVEPKSCDKTHTC
TH-69 LC (SEQ ID NO: 15):
DIQMTQTTSSLSASLGDRVTISCSASQGISNYLNWYQQKPDGTVKLLIYYT
SSLHSGVPSRFSGSGSGTDYSLTISNLEPEDIATYYCQQYSKLPYTFGGGT
KLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC
anti-CD2 TS2/18.1-Fab sequences:
TS2/18.1 HC (SEQ ID NO: 16):
EVQLVESGGGLVMPGGSLKLSCAASGFAFSSYDMSWVRQTPEKRLEWVAYI
SGGGFTYYPDTVKGRFTLSRDNAKNTLYLQMSSLKSEDTAMYYCARQGANW
ELVYWGQGTLVTVSAASTKGPSVFPLAPSSKSTSGGTAALGCLVKDYFPEP
VTVSWNSGALTSGVHTFPAVLQSSGLYSLSSVVTVPSSSLGTQTYICNVNH
KPSNTKVDKKVEPKSSDKTHTC
TS2/18.1 LC (SEQ ID NO: 17):
DIVMTQSPATLSVTPGDRVFLSCRASQSISDFLHWYQQKSHESPRLLIKYA
SQSISGIPSRFSGSGSGSDFTLSINSVEPEDVGVYFCQNGHNFPPTFGGGT
KLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES
anti-CD2 9.6-Fab sequences:
9.6 HC (SEQ ID NO: 18):
QVQLQQPGAELVRPGSSVKLSCKASGYTFTRYWIHWVKQRPIQGLEWIGNI
DPSDSETHYNQKFKDKATLTVDKSSGTAYMQLSSLTSEDSAVYYCATEDLY
YAMEYWGQGTSVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSSDKTHTC
9.6 LC (SEQ ID NO: 19):
NIMMTQSPSSLAVSAGEKVTMTCKSSQSVLYSSNQKNYLAWYQQKPGQSPK
LLIYWASTRESGVPDRFTGSGSGTDFTLTISSVQPEDLAVYYCHQYLSSHT
FGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGES
anti-CD2 9-1-Fab sequences:
9-1 HC (SEQ ID NO: 20):
QVQLQQPGTELVRPGSSVKLSCKASGYTFTSYWVNWVKQRPDQGLEWIGRI
DPYDSETHYNQKFTDKAISTIDTSSNTAYMQLSTLTSDASAVYYCSRSPRD
SSTNLADWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICN
VNHKPSNTKVDKKVEPKSSDKTHTC
9-1 LC (SEQ ID NO: 21):
DIVMTQSPATLSVTPGDRVSLSCRASQSISDYLHWYQQKSHESPRLLIKYA
SQSISGIPSRFSGSGSGSDFTLSINSVEPEDVGVYYCQNGHSFPLTFGAGT
KLELRRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES
mutOKT8-Fab sequences:
mutOKT8 HC (SEQ ID NO: 22):
QVQLVQSGAEDKKPGASVKVSCKASGFNIKDTYIHWVRQAPGQGLEWMGRI
DPANDNTLYASKFQGRVTITADTSSNTAYMELSSLRSEDTAVYYCGRGAGA
YVFDHWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSSDKTHTC
mutOKT8 LC (SEQ ID NO: 23):
DIVMTQSPSSLSASVGDRVTITCRTSRSISAALAWYQEKPGKAPKLLIYSG
STLQSGVPSRFSGSGSGTDFTLTISSLQPEDFATYYCQQHNENPLTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES.

In certain embodiments, the targeting group or immune cell targeting group (e.g., T cell-targeting agent, B cell-targeting agent, or NK-cell targeting agent) may be covalently coupled to a lipid via a polyethylene glycol (PEG) containing linker.

In other embodiments, the lipid used to create a conjugate may be selected from

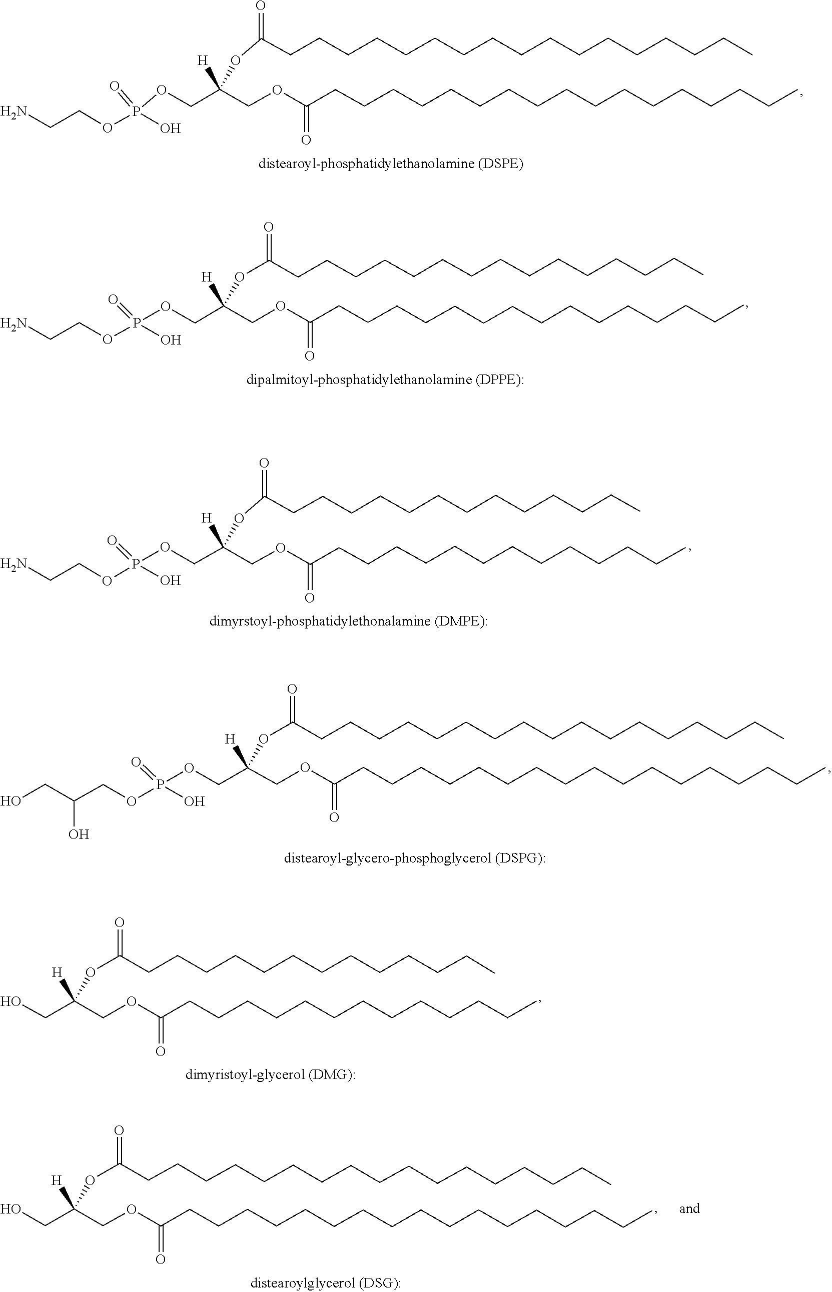 [SMILES: CCCCCCCCCCCCCCCCCC(=O)OC[C@H](COP(=O)(O)OCCN)OC(=O)CCCCCCCCCCCCCCCCC ; CCCCCCCCCCCCCCCC(=O)OC[C@H](COP(=O)(O)OCCN)OC(=O)CCCCCCCCCCCCCCC ; CCCCCCCCCCCCCC(=O)OC[C@H](COP(=O)(O)OCCN)OC(=O)CCCCCCCCCCCCC ; CCCCCCCCCCCCCCCCCC(=O)OC[C@H](COP(=O)(O)OCC(O)CO)OC(=O)CCCCCCCCCCCCCCCCC ; CCCCCCCCCCCCCC(=O)OC[C@H](CO)OC(=O)CCCCCCCCCCCCC ; CCCCCCCCCCCCCCCCCC(=O)OC[C@H](CO)OC(=O)CCCCCCCCCCCCCCCCC]

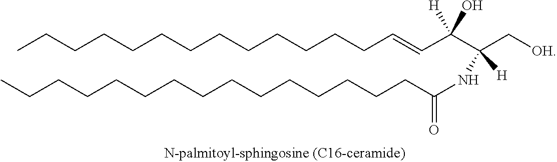 [SMILES: CCCCCCCCCCCCC/C=C/[C@@H](O)[C@H](CO)NC(=O)CCCCCCCCCCCCCCC]

The immune cell targeting group can be covalently coupled to a lipid either directly or via a linker, for example, a polyethylene glycol (PEG) containing linker. In certain embodiments, the PEG is PEG 1000, PEG 2000, PEG 3400, PEG 3000, PEG 3450, PEG 4000, or PEG 5000. In certain, embodiments, the PEG is PEG 2000.

In some embodiments, the lipid-immune cell targeting group conjugate is present in the lipid blend in a range of 0.001-0.5 mole percent, 0.001-0.3 mole percent, 0.002-0.2 mole percent, 0.01-0.1 mole percent, 0.1-0.3 mole percent, or 0.1-0.2 mole percent.

In certain embodiments, the lipid immune-cell targeting agent conjugate comprises DSPE, a PEG component and a targeting antibody. In certain embodiments, the antibody is a T-cell targeting agent, for example, an anti-CD2 antibody, an anti-CD3 antibody, an anti-CD4 antibody, an anti-CD5 antibody, an anti-CD7 antibody, an anti CD8 antibody, or an anti-TCR (3 antibody.

An exemplary lipid-immune cell targeting group conjugate comprises DSPE and PEG 2000, for example, as described in Nellis et al. (2005) BIOTECHNOL. PROG. 21, 205-220. An exemplary conjugate comprises the structure of Formula V, where the scFv represents an engineered antibody binding site that binds to a target of interest. In certain embodiments, the engineered antibody binding site binds to any of the targets described hereinabove. In certain embodiments, the engineered antibody binding site can be, for example, an engineered anti-CD3 antibody or an engineered anti-CD8 antibody. In certain embodiments, the engineered antibody binding site can be, for example, an engineered anti-CD2 antibody or an engineered anti-CD7 antibody.

An example of a compound of Formula (V) is as shown below:

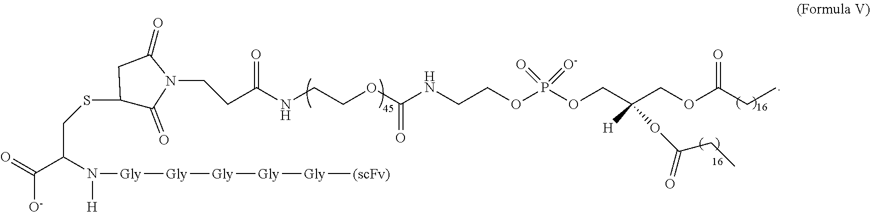

It is contemplated that the scFv in Formula V may be replaced with an intact antibody or an antigen fragment thereof (e.g., an Fab).

Another example of a compound of Formula (VI) is as shown below:

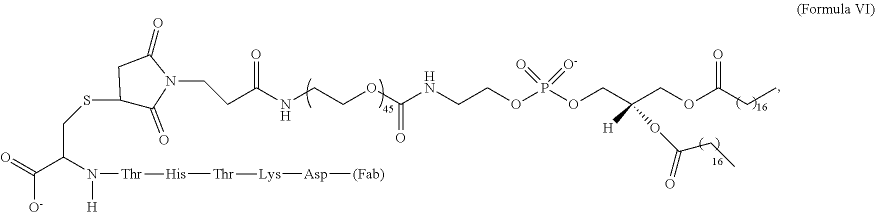

the production of which is described in Nellis et al. (2005) supra, or U.S. Pat. No. 7,022,336. It is contemplated that the Fab in Formula VI may be replaced with an intact antibody or an antigen fragment thereof (e.g., an (Fab′)₂ fragment) or an engineering antibody binding site (e.g., an scFv).

Other lipid immune cell target group conjugates are described, for example, in U.S. Pat. No. 7,022,336, where the targeting group may be replaced with a targeting group of interest, for example, a targeting group that binds an T-cell or NK cell surface antigen as described hereinabove.

In certain embodiments, the lipid component of an exemplary conjugate of Formula IV can be based on an ionizable, cationic lipid described herein, for example, an ionizable, cationic lipid of Formula I, Formula II, or Formula III. For example, an exemplary ionizable, cationic lipid can be selected from the group consisting of:

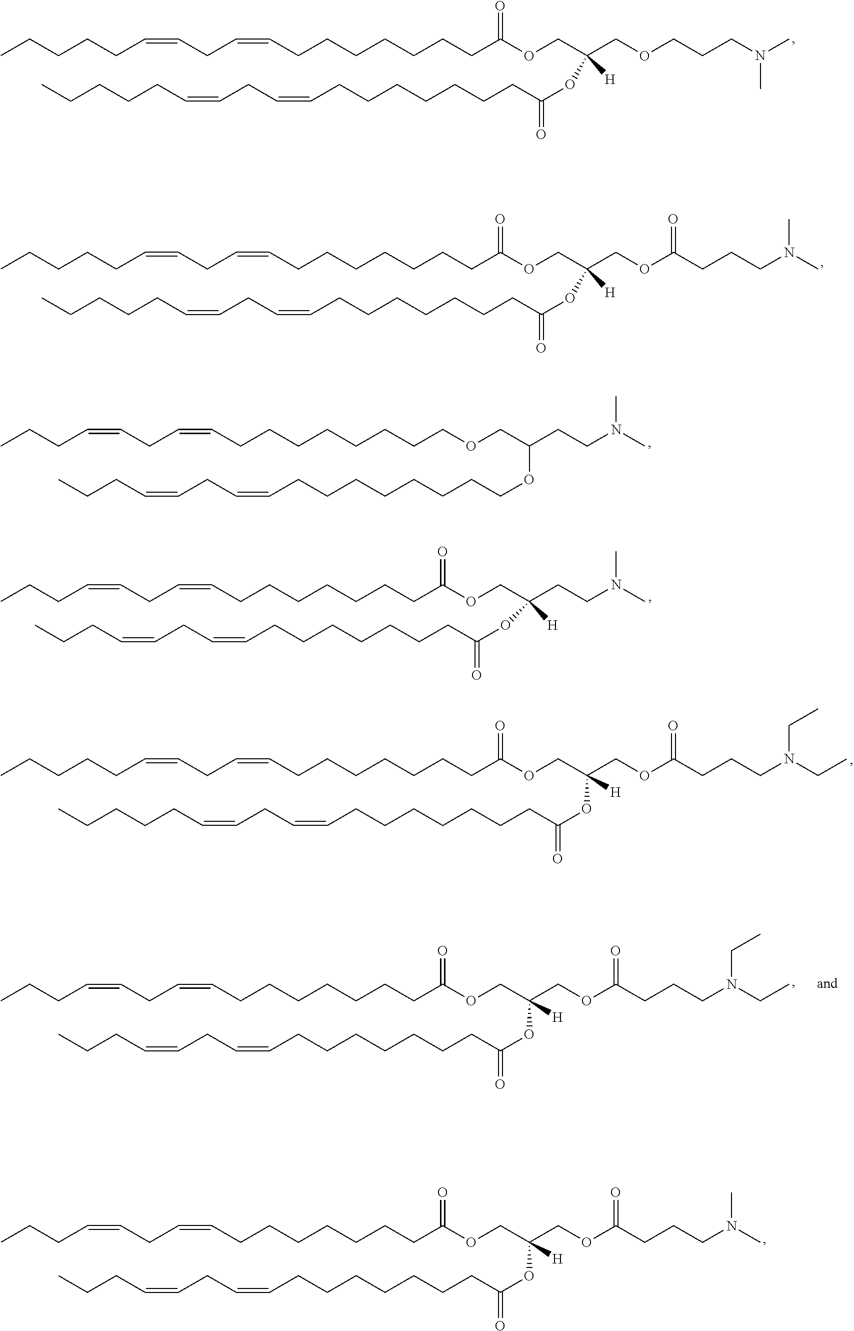

or a salt thereof.

In certain embodiments, an exemplary ionizable, cationic lipid can be a compound of the formula:

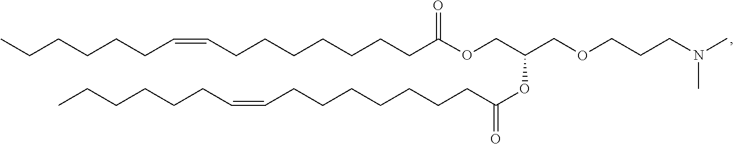 [SMILES: CCCCCC/C=C\CCCCCCCC(=O)OC[C@H](COCCCN(C)C)OC(=O)CCCCCCC/C=C\CCCCCC]

or a salt thereof.

In some embodiments, an exemplary ionizable, cationic lipid can be a compound of the formula:

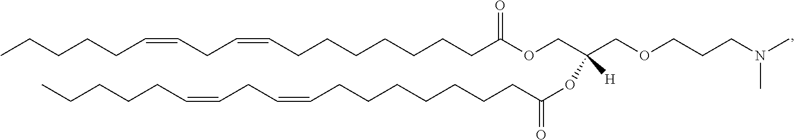 [SMILES: CCCCC/C=C\C/C=C\CCCCCCCC(=O)OC[C@H](COCCCN(C)C)OC(=O)CCCCCCC/C=C\C/C=C\CCCCC]

or a salt thereof.

In other embodiments, an exemplary ionizable, cationic lipid can be a compound of the formula:

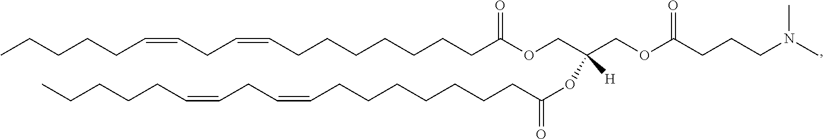 [SMILES: CCCCC/C=C\C/C=C\CCCCCCCC(=O)OC[C@H](COC(=O)CCCN(C)C)OC(=O)CCCCCCC/C=C\C/C=C\CCCCC]

or a salt thereof.

In certain embodiments, an exemplary ionizable, cationic lipid can be a compound of the formula:

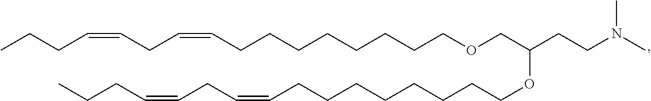 [SMILES: CCC/C=C\C/C=C\CCCCCCCCOCC(CCN(C)C)OCCCCCCCC/C=C\C/C=C\CCC]

or a salt thereof.

In some embodiments, an exemplary ionizable, cationic lipid can be a compound of the formula:

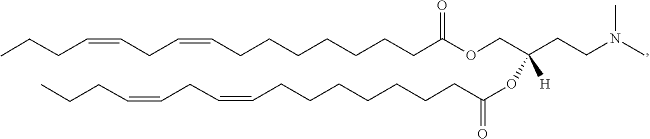 [SMILES: CCC/C=C\C/C=C\CCCCCCCC(=O)OC[C@H](CCN(C)C)OC(=O)CCCCCCC/C=C\C/C=C\CCC]

or a salt thereof.

In other embodiments, an exemplary ionizable, cationic lipid can be a compound of the formula:

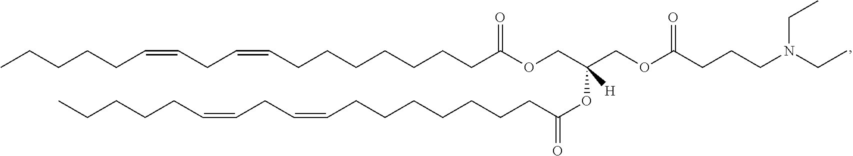 [SMILES: CCCCC/C=C\C/C=C\CCCCCCCC(=O)OC[C@H](COC(=O)CCCN(CC)CC)OC(=O)CCCCCCC/C=C\C/C=C\CCCCC]

or a salt thereof.

In certain embodiments, an exemplary ionizable, cationic lipid can be a compound of the formula:

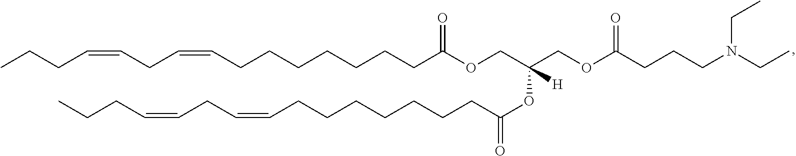 [SMILES: CCC/C=C\C/C=C\CCCCCCCC(=O)OC[C@H](COC(=O)CCCN(CC)CC)OC(=O)CCCCCCC/C=C\C/C=C\CCC]

or a salt thereof.

In some embodiments, an exemplary ionizable, cationic lipid can be a compound of the formula:

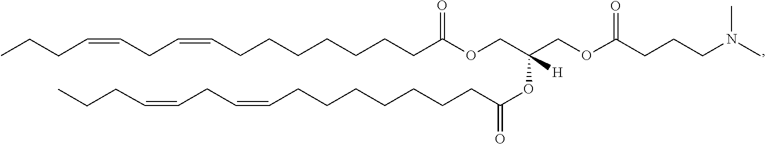 [SMILES: CCC/C=C\C/C=C\CCCCCCCC(=O)OC[C@H](COC(=O)CCCN(C)C)OC(=O)CCCCCCC/C=C\C/C=C\CCC]

or a salt thereof.

In certain embodiments, the conjugate based on a lipid of Formula III may include:

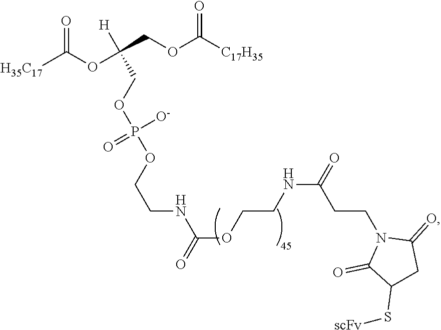

where scFv represents an engineered antibody binding site that binds a target described hereinabove, e.g., CD2, CD3, CD7, or CD8.

In certain embodiments, the lipid blend may further comprise free PEG-lipid so as to reduce the amount of non-specific binding via the targeting group. The free PEG-lipid can be the same or different from the PEG-lipid included in the conjugate. In certain embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl[methoxy(polyethylene glycol)] (PEG-ceramide), DSPE-PEG-cysteine, or a derivative thereof, all with average PEG lengths between 2000-5000, with 2000, 3400, or 5000. A final composition may contain a mixture of two or more of these pegylated lipids. In certain embodiments, the LNP composition comprises a mixture of PEG-lipids with myristoyl and stearic acyl chains.

In certain embodiments, the derivative of the PEG-lipid has a hydroxyl or a carboxylic acid end group at the PEG terminus.

The lipid-immune cell targeting group conjugate can be incorporated into LNPs as described below, for example, in LNPs containing, for example, an ionizable cationic lipid, a sterol, a neutral phospholipid and a PEG-lipid. It is contemplated that, in certain embodiments, the LNPs containing the lipid-immune cell targeting group can contain an ionizable cationic lipid described herein or a cationic lipid described, for example, in U.S. Pat. Nos. 10,221,127, 10,653,780 or U.S. Published application No. US2018/0085474, US2016/0317676, International Publication No. WO2009/086558, or Miao et al. (2019) NATURE BIOTECH 37:1174-1185, or Jayaraman et al. (2012) ANGEW CHEM INT. 51: 8529-8533. In other embodiments, the cationic lipid can be selected from an ionizable cationic lipid set forth in the Table 1.

TABLE 1

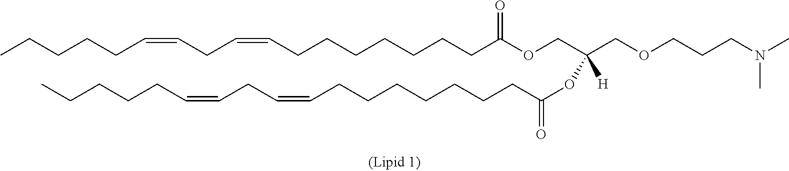 [SMILES: CCCCC/C=C\C/C=C\CCCCCCCC(=O)OC[C@H](COCCCN(C)C)OC(=O)CCCCCCC/C=C\C/C=C\CCCCC]

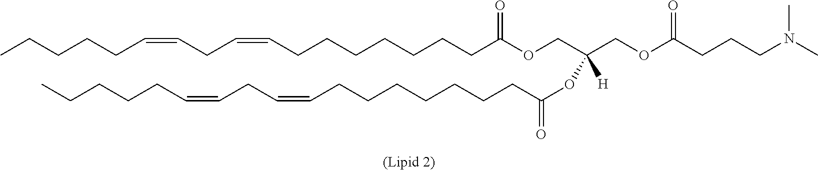 [SMILES: CCCCC/C=C\C/C=C\CCCCCCCC(=O)OC[C@H](COC(=O)CCCN(C)C)OC(=O)CCCCCCC/C=C\C/C=C\CCCCC]

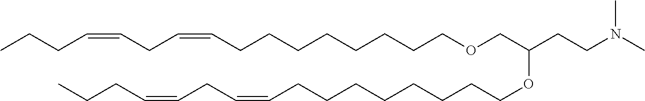 [SMILES: CCC/C=C\C/C=C\CCCCCCCCOCC(CCN(C)C)OCCCCCCCC/C=C\C/C=C\CCC]

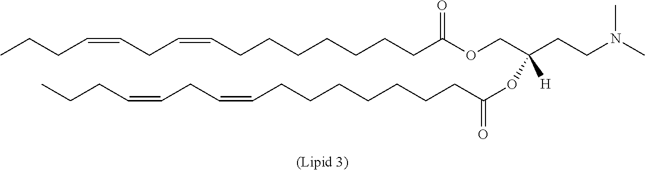 [SMILES: CCC/C=C\C/C=C\CCCCCCCC(=O)OC[C@H](CCN(C)C)OC(=O)CCCCCCC/C=C\C/C=C\CCC]

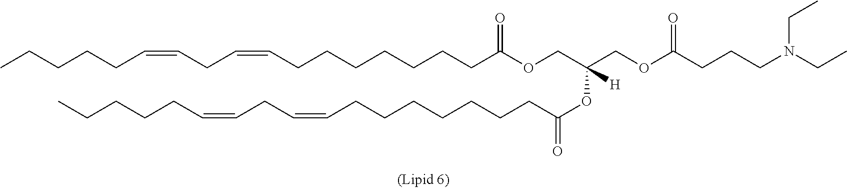 [SMILES: CCCCC/C=C\C/C=C\CCCCCCCC(=O)OC[C@H](COC(=O)CCCN(CC)CC)OC(=O)CCCCCCC/C=C\C/C=C\CCCCC]

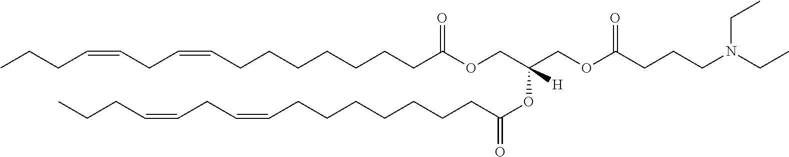 [SMILES: CCC/C=C\C/C=C\CCCCCCCC(=O)OC[C@H](COC(=O)CCCN(CC)CC)OC(=O)CCCCCCC/C=C\C/C=C\CCC]

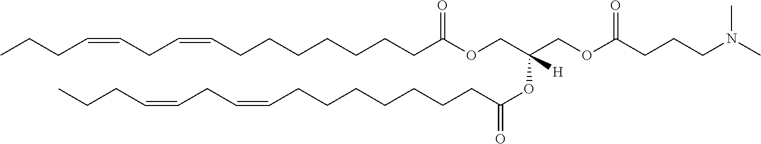 [SMILES: CCC/C=C\C/C=C\CCCCCCCC(=O)OC[C@H](COC(=O)CCCN(C)C)OC(=O)CCCCCCC/C=C\C/C=C\CCC]

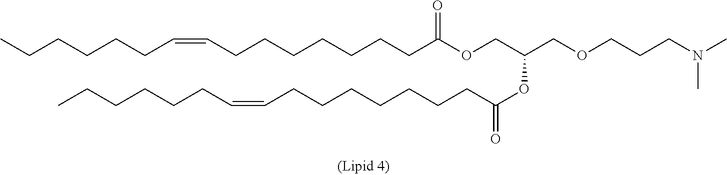 [SMILES: CCCCCC/C=C\CCCCCCCC(=O)OC[C@H](COCCCN(C)C)OC(=O)CCCCCCC/C=C\CCCCCC]

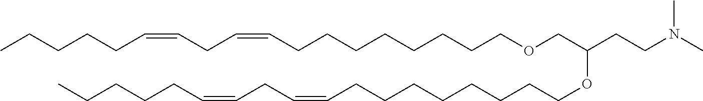 [SMILES: CCCCC/C=C\C/C=C\CCCCCCCCOCC(CCN(C)C)OCCCCCCCC/C=C\C/C=C\CCCCC]

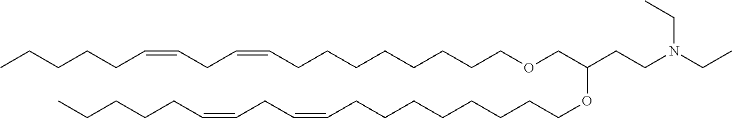 [SMILES: CCCCC/C=C\C/C=C\CCCCCCCCOCC(CCN(CC)CC)OCCCCCCCC/C=C\C/C=C\CCCCC]

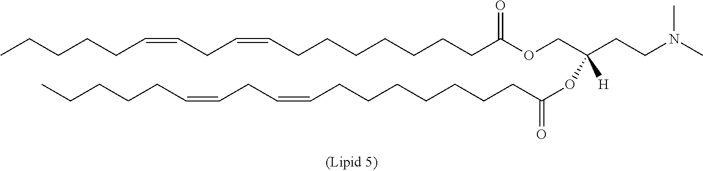 [SMILES: CCCCC/C=C\C/C=C\CCCCCCCC(=O)OC[C@H](CCN(C)C)OC(=O)CCCCCCC/C=C\C/C=C\CCCCC]

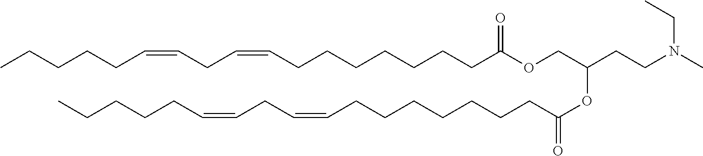 [SMILES: CCCCC/C=C\C/C=C\CCCCCCCC(=O)OCC(CCN(C)CC)OC(=O)CCCCCCC/C=C\C/C=C\CCCCC]

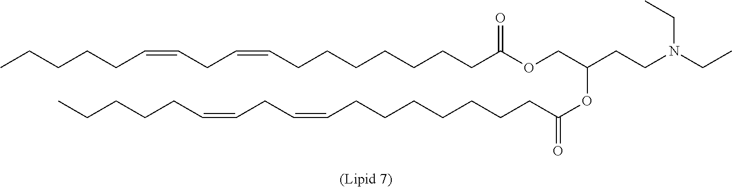 [SMILES: CCCCC/C=C\C/C=C\CCCCCCCC(=O)OCC(CCN(CC)CC)OC(=O)CCCCCCC/C=C\C/C=C\CCCCC]

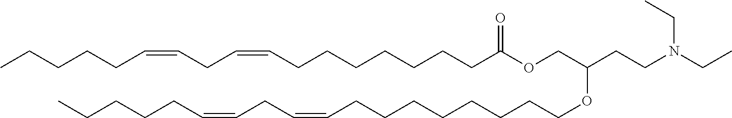 [SMILES: CCCCC/C=C\C/C=C\CCCCCCCCOC(CCN(CC)CC)COC(=O)CCCCCCC/C=C\C/C=C\CCCCC]

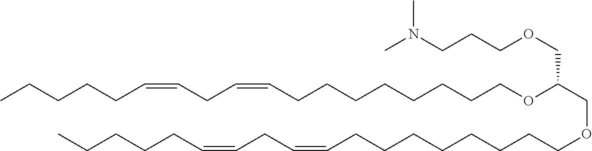 [SMILES: CCCCC/C=C\C/C=C\CCCCCCCCOC[C@@H](COCCCN(C)C)OCCCCCCCC/C=C\C/C=C\CCCCC]

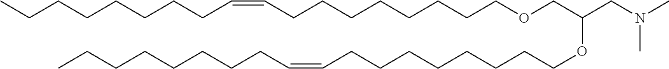 [SMILES: CCCCCCCC/C=C\CCCCCCCCOCC(CN(C)C)OCCCCCCCC/C=C\CCCCCCCC]

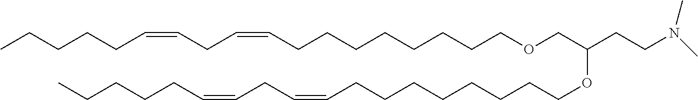 [SMILES: CCCCC/C=C\C/C=C\CCCCCCCCOCC(CCN(C)C)OCCCCCCCC/C=C\C/C=C\CCCCC]

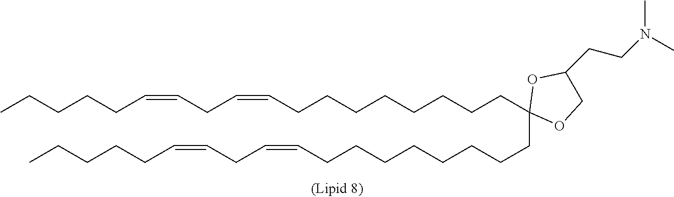 [SMILES: CCCCC/C=C\C/C=C\CCCCCCCCC1(CCCCCCCC/C=C\C/C=C\CCCCC)OCC(CCN(C)C)O1]

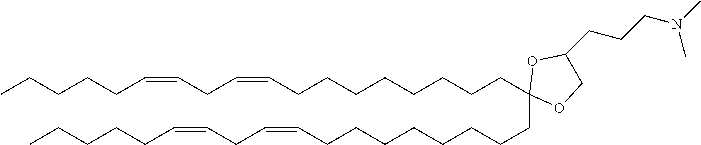 [SMILES: CCCCC/C=C\C/C=C\CCCCCCCCC1(CCCCCCCC/C=C\C/C=C\CCCCC)OCC(CCCN(C)C)O1]

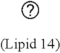

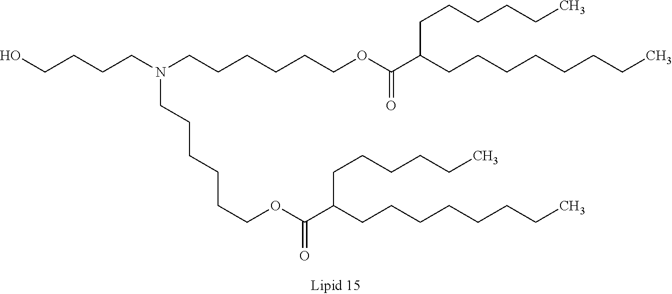 [SMILES: CCCCCCCCC(CCCCCC)C(=O)OCCCCCCN(CCCCO)CCCCCCOC(=O)C(CCCCCC)CCCCCCCC]

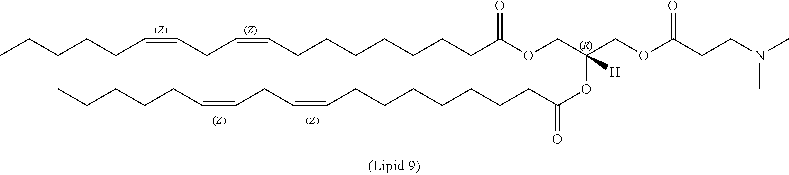

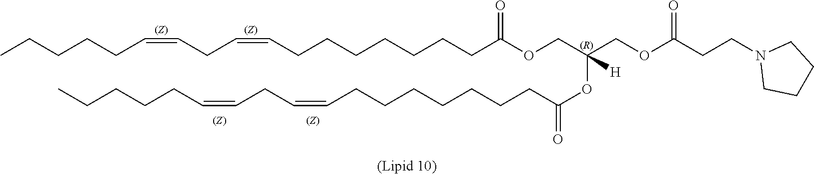

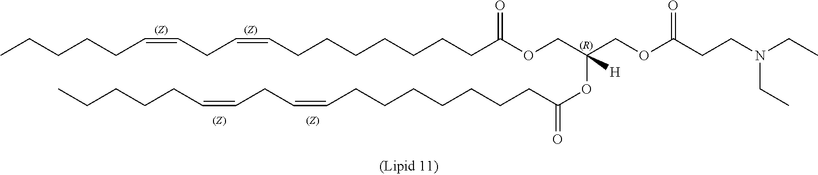

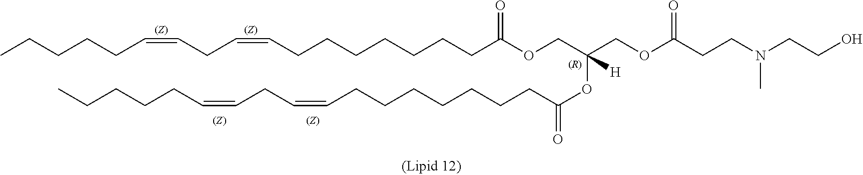

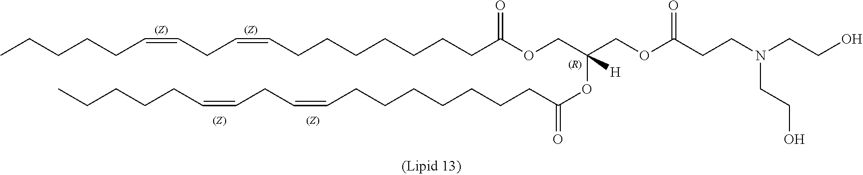

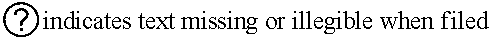

The LNPs can be formulated using the methods and other components described below in the following sections.

### IV. Lipid Nanoparticle Compositions

The invention provides a lipid nanoparticle (LNP) composition comprising a lipid blend that contains an ionizable cationic lipid described herein and/or a lipid-immune cell targeting agent conjugate described herein. In certain embodiments, the lipid blend may comprise an ionizable, cationic lipid described herein and one or more of a sterol, a neutral phospholipid, a PEG-lipid, and a lipid-immune cell targeting group conjugate.

In certain embodiments, the ionizable, cationic lipid described herein may be present in the lipid blend in a range of 30-70 mole percent, 30-60 mole percent 30-50 mole percent, 40-70 mole percent, 40-60 mole percent, 40-50 mole percent, 50-70 mole percent, 50-60 mole percent, or of about 30 mole percent, about 35 mole percent, about 40 mole percent, about 45 mole percent, about 50 mole percent, about 55 mole percent, about 60 mole percent, about 65 mole percent, or about 70 mole percent.

### Sterol

In certain embodiments, the lipid blend of the lipid nanoparticle may comprise a sterol component, for example, one or more sterols selected from the group consisting of cholesterol, fecosterol, β-sitosterol, ergosterol, campesterol, stigmasterol, stigmastanol, brassicasterol. In certain embodiments, the sterol is cholesterol.

The sterol (e.g., cholesterol) may be present in the lipid blend in a range of 20-70 mole percent, 20-60 mole percent, 20-50 mole percent, 30-70 mole percent, 30-60 mole percent, 30-50 mole percent, 40-70 mole percent, 40-60 mole percent, 40-50 mole percent, 50-70 mole percent, 50-60 mole percent, or about 20 mole percent, about 25 mole percent, about 30 mole percent, about 35 mole percent, about 40 mole percent, about 45 mole percent, about 50 mole percent, about 55 mole percent, about 60 mole percent or about 65 mole percent.

### Neutral Phospholipid

In certain embodiments, the lipid blend of the lipid nanoparticle may contain one or more neutral phospholipids. The neutral phospholipid can be selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), sphingomyelin (SM).

Other neutral phospholipids can be selected from the group consisting of distearoyl-phosphatidylethanolamine (DSPE), dimyrstoyl-phosphatidylethanolamine (DMPE), distearoyl-glycero-phosphocholine (DSPC), dioleoyl-glycero-phosphoethanolamine (DOPE), dilinoleoyl-glycero-phosphocholine (DLPC), dimyristoyl-glycero-phosphocholine (DMPC), dioleoyl-glycero-phosphocholine (DOPC), dipalmitoyl-glycero-phosphocholine (DPPC), diundecanoyl-glycero-phosphocholine (DUPC), palmitoyl-oleoyl-glycero-phosphocholine (POPC), dioctadecenyl-glycero-phosphocholine, oleoyl-cholesterylhemisuccinoyl-glycero-phosphocholine, hexadecyl-glycero-phosphocholine, dilinolenoyl-glycero-phosphocholine, diarachidonoyl-glycero-3-phosphocholine, didocosahexaenoyl-glycero-phosphocholine, or sphingomyelin.

The neutral phospholipid may be present in the lipid blend in a range of 1-10 mole percent, 1-15 mole percent, 1-12 mole percent, 1-10 mole percent, 3-15 mole percent, 3-12 mole percent, 3-10 mole percent, 4-15 mole percent, 4-12 mole percent, 4-10 mole percent, 4-8 mole percent, 5-15 mole percent, 5-12 mole percent, 5-10 mole percent, 6-15 mole percent, 6-12 mole percent, 6-10 more percent, or about 1 mole percent, about 2 mole percent, about 3 mole percent, about 4 mole percent, about 5 mole percent, about 6 mole percent, about 7 mole percent, about 8 mole percent, about 9 mole percent, about 10 mole percent, about 11 mole percent, about 12 mole percent, about 13 mole percent, about 14 mole percent, or about 15 mole percent.

### Peg-Lipid

The lipid blend of the lipid nanoparticle may include one or more PEG or PEG-modified lipids. Such species may be alternately referred to as PEGylated lipids. A PEG lipid is a lipid modified with polyethylene glycol. As noted above, free PEG-lipids can be included in the lipid blend to reduce or eliminate non-specific binding via a targeting group when a lipid-immune cell targeting group is included in the lipid blend.

A PEG lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

In certain embodiments, the blend may contain a free PEG-lipid that can be selected from the group consisting of PEG-distearoylglycerol (PEG-DSG), PEG-diacylglycerol (PEG-DAG, e.g., PEG-DMG, PEG-DPG, and PEG-DSG), PEG-dimyristoyl-glycerol (PEG-DMG), PEG-distearoyl-phosphatidylethanolamine (PEG-DSPE) and PEG-dimyrstoyl-phosphatidylethanolamine (PEG-DMPE). In some embodiments, the free PEG-lipid comprises a diacylphosphatidylcholines comprising Dipalmitoyl (C16) chain or Distearoyl (C18) chain.

The PEG-lipid may be present in the lipid blend in a range of 1-10 mole percent, 1-8 mole percent, 1-7 mole percent, 1-6 mole percent, 1-5 mole percent, 1-4 mole percent, 1-3 mole percent, 2-8 mole percent, 2-7 mole percent, 2-6 mole percent, 2-5 mole percent, 2-4 mole percent, 2-3 mole percent, or about 1 mole percent, about 2 mole percent, about 3 mole percent, about 4 mole percent, or about 5 mole percent. In some embodiments, the PEG-lipid is a free PEG-lipid.

In some embodiments, the PEG-lipid may be present in the lipid blend in the range of 0.01-10 mole percent, 0.01-5 mole percent, 0.01-4 mole percent, 0.01-3 mole percent, 0.01-2 mole percent, 0.01-1 mole percent, 0.1-10 mole percent, 0.1-5 mole percent, 0.1-4 mole percent, 0.1-3 mole percent, 0.1-2 mole percent, 0.1-1 mole percent, 0.5-10 mole percent, 0.5-5 mole percent, 0.5-4 mole percent, 0.5-3 mole percent, 0.5-2 mole percent, 0.5-1 mole percent, 1-2 mole percent, 3-4 mole percent, 4-5 mole percent, 5-6 mole percent, or 1.25-1.75 mole percent. In some embodiments, the PET-lipid may be about 0.5 mole percent, about 1 mole percent, about 1.5 mole percent, about 2 mole percent, about 2.5 mole percent, about 3 mole percent, about 3.5 mole percent, about 4 mole percent, about 4.5 mole percent, about 5 mole percent, or about 5.5 mole percent of the lipid blend. In some embodiments, the PEG-lipid is a free PEG-lipid.

In some embodiments, the lipid anchor length of PEG-lipid is C14 (as in PEG-DMG). In some embodiments, the lipid anchor length of PEG-lipid is C16 (as in DPG). In some embodiments, the lipid anchor length of PEG-lipid is C18 (as in PEG-DSG). In some embodiments, the back bone or head group of PEG-lipid is diacyl glycerol or phosphoethanolamine. In some embodiments, the PEG-lipid is a free PEG-lipid.

A LNP of the present disclosure may comprise one or more free PEG-lipid that is not conjugated to an immune cell targeting group, and a PEG-lipid that is conjugated to immune cell targeting group. In some embodiments, the free PEG-lipid comprises the same or a different lipid as the lipid in the lipid-immune cell targeting group conjugate.

### Immune Cell Targeting Group Conjugate

In certain embodiments, the lipid blend can also include a lipid-immune cell targeting group conjugate as described in Section III above.

The lipid-immune cell targeting group conjugate may be present in the lipid blend in a range of 0.001-0.5 mol percent, 0.001-0.1 mole percent, 0.01-0.5 mole percent, 0.05-0.5 mole percent, 0.1-0.5 mole percent, 0.1-0.3 mole percent, 0.1-0.2 mole percent, 0.2-0.3 mole percent, of about 0.01 mole percent, about 0.05 mole percent, about 0.1 mole percent, about 0.15 mole percent, about 0.2 mole percent, about 0.25 mole percent, about 0.3 mole percent, about 0.35 mole percent, about 0.4 mole percent, about 0.45 mole percent, or about 0.5 mole percent.

In addition to the lipids present in the lipid blend, the LNP compositions may further comprise a payload, for example, a payload described hereinbelow. In certain embodiments, the payload is a nucleic acid, for example, DNA or RNA, for example, an mRNA, transfer RNA (tRNA), a microRNA, or small interfering RNA (siRNA).

In certain embodiments, the number of the nucleotides in the nucleic acid is from about 400 to about 6000.

### Production of Lipid Nanoparticles

In general the LNPs are produced by using either rapid mixing by an orbital vortexer or by microfluidic mixing. Orbital vortexer mixing is accomplished by rapid addition of lipids solution in ethanol to the aqueous solution of a nucleic acid of interest followed immediately by vortexing at 2,500 rpm. Microfluidic mixing is achieved mixing the aqueous and organic streams at a controlled flow rates in a microfluidic channel using, e.g., a NanoAssemblr device and microfluidic chips featuring optimized mixing chamber geometry (Precision Nanosystems, Vancouver, BC).

In certain embodiments, the resulting LNP compositions comprise a lipid blend containing, for example, from about 40 mole percent to about 60 mole percent of one or more ionizable cationic lipids described herein, from about 35 mole percent to about 50 mole percent of one or more sterols, from about 5 mole percent to about 15 mole percent of one or more neutral lipids, and from about 0.5 mole percent to about 5 mole percent of one or more PEG-lipids.

### Physical Properties of Lipid Nanoparticles

The characteristics of an LNP composition may depend on the components, their absolute or relative amounts, contained in a lipid nanoparticle (LNP) composition. Characteristics may also vary depending on the method and conditions of preparation of the LNP composition.

LNP compositions may be characterized by a variety of methods. For example, microscopy (e.g., transmission electron microscopy or scanning electron microscopy) may be used to examine the morphology and size distribution of an LNP composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of an LNP composition, such as particle size, polydispersity index, and zeta potential. RNA encapsulated efficiency is determined by a combination of methods relying on RNA binding dyes (ribogreen, cybergreen to determine dye accessible RNA fraction) and LNP de-formulation followed by HPLC analysis for total RNA content.

In some embodiments, the LNP may have a mean diameter in the range of 1-250 nm, 1-200 nm, 1-150 nm, 1-100 nm, 50-250 nm, 50-200 nm, 50-150 nm, 50-100 nm, 75-250 nm, 75-200 nm, 75-150 nm, 75-100 nm, 100-250 nm, 100-200 nm, 100-150 nm. In certain embodiments, the LNP compositions may have a mean diameter of about 1 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the LNP has a mean diameter of about 100 nm.

Alternatively or in addition, the LNP compositions may have a polydispersity index in a range from 0.05-1, 0.05-0.75, 0.05-0.5, 0.05-0.4, 0.05-0.3, 0.05-0.2, 0.08-1, 0.08-0.75, 0.08-0.5, 0.08-0.4, 0.08-0.3, 0.08-0.2, 0.1-1, 0.1-0.75, 0.1-0.5, 0.1-0.4, 0.1-0.3, 0.1-0.2. In certain embodiments, the polydispersity index is in the range of 0.1-0.25, 0.1-0.2, 0.1-0.19, 0.1-0.18, 0.1-0.17, 0.1-0.16, or 0.1-0.15.

Alternatively or in addition, the LNP compositions may have a zeta potential of about −30 mV to about +30 mV. In certain embodiments, the LNP composition has a zeta potential of about −10 mV to about +20 mV. The zeta potential may vary as a function of ph. As a result, in certain embodiments, the LNP compositions may have a zeta potential of about −10 mV to about +30 mV or about 0 mV to +30 mV or about +5 mV to about +30 mV at pH 5.5 or pH 5, and/or a zeta potential of about −30 mV to about +5 mV or about −20 mV to about +15 mV at pH 7.4.

### V. Payloads

The LNP compositions may comprise an agent, for example, a nucleic acid molecule for delivery to a cell (e.g., an immune cell) or tissue, for example, a cell (e.g., an immune cell) or tissue in a subject.

The LNP compositions of the present invention may include a nucleic acid, for example, a DNA or RNA, such as an mRNA, tRNA, microRNA, siRNA, or dicer substrate siRNA. It is contemplated that nucleic acids can contain naturally occurring components, such as, naturally occurring bases, sugars or linkage groups (e.g., phosphodiester linkage groups) or may contain non-naturally occurring components or modifications, (e.g., thioester linkage groups). For example, the nucleic acid can be synthesized to contain base, sugar, linker modifications known to those skilled in the art. Furthermore, the nucleic acids can be linear or circular, or have any desired configuration. The LNP compositions can include multiple nucleic acid molecules, for example, multiple RNA molecules, which can be the same or different.

In certain embodiments, the payload is an mRNA. In certain embodiments, a particular LNP composition may contain a number mRNA molecules that can be the same or different. In certain embodiments, one or more LNP compositions including one or more different mRNAs may be combined, and/or simultaneously contacted, with a cell. It is contemplated that an mRNA may include one or more of a stem loop, a chain terminating nucleoside, a polyA sequence, a polyadenylation signal, and/or a 5′ cap structure. The mRNA may encode a receptor, such as a chimeric antigen receptor (CAR), for use in for example, an immune disorder, inflammatory disorder or cancer. In addition, the mRNA may encode an antigen for use in a therapeutic or prophylactic vaccine, for example, for treating or preventing an infection by a pathogen, for example, a microbial or viral pathogen, or for reducing or ameliorating the side effects caused directly or indirectly by such an infection.

In certain embodiments, the LNP composition may include one or more other components including, but not limited to, one or more pharmaceutically acceptable excipients, small hydrophobic molecules, therapeutic agents, carbohydrates, polymers, permeability enhancing molecules, and surface altering agents.

In some embodiments, the wt/wt ratio of the lipid component to the payload (e.g., mRNA) in the resulting LNP composition is from about 1:1 to about 50:1. In certain embodiments, the wt/wt ratio of the lipid component to the payload (e.g., mRNA) in the resulting composition is from about 5:1 to about 50:1. In certain embodiments, the wt/wt ratio is from about 5:1 to about 40:1. In certain embodiments, the wt/wt ratio is from about 10:1 to about 40:1. In certain embodiments, the wt/wt ratio is from about 15:1 to about 25:1.

In certain embodiments, the encapsulation efficiency of the payload (e.g., mRNA) in the lipid nanoparticles is at least 50%. In certain embodiments, the encapsulation efficiency is at least 80%, at least 90% or, or greater than 90%.

### RNA Payload

In certain embodiments, the RNA payload is an mRNA, tRNA, microRNA, or siRNA payload.

In certain embodiments, the lipid nanoparticle compositions are optimized for the delivery of RNA, e.g., mRNA, to a target cell for translation within the cell. An mRNA may be a naturally or non-naturally occurring mRNA. An mRNA may include one or more modified nucleobases, nucleosides, or nucleotides.

The nucleobases may be selected from the non-limiting group consisting of adenine, guanine, uracil, cytosine, 7-methylguanine, 5-methylcytosine, 5-hydroxymethylcytosine, thymine, pseudouracil, dihydrouracil, hypoxanthine, and xanthine.

A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications.

A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein.

An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine.

In certain embodiments, an mRNA may include a 5′ cap structure, a chain terminating nucleotide, a stem loop, a polyA sequence, and/or a polyadenylation signal.

A cap structure or cap species is a compound including two nucleoside moieties joined by a linker and may be selected from a naturally occurring cap, a non-naturally occurring cap or a cap analog. A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m7Gpppm7G, m73′dGpppG, and m27 02′GppppG.

Alternatively or in addition, an mRNA may include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

Alternatively or in addition, an mRNA may include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

Alternatively or in addition, an mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA.

An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell. In some embodiments, the mRNA may encode an antibody, enzyme, growth factor, hormone, cytokine, viral protein (e.g., a viral capsid protein), antigen, vaccine, or receptor. In some embodiments, the mRNA may encode an engineered receptor such as a CAR or an antigen for use in a therapeutic vaccine (e.g., a cancer vaccine) or a prophylactic vaccine (e.g., a vaccine for minimizing the risk or severity of an infection by a microbial or viral pathogen). In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

A lipid composition may be designed for one or more specific applications or targets. For example, an LNP composition may be designed to deliver mRNA to a particular cell, tissue, organ, or system or group thereof in a mammal's body, such as the renal system. Physiochemical properties of LNP compositions may be altered in order to increase selectivity for particular target site within a subject. For instance, particle sizes may be adjusted based on the fenestration sizes of different organs. The mRNA included in an LNP composition may also depend on the desired delivery target or targets. For example, an mRNA may be selected for a particular indication, condition, disease, or disorder and/or for delivery to a particular cell, tissue, organ, or system or group thereof (e.g., localized or specific delivery).

The amount of mRNA in a lipid composition may depend on the size, sequence, and other characteristics of the mRNA. The amount of mRNA in an LNP may also depend on the size, composition, desired target, and other characteristics of the LNP composition. The relative amounts of mRNA and other elements (e.g., lipids) may also vary. The amount of mRNA in an LNP composition may, for example, be measured using absorption spectroscopy (e.g., ultraviolet-visible spectroscopy).

In some embodiments, the one or more mRNAs, lipids, and polymers and amounts thereof may be selected to provide a specific N:P ratio (the ratio of positively-chargeable lipid or polymer amine (N=nitrogen) groups to negatively-charged nucleic acid phosphate (P) groups). The N:P ratio of the composition refers to the molar ratio of nitrogen atoms in one or more lipids to the number of phosphate groups in an mRNA. In general, a lower N:P ratio is preferred. A N:P ratio may be dependent on a specific lipid and its pKa. In certain embodiments, the mRNA and LNP composition, and/or their relative amounts may be selected to provide an N:P ratio from about 1:1 to about 30:1, or from about 1:1 to about 20:1. In certain embodiments, the N:P ratio can be, for example, 1:1, 2:1, 3:1, 4:1, 5:1, 6:1, 7:1, or 8:1. In certain embodiments, the N:P ratio may be from about 2:1 to about 5:1. In certain embodiments, the N:P ratio may be about 4:1. In other embodiments, the N:P ratio is from about 4:1 to about 8:1. For example, the N:P ratio may be about 4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 6.0:1, about 6.5:1, or about 7.0:1.

The amount of mRNA in a nanoparticle composition may depend on the size, sequence, and other characteristics of the mRNA. The amount of mRNA in a nanoparticle composition may also depend on the size, composition, desired target, and other characteristics of the nanoparticle composition. The relative amounts of mRNA and other elements (e.g., lipids) may also vary. In some embodiments, the wt/wt ratio of the lipid component to an mRNA in a nanoparticle composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of the lipid component to an mRNA may be from about 10:1 to about 40:1. The amount of mRNA in a nanoparticle composition may, for example, be measured using absorption spectroscopy (e.g., ultraviolet-visible spectroscopy).

The efficiency of encapsulation of an mRNA describes the amount of mRNA that is encapsulated or otherwise associated with a lipid composition after preparation, relative to the initial amount provided. The encapsulation efficiency is desirably high (e.g., close to 100%). The encapsulation efficiency may be measured, for example, by comparing the amount of mRNA in a solution containing the lipid composition before and after breaking up the LNP composition with one or more organic solvents or detergents. Fluorescence may be used to measure the amount of free mRNA in a solution. For the LNP compositions of the invention, the encapsulation efficiency of an mRNA may be at least 50%, for example 50%, 55%, 60%, 65%, 70%, 75%, 80%, 85%, 90%, 91%, 92%, 93%, 94%, 95%, 96%, 97%, 98%, 99%, or 100%. In certain embodiments, the encapsulation efficiency may be at least 80%.

### VI. Formulation and Mode of Delivery

LNP compositions of the invention may be formulated in whole or in part as a pharmaceutical composition. The pharmaceutical compositions may further include one or more pharmaceutically acceptable excipients or accessory ingredients such as those described herein. General guidelines for the formulation and manufacture of pharmaceutical compositions and agents are available, for example, in Remington's (2006) supra. Conventional excipients and accessory ingredients may be used in any pharmaceutical composition of the invention, except insofar as any conventional excipient or accessory ingredient may be incompatible with one or more components of an LNP composition of the invention. An excipient or accessory ingredient may be incompatible with a component of an LNP composition if its combination with the component may result in any undesirable biological effect or otherwise deleterious effect.

In some embodiments, one or more excipients or accessory ingredients may make up greater than 50% of the total mass or volume of a pharmaceutical composition including an LNP composition of the invention. For example, the one or more excipients or accessory ingredients may make up 30%, 40%, 50%, 60%, 70%, 80%, 90%, or more of a pharmaceutical composition. In certain embodiments, the excipient is approved for use in humans and for veterinary use, for example, by United States Food and Drug Administration. In certain embodiments, the excipient is pharmaceutical grade. In certain embodiments, an excipient meets the standards of the United States Pharmacopoeia (USP), the European Pharmacopoeia (EP), the British Pharmacopoeia, and/or the International Pharmacopoeia.

Relative amounts of the one or more lipids or LNPs, one or more pharmaceutically acceptable excipients, and/or any additional ingredients in a pharmaceutical composition will vary, depending upon the identity, size, and/or condition of the subject treated and further depending upon the route by which the composition is to be administered.

Lipid compositions and/or pharmaceutical compositions including one or more LNP compositions may be administered to any subject, including a human patient that may benefit from a therapeutic effect provided by the delivery of a nucleic acid, e.g., an RNA (e.g., mRNA, tRNA or siRNA) to one or more particular cells, tissues, organs, or systems or groups thereof, such as the renal system. Although the descriptions provided herein of LNP compositions and pharmaceutical compositions including LNP compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is understood.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., the payload).

Pharmaceutical compositions of the invention may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Other Components

In addition, it is contemplated that the pharmaceutical compositions may include one or more components in addition to those described hereinabove.

The pharmaceutical compositions may also include one or more permeability enhancer molecules, carbohydrates, polymers, therapeutic agents, surface altering agents, or other components. A permeability enhancer molecule may be a molecule described, for example, in U.S. patent application publication No. 2005/0222064. Carbohydrates may include simple sugars (e.g., glucose) and polysaccharides (e.g., glycogen and derivatives and analogs thereof).

The pharmaceutical compositions may also contain a surface altering agent, including for example, anionic proteins (e.g., bovine serum albumin), surfactants (e.g., cationic surfactants such as dimethyldioctadecyl-ammonium bromide), sugars or sugar derivatives (e.g., cyclodextrin), polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within and/or upon the surface of a composition described herein.

In addition to these components, a pharmaceutical composition containing an LNP composition of the invention may include any substance useful in pharmaceutical compositions. For example, the pharmaceutical composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see, e.g., Remington's (2006) supra).

Dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof.

In certain embodiments, the lipid nanoparticle compositions and formulations thereof are adapted for administration intravenously, intramuscularly, intradermally, subcutaneously, intra-arterially, intra-tumor, or by inhalation. In certain embodiments, a dose of about 0.001 mg/kg to about 10 mg/kg is administered to a subject. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

The specific therapeutically effective, prophylactically effective, or otherwise appropriate dose level (e.g., for imaging) for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

### VII. Methods

The present disclosure provides methods of delivering a payload to a target cell or tissue, for example, a target cell or tissue in a subject, and LNPs or pharmaceutical compositions containing the LNPs for use in such methods.

In certain embodiments, the invention provides a method of producing a polypeptide of interest (e.g., a protein of interest) in a mammalian cell, and LNPs or pharmaceutical compositions containing the LNPs for use in such methods. Methods of producing polypeptides in such a cell involve contacting a cell with an LNP composition comprising an RNA of interest (e.g., an mRNA encoding the polypeptide of interest (e.g., a protein of interest). Upon contacting the cell with the LNP composition, the mRNA may be taken up and translated in the cell to produce the polypeptide of interest.

In general, the step of contacting a mammalian cell with an LNP composition including an mRNA encoding a polypeptide of interest may be performed in vivo, ex vivo, or in vitro. The amount of an LNP composition contacted with a cell, and/or the amount of mRNA therein, may depend on the type of cell or tissue being contacted, the means of administration, the physiochemical characteristics of the LNP composition and the mRNA (e.g., size, charge, and chemical composition) therein, and other factors. In general, an effective amount of the LNP composition will allow for efficient polypeptide production in the cell. Metrics for efficiency may include polypeptide translation (indicated by polypeptide expression), level of mRNA degradation, and immune response indicators.

The step of contacting an LNP composition including an mRNA with a cell may involve or cause transfection where the LNP composition may fuse with the membrane of cell to permit the delivery of the mRNA into the cell. Upon introduction into the cytoplasm of the cell, the mRNA is then translated into a protein or peptide via the protein synthesis machinery within the cytoplasm of the cell.

In certain embodiments, the LNP compositions described herein may be used to deliver therapeutic or prophylactic agents to a subject. For example, an mRNA included in an LNP composition may encode a polypeptide and produce the therapeutic or prophylactic polypeptide upon contacting and/or entry (e.g., transfection) into a cell. In certain embodiments, an mRNA included in an LNP composition of the invention may encode a polypeptide that may improve or increase the immunity of a subject.

In certain embodiments, contacting a cell with an LNP composition including an mRNA may reduce the innate immune response of a cell to an exogenous nucleic acid. A cell may be contacted with a first LNP composition including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times.

Additionally, efficiency of polypeptide production in the cell may be optionally determined, and the cell may be re-contacted with the first and/or second composition repeatedly until a target protein production efficiency is achieved.

The present disclosure provides methods of delivering a nucleic acid (e.g., an mRNA) to a mammalian cell or tissue, for example, a mammalian cell or tissue in a subject. Delivery of an mRNA to such a cell or tissue involves administering an LNP composition including the mRNA to a subject, for example, by injection, e.g., via intramuscular injection or intravascular delivery into the subject. After administration, the LNP can target and/or contact a cell, for example, an immune cell, such as a T-cell. Upon contacting the cell with the LNP composition, a translatable mRNA may be translated in the cell to produce a polypeptide of interest.

In certain embodiments, an LNP composition of the invention may target a particular type or class of cells. This targeting may be facilitated using the lipids described herein to form LNPs, which may also include a targeting group for targeting cells of interest. In certain, embodiments, specific delivery may result in a greater than 2 fold, 5 fold, 10 fold, 15 fold, or 20 fold increase in the amount of mRNA to the targeted destination (e.g., cells that express or express at high levels the receptor of interest which binds to the immune cell targeting group of the LNPs) as compared to another destinations (e.g., cells that either do not express or only express at low levels the receptor of interest).

LNP compositions of the invention may be useful for treating a disease, disorder, or condition characterized by missing or aberrant protein or polypeptide activity. Upon delivery of an mRNA encoding the missing or aberrant polypeptide to a cell, translation of the mRNA may produce the polypeptide, thereby reducing or eliminating an issue caused by the absence of or aberrant activity caused by the polypeptide. Because translation may occur rapidly, the methods and compositions of the invention may be useful in the treatment of acute diseases, disorders, or conditions such as sepsis, stroke, and myocardial infarction. An mRNA included in an LNP composition of the invention may also be capable of altering the rate of transcription of a given species, thereby affecting gene expression.

Diseases, disorders, and/or conditions characterized by dysfunctional or aberrant protein or polypeptide activity for which a composition of the invention may be administered include, but are not limited to, cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis. The present disclosure provides a method for treating such diseases, disorders, and/or conditions in a subject by administering an LNP composition including an mRNA and a lipid component including KL10, a phospholipid (optionally unsaturated), a PEG lipid, and a structural lipid, wherein the m RNA encodes a polypeptide that antagonizes or otherwise overcomes an aberrant protein activity present in the cell of the subject.

The therapeutic and/or prophylactic compositions described herein may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject, the purpose of the administration, the particular composition, the mode of administration, and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

A LNP composition including one or more mRNAs may be administered by a variety of routes, for example, orally, intravenously, intramuscularly, intra-arterially, intramedullary, intrathecally, subcutaneously, intraventricularly, trans- or intra-dermally, intradermally, rectally, intravaginally, intraperitoneally, topically, mucosally, nasally, intratumorally. In certain embodiments, an LNP composition may be administered intravenously, intramuscularly, intradermally, intra-arterially, intratumorally, or subcutaneously. However, the present disclosure encompasses the delivery of LNP compositions of the invention by any appropriate route taking into consideration likely advances in the sciences of drug delivery. In general, the most appropriate route of administration will depend upon a variety of factors including the nature of the LNP composition including one or more mRNAs (e.g., its stability in various bodily environments such as the bloodstream and gastrointestinal tract), the condition of the patient (e.g., whether the patient is able to tolerate particular routes of administration), etc.

In certain embodiments, compositions in accordance with the present disclosure may be administered at dosage levels sufficient to deliver from about 0.0001 mg/kg to about 1 0 mg/kg, from about 0.001 mg/kg to about 1 0 mg/kg, from about 0.005 mg/kg to about 1 0 mg/kg, from about 0.01 mg/kg to about 1 0 mg/kg, from about 0.05 mg/kg to about 1 0 mg/kg, from about 0.1 mg/kg to about 1 0 mg/kg, from about 1 mg/kg to about 1 0 mg/kg, from about 2 mg/kg to about 1 0 mg/kg, from about 5 mg/kg to about 1 0 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a composition in a given dose, where a dose of 1 mg/kg provides 1 mg of a composition per 1 kg of subject body weight.

In particular embodiments, a dose of about 0.001 mg/kg to about 1 0 mg/kg of an LNP composition of the invention may be administrated. In other embodiments, a dose of about 0.005 mg/kg to about 2.5 mg/kg of an LNP composition may be administered. In certain embodiments, a dose of about 0.1 mg/kg to about 1 mg/kg may be administered. In other embodiments, a dose of about 0.05 mg/kg to about 0.25 mg/kg may be administered. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

LNP compositions including one or more mRNAs may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more LNP compositions including one or more different m RNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

In some embodiments, no more than 1%, no more than 2%, no more than 3%, no more than 4%, no more than 5%, no more than 6%, no more than 7%, no more than 8%, no more than 9%, no more than 10%, no more than 15%, no more than 20%, no more than 25%, no more than 30%, no more than 35%, no more than 40%, no more than 45%, or no more than 50% of cells that are not meant to be the destination of the delivery are transfected by the LNP. In some embodiments, the cells that are not meant to be the destination of the delivery are subject's non-immune cells. In some embodiments, the cells that are not meant to be the destination of the delivery are cells not targeted by the method. In some embodiments, the cells that are not meant to be the destination of the delivery are subject's cells not targeted by the method.

In some embodiments, the half-life of the nucleic acid delivered by the LNP described herein to the immune cell or a polypeptide encoded by the nucleic acid delivered by the LNP and expressed in the immune cell is at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, or at least 5 times longer than the half-life of the nucleic acid delivered by a reference LNP to the immune cells or a polypeptide encoded by the nucleic acid delivered by the reference LNP and expressed in the immune cell.

In some embodiments, the composition of the LNP differs from the composition of the reference LNP in the type of ionizable cationic lipid, relative amount of ionizable cationic lipid, length of the lipid anchor in PEG lipid, back bone or head group of the PEG lipid, relative amount of PEG lipid, or type of immune cell targeting group, or any combination thereof. In some embodiments, the composition of the LNP differs from the composition of the reference LNP only in the type of ionizable cationic lipid. In some embodiments, the composition of the LNP differs from the composition of the reference LNP only in the amount of PEG lipid. In some embodiments, the reference LNP comprises cationic Lipid DLin-MC3-DMA or Lipid 7, but otherwise as the same as a tested LNP. In some embodiments, PEG lipid is a free PEG lipid.

In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the immune cells are transfected by the LNP. In some embodiments, the immune cells are subject's immune cells. In some embodiments, the immune cells are immune cells targeted by the method. In some embodiments, the immune cells are subject's immune cells targeted by the method.

In some embodiments, the expression level of the nucleic acid delivered by the LNP is at least at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, at least 5 times, at least 6 times, at least 7 times, at least 8 times, at least 9 times, or at least 10 times higher than the expression level of the nucleic acid delivered by a reference LNP. In some embodiments, the expression level is measured and compared with a method described herein. In some embodiments, the expression level is measured by the ratio of cells expressing the encoded polypeptide. In some embodiments, the expression level is measured with FACS. In some embodiments, the expression level is measured by the average amount of the encoded polypeptide expressed in cells. In some embodiments, the expression level is measured as mean fluorescence intensity. In some embodiments, the expression level is measured by the amount of the encoded polypeptide or other materials secreted by cells.

In another aspect, provided herein are methods of targeting the delivery of a nucleic acid to an immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the compound of the following formula: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, an aspect of the disclosure relates to an LNP or a pharmaceutical composition containing thereof, as disclosed herein, for use in a method of targeting the delivery of a nucleic acid to an immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP). In some embodiments, the LNP is an LNP as described herein in the present disclosure.

In some embodiments, the LNP provides at least one of the following benefits:

(i) increased specificity of targeted delivery to the immune cell compared to a reference LNP;
(ii) increased half-life of the nucleic acid or a polypeptide encoded by the nucleic acid in the immune cell compared to a reference LNP;
(iii) increased transfection rate compared to a reference LNP; and
(iv) a low level of dye accessible mRNA (<15%) and high RNA encapsulation efficiencies, wherein at least 80% mRNA was recovered in final formulation relative to the total RNA used in LNP batch preparation.

In some aspect, provided are methods of expressing a polypeptide of interest in a targeted immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition containing thereof, as disclosed herein, for use in a method of expressing a polypeptide of interest in a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:

(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP; and
(v) a low level of dye accessible mRNA (<15%) and high RNA encapsulation efficiencies, wherein at least 80% mRNA was recovered in final formulation relative to the total RNA used in LNP batch preparation.

In some aspects, provided are methods of modulating cellular function of a target immune cell of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the immune cell. In some embodiments, an aspect of the disclosure relates to an LNP or a pharmaceutical composition containing thereof, as disclosed herein, for use in a method of modulating cellular function of a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:

(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP;
(v) the LNP can be administered at a lower dose compared to a reference LNP to reach the same biologic effect in the immune cell; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the modulation of cell function comprises reprogramming the immune cells to initiate an immune response. In some embodiments, the modulation of cell function comprises modulating antigen specificity of the immune cell.

In some aspect, provided are methods of treating, ameliorating, or preventing a symptom of a disorder or disease in a subject in need thereof. In some embodiments, the method comprises administering to the subject a lipid nanoparticle (LNP) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the nucleic acid modulates the immune response of the immune cell, therefore to treat or ameliorate the symptom. In some embodiments, an aspect of the disclosure relates to an LNP or a pharmaceutical composition containing thereof, as disclosed herein, for use in a method of treating, ameliorating, or preventing a symptom of a disorder or disease in a subject in need thereof. A disease or disorder may be as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:

(i) increased specificity of delivery of the nucleic acid into the immune cell compared to a reference LNP;
(ii) increased half-life of the nucleic acid or a polypeptide encoded by the nucleic acid in the immune cell compared to a reference LNP;
(iii) increased transfection rate compared to a reference LNP;
(iv) the LNP can be administered at a lower dose compared to a reference LNP to reach the same treatment efficacy;
(v) increased level of gain of function by an immune cell compared to a reference LNP; and (vi) a low level of dye accessible mRNA (<15%) and high RNA encapsulation efficiencies, wherein at least 80% mRNA was recovered in final formulation relative to the total RNA used in LNP batch preparation.

In some embodiments, the disorder is an immune disorder, an inflammatory disorder, or cancer. In some embodiments, the nucleic acid encodes an antigen for use in a therapeutic or prophylactic vaccine for treating or preventing an infection by a pathogen.

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In some aspect, provided are methods of targeting the delivery of a nucleic acid to an immune cell of a subject. In some embodiments, the method comprises contacting the immune cell with a lipid nanoparticle (LNP) provided herein. In some embodiments, the method is for targeting NK cells. In some embodiments, the immune cell targeting group binds to CD56. In some embodiments, the method is for targeting both T cells and NK cells simultaneously. In some embodiments, the immune cell targeting group binds to CD7, CD8, or both CD7 and CD8. In some embodiments, the method is for targeting both CD4+ and CD8+ T cells simultaneously. In some embodiments, the immune cell targeting group comprises a polypeptide that binds to CD3 or CD7.

In some aspect, provided are methods of expressing a polypeptide of interest in a targeted immune cell of a subject. In some embodiments, the method comprises contacting the immune cell with a lipid nanoparticle (LNP) provided herein.

In some aspect, provided are method of modulating cellular function of a target immune cell of a subject. In some embodiments, the method comprises administering to the subject a lipid nanoparticle (LNP) provided herein.

In some aspect, provided are method of treating, ameliorating, or preventing a symptom of a disorder or disease in a subject in need thereof. In some embodiments, the method comprises administering to the subject a lipid nanoparticle (LNP) provided herein.

In some aspect, provided are methods of treating a disease or disorder related to CD8 in a subject. In some embodiments, the method comprises administering a pharmaceutical composition described herein to the subject. In some embodiments, the disease or disorder is cancer.

LNPs disclosed in the present disclosure and as claimed are suitable for the methods described above.

### VIII. Kits for Use in Medical Applications

Another aspect of the invention provides a kit for treating a disorder. The kit comprises: an ionizable cationic lipid, a lipid-immune cell targeting group conjugate, a lipid nanoparticle composition comprising an ionizable cationic lipid and/or a lipid-immune cell targeting group conjugate with or without an encapsulated payload (e.g., an mRNA), and instructions for treating a medical disorder, such as, cancer or a microbial or viral infection.

### EXAMPLES

The invention now being generally described, will be more readily understood by reference to the following examples, which are included merely for purposes of illustration of certain aspects and embodiments of the present invention, and are not intended to limit the invention.

### Example 1—Preparation of Ionizable Cationic Lipids

This Example describes the synthesis of various cationic lipids.

### Cationic Lipid 1

The synthesis of Cationic Lipid 1, shown in the following formula,

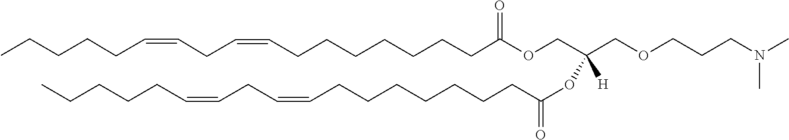 [SMILES: CCCCC/C=C\C/C=C\CCCCCCCC(=O)OC[C@H](COCCCN(C)C)OC(=O)CCCCCCC/C=C\C/C=C\CCCCC]

was prepared as described in the following scheme 1.

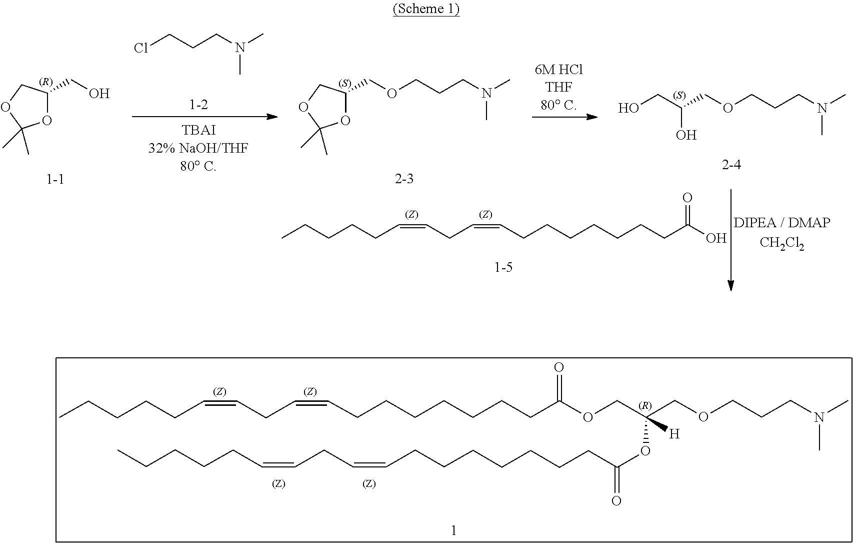

Ether intermediate 1-3 was prepared by reacting hydroxy-functional, protected 1,2-diol starting material (1-1) (0.151 mol, 24 g) with dimethylaminopropylchloride, compound 1-2(0.051 mol, 24 g, 1 equiv.) and TBAI (0.0015 mol, 554 mg, 0.01 equiv.) in the presence of NaOH (32%)/THF at 80° C. overnight to afford ether intermediate compound 1-3 (20.1 g, 0.1 mol). Thereafter, compound 1-3 (2.2 g, 0.01 mol) was deprotected (THF, 6 M HCl, 4 hours) to obtain vicinal diol intermediate compound 1-4 (1.6 g, 0.009 mol) in quantitative yield. Compound 1-4 (1.12 g, 0.006 mol) was bis-acylated using fatty acid 1-5 (5.9 mL 0.018 mol, 3 equiv.) using 10 equiv. EQ DIPEA, in DCM using EDC (3.8 g, 0.019 mol, 3.2 equiv.) to afford Lipid Compound 1 (238 mg, 0.0034 mol). Lipid Compound 1 was purified by preparatory HPLC (CombiFlash Nextgen 300+Teledyne ISCO), and final product purity was 98% (RP-HPLC-ELSD, using Durashell-C18, 4.6×50 mm, 3 uM, Cat# DC930505-0).

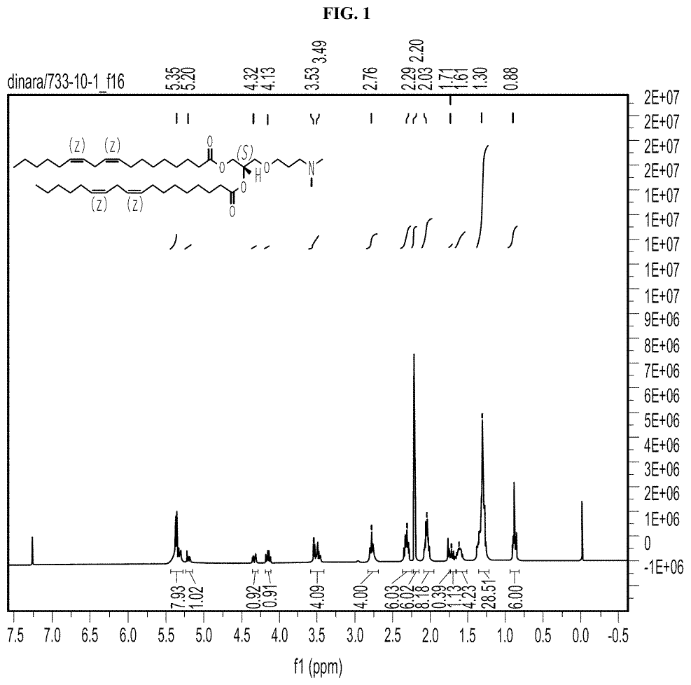

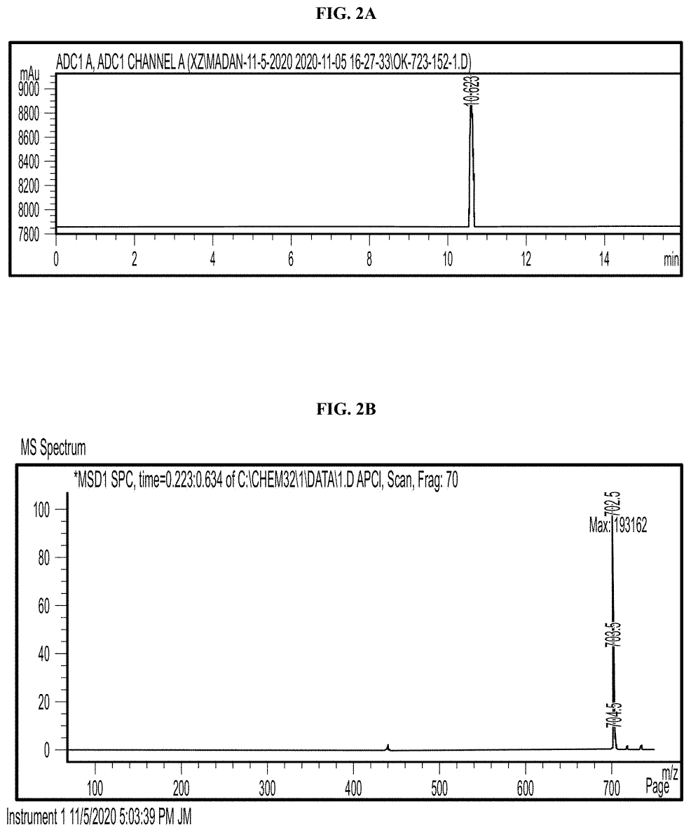

Purified Lipid 1 free base (C44H79N05, molecular weight 702.12 g/mol.) was characterized by proton NMR Spectroscopy (400 MHz) in CDCl3 as shown in FIG. 1, and by LC-MS to confirm structure (NMR, m/z) and purity (NMR, LC) as shown in FIGS. 2A and 2B.

### Cationic Lipid 2

The synthesis of Cationic Lipid 2, shown in the following formula,

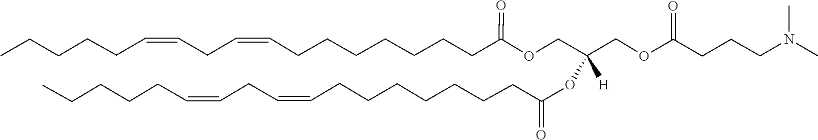 [SMILES: CCCCC/C=C\C/C=C\CCCCCCCC(=O)OC[C@H](COC(=O)CCCN(C)C)OC(=O)CCCCCCC/C=C\C/C=C\CCCCC]

was prepared as described in the following scheme 2.

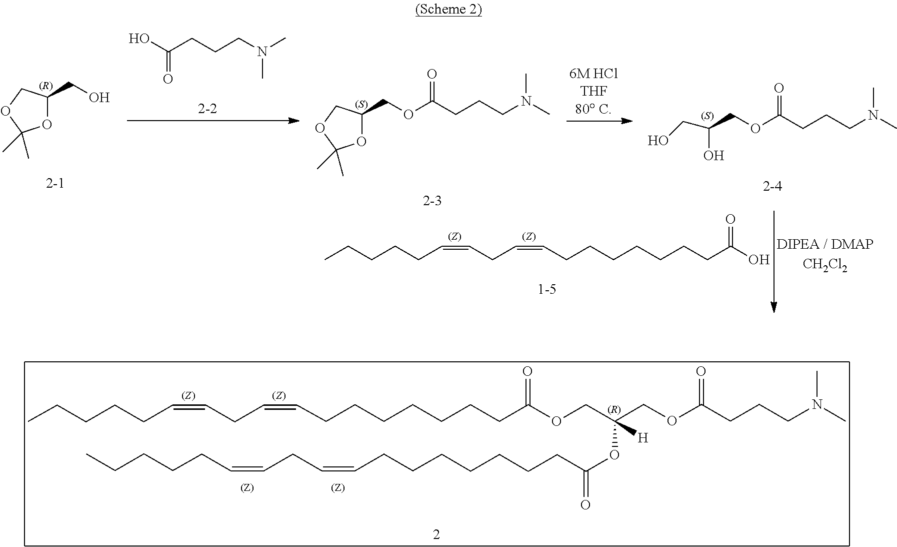

Ester intermediate 2-3 was prepared by acylating protected 1,2-diol (1,2-O-isopropylidene-D-glycerol) starting material (2-1) with dimethylaminobutanoic acid compound 2-2 (0.03 mol, 4.19 g, 0.03 eq.) using EDCl (0.03 mol, 5.73 g, 1 eq.), DIPEA (0.12 mol, 12.9 g, 4.0 eq.) and DMAP (0.006 mol, 733 mg, 0.2 eq.) in DCM (300 mL) solution to obtain 5.44 g (0.02 mol) of 2-3. Intermediate 2-3 (670 mg, 0.0027 mol, 1.0 eq.) was deprotected in a mixture of 10 mL TFA/water (50:50 v/v) and 10 mL 6 M HCl at room temperature to afford 560 mg (0.0027 mol) of compound 2-4. Compound 2-4 (0.003 mol, 641 mg, 1 eq.) was esterified with fatty acid 1-5 (0.009 mol, 2.52 g, 3 eq.) in 50 mL of DCM, using EDC (0.009 mol, 1.72 g, 1 eq.), DIPEA(0.001 mol, 122 mg, 0.33 eq.) to afford cationic lipid 2 (260 mg, 0.0003 mol).

Lipid Compound 2 was purified by preparatory HPLC. The resulting product was greater than 99% pure (Reverse phase HPLC-ELSD using Durashell-C18, 4.6×50 mm, 3 uM, Cat# DC930505-0).

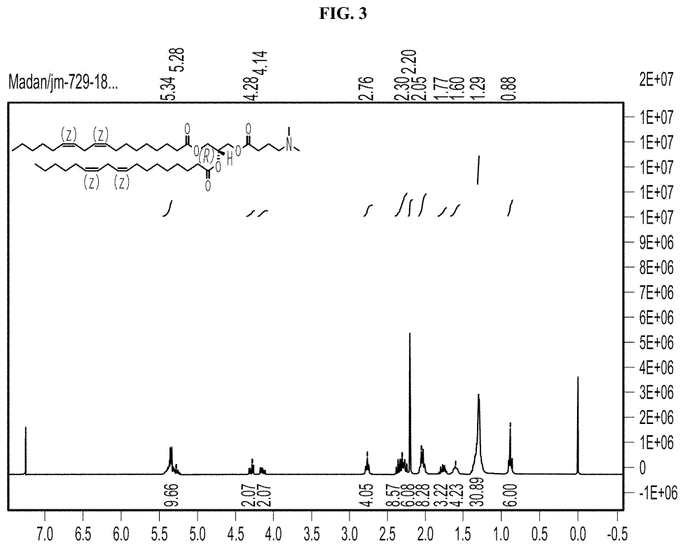

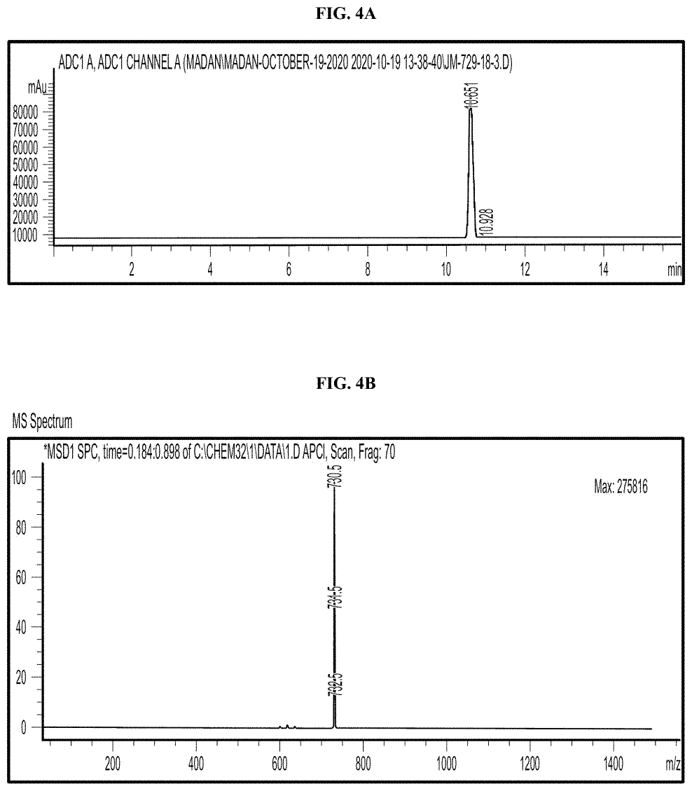

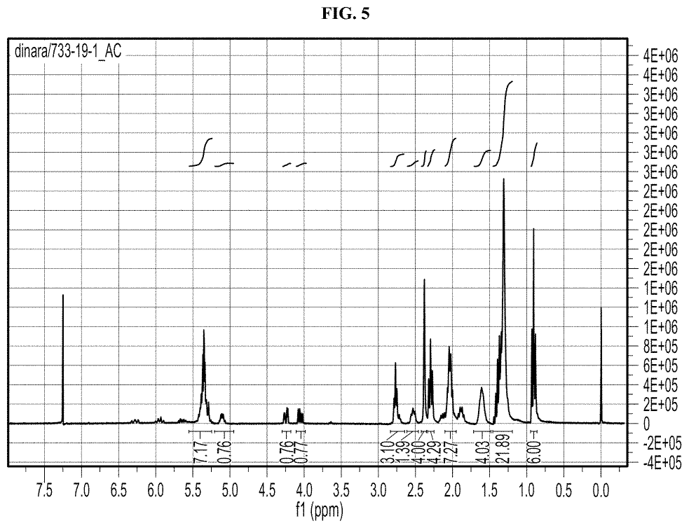

Purified Lipid 2 free base (C45H79N06, molecular weight 730.10 g/mol.) was characterized by proton NMR Spectroscopy (400 MHz) in CDCl3 as shown in FIG. 3 and by LC-MS to confirm structure (NMR, m/z) and purity (NMR, LC) as shown in FIGS. 4A and 4B.

Lipid 6 was synthesized using a method analogous to the synthesis of Lipid 2 except that diethylaminobutanoic acid was used to generate the tertiary amine head group instead of dimethylaminobutanoic acid used for Lipid 2.

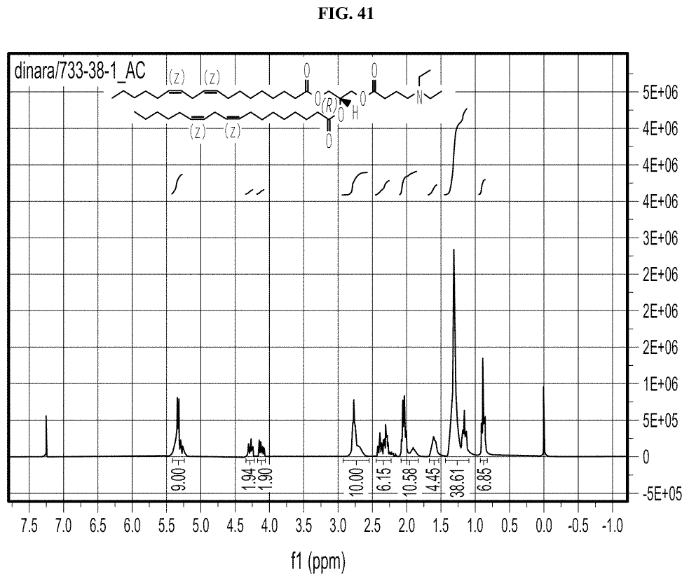

Purified Lipid 6 was characterized by proton NMR spectroscopy as shown in FIG. 41 and mass spectrometry and reverse phase HPLC as shown in FIGS. 42A and 42B.

### Cationic Lipid 3

The synthesis of Cationic Lipid 3, shown in the following formula,

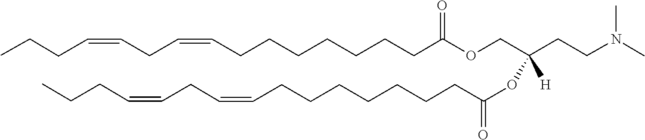 [SMILES: CCC/C=C\C/C=C\CCCCCCCC(=O)OC[C@H](CCN(C)C)OC(=O)CCCCCCC/C=C\C/C=C\CCC]

was prepared as described in the following scheme 3.

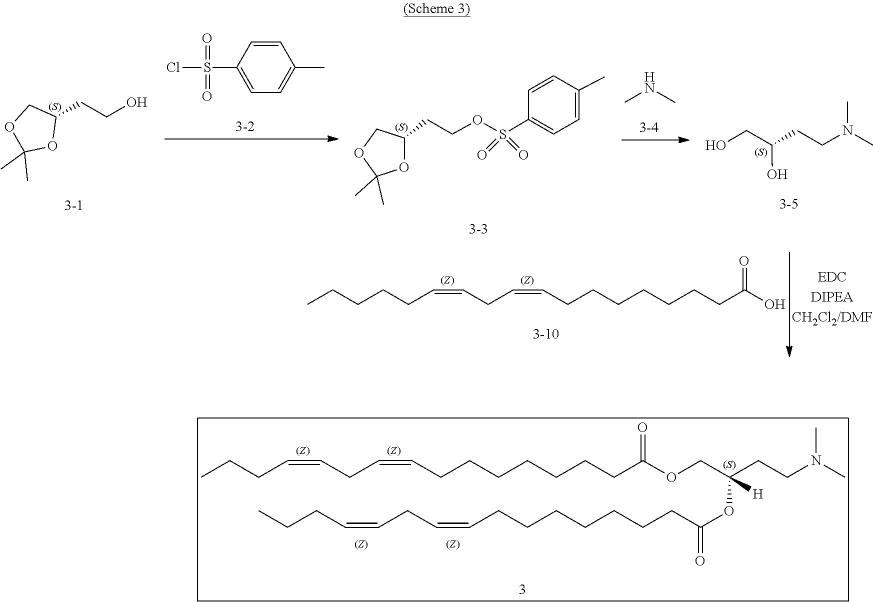

Fatty acid 3-10 was prepared using the following scheme 3a:

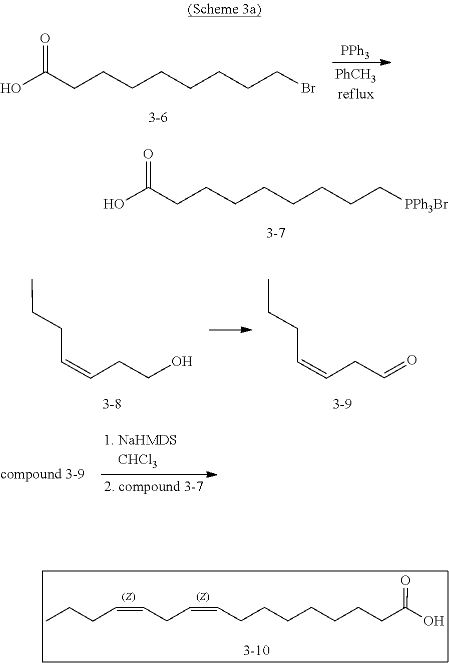

Fatty acid 3-10 (9Z,12Z)-hexadeca-9,12-dienoic acid was synthesized using a Wittig reaction approach as shown in Scheme 3a. In order to produce compound 3-7, 9-bromononanoic acid (0.0148 mol, 3.50 g) was combined with PPh₃ (0.0148 mol, 3.87 g, 1 eq.) in 5 mL toluene and refluxed for 48 hours to afford 7.23 g of phosphonium bromide 3-7. In order to produce compound 3-9, compound 3-8 (0.0076 mol, 0.87 g) was oxidized using Dess-Martin periodinane (0.0082 mol, 3.48 g, 1.1 eq.) in 20 mL DCM to afford aldehyde 0.85 g of 3-9. Compound 3-9 (0.0076 mol, 085 g) was reacted with compound 3-7 (0.0076 mol, 3.78 g, 1 eq.) in 7.6 mL of 40% NAHMDS in 60 mL THF to afford 400 mg of fatty acid 3-10.

As shown in Scheme 3, intermediate 3-3 was produced by the tosylation of the free hydroxyl on a protected 1,2,4-butanetriol starting material (3-1) (0.0342 mol, 5.0 g, 1 eq.) using tosylchloride (3-2) (0.034 mol, 6.5 g, 1 eq.) and triethylamine (0.034 mol, 4.9 mL, 1 eq.), DMAP (0.011 mol, 200 mg, 0.3 eq.) in 250 mL DCM at room temperature. Nucleophilic displacement of compound 3-3 (0.0032 mol, 1.0 g, 1 eq.) with dimethylamine (0.03 mol, 1.5 g, 10 eq.) in 16.6 mL of THF to afford 400 mg of tertiary amine 3-4. Compound 3-4 was deprotected with 2 M HCl in MeOH to afford 410 mg of compound 3-5. Compound 3-5 (90 mg) was esterified with fatty acid 3-10 (0.0016 mol, 400 mg, 3 eq.) using EDC (0.0018 mol, 306 mg, 3 eq.), DIPEA (0.0024 mol, 8.8 mL, 4.5 eq.) in 5.4 mL of DCM, 2 hours) to afford 12 mg of ionizable lipid 3. Lipid Compound 3 was purified by preparatory HPLC.

Lipid Compound 3 was purified by preparatory HPLC (CombiFlash Nextgen 300+Teledyne ISCO). Product purity of 99% was determined by reverse phase HPLC-ELSD (using Durashell-C18, 4.6×50 mm, 3 uM, Cat# DC930505-0).

Cationic Lipid 4

The synthesis of Cationic Lipid 4, shown in the following formula,

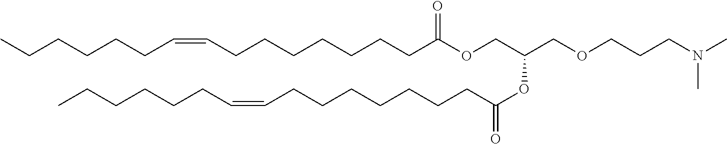 [SMILES: CCCCCC/C=C\CCCCCCCC(=O)OC[C@H](COCCCN(C)C)OC(=O)CCCCCCC/C=C\CCCCCC]

was prepared as described in the following Scheme 4.

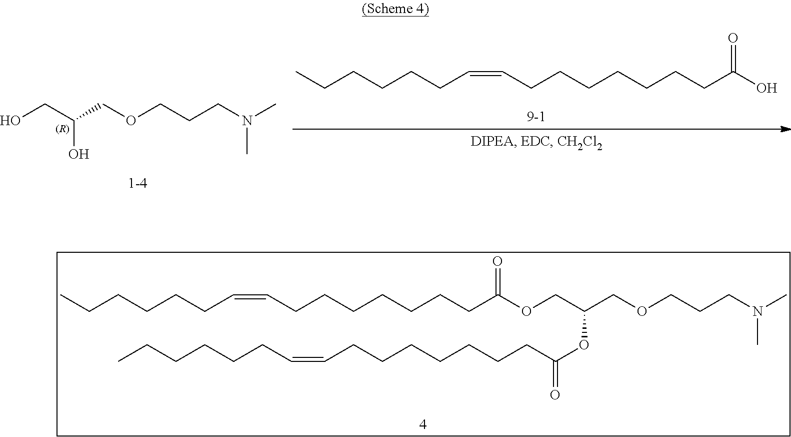

Fatty acid 9-1 was synthesized using a protocol analogous to that used for synthesis of fatty acid 4-6 above using 9-bromononanoic acid heptanal starting materials.

The bis-acylation of 1-4 (0.003 mol, 600 mg) by fatty acid 9-1 (0.01 mol, 2.14 g, 3 eq.) proceeded using EDC (0.009 mol, 1.7 g, 3.2 eq.), DIPEA (0.009 mol, 2.45 mL, 3.2 eq.) in 7 mL of DCM provided 203 mg of ionizable lipid 4 (mass, yield). Lipid Compound 4 was purified by preparatory HPLC (CombiFlash Nextgen 300+Teledyne ISCO) and yield 99% pure product (HPLC-ELSD using Durashell-C18, 4.6×50 mm, 3 uM, Cat# DC930505-0).

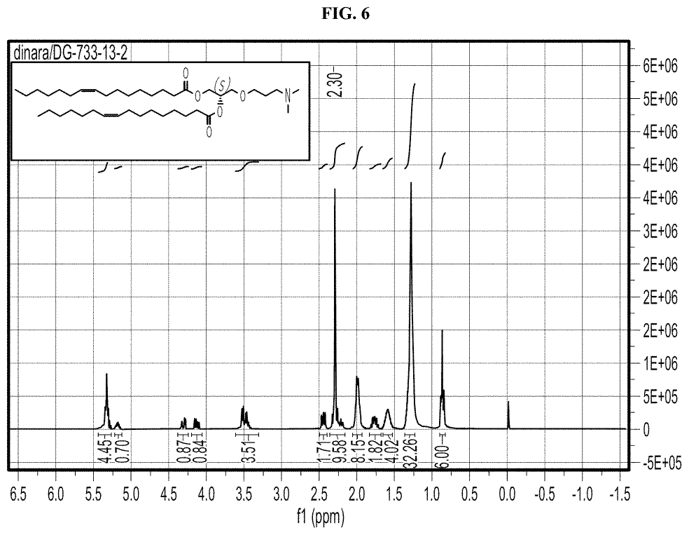

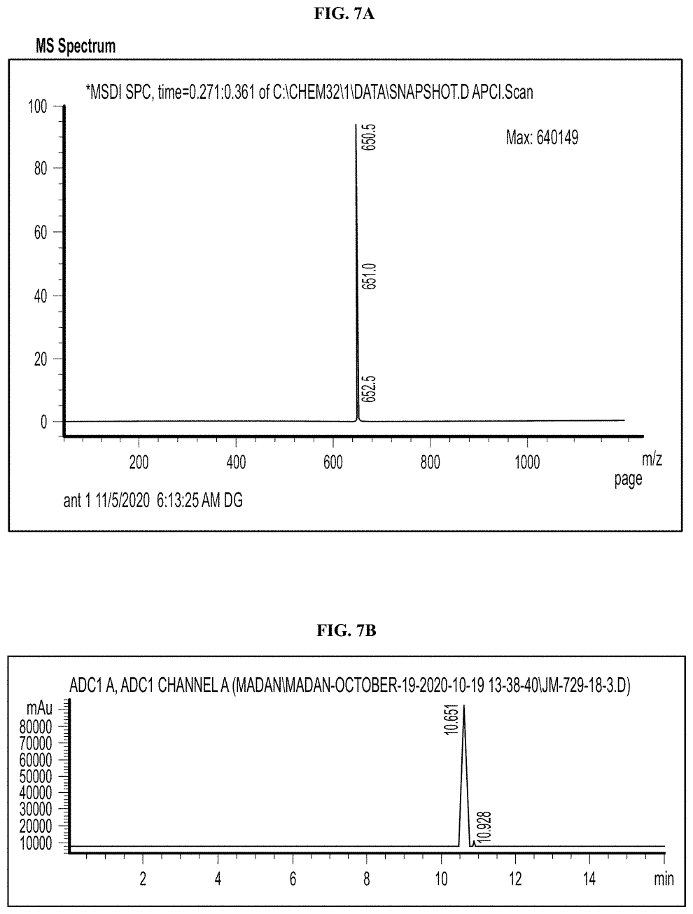

Purified Lipid 4 was characterized by proton NMR spectroscopy as shown in FIG. 6 and mass spectrometry and reverse phase HPLC as shown in FIGS. 7A and 7B.

Cationic Lipid 5

The synthesis of Cationic Lipid 5, shown in the following formula,

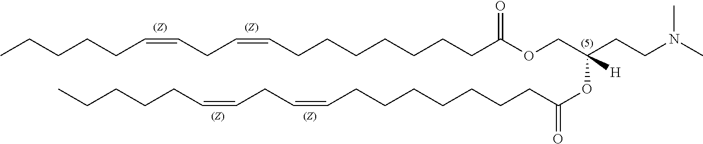 [SMILES: CCCCC/C=C\C/C=C\CCCCCCCC(=O)OC[C@H](CCN(C)C)OC(=O)CCCCCCC/C=C\C/C=C\CCCCC]

was prepared as described in the following scheme 5.

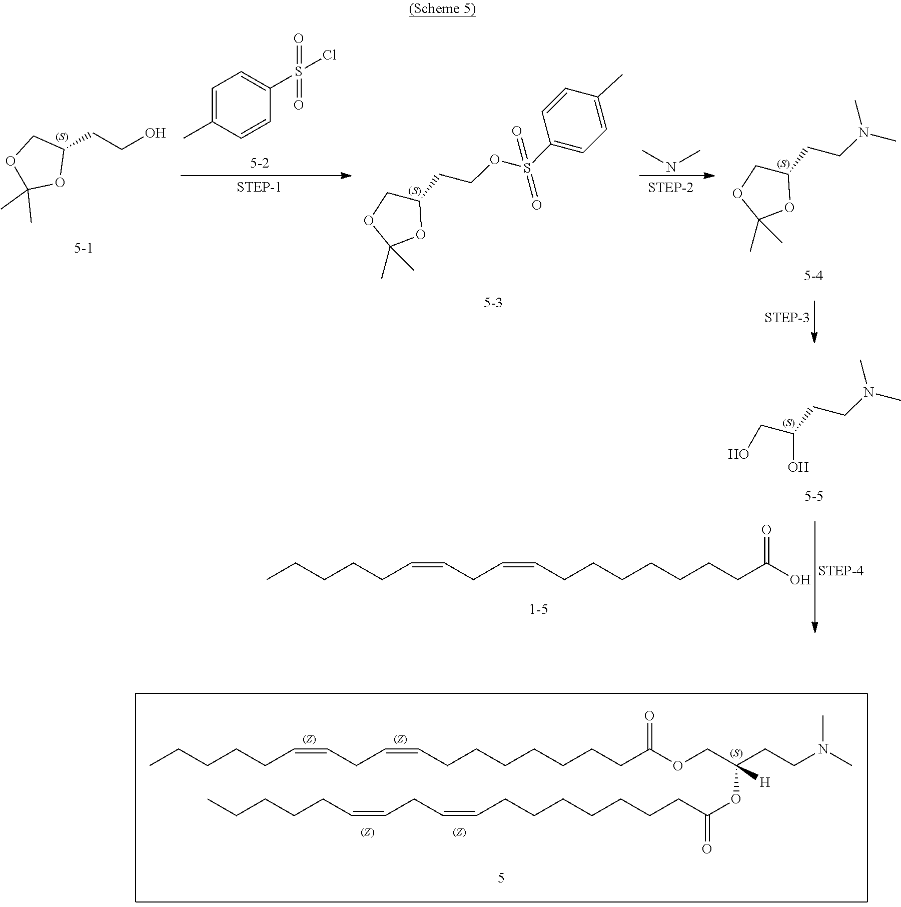

As shown in Scheme 5, intermediate 5-3 was produced by the tosylation of the free hydroxyl on a protected 1,2,4-butanetriol starting material (5-1) (0.0342 mol, 5.0 g, 1 eq.) using tosylchloride (5-2) (0.039 mol, 7.5 g, 1 eq.) and triethylamine (0.039 mol, 5.6 mL, 1 eq.), DMAP (0.013 mol, 230 mg, 0.3 eq.) in 250 mL DCM at room temperature. Nucleophilic displacement of compound 5-3 (0.0032 mol, 1.0 g, 1 eq.) with dimethylamine (0.03 mol, 1.5 g, 10 eq.) in 16.6 mL of THF overnight to afford 1.1 g of tertiary amine 5-4. Compound 5-4 (712 mg) was deprotected in 6 M HCl in water (5 mL) to afford 551 mg of compound 5-5. Compound 5-5 (2 g) was esterified with fatty acid 1-5 (35.5 mmol, 8.88 g, 3 eq.) using EDC.HCl (35.5 mol, 6.7 g, 3 eq.), DIPEA (47 mmol, 6.7 mL, 4 eq.) in 55 mL of DCM, 2 hours) to afford 1.09 g of ionizable lipid 5. Lipid Compound 5 was purified by preparatory HPLC.

Lipid Compound 5 was purified by preparatory HPLC (CombiFlash Nextgen 300+Teledyne ISCO). Product purity of 99% was determined by reverse phase HPLC-ELSD (using Durashell-C18, 4.6×50 mm, 3 uM, Cat# DC930505-0).

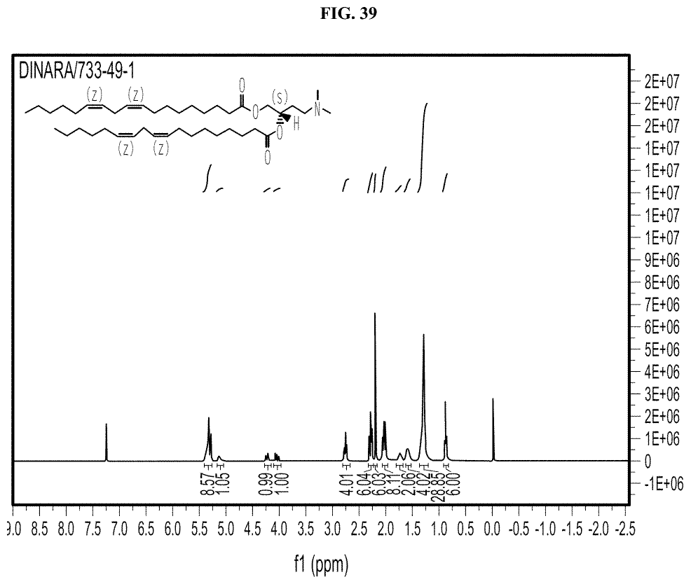

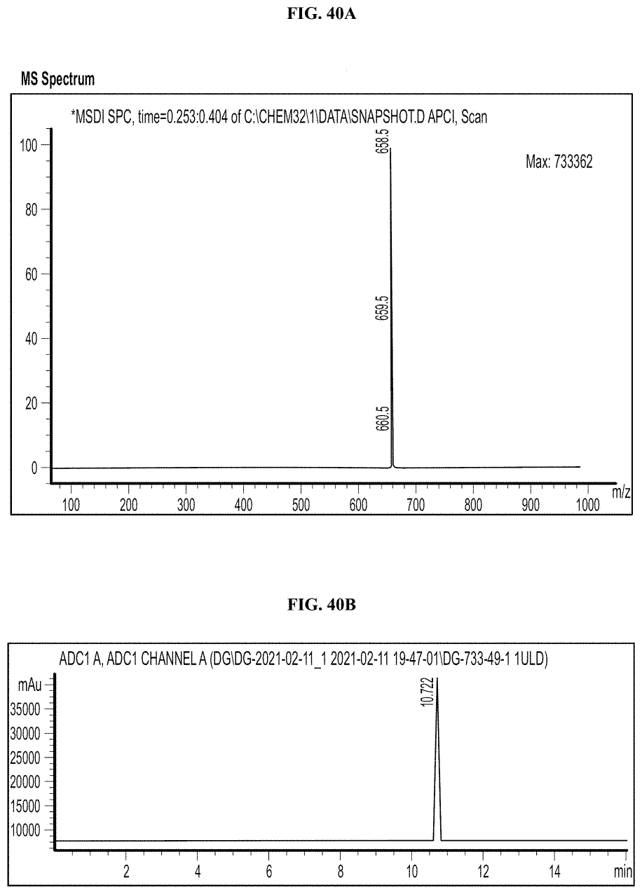

Purified Lipid 5 free base (C42H74N04, molecular weight 657.57 g/mol.) was characterized by proton NMR Spectroscopy (400 MHz) in CDCl3 as shown in FIG. 39A and by mass spectrometry to confirm structure (NMR, m/z) and purity (LC-ELSD) as shown in FIGS. 40A and 40B.

Lipid 7 was synthesized using a method analogous to the synthesis of Lipid 5 except using diethyl amine instead of dimethyl amine to incorporate the tertiary amine head group.

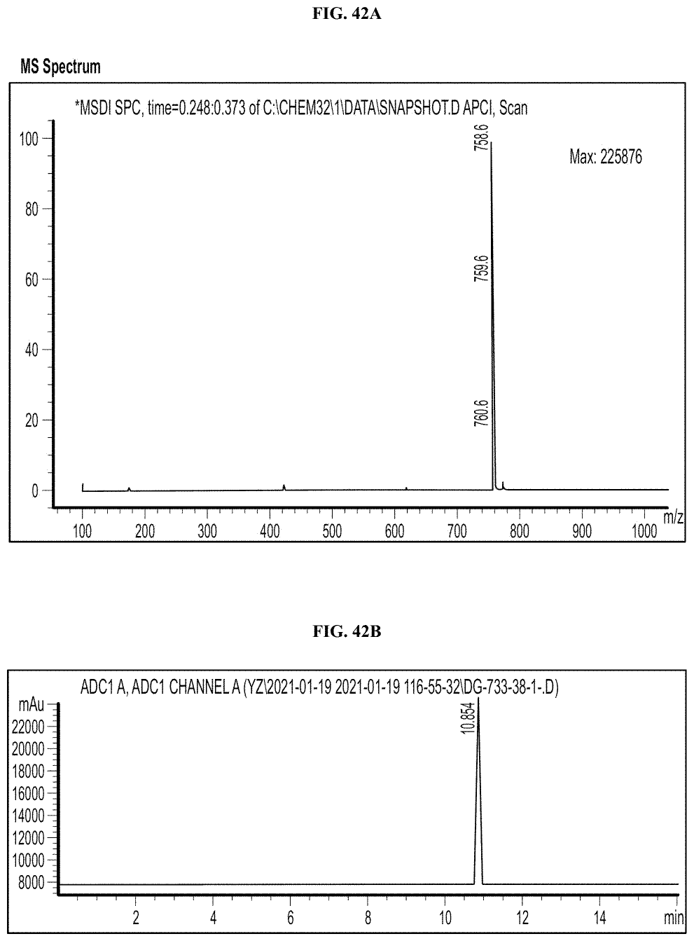

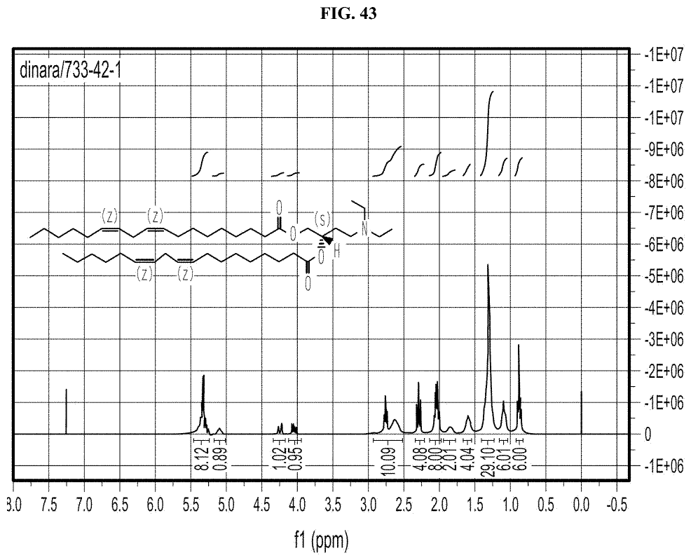

Purified Lipid 7 was characterized by proton NMR spectroscopy as shown in FIG. 43 and mass spectrometry and reverse phase HPLC as shown in FIGS. 44A and 44B.

Scheme 6 below depicts the synthesis of Lipid 9, Lipid 10, and Lipid 11:

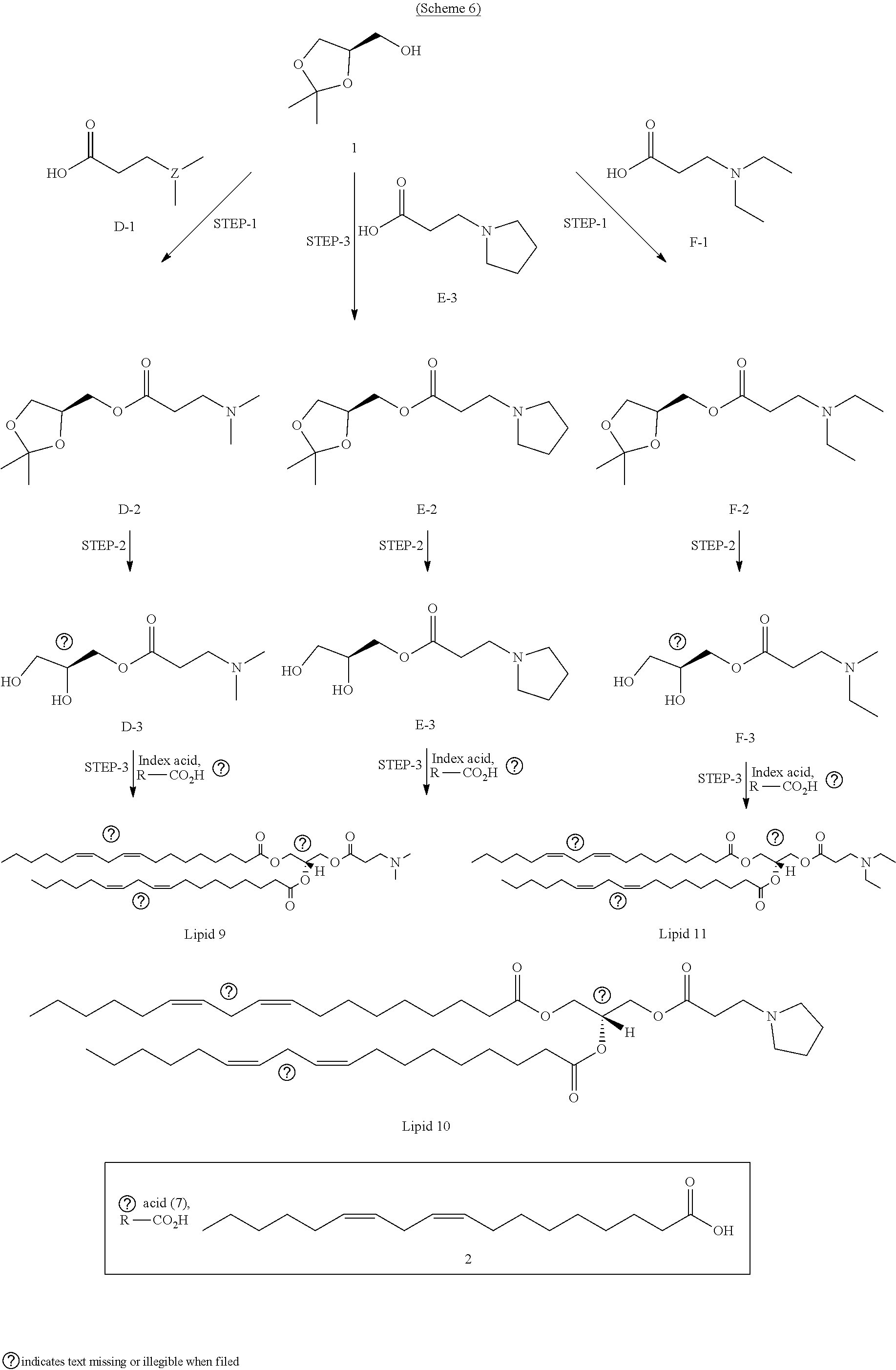

### Cationic Lipid 9

Ester intermediate D-2 was prepared by acylating 4 g (30.2 mmol) of protected 1,2-diol (1,2-O-isopropylidene-D-glycerol) starting material (1) with dimethylaminopropanoic acid compound D-1 (33.3 mmol, 6.06 g, 1.71 eq), EDCI (33.2 mmol, 6.28 g, 1.1 eq), DIPEA (121.0 mmol, 21.08 mL, 4.0 eq), and DMAP (6 mmol, 740 mg, 0.2 eq) in DCM (100 mL) solution to obtain 2.6 g (0.02 mol) in 37% yield of D-2. Intermediate D-2 (2.5 g, 10.68 mmol, 1.0 eq.) was deprotected in a 1:3 (v/v) mixture of 1M aq. HCl and THF (total volume 20 mL) at room temperature to afford 2.4 g (12.6 mmol) of crude compound D-3. Crude compound D-3 (12.6 mmol, 2.4 g, 1 eq.) was esterified with fatty acid 2 (8.9 g, 2.5 eq, 31.8 mmol), EDCI (6.1 g, 2.5 eq, 31.8 mmol), DIPEA (5.53 mL, 2.5 eq, 31.8 mmol), DMAP (285 mg, 0.2 eq, 2.6 mmol), DCM (100 mL) to afford 7 g of crude ionizable lipid 9. Crude product (3g) was purified by preparatory HPLC to obtain 100 mg of pure Lipid (>99% pure by Reverse phase HPLC-ELSD using Durashell-C18, 4.6×50 mm, 3 uM, Cat# DC930505-0).

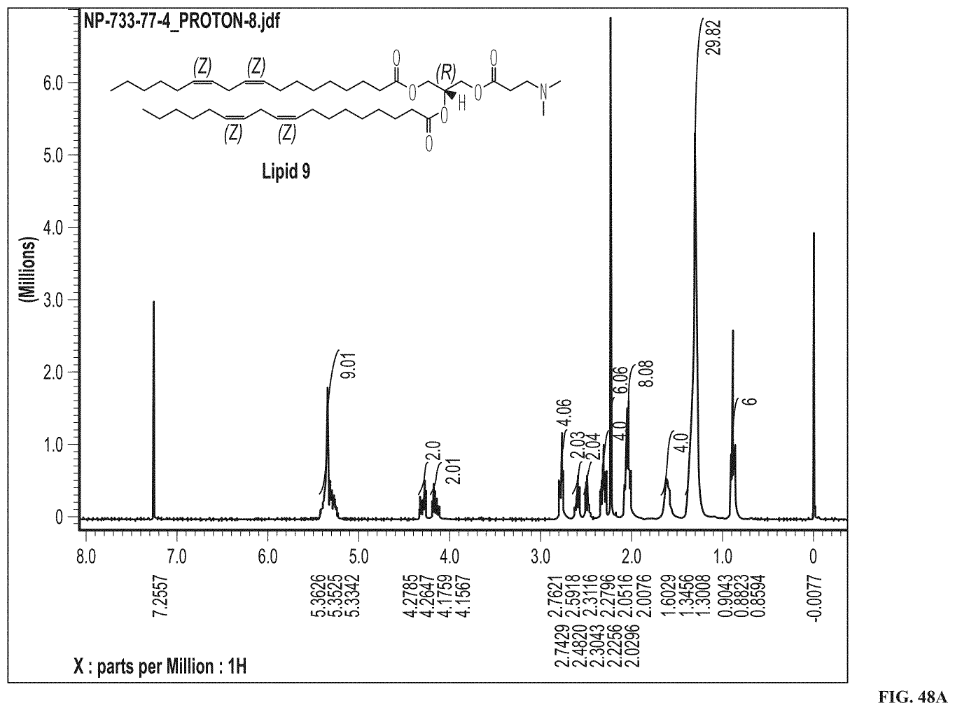

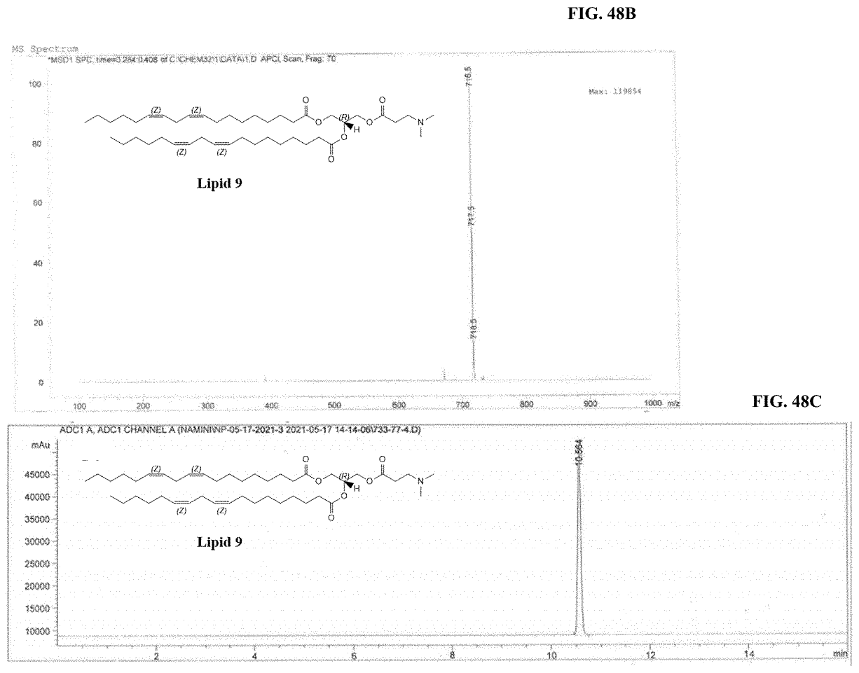

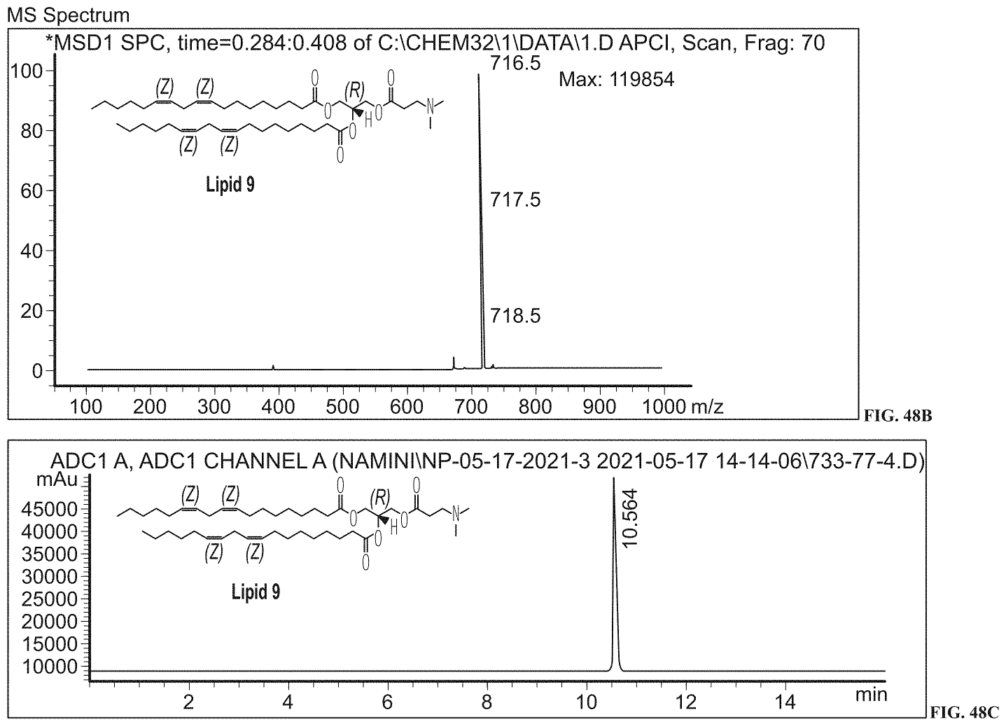

Purified Lipid 9 free base (C44H77N06, molecular weight 716.10 g/mol.) was characterized by proton NMR Spectroscopy (400 MHz) in CDCl3 and Mass Spectrometry to confirm structure (FIG. 48A and FIG. 48B) and by LC-ELSD to determine purity (FIG. 48C).

### Cationic Lipid 10

Ester intermediate E-2 was prepared by acylating 4 g (30.2 mmol) of protected 1,2-diol (1,2-O-isopropylidene-D-glycerol) starting material (1) with dimethylaminopropanoic acid compound E-1 (4.76 g, 1.1 eq, 33 mmol), EDCI (6.38 g, 1.1 eq, 33 mmol), DIPEA (21.08 mL, 4.0 eq, 120 mmol), and DMAP (680 mg, 0.2 eq, 6 mmol), in DCM (150 mL) solution to obtain 1.9 g (7.38 mmol) in 25% yield of E-2. Intermediate E-2 (1.9 g, 7.38 mmol, 1 eq) was deprotected in a 1:3 (v/v) mixture of 1M aq. HCl and THF (total volume 80 mL) at room temperature to afford 1.58 g (7.27 mmol) of crude compound E-3. Crude compound E-3 (7.27 mmol, 1.58 g, 1 eq.) was esterified with fatty acid 2 (18.2 mmol, 5.1 g, 2.5 eq), EDCI (18.2 mmol, 3.48 g, 2.5 eq), DIPEA (18.2 mmol, 3.16 mL, 2.5 eq), and DMAP (1.4 mmol, 160 mg, 0.2 eq) in DCM (80 mL) solution to afford ˜7.5 g of crude ionizable lipid 10. Crude product (3g) was purified by preparatory HPLC to obtain 105 mg of pure Lipid (>99% pure by Reverse phase HPLC-ELSD using Durashell-C18, 4.6×50 mm, 3 uM, Cat# DC930505-0)

Purified Lipid 10 free base (C46H79N06, molecular weight 742.14 g/mol.) was characterized by proton NMR Spectroscopy (400 MHz) in CDCl3 and Mass Spectrometry to confirm structure (FIG. 49A and FIG. 49B) and by LC-ELSD to determine purity (FIG. 49C).

### Cationic Lipid 11

Ester intermediate F-2 was prepared by acylating 4 g (30.2 mmol) of protected 1,2-diol (1,2-O-isopropylidene-D-glycerol) starting material (1) with dimethylaminopropanoic acid compound F-1 (6.06 g, 1.38 eq, 41.7 mmol), EDCI (6.38 g, 1.1 eq, 33.3 mmol), DIPEA (21.08 mL, 4.0 eq, 121.0 mmol), and DMAP (740 mg, 0.2 eq, 6 mmol), in DCM (100 mL) solution to obtain 3.3 g (12.7 mmol) in 41.7% yield of F-2. Intermediate F-2 (3.2 g, 12.3 mmol, 1 eq) was deprotected in a 1:3 (v/v) mixture of 1M aq. HCl and THF (total volume 80 mL) at room temperature to afford 3.1 g (14.1 mmol) of crude compound F-3. Crude compound F-3 (14.13 mmol, 3.1 g, 1 eq.) was esterified with fatty acid 2 (35.3 mmol, 9.9 g, 2.5 eq), EDCI (6.8 g, 2.5 eq, 35.3 mmol), DIPEA (6.2 mL, 2.5 eq, 35.3 mmol), and DMAP (316 mg, 0.2 eq, 2.8 mmol) in DCM (100 mL) solution to afford ˜9 g of crude ionizable lipid 11. Crude product (3g) was purified by preparatory HPLC to obtain 55 mg of pure Lipid 11 (>99% pure by Reverse phase HPLC-ELSD using Durashell-C18, 4.6×50 mm, 3 uM, Cat# DC930505-0).

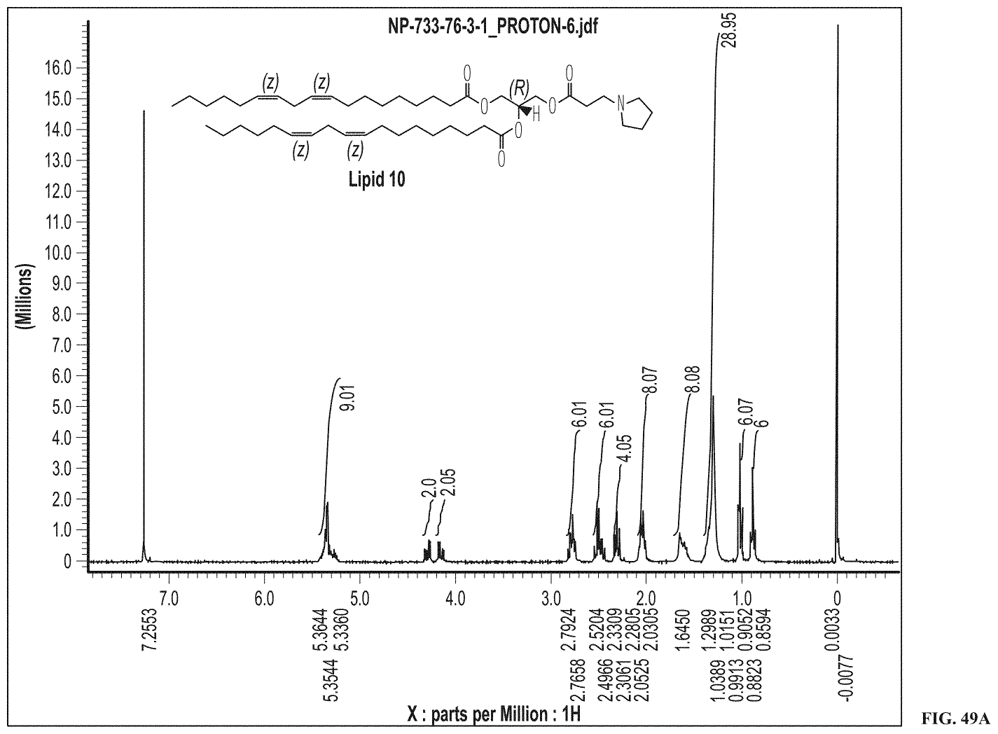

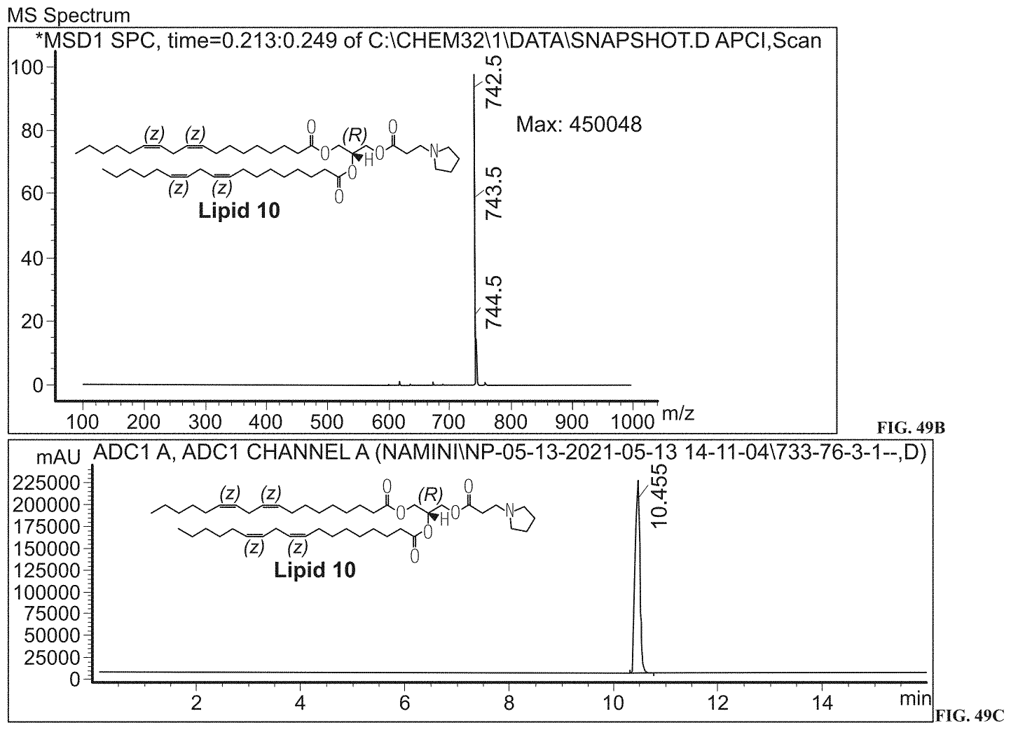

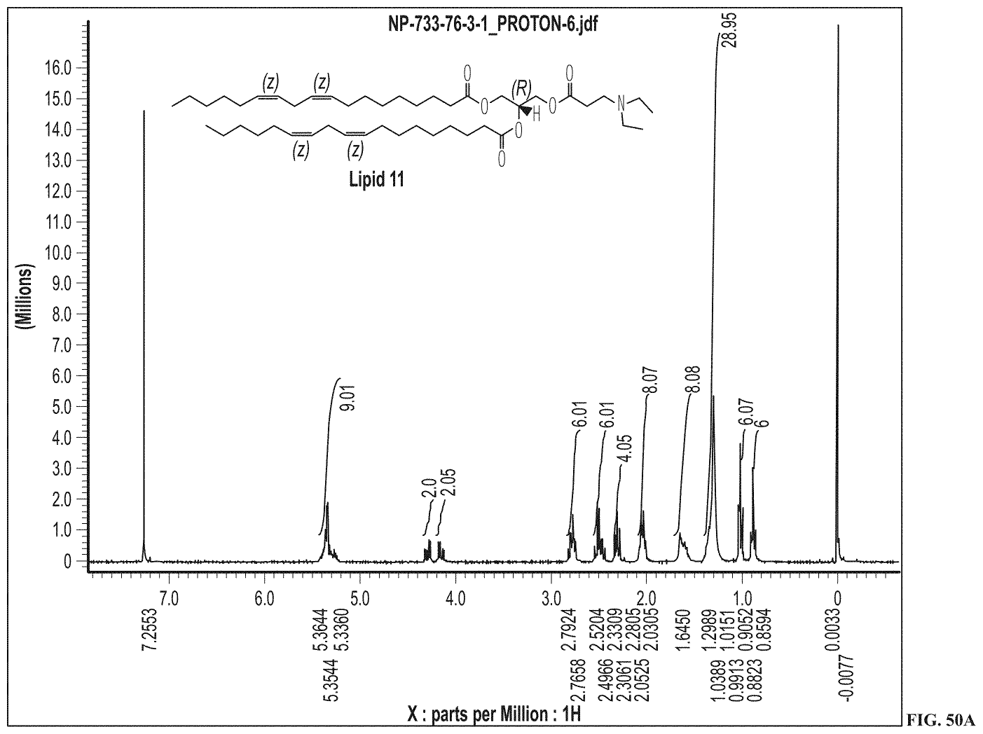

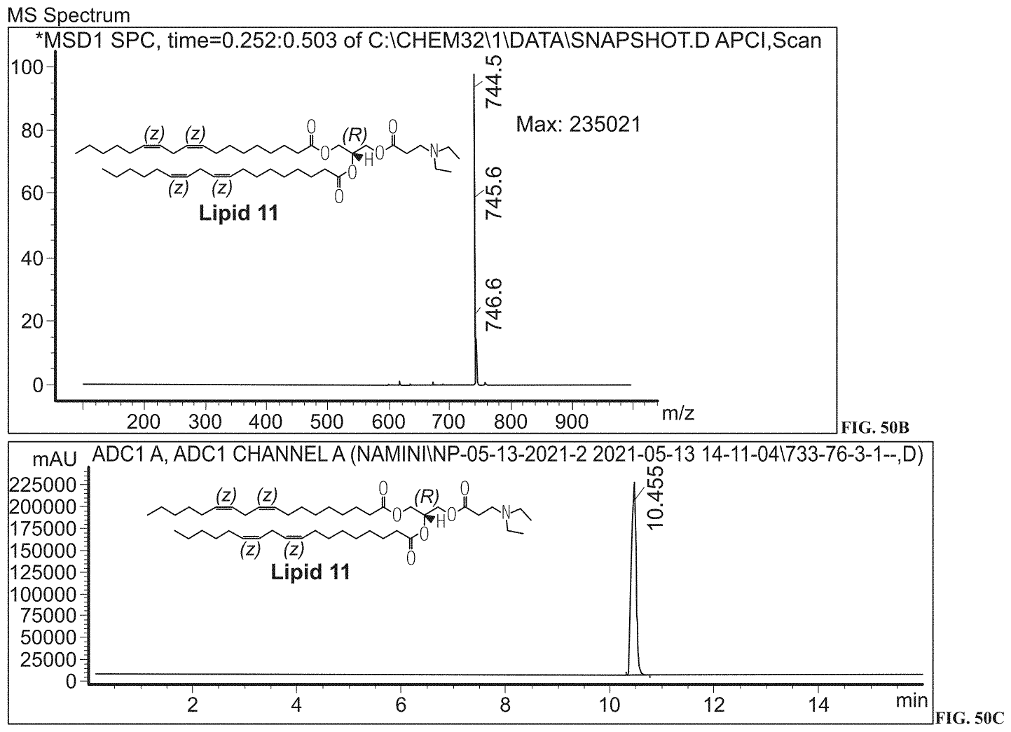

Purified Lipid 11 free base (C46H81N06, molecular weight 744.16 g/mol.) was characterized by proton NMR Spectroscopy (400 MHz) in CDCl3 and Mass Spectrometry to confirm structure (FIG. 50A and FIG. 50B) and by LC-ELSD to determine purity (FIG. 50C).

Scheme 7 and Scheme 8 below depict the synthesis of Lipid 12 and Lipid 13:

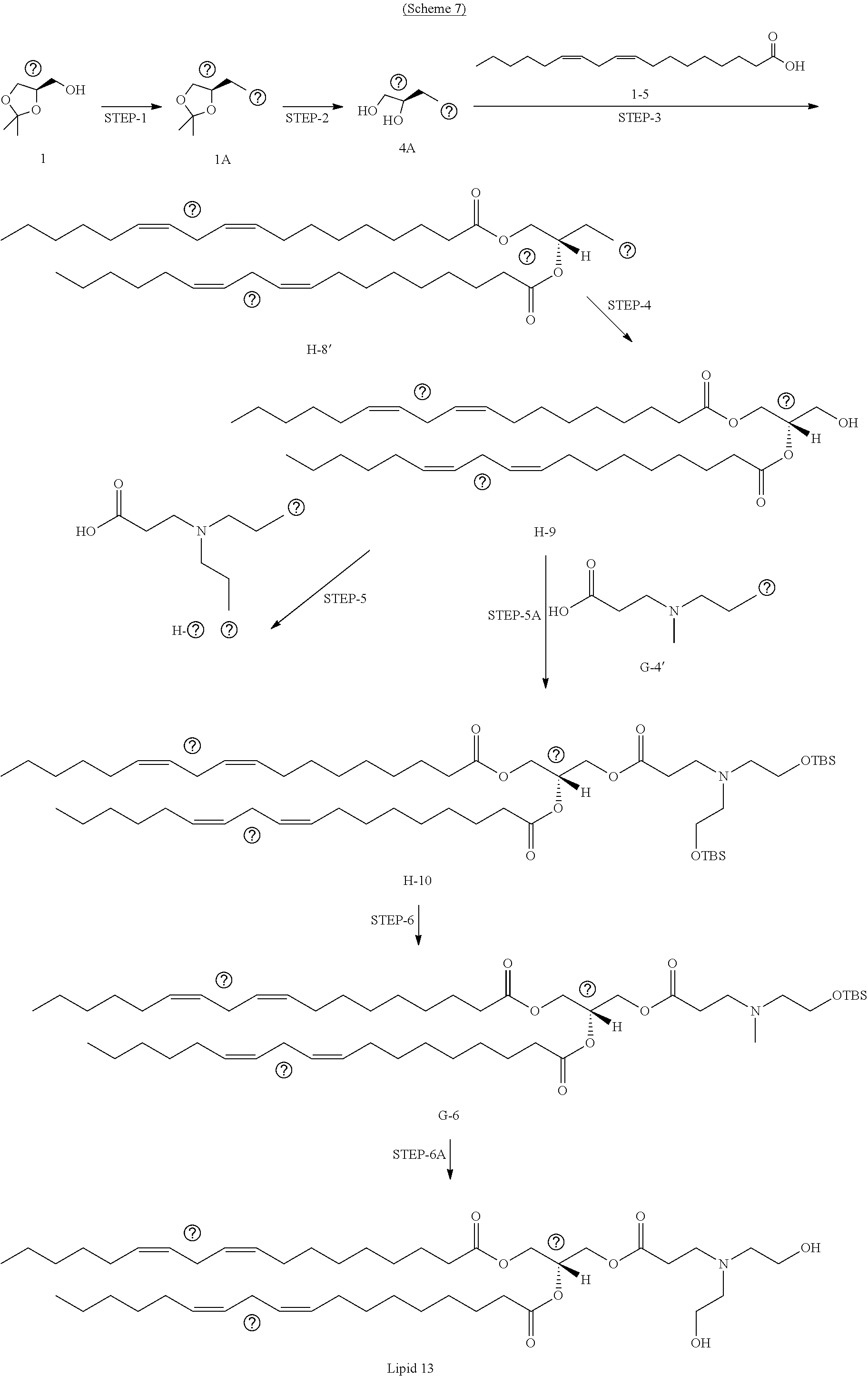

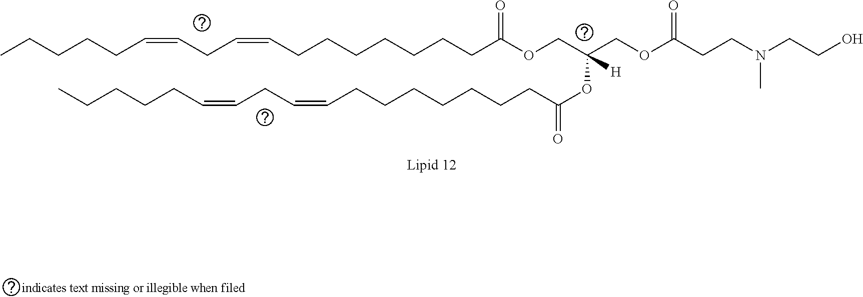 [SMILES: CCCCC/C=C\C/C=C\CCCCCCCC(=O)OC[C@H](COC(=O)CCN(C)CCO)OC(=O)CCCCCCC/C=C\C/C=C\CCCCC ; "Lipid 12"]

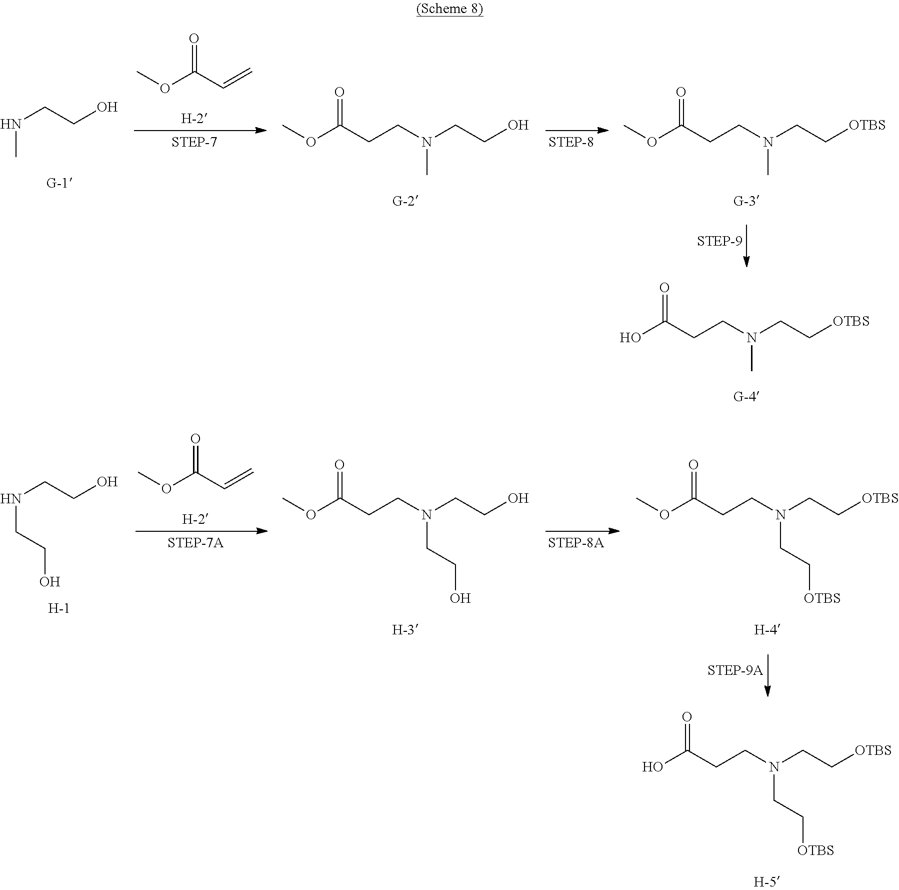

### Cationic Lipid 12

Fmoc protected intermediate 3A was produced from (R)-(2,2-dimethyl-1,3-dioxolan-4-yl)methanol (1), 2.0 g (1.0 eq, 15.2 mmol) using Fmoc chloride (30 mmol, 7.9 g, 2.0 eq) in pyridine (20 mL) to afford 3.8 g of 3A in 71% yield (Step 1, Scheme 7). 3A, 2.3 g (1.0 eq, 6.5 mmol) was selectively deprotected in 1M HCl: THF (1:3, 20 mL) and 0.5 mL methanol to afford g of 4A (Step 2, Scheme 7). O-acylation of 4A, 1.3 g (1.0 eq, 4.2 mmol) with linoleic acid, 1-5 (9.2 mmol, 2.9 mL, 2.2 eq) using PyBOP (9.2 mmol, 4.7 g, 2.2 eq) and DIPEA (9.2 mmol, 1.6 mL, 2.2 eq,) in 3 mL DMF. Combined product from two batches afforded a total of 1.6 g (21%) of pure intermediate H-8′ (Step 3, Scheme 7). Fmoc removal (Step 4, Scheme 7) from H-8′, 2.05 g (1.0 eq, 2.44 mmol) using 1% piperidine in THF (25 mL), 0° C., 4 hours, yielded 680 mg of purified intermediate H-9 in 45% yield. Key intermediate H-9 was used in subsequent steps for production of Lipid 12 (via O-acylation of G-4′) as well as for production of Lipid 13 (via O-acylation of H-5′ as described in relevant section below).

Protected compound G-4′, 3-((2-((tert-butyldimethylsilyl) oxy) ethyl) (methyl) amino) propanoic acid, was prepared using starting materials, methyl acrylate, H-2′, and 2-(methylamino) ethan-1-ol, G-1′ via Michael addition. Methyl acrylate (H-2′), 1.6 ml (1 eq, 17.8 mmol) was reacted with G-1′ (2 g, 1.5 eq, 26.6 mmol) and Aluminum oxide (904 mg, 0.5 eq, 8.9 mmol) under solvent free conditions at room temperature for 3 hours to afford 2.58 g (91%) of G-2′ (Step 7, Scheme 8). G-2′, 1.33 g (1 eq, 8.26 mmol) was converted to the tertiary butyl dimethylsilyl protected intermediate G-3′ (Step 8, Scheme 8) using tert-butyldimethylsilyl chloride, TBDMSC1 (1.62 g, 1.3 eq, 10.74 mmol) and TEA (2.3 ml, 2 eq, 16.52 mmol) in 3 ml DCM at room temperature, overnight, resulting in recovery of about 1.13 g (50%) for purified G-3′. Subsequent selective deprotection of G-3′, 1.14 g (1 eq, 4 mmol) in THF/MeOH/1 M HCl (3/2/1 (v/v); total volume of 6 ml) at room temperature, overnight, yielded 1.13 g of G-4′ (Step 9, Scheme 8). G-4′ and H-9 were combined to produce intermediate G-6 (Step 5A, Scheme 8). H-9, 400 mg (1.0 eq, 0.65 mmol) was acylated with G-4′ (0.98 mmol, 268 mg, 1.5 eq) using EDCI (198 mg, 1.5 eq, 0.98 mmol), DIPEA (167 μL, 1.5 eq, 0.98 mmol), DMAP (15.9 mg, 0.2 eq, 0.13 mmol) in 2.0 mL DCM to afford 308 mg (55%) of crude G-6. Crude G-6 (308 mg) was deprotected in HF.pyridine (9.0 mmol, 641 μL, 25 eq) in 6.0 mL of THF (Step 6A, Scheme 7) yielding 308 mg of crude Lipid 12. Crude product was purified twice using preparatory HPLC to isolate 213 mg (79%) of purified Lipid 12. (>99% pure by Reverse phase HPLC-ELSD using Durashell-C18, 4.6×50 mm, 3 uM, Cat# DC930505-0).

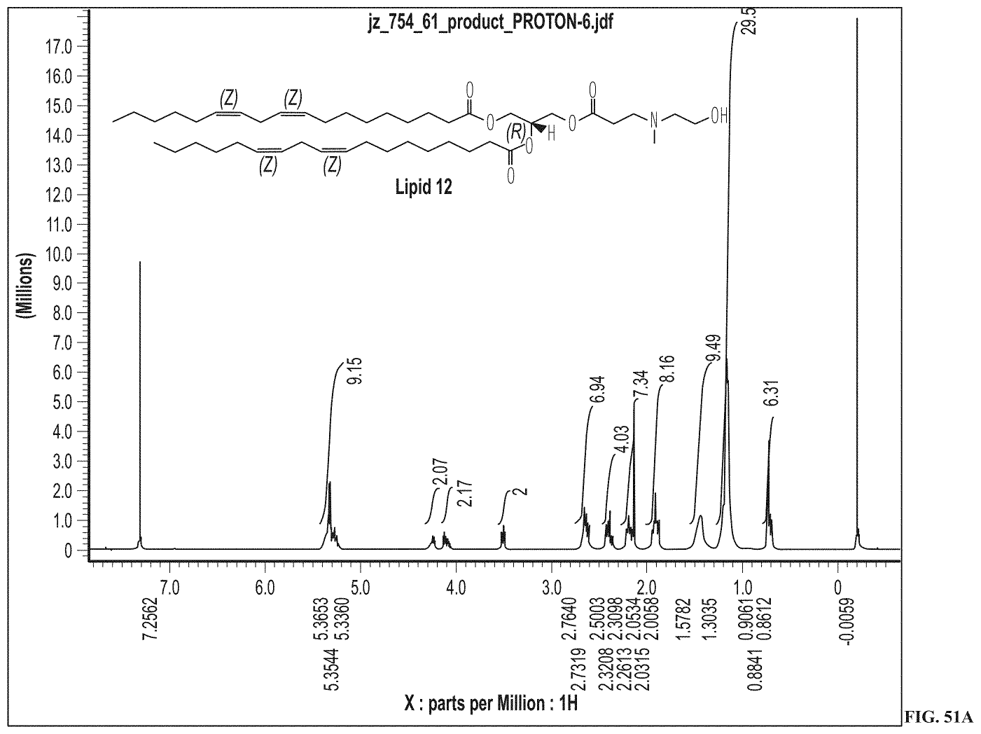

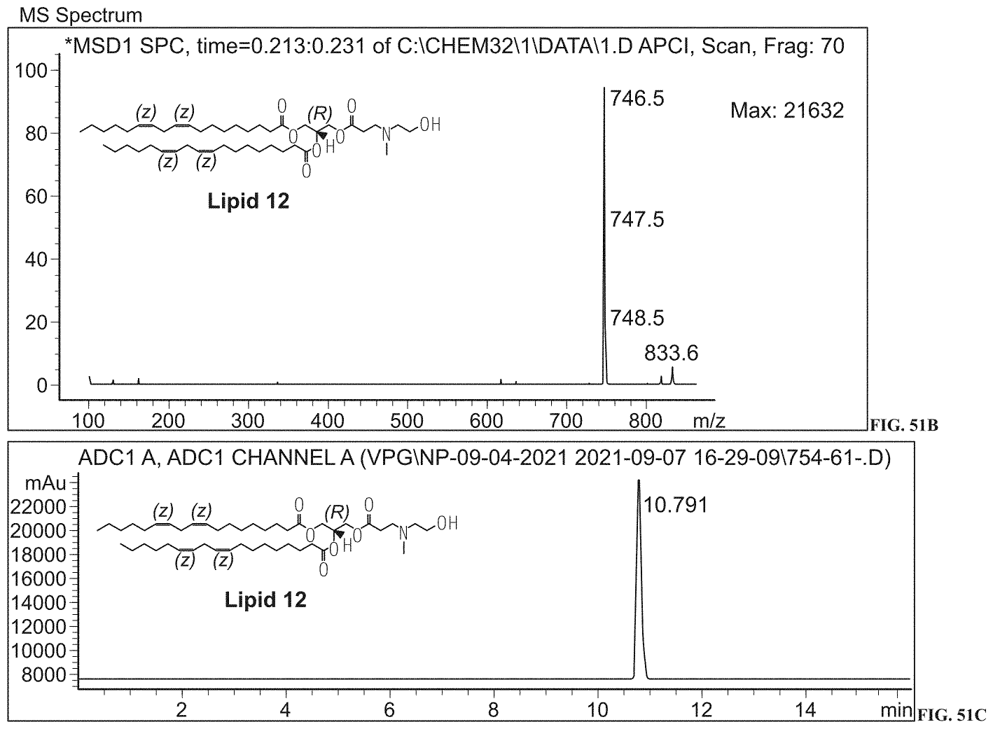

Purified Lipid 12 free base (C45H79N07, molecular weight 746.13 g/mol.) was characterized by proton NMR Spectroscopy (400 MHz) in CDCl3 and Mass Spectrometry to confirm structure (FIG. 51A and FIG. 51B) and by LC-ELSD to determine purity (FIG. 51C).

### Cationic Lipid 13

Protected compound 3-(bis(2-((tert-butyldimethylsilyl)oxy)ethyl)amino)propanoic acid (H5′) was prepared using starting materials, methyl acrylate, H-2′, and 2,2′-azanediylbis(ethan-1-ol), H-1′ via Michael addition. Methyl acrylate (H-2′), 1.65 g (1 eq, 19.2 mmol) was reacted with H-1′ (28.5 mmol, 3.0 g, 1.5 eq,), Aluminum oxide (960 mg, 0.5 eq, 9.6 mmol) under solvent free conditions at room temperature for 3 hours to afford 3.53 g (97%) of H-3′. H-3′, 830 mg (1 eq, 4.3 mmol) was converted to the tertiary butyl dimethylsilyl protected intermediate H-4′ using tert-butyldimethylsilyl chloride, TBDMSC1 (1.56 g, 2.5 eq, 10.9 mmol), TEA (1.16 mL, 2.0 eq, 10.9 mmol in 8 mL DCM at room temperature, overnight, resulting in recovery of about 1.51 g (83%) for purified H-4′. Subsequent selective deprotections of H-4′, 600 mg (1 eq, 1.4 mmol) in THF (3 ml)/MeOH (2 ml)/1M LiOH (1 ml) at room temperature, overnight yielded −500 mg of H-5′. Intermediate H-10 was produced by O-acylation of H-9 using 3-(bis(2-((tert-butyldimethylsilyl)oxy)ethyl)amino)propanoic acid (H5′). H-5′ and H-9 were combined to produce intermediate H-10 (Step 5, Scheme 7). H-9, 150 mg (1.0 eq, 0.24 mmol) was acylated with H-5′ (0.36 mmol, 150 mg, 1.5 eq) using EDCI (0.36 mmol, 74 mg, 1.5 eq), DIPEA (0.36 mmol, 62 4, 1.5 eq), DMAP (0.048 mmol, 6 mg, 0.2 eq) in 1.0 mL DCM to afford 108 mg (44%) of crude H-10. Crude H-10, 108 mg (1.0 eq, 0.11 mmol) was deprotected in HF.pyridine (2.75 mmol, 200 4, 25 eq), in 2.0 mL THF yielding 41 mg (48%) of crude Lipid 13. Crude product from two batches was combined and purified twice using preparatory HPLC to isolate 71 mg of purified Lipid 13. (>99% pure by Reverse phase HPLC-ELSD using Durashell-C18, 4.6×50 mm, 3 uM, Cat# DC930505-0).

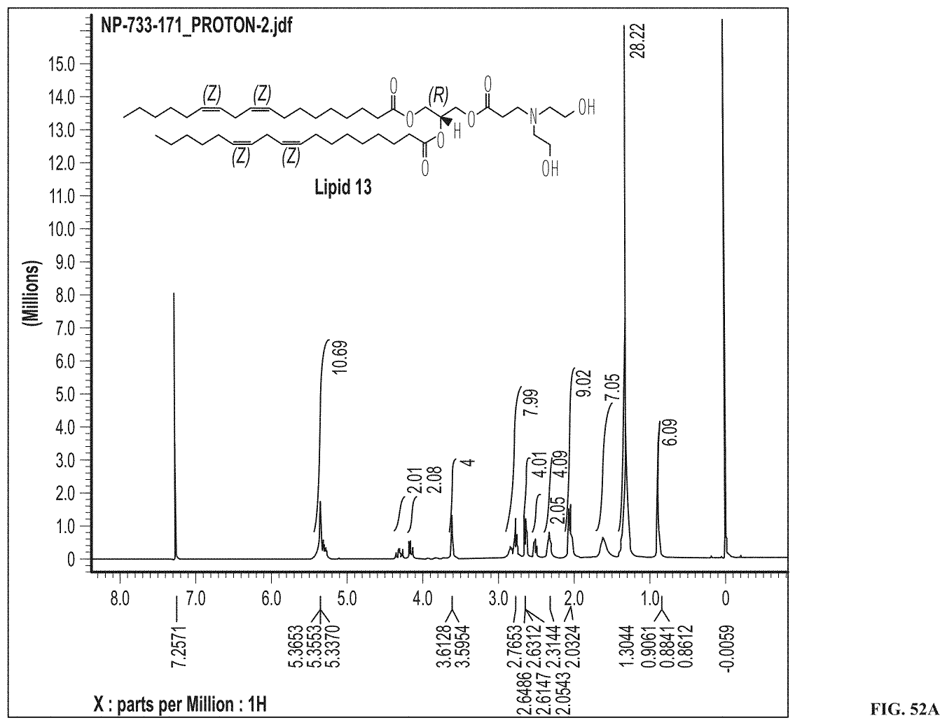

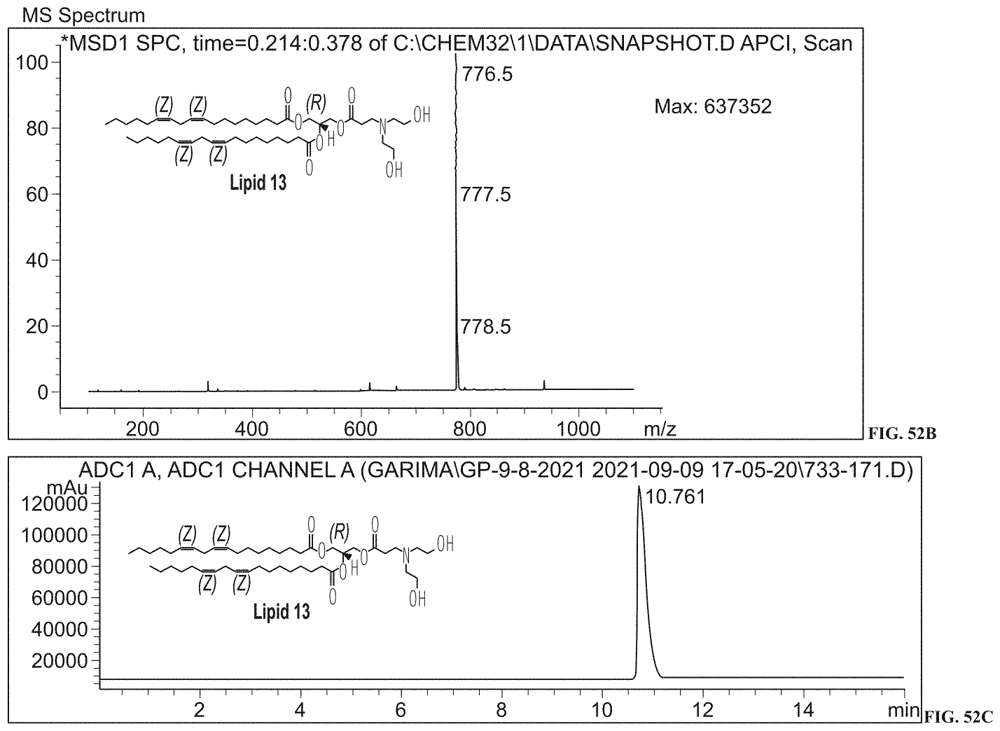

Purified Lipid 13 free base (C46H81N08, molecular weight 776.15 g/mol.) was characterized by proton NMR Spectroscopy (400 MHz) in CDCl3 and Mass Spectrometry to confirm structure (FIG. 52A and FIG. 52B) and by LC-ELSD to determine purity (FIG. 52C).

### Example 2—Preparation of LNPs by Vortex Mixing Using Exemplary Ionizable Lipids

Exemplary LNPs were created using cationic Lipid 2 and cationic Lipid 5 as synthesized in Example 1 as well as commercially available cationic Lipid 8 and cationic Lipid DLin-MC3-DMA (MedChemExpress, New Jersey, US; Catalog #HY-112758).

LNPs were created with an encapsulated mRNA payload and lipid blend by vortex mixing an aqueous mRNA solution and an ethanolic lipid solution. The mRNA (a 9:1 w/w mix of mRNA encoding eGFP and eGFP mRNA labeled with Cy5, TriLink Biotechnologies, California, US) was mixed with pH 4 acetate buffer to provide a final aqueous mRNA solution containing 133 pg/mL mRNA and 21.7 mM acetate buffer. The lipid components were dissolved in anhydrous ethanol at the relative ratios set forth in TABLE 3 below.

TABLE 3

| | | Ratio of Lipid to | Concentration | Theoretical |
|---|---|---|---|---|
| | | mRNA (nmol | in Lipid | LNP Lipid |
| | | lipid/100 μg | Solution | Composition |
| Lipid | Source | mRNA) | (mM) | (mol %) |
| Ionizable Cationic | — | 1,500 | 6 | 48.8 |
| Lipid | | | | |
| Cholesterol | Dishman | 1,200 | 4.8 | 39.0 |
| | Netherlands | | | |
| DSPC | Avanti Polar | 300 | 1.2 | 9.8 |
| | Lipids, Alabama, U.S. | | | |
| DMG-PEG | Avanti Polar | 75 | 0.3 | 2.4 |
| (2000) | Lipids, Alabama, U.S. | | | |

Briefly, the mRNA solution (375 μL) was transferred into a conical bottom centrifuge tube, and the lipid solution (125 μL) was rapidly added into the tube containing the mRNA solution (3:1 v/v ratio of mRNA solution to lipid solution). The tube containing the mixture was immediately capped and vortexed for 15 s at 2,500 rpm, followed by incubation at room temperature for not less than 5 min before proceeding to ethanol removal and buffer exchange.

Following mixing, ethanol removal and buffer exchange was performed on the resulting LNP suspension using a Sephadex G-25 resin packed SEC column (PD MiniTrap G-25, Cytiva, Massachusetts, U.S.), by gravity flow. Briefly, the SEC column was rinsed five times with 2.5 mL of exchange buffer (25 mM pH 7.4 HEPES buffer with 150 mM NaCl) before then loading 425 μL of LNP suspension. Once the LNP suspension fully moved into the resin bed, a 75 μL stacker volume of exchange buffer was applied to the column to achieve the specified target load volume of the column and maximize recovery, according to manufacturer specifications. After the stacker fully moved into the resin bed, the SEC column was transferred to a new centrifuge tube, and the LNP suspension was eluted by adding 1.0 mL of exchange buffer to the column. Eluate containing the LNPs in the exchange buffer was recovered and stored at 4° C. until further use.

### Example 3—Characterization of LNPs

This Example describes the characterization of LNPs produced in Example 2.

Samples of the LNPs produced in Example 2 were characterized to determine the average hydrodynamic diameter, zeta potential, and mRNA content (total and dye-accessible). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer.

RNA content of the nanoparticles is measured using Thermo Fisher Quant-iT RiboGreen RNA Assay Kit. Dye accessible RNA, which includes both non-incorporated RNA and RNA that is near the surface of the nanoparticle, is measured by diluting the nanoparticles to approximately 1 μg/mL mRNA using HEPES buffered saline, and then adding Quant-iT reagent to the mixture. Total RNA content is measured by diluting the particles to 1 μg/mL mRNA using HEPES buffered saline, disrupting the nanoparticles by heating them to 60° C. for 30 minutes in HEPES buffered saline containing 0.5% Triton, and then adding Quant-It reagent. RNA is quantified by measuring fluorescence at 485/535 nm, and concentration is determined relative to a contemporaneously run RNA standard curve. The results are set forth in TABLE 5.

TABLE 5

| | | | | Zeta | Zeta | Dye- |
|---|---|---|---|---|---|---|
| | | DLS Z-Avg. | | Potential | Potential | Accessible |
| Formulation | Ionizable | Diameter | | at pH 5.5 | at pH 7.4 | mRNA |
| No. | Lipid | (nm) | DLS PDI | (mV) | (mV) | (%) |
| 1 | Cationic | 120 | 0.19 | 21 | 5.4 | 16 |
| | Lipid 2 | | | | | |
| 2 | Cationic | 109 | 0.14 | 23 | 0.3 | 13 |
| | Lipid 5 | | | | | |
| 3 | Cationic | 91 | 0.12 | 20 | 0.9 | 6.3 |
| | Lipid 8 | | | | | |
| 4 | Cationic | 96 | 0.14 | 16 | −0.4 | 9.4 |
| | Lipid DLn- | | | | | |
| | MC3-DMA | | | | | |

### Example 4—Preparation of Fab Conjugates to Enable T-Cell Targeting

This Example describes the production of an exemplary lipid-immune cell targeting group conjugate.

An anti-CD3 Fab (hSP34 with mouse lambda and human lambda) (see amino acid sequences below) was conjugated to DSPE-PEG(2k)-maleimide via covalent coupling between the maleimide group and a C-terminal cysteine in the heavy chain (HC). Anti-CD3 Fab clone Hu291, anti-CD8 Fab clone TRX2, anti-CD8 Fab clone OKT8, a non-functional mutated OKT8 (mutOKT8), anti-CD4 Fab from Ibalizumab sequence, anti-CD5 Fab clone He3, anti-CD7 Fab clone TH-69, anti-CD2 Fab clone TS2/18.1, anti-CD2 Fab clone 9.6, anti-CD2 Fab clone 9-1 with human kappas were also conjugated using similar methods described herein. The protein (3-4 mg/mL), after buffer exchange into oxygen free, pH 7 phosphate buffer, was reduced in 2 mM TCEP in oxygen free pH 7 phosphate buffer for 1 hour at room temperature. The reduced protein was isolated using a 7 kDa SEC column to remove TCEP and buffer exchanged into fresh oxygen free pH 7 phosphate buffer.

The conjugation reaction was initiated by addition of a 10 mg/mL micellar suspension of DSPE-PEG-maleimide (Avanti Polar Lipids, Alabama, US) and 30 mg/mL DSPE-PEG-OCH3 (Avanti Polar Lipids, Alabama, U.S.) (1:1 to 1:3 weight ratio is used depending on protein) in oxygen free pH 5.7 citrate buffer (1 mM Citrate). Protein solution is concentrated to 3-4 mg/mL using a 10 kDa Regenerated Cellulose Membrane and subsequently buffer exchanged in oxygen free pH 7 phosphate buffer using a 40 kDa Size Exclusion Column. The conjugation reaction is carried out using 2-4 mg/mL protein and a 3.5 molar excess of maleimide at 37° C. for 2 hours followed by incubation at room temperature for an additional 12-16 hours.

The production of the resulting conjugate was monitored by HPLC and the reaction quenched in 2 mM cysteine. The resulting conjugate (DSPE-PEG(2k)-anti-hSP34 Fab) is isolated using a 100 kDa Millipore Regenerated Cellulose membrane filtration using pH 7.4 HEPES buffer saline (25 mM HEPES, 150 mM NaCl) buffer and stored at 4° C. prior to use. After quenching, the final micelle composition consists of a mixture of DSPE-PEG-Fab, DSPE-PEG-maleimide(cysteine terminated), and DSPE-PEG-OCH3. The ratio of the three components is DSPE-PEG-Fab: DSPE-PEG-maleimide(cysteine terminated): DSPE-PEG-OCH₃=1: 2.45: 3.45-10.35 (by mole)).

The resulting conjugate displayed comparable binding to recombinant Rhesus CD3 epsilon as the unconjugated anti-CD3 Fab by ELISA assay.

Anti-CD3 hSP34-Fab sequence:
hSP34 HC (SEQ ID NO: 1):
EVQLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRHG
NFGNSYISYWAYWGQGTLVTVSSASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSSDKTHTC
hSP34-mlam LC (mouse lambda) (SEQ ID NO: 2):
QTVVTQEPSLTVSPGGTVTLTCGSSTGAVTSGNYPNWVQQKPGQAPRGLIG
GTKFLAPGTPARFSGSLLGGKAALTLSGVQPEDEAEYYCVLWYSNRWVFGG
GTKLTVLGQPKSSPSVTLFPPSSEELETNKATLVCTITDFYPGVVTVDWKV
DGTPVTQGMETTQPSKQSNNKYMASSYLTLTARAWERHSSYSCQVTHEGHT
VEKSLSRADSS
SP34-hlam LC (human lambda) (SEQ ID NO: 3):
QTVVTQEPSLTVSPGGTVTLTCGSSTGAVTSGNYPNWVQQKPGQAPRGLIG
GTKFLAPGTPARFSGSLLGGKAALTLSGVQPEDEAEYYCVLWYSNRWVFGG
GTKLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGST
VEKTVAPAESS
Anti-CD3 Hu291-Fab sequence:
Hu291 HC (SEQ ID NO: 4):
QVQLVQSGAEVKKPGASVKVSCKASGYTFISYTMHWVRQAPGQGLEWMGYI
NPRSGYTHYNQKLKDKATLTADKSASTAYMELSSLRSEDTAVYYCARSAYY
DYDGFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICN
VNHKPSNTKVDKKVEPKSSDKTHTC
Hu 291 LC (SEQ ID NO: 5):
MDMRVPAQLLGLLLLWLPGAKCDIQMTQSPSSLSASVGDRVTITCSASSSV
SYMNWYQQKPGKAPKRLIYDTSKLASGVPSRFSGSGSGTDFTLTISSLQPE
DFATYYCQQWSSNPPTFGGGTKVEIKRTVAAPSVFIFPPSDEQLKSGTASV
VCLLNNFYPREAKVQWKVDNALQSGNSQESVTEQDSKDSTYSLSSTLTLSK
ADYEKHKVYACEVTHQGLSSPVTKSFNRGES
Anti-CD8 TRX2-Fab sequence:
TRX2 HC (SEQ ID NO: 6):
QVQLVESGGGVVQPGRSLRLSCAASGFTFSDFGMNWVRQAPGKGLEWVALI
YYDGSNKFYADSVKGRFTISRDNSKNTLYLQMNSLRAEDTAVYYCAKPHYD
GYYHFFDSWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYIC
NVNHKPSNTKVDKKVEPKSSDKTHTC
TRX2 LC (SEQ ID NO: 7):
DIQMTQSPSSLSASVGDRVTITCKGSQDINNYLAWYQQKPGKAPKLLIYNT
DILHTGVPSRFSGSGSGTDFTFTISSLQPEDIATYYCYQYNNGYTFGQGTK
VEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPV
TKSFNRGES
Anti-CD8 OKT8-Fab sequence:
OKT8 HC (SEQ ID NO: 8):
QVQLVQSGAEDKKPGASVKVSCKASGFNIKDTYIHWVRQAPGQGLEWMGRI
DPANDNTLYASKFQGRVTITADTSSNTAYMELSSLRSEDTAVYYCGRGYGY
YVFDHWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSSDKTHTC
OKT8 LC (SEQ ID NO: 9):
DIVMTQSPSSLSASVGDRVTITCRTSRSISQYLAWYQEKPGKAPKLLIYSG
STLQSGVPSRFSGSGSGTDFTLTISSLQPEDFATYYCQQHNENPLTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES
Anti-CD4 Ibalizumab-Fab sequence:
Ibalizumab HC (SEQ ID NO: 10):
QVQLQQSGPEVVKPGASVKMSCKASGYTFTSYVIHWVRQKPGQGLDWIGYI
NPYNDGTDYDEKFKGKATLTSDTSTSTAYMELSSLRSEDTAVYYCAREKDN
YATGAWFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYI
CNVNHKPSNTKVDKKVEPKSSDKTHTC
Ibalizumab LC (SEQ ID NO: 11):
DIVMTQSPDSLAVSLGERVTMNCKSSQSLLYSTNQKNYLAWYQQKPGQSPK
LLIYWASTRESGVPDRFSGSGSGTDFTLTISSVQAEDVAVYYCQQYYSYRT
FGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGES
anti-CD5 He3-Fab sequence:
He3 HC (SEQ ID NO: 12):
EIQLVQSGGGLVKPGGSVRISCAASGYTFTNYGMNWVRQAPGKGLEWMGWI
NTHTGEPTYADSFKGRFTFSLDDSKNTAYLQINSLRAEDTAVYFCTRRGYD
WYFDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSSDKTHTC
He3 LC (SEQ ID NO: 13):
DIQMTQSPSSLSASVGDRVTITCRASQDINSYLSWFQQKPGKAPKTLIYRA
NRLESGVPSRFSGSGSGTDYTLTISSLQYEDFGIYYCQQYDESPWTFGGGT
KLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES
anti-CD7 TH-69-Fab sequence:
TH-69 HC (SEQ ID NO: 14):
EVQLVESGGGLVKPGGSLKLSCAASGLTFSSYAMSWVRQTPEKRLEWVASI
SSGGFTYYPDSVKGRFTISRDNARNILYLQMSSLRSEDTAMYYCARDEVRG
YLDVWGAGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNH
KPSNTKVDKKVEPKSCDKTHTC
TH-69 LC (SEQ ID NO: 15):
DIQMTQTTSSLSASLGDRVTISCSASQGISNYLNWYQQKPDGTVKLLIYYT
SSLHSGVPSRFSGSGSGTDYSLTISNLEPEDIATYYCQQYSKLPYTFGGGT
KLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC
anti-CD2 TS2/18.1-Fab sequence:
TS2/18.1 HC (SEQ ID NO: 16):
EVQLVESGGGLVMPGGSLKLSCAASGFAFSSYDMSWVRQTPEKRLEWVAYI
SGGGFTYYPDTVKGRFTLSRDNAKNTLYLQMSSLKSEDTAMYYCARQGANW
ELVYWGQGTLVTVSAASTKGPSVFPLAPSSKSTSGGTAALGCLVKDYFPEP
VTVSWNSGALTSGVHTFPAVLQSSGLYSLSSVVTVPSSSLGTQTYICNVNH
KPSNTKVDKKVEPKSSDKTHTC
TS2/18.1 LC (SEQ ID NO: 17):
DIVMTQSPATLSVTPGDRVFLSCRASQSISDFLHWYQQKSHESPRLLIKYA
SQSISGIPSRFSGSGSGSDFTLSINSVEPEDVGVYFCQNGHNFPPTFGGGT
KLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES
anti-CD2 9.6-Fab sequence:
9.6 HC (SEQ ID NO: 18):
QVQLQQPGAELVRPGSSVKLSCKASGYTFTRYWIHWVKQRPIQGLEWIGNI
DPSDSETHYNQKFKDKATLTVDKSSGTAYMQLSSLTSEDSAVYYCATEDLY
YAMEYWGQGTSVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSSDKTHTC
9.6 LC (SEQ ID NO: 19):
NIMMTQSPSSLAVSAGEKVTMTCKSSQSVLYSSNQKNYLAWYQQKPGQSPK
LLIYWASTRESGVPDRFTGSGSGTDFTLTISSVQPEDLAVYYCHQYLSSHT
FGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGES
anti-CD2 9-1-Fab sequence:
9-1 HC (SEQ ID NO: 20):
QVQLQQPGTELVRPGSSVKLSCKASGYTFTSYWVNWVKQRPDQGLEWIGRI
DPYDSETHYNQKFTDKAISTIDTSSNTAYMQLSTLTSDASAVYYCSRSPRD
SSTNLADWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICN
VNHKPSNTKVDKKVEPKSSDKTHTC
9-1 LC (SEQ ID NO: 21):
DIVMTQSPATLSVTPGDRVSLSCRASQSISDYLHWYQQKSHESPRLLIKYA
SQSISGIPSRFSGSGSGSDFTLSINSVEPEDVGVYYCQNGHSFPLTFGAGT
KLELRRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES
mutOKT8-Fab sequence:
mutOKT8 HC (SEQ ID NO: 22):
QVQLVQSGAEDKKPGASVKVSCKASGFNIKDTYIHWVRQAPGQGLEWMGRI
DPANDNTLYASKFQGRVTITADTSSNTAYMELSSLRSEDTAVYYCGRGAGA
YVFDHWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSSDKTHTC
mutOKT8 LC (SEQ ID NO: 23):
DIVMTQSPSSLSASVGDRVTITCRTSRSISAALAWYQEKPGKAPKLLIYSG
STLQSGVPSRFSGSGSGTDFTLTISSLQPEDFATYYCQQHNENPLTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGES

### Example 5—Preparation of LNPs Containing T Cell Targeting Group

This Example describes the incorporation of an exemplary immune cell targeting conjugate into a preformed LNP.

LNPs from Example 3 and the conjugate from Example 4 were combined as shown in Table 6 in an Eppendorf tube and vortexed for 10 seconds at 2,500 rpm. The Eppendorf tubes were placed in the ThermoMixer at 37° C. at 300 rpm for 14 hours, and then stored at 4° C. until use.

TABLE 6

| nmol total | FAb | FAb | RNA | FAb | Fab | LNP |
|---|---|---|---|---|---|---|
| lipid/mg | target | mg/mg | mg/mL | mg/mL | mg/mL | mL/mL |
| RNA | g/mol lipid | RNA | LNP | Fab | LNP | Fab |
| 30,750 | 17 | 0.52275 | 1 | 1 | 0.52 | 1.91 |

This Example describes the incorporation of an immune cell targeting conjugate into a preformed LNP.

LNPs from Example 2 and conjugates (anti-CD3 (hSP34) and anti-CD8 (TRX-2) conjugates) were prepared using methods described in Example 4 were combined as shown in Table 6A in an Eppendorf tube and vortexed for 10 seconds at 2,500 rpm. The Eppendorf tubes were placed in the ThermoMixer at 37° C. at 300 rpm for 14 hours, and then stored at 4° C. until use. Alternatively, the Eppendorf tubes were placed in the ThermoMixer at 60° C. at 300 rpm for 30 minutes to 3 hours, followed by continued mixing at 4° C. and 300 rpm for an additional 12-24 hr and then stored at 4° C. until use.

TABLE 6A

| nmol | FAb | | | | | | Mol % |
|---|---|---|---|---|---|---|---|
| total | target | FAb | RNA | FAb | Fab | LNP | DSPE- |
| lipid/mg | g/mol | mg/mg | mg/mL | mg/mL | mg/mL | mL/mL | PEG |
| RNA | lipid | RNA | LNP | Fab | LNP | Fab | In LNP |
| 30,750 | 17 | 0.52275 | 0.45 | 1.46 | 0.235 | 6.2 | 0.47 |

### Example 6—Preparation of LNPs by Microfluidic Mixing Using Exemplary Ionizable Lipids

This example describes the preparation of LNPs using cationic Lipid 5 and cationic Lipid 8 by a microfluidic mixing method.

LNPs were created with an encapsulated mRNA payload and lipid blend by mixing an aqueous mRNA solution and an ethanolic lipid solution using an in-line microfluidid mixing process. The mRNA (a 9:1 w/w mix of mRNA encoding eGFP and eGFP mRNA labeled with Cy5, TriLink Biotechnologies, California, US) was mixed with pH 4 acetate buffer to provide a final aqueous mRNA solution containing 133 μg/mL mRNA and 21.7 mM acetate buffer. The lipid components were dissolved in anhydrous ethanol at the relative ratios set forth in TABLE 7 below.

TABLE 7

| | | Ratio of Lipid to | Concentration | Theoretical |
|---|---|---|---|---|
| | | mRNA (nmol | in Lipid | LNP Lipid |
| | | lipid/100 μg | Solution | Composition |
| Lipid | Source | mRNA) | (mM) | (mol %) |
| Ionizable Cationic | — | 1,500 | 6 | 48.8 |
| Lipid | | | | |
| Cholesterol | Dishman | 1,200 | 4.8 | 39.0 |
| | Netherlands | | | |
| DSPC | Avanti Polar | 300 | 1.2 | 9.8 |
| | Lipids, Alabama, US | | | |
| DMG-PEG | Avanti Polar | 75 | 0.3 | 2.4 |
| (2000) | Lipids, Alabama, US | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were then attached to the cartridge. The two solutions were then mixed at a 3:1 v/v ratio of mRNA solution (975 μL) to lipid solution (325 μL) at a total flow rate of 9 mL/min using the NanoAssemblr Ignite. The resulting suspension was incubated at room temperature for not less than 5 min before proceeding to ethanol removal and buffer exchange.

Following mixing, ethanol removal and buffer exchange was performed on the resulting LNP suspension using two Sephadex G-25 resin packed SEC columns (PD MiniTrap G-25, Cytiva, Massachusetts, US), by gravity flow. Briefly, the SEC columns were each rinsed five times with 2.5 mL of exchange buffer (25 mM pH 7.4 HEPES buffer with 150 mM NaCl) before then loading 450 μL of LNP suspension per column. Once the LNP suspension fully moved into the resin bed, a 50 μL stacker volume of exchange buffer was applied to each column to achieve the specified target load volume of the column and maximize recovery, according to manufacturer specifications. After the stacker fully moved into the resin bed, the SEC columns were transferred to new centrifuge tubes, and the LNP suspension was eluted by adding 1.0 mL of exchange buffer to each column. Eluate containing the LNPs in the exchange buffer was recovered from each column, combined into a single LNP batch, and stored at 4° C. until further use.

The resulting LNPs were characterized as described in Example 3. The results are summarized in TABLE 8 below. As seen in Table 8, the microfluidic process results in sub-100 nm particles exhibiting narrow polydispersity and good mRNA encapsulation (<20% dye accessible RNA).

TABLE 8

| | | | | Zeta | Zeta | Dye- |
|---|---|---|---|---|---|---|
| | | DLS Z-Avg. | | Potential at | Potential at | Accessible |
| Formulation | Ionizable | Diameter | | pH 5.5 | pH 7.4 | mRNA |
| No. | Lipid | (nm) | DLS PDI | (mV) | (mV) | (%) |
| 5 | Cationic | 52 | 0.11 | 24 | 3.8 | 19 |
| | Lipid 5 | | | | | |
| 6 | Cationic | 55 | 0.15 | 24 | 4.0 | 11 |
| | Lipid 8 | | | | | |

### Example 7—Characterization of LNPs pKa Using Toluidinyl-Naphthalene Sulfonate (TNS) Fluorescent Probe

This example describes the fluorescent dye based method used for measurement of the apparent pKa of the lipid nanoparticles. Apparent pKa determines the nanoparticle surface charge under physiological pH conditions, typically a pKa value in the endosomal pH range (6-7.4) results in LNPs that are neutral or slightly charged at plasma or the extracellular space (pH 7.4) and become strongly positive under acidic endosomal environments. This positive surface charge drives fusion of the LNP surface with negatively charged endosomal membranes resulting in destabilization and rupture of the endosomal compartment and LNP escape into the cytosolic compartment, a critical step in cytosolic delivery of mRNA and protein expression via engagement of the cells ribosomal machinery.

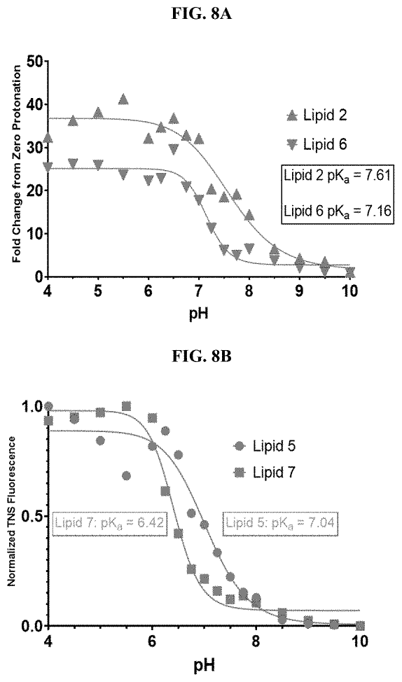

The apparent pKa of LNPs made using ionizable Lipids 2, 5 (synthesized as described in example 1), 6 and 7 (synthesized using method analogous to Lipid 2, 5, respectively, except using diethyl amine instead of dimethyl amine to incorporate the tertiary amine head group) were determined by 6-(p-Toluidino)-2-naphthalenesulfonic acid (TNS) fluorescence measurement in aqueous buffers covering a range of pH values (pH 4-pH 10). TNS is non-fluorescent when free in solution, but which fluoresces strongly when associated with a positively charged lipid nanoparticle. At a pH values below the pKa of the nanoparticle, positive LNP surface charge results in dye recruitment at the particle interface resulting in TNS fluorescence. At pH values above the LNP pKa no fluorescence is observed. The apparent pKa of the LNP is reported as the pH at which the fluorescence is at 50% of its maximum, as determined using a four-point logistic curve fit. Lipids 2 exhibited an apparent pKa of 7.5 and chemical modification of tertiary amine head group in Lipid 6 resulted in a pKa shift to lower values (Lipid 6 pKa˜7, FIG. 8A). Similarly, Lipid 5 exhibited an apparent pKa of 6.9 and chemical modification of tertiary amine head group in Lipid 7 resulted in a pKa shift to lower values (Lipid 7 pKa ˜6.3, FIG. 8B). As a result, both modifications were found to create LNPs potentially capable of improved ability to fuse with negatively charged endosomal membranes and result in improved cytosolic delivery of the mRNA payload.

### Example 8—In Vitro Transfection Protocol in Primary Human T-cells

This example describes the protocol used for in vitro LNP transfections in primary human T-cells. This method is used to assess LNP in vitro efficacy in relevant target cells (CD3+ T cells) by first transfecting the cells using LNPs loaded with mRNA encoding for a reporter gene, such as GFP mRNA, and then assessing transfection by measuring reporter gene expression by fluorescence-actuated cell sorting (FACS). Additionally, particle association with cells may be observed by in the same assay by labeling individual nanoparticle components, such as the mRNA, with a fluorescent dye, such as Cy5, and then observing cell/dye association by FACS.

CD3+ T cells were isolated from frozen peripheral blood mononuclear cells using an EasySep Human T Cell Isolation Kit on a RoboSep automated cell isolation system from STEMCELL. T cells were plated into a flat bottom 96-well plate in RPMI cell culture media supplemented with glutamax, 10% fetal bovine serum, and 40 ng/mL IL2. 100 μL of cell suspension was seeded per well at a density of 1M T cells/mL (100K T cells/well). Cells were allowed to rest for two hours in a 37° C. incubator, and then were transfected by gently adding 10 μL of a 22 μg/mL (by mRNA) nanoparticle suspension, resulting in a final mRNA concentration of 2 μg/mL. Cells were gently mixed with a pipette and then incubated for 24 hours in a 37° C. incubator. After incubation the cells were analyzed using an ThermoFisher Attune NXT flow cytometer. Cy5 was detected using a 638 nm laser with 670/14 nm filter. eGFP was detected using a 488 nm laser and a 530/30 nm filter. Data were analyzed using FlowJo software from BD biosciences. FACS data were first gated to exclude doublets and dead cells and then gated for GFP and Cy5. Gates for GFP+ and Cy5+were set such that a control sample (PBS treated T cells) was <0.1% positive.

### Example 9—Lipid 2, Lipid 6 LNP Properties and In Vitro Protein Expression in Primary Human T-Cells

This example describes the transfection ability of LNPs derived from Lipid 2 and Lipid 6. Nanoparticles are first produced using a mixing process followed by buffer exchange. Particles thus produced were subsequently tested in vitro in human CD3+ T cells to assess LNP association with cells, and expression of a reporter gene.

Lipid 2 and Lipid 6 LNPs encapsulating a 90-10 (w/w) mixture of GFP-mRNA and Cyanine-5 dye labelled mRNA (TriLink Biotechnologies Inc.) were prepared using the mixing process described in Example 6, the buffer exchange process described below in Example 21. Both formulations resulted in particles exhibiting hydrodynamic diameters in the sub-150 nm range and moderate polydispersities, as well as good mRNA encapsulation and recovery (<25% dye accessible mRNA and >80% encapsulated mRNA was recovered using the Triton-deformulation procedure described in Example 3).

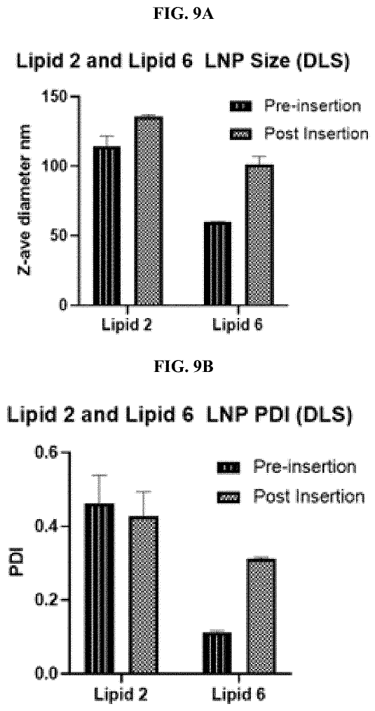

As seen in FIGS. 9A and 9B and Table 9, moderate changes in particle size and PDI were observed upon insertion of an anti-CD3 hSP34-PEG2k-DSPE conjugation using the insertion procedure described in Example 4. The resulting targeted LNPs were evaluated in primary human T-cells using the in vitro transfection protocol described in example 8. As seen in FIG. 11, both formulations were well tolerated by T-cells below 0.5 μg/mL dose (<40% drop in cell viability relative to PBS control) with Lipid 6 LNPs resulting in moderately higher viability at higher dose of 2 μg/mL. Dose dependent expression of GFP protein was observed with both ionizable lipids (2 and 6) as illustrated by high percentage of GFP+ cells and strong GFP MFI values. As illustrated by the Cy5+ and Cy5 MFI values, both formulations were equally associated with cells suggesting the conjugate insertion process was not dependent of the ionizable lipid chemistry. Both ionizable lipids (2 and 6) resulted in acceptable levels of mRNA encapsulation (<30% dye accessible RNA and >60% total mRNA recovery).

TABLE 9
Lipid 2 and Lipid 6 LNP mRNA content

| | Thoretical | Measured total | Ribogreen Dye | % |
|---|---|---|---|---|
| | total | (Triton method) | Accessible | Dye |
| Ionizable | mRNA | mRNA | mRNA | Accessible |
| Lipid | (μg/mL) | (μg/mL) | (μg/mL) | mRNA |
| Lipid 2 | 45 | 30 | 6 | 20% |
| Lipid 6 | 45 | 40 | 6 | 15% |

These findings demonstrate that lipid nanoparticles made using alternative ionizable lipids 2 and 6 may effectively encapsulate mRNA and transfect T cells in vitro.

### Example 10—Lipid 5, Lipid 7 LNP Properties and In Vitro Protein Expression in Primary Human T-Cells

This example describes the transfection ability of LNPs derived from Lipid 5 and Lipid 7. Nanoparticles are first produced using a mixing process followed by buffer exchange. Particles thus produced were subsequently tested in vitro in human CD3+ T cells to assess LNP association with cells, and expression of a reporter gene.

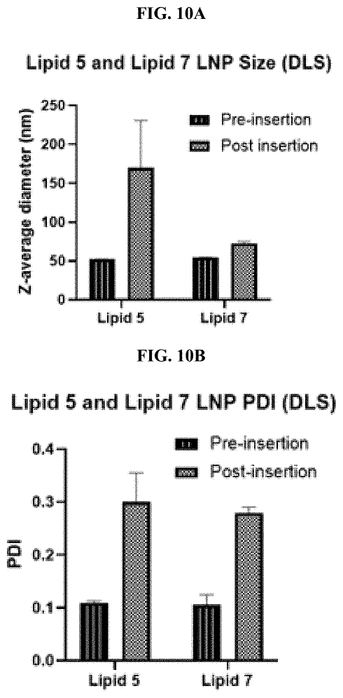

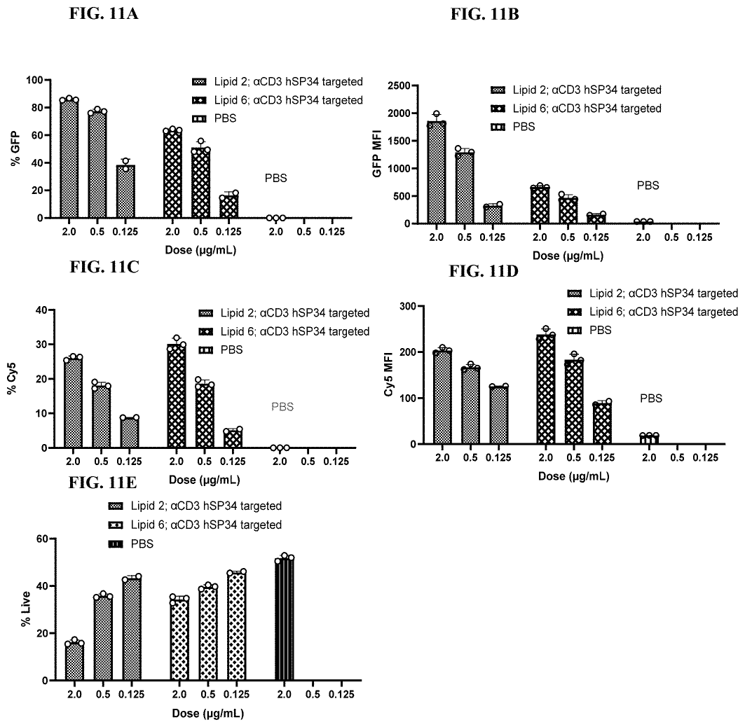

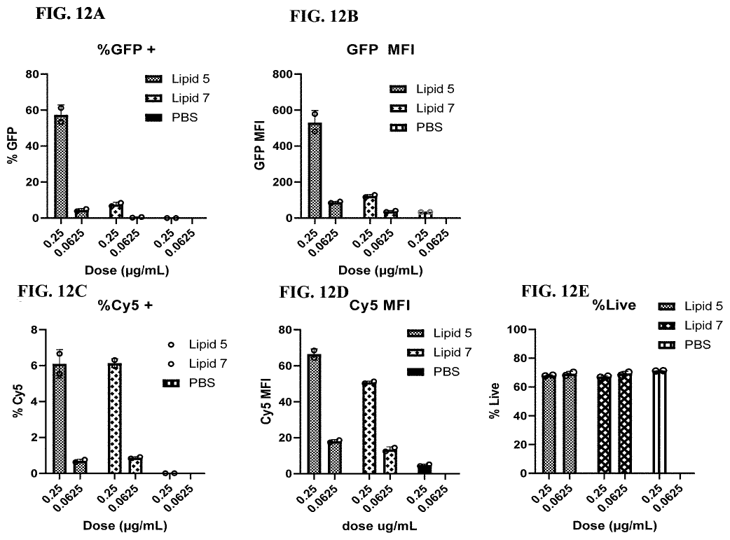

Lipid 5 and Lipid 7 LNPs encapsulating a 90-10 (w/w) mixture of GFP-mRNA and Cyanine-5 dye labelled mRNA (TriLink Biotechnologies Inc.) were prepared using the mixing process described in Example 6, the buffer exchange process described below in Example 21. Both formulations resulted in particles exhibiting hydrodynamic diameters in the sub-150 nm range and moderate polydispersities, as well as good mRNA encapsulation and recovery (<25% dye accessible mRNA and >80% encapsulated mRNA was recovered using the Triton-deformulation procedure described in Example 3). As seen in FIGS. 10A and 10B and Table 10, Lipid 5 LNP exhibited a larger change in hydrodynamic diameter (relative to Lipid 7 LNP) upon insertion of an anti-CD3 hSP34-PEG2k-DSPE conjugate using the insertion procedure described in Example 4. The resulting targeted LNPs were evaluated in primary human T-cells using the in vitro transfection protocol described in example 8. As seen in FIGS. 12A-12E, both formulation were well tolerated by T-cells at and below 0.5 μg/mL dose (minimal drop in cell viability was observed relative to the PBS control). As illustrated by the Cy5+ and Cy5 MFI values, both formulations were equally associated with cells suggesting the conjugate insertion process is not dependent on the ionizable lipid chemistry.

Dose dependent expression of GFP protein was observed with both ionizable lipids (5 and 7) as illustrated by similar % GFP+ and GFP MFI values (FIGS. 12A and B). Both formulations resulted in similar levels of cell association as illustrated by similar % Cy5+ and Cy5 MFI values (FIGS. 12C and D). However, Lipid 7 LNP formulation (apparent pKa ˜6.4) exhibited significantly lower level of GFP protein expression relative to Lipid 5 LNP formulation (apparent pKa ˜7) suggesting relatively poor cytosolic access with Lipid 7 LNPs. Both ionizable lipids (5 and 7) resulted in acceptable levels of mRNA encapsulation (<30% dye accessible RNA and >60% total mRNA recovery).

TABLE 10
Lipid 5 and Lipid 7 LNP mRNA content

| | Theoretical | Measured total | Ribogreen Dye | % |
|---|---|---|---|---|
| | total | (Triton method) | Accessible | Dye |
| Ionizable | mRNA | mRNA | mRNA | Accessible |
| Lipid | (μg/mL) | (μg/mL) | (μg/mL) | mRNA |
| Lipid 5 | 50 | 43 | 10 | 23% |
| Lipid 7 | 50 | 43 | 6 | 14% |

### Example 11—Lipid 5, Lipid 8 and DLn-MC3-DMA LNP Properties In Vitro Protein Expression in Primary Human T-Cells

This example compares the GFP protein expression resulting from LNP's derived from Lipid 5 and Lipid 8 to LNPs made using DLn-MC3-DMA. Nanoparticles are first produced using a mixing process followed by buffer exchange. Particles thus produced were subsequently tested in vitro in human CD3+ T cells to assess LNP association with cells, and expression of a reporter gene.

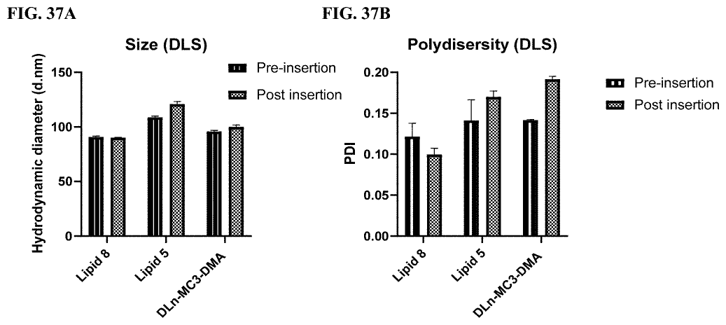

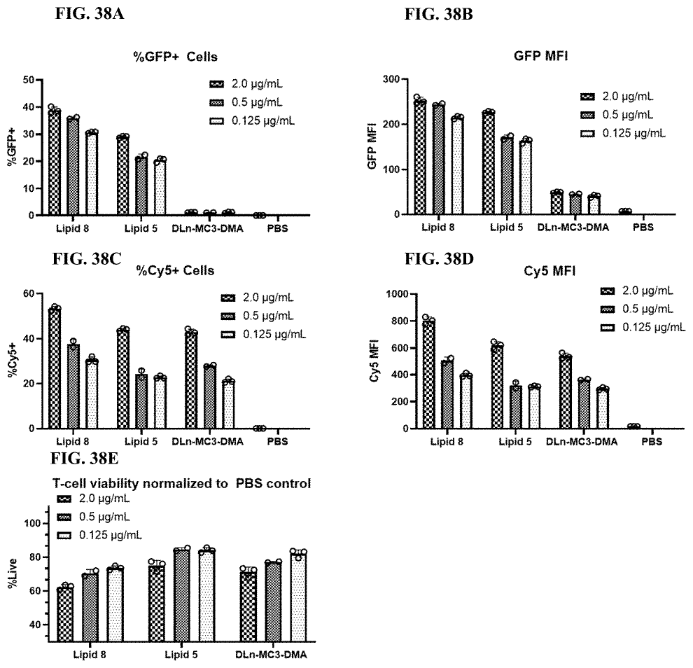

Lipid 5, Lipid 8 and DLn-MC3-DMA LNPs encapsulating a 90-10 (w/w) mixture of GFP-mRNA and Cyanine-5 dye labelled mRNA (TriLink Biotechnologies Inc.) were prepared using the vortexer mixing and buffer exchange process described in Example 4. All three formulations resulted in particles exhibiting hydrodynamic diameters in the sub-150 nm range and moderate polydispersities (FIG. 37A and B). Additionally, all three formulations made using the vortexer method using Lipids 5, 8 and DLn-MC3-DMA exhibited acceptable mRNA encapsulation (<30% dye accessible mRNA) and moderate mRNA recovery (>60% encapsulated mRNA recovered using the Triton-deformulation procedure described in Example 3). As seen in FIG. 37, the insertion of an anti-CD3 hSP34-PEG2k-DSPE conjugation using the insertion procedure described in Example 4 resulted in only slight increases in hydrodynamic diameter and PDI. The resulting targeted LNPs were evaluated in primary human T-cells using the in vitro transfection protocol described in example 8. As seen in FIG. 38, all three formulations were well tolerated by T-cells below 0.5 μg/mL dose (<40% drop in cell viability relative to PBS control) with Lipid 8 LNPs exhibiting slightly lower viability at higher dose of 2 μg/mL (FIG. 38E). Dose dependent expression of GFP protein was observed with ionizable Lipids 2 and 8 as illustrated by high percentage of GFP+ cells and strong GFP MFI values (FIGS. 38A, B). However, DLn-MC3-DMA (also shown in FIGS. 38A, B) LNPs failed to express GFP protein. Comparison of the Cy5+ and Cy5 MFI values in Lipid 2 or Lipid 8 formulations with the corresponding levels in the DLn-MC3-DMA LNP transfections (FIGS. 38C and D) indicates that all three formulation associated equally with T-cells suggesting that the efficiency of the antibody insertion process is independent of ionizable lipid chemistry. Poor performance of DLn-MC3-DMA LNPs may be attributed to this formulation resulting in poor cytosolic availability of mRNA in primary human T-cells.

TABLE 11A
Lipid 5, Lipid 8 and DLn-MC3-DMA LNP mRNA content

| | Theoretical | Measured total | Ribogreen Dye |
|---|---|---|---|
| | total | (Triton method) | Accessible |
| | mRNA | mRNA | mRNA |
| Ionizable Lipid | (ug/mL) | (ug/mL) | (ug/mL) |
| Lipid 8 | 42.5 | 30 | 3 |
| DLn-MC3-DMA | 42.5 | 33 | 2 |
| Lipid 5 | 42.5 | 27 | 4 |

### Example 12—In Vitro Protein Expression—CD3 and Cd8 Targeted Cy5/GFP LNP with Various Densities

This Example describes targeting human CD8 T cells with either anti-CD3 or anti-CD8 Fabs post-inserted into Cy5/GFP mRNA LNPs at various Fab densities and their effect on particle binding, transfection, viability, CD69 upregulation and IFNγ secretion.

LNPs were prepared using the mixing process described in Example 6, the buffer exchange process described in Example 21. hSP34 and TRX2 Fab-lipid conjugates generated from methods described in Example 4 and a non-T cell specific anti-HER2 lipid-conjugate (Nellis D F, Ekstrom D L, Kirpotin D B, Zhu J, Andersson R, Broadt T L, Ouellette T F, Perkins S C, Roach J M, Drummond D C, Hong K, Marks J D, Park J W and Giardina S L (2005) Preclinical manufacture of an anti-HER2 scFv-PEG-DSPE, liposome-inserting conjugate. 1. Gram-scale production and purification. Biotechnol Prog 21:205-220) were post-inserted at various densities (SP34 0.6-17 g/mol; TRX2 3-9 g/mol; anti-HER2 17 g/mol) into LNPs containing Lipid 8, Cy5 labeled GFP mRNA and GFP mRNA (1:9 Cy5:GFP mass ratio) by adding conjugate and LNPs together and heating the solution without mixing in a thermal cycler at 60 C for 60 min and cooled to 4° C. for 3-5 min. Particles were then diluted to 25 μg/mL mRNA with Hepes Buffered Saline pH 7.4 prior to transfection of human CD8 T cells using a method similar to Example 8 where the final concentration was approximately 2.5 μg/mL mRNA for approximately 24 hr (or 10 μL of Hepes Buffered Saline pH 7.4 buffer was added as a mock transfection). After transfection, LNP binding efficiency (Cy5) and transfection efficiency (GFP) was evaluated by flow cytometry. Supernatants were measured for human IFNγ concentration using a commercially available ELISA kit under manufacturers recommended conditions (R&D Systems Duoset).

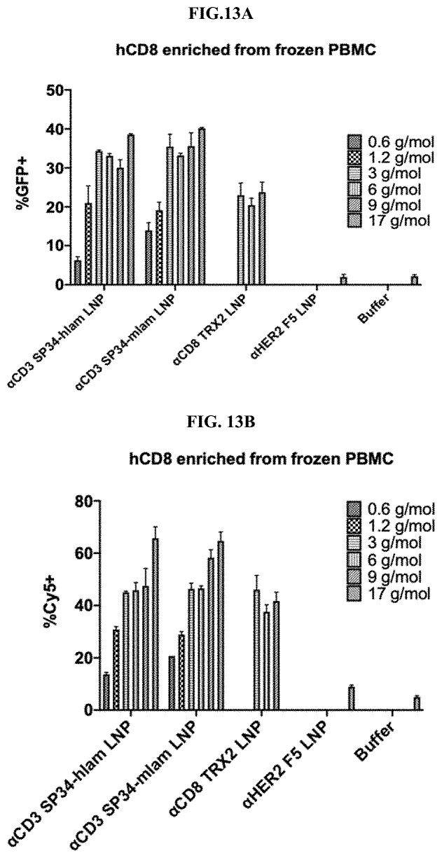

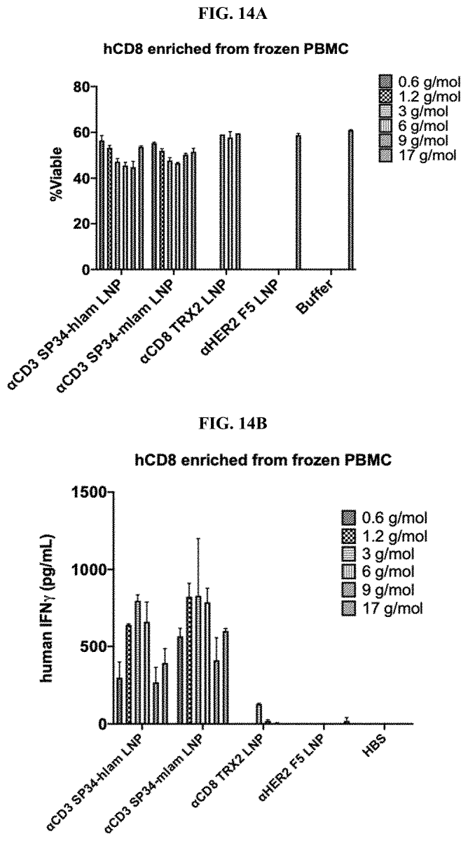

High transfection (FIG. 13A) and binding (FIG. 13B) was observed for SP34 and TRX2 Fab post-inserted LNPs with a broad range of Fab densities mediating transfection while non-specific HER2 targeted LNPs exhibited low binding and transfection. Some loss in cell viability was observed (FIG. 14A) using hSP34 CD3 targeted LNPs while TRX2 CD8 targeted LNPs had similar viabilities to non-specific HER2 targeted LNPs and untransfected (mock buffer added) T cells. Additionally, hSP34 (with mouse or human lambda) CD3-targeted LNPs mediated high IFNγ secretion (FIG. 14B) relative to TRX2 CD8-targeted, HER2-targeted LNPs and the mock T cell transfection conditions.

This study shows that CD8 T cells can be efficiently transfected with CD3 and/or CD8 targeted LNPs using a broad range of Fab densities in all cases. Additionally, using anti-CD8 Fab can mediate efficient LNP transfection while avoiding high CD69 upregulation and IFNγ secretion.

### Example 13—In Vitro Protein Expression—CD3, CD8 and CD3/CD8 Targeted TTR-023 LNP with Various Densities

This example describes targeting human CD3 T cells with either anti-CD3 or anti-CD8 Fabs post-inserted into anti-CD20 CAR (TTR-023) mRNA LNPs at various Fab densities and their effect on transfection, viability, CD69 upregulation and IFNγ secretion.

LNPs were prepared using the mixing process described in Example 6, the buffer exchange process described below in Example 21. Using methods similar to Example 12, hSP34 and TRX2 Fab-lipid conjugates and a non-T cell specific anti-HER2 lipid-conjugate (Nellis D F, Ekstrom D L, Kirpotin D B, Zhu J, Andersson R, Broadt T L, Ouellette T F, Perkins S C, Roach J M, Drummond D C, Hong K, Marks J D, Park J W and Giardina S L (2005) Preclinical manufacture of an anti-HER2 scFv-PEG-DSPE, liposome-inserting conjugate. 1. Gram-scale production and purification. Biotechnol Prog 21:205-220) were post-inserted at various densities (SP34 0.25-17 g/mol; TRX2 0.25-9 g/mol; SP34+TRX2 0.25-9 g/mol each conjugate; anti-HER2 17 g/mol) into LNPs containing Lipid 8 and anti-CD20 targeting CART mRNA. Transfections were performed with human CD3 T cells at approximately 2.5 μg/mL mRNA for approximately 24 hr. For FACS analysis T cells were stained with M1 antibody (Sigma, F3040) that bind a N-terminus FLAG-tag variant sequence on the TTR-023 CAR (sequence provided below) in addition to staining for CD69 (Biolegend, 310930) and CD4 (Biolegend, 344648) to differentiate CD8 from CD4 cells.

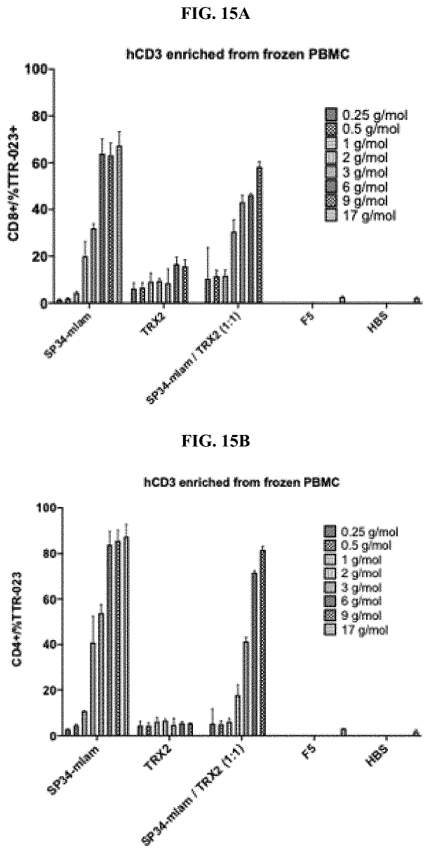

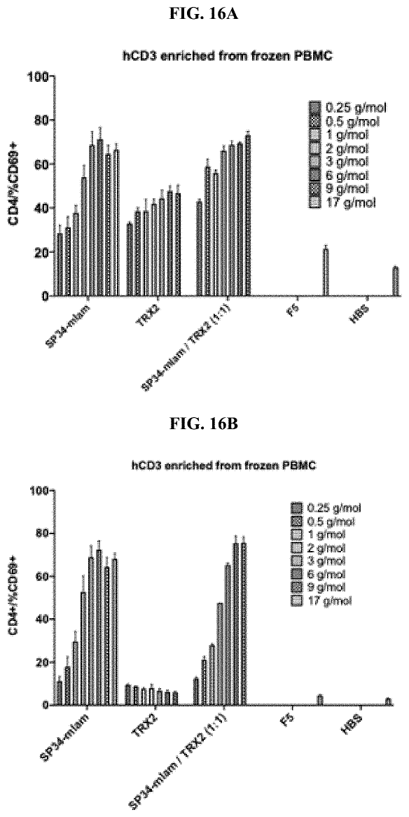

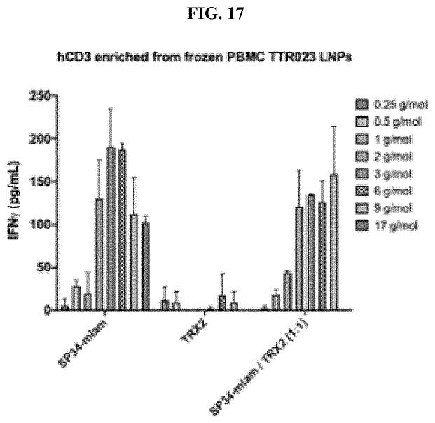

High transfection efficiency (FIGS. 15A and 15B) was observed between 2-17 g/mol Fab for hSP34 alone or co-targeted with TRX2 and transfection was detected over background for TRX2 at 6-9 g/mol Fab. Consistent with the transfection results, CD69 was upregulated in a target specific manner where CD3 targeting by hSP34 Fab induced CD69 expression on both CD8 and CD4 cells while CD8 targeting by TRX2 mediated CD69 expression on CD8 cells only (FIGS. 16A and 16B). CD8-targeted LNPs with TRX2 Fab induced low levels of IFNγ secretion (FIG. 17) relative to CD3 and CD3/CD8 targeted LNPs despite observable CD69 upregulation on CD8 T cells.

This study shows that both CD4 and CD8 T cells can be efficiently transfected with CD3-targeted and CD3/CD8-targeted LNPs and CD8 cells can be specifically (avoidance of CD4 transfection) transfected with CD8-targeted LNPs using a broad range of Fab densities in all cases. Additionally, using anti-CD8 Fab can mediate efficient transfection with CAR mRNA while avoiding high CD69 upregulation and IFNγ secretion.

TTR-023 anti-CD20 (Leu-16) CAR sequence
(including leader) (SEQ ID NO: 24):
METDTLLLWVLLLWVPGSTGDYKAKEVQLQQSGAELVKPGASVKMSCKASG
YTFTSYNMHWVKQTPGQGLEWIGAIYPGNGDTSYNQKFKGKATLTADKSSS
TAYMQLSSLTSEDSADYYCARSNYYGSSYWFFDVWGAGTTVTVSSGGGSGG
GSGGGGSSDIVLTQSPAILSASPGEKVTMTCRASSSVNYMDWYQKKPGSSP
KPWIYATSNLASGVPARFSGSGSGTSYSLTISRVEAEDAATYYCQQWSFNP
PTFGGGTKLEIKGGGGSAAAIEVMYPPPYLDNEKSNGTIIHVKGKHLCPSP
LFPGPSKPFWVLVVVGGVLACYSLLVTVAFIIFWVRSKRSRLLHSDYMNMT
PRRPGPTRKHYQPYAPPRDFAAYRSRVKFSRSAEPPAYQQGQNQLYNELNL
GRREEYDVLDKRRGRDPEMGGKPRRKNPQEGLYNELQKDKMAEAYSEIGMK
GERRRGKGHDGLYQGLSTATKDTYDALHMQALPPR
Corresponding nucleic acid sequence (SEQ ID NO:
25):
atggagaccgacaccctgttgctttgggtactgttactttgggtgcccgga
tctaccggtgattacaaggccaaggaggtgcagctgcagcagagcggagcc
gagctggtgaagccaggcgcttccgtgaagatgtcttgtaaggcctccggc
tacacattcaccagctacaatatgcactgggtaaagcagactccggggcag
ggcctggagtggataggtgccatctaccctggcaacggcgacaccagctac
aaccagaagtttaaggggaaggctactctaacagcggacaagtcgtcctct
accgcctacatgcaactcagctccctgacgagcgaggactccgcggactac
tactgtgcccgctccaactactacggctctagctattggttcttcgacgtg
tggggcgctggaacgaccgtgaccgtgtcttccggtggaggttccgggggc
ggaagcggcggtggcggcagttcggacategtgctgacccagagccctgcc
atcctgtccgcttccccgggggagaaagttacgatgacctgccgagcgagc
tccagtgtcaactacatggattggtaccagaagaagcccggcagcagtccc
aagccgtggatttacgctactagcaacctggcgtccggtgtcccggctcgc
ttctcaggttctggctcgggtactagttattcattaaccatttctcgcgtg
gaggctgaggacgctgccacctactactgccaacagtggtctttcaaccct
cccactttcggaggcggcaccaagctcgagatcaagggcgggggtggctcc
gcagcagccattgaggtgatgtatcctcctccctatttggacaacgagaag
tcaaatggcaccatcatccacgttaagggcaagcacctgtgcccatctccc
ctgttcccaggcccctctaagcccttctgggtcctggtggtggtcggcggc
gtcctggcatgttactctctgctggtgaccgtcgcgttcatcatcttttgg
gtccggtccaagcgcagccgcctgctccactccgactacatgaatatgact
cctcgtaggcccggtccaacccgcaagcactaccagccgtacgcgccgccc
agagactttgctgcttaccgatccagagtgaaattttctaggtcggccgaa
cctcccgcatatcagcagggccagaaccagctgtacaacgaactcaacttg
ggacggcgcgaggaatacgatgtgctggataaacgccgtggccgcgatccc
gagatgggcgggaagccacgtcgcaaaaaccctcaggagggcctttacaac
gagttgcagaaggacaaaatggcggaggcctactccgagatcggaatgaag
ggggagcgccggcgcggcaaagggcatgacggcctctaccagggcctgtcc
acagccacgaaagacacctatgacgccctgcatatgcaggccctgcccccg
cgctgataatga

### Example 14—In Vitro Protein Expression—Cd3 and Cd8 Targeted with Other Clones

This example describes targeting human CD8 T cells with either anti-CD3 or anti-CD8 Fabs post-inserted into Cy5/GFP mRNA LNPs at various Fab densities and their effect on particle binding, transfection, viability, CD69 upregulation and IFNγ secretion.

LNPs were prepared using the mixing process described in Example 6, the buffer exchange process described in Example 21. Using methods similar to Example 12, hSP34, Hu291, TRX2, OKT8 Fab-lipid conjugates and a non-T cell specific anti-HER2 lipid-conjugate (Nellis D F, Ekstrom D L, Kirpotin D B, Zhu J, Andersson R, Broadt T L, Ouellette T F, Perkins S C, Roach J M, Drummond D C, Hong K, Marks J D, Park J W and Giardina S L (2005) Preclinical manufacture of an anti-HER2 scFv-PEG-DSPE, liposome-inserting conjugate. 1. Gram-scale production and purification. Biotechnol Prog 21:205-220) were post-inserted at various densities (Table 11) into LNPs containing Lipid 8 and Cy5/GFP mRNA. Transfections were performed with human CD8 T cells at approximately 2.5 μg/mL mRNA for approximately 24 hr.

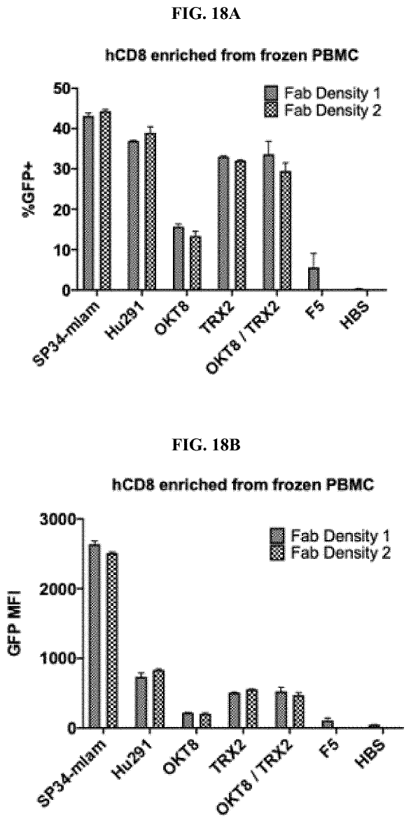

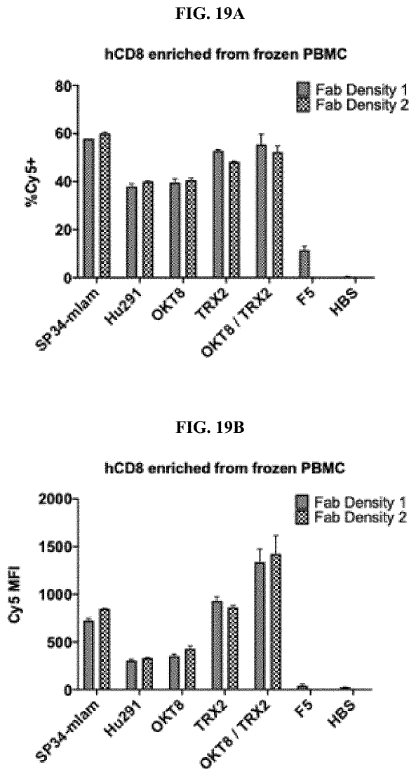

SP34 mediated higher % transfection (FIG. 18A) and substantially higher GFP expression levels (FIG. 18B) quantified by mean fluorescence intensity, MFI) than Hu291 despite both clones showing high levels of % Cy5+ T cells and binding the same target, CD3 (FIGS. 19A and 19B). Similarly for CD8 targeting, TRX2 mediated higher transfection than OKT8 by both metrics of % GFP+ and MFI (FIGS. 18A and 18B) despite both clones mediating high levels of particle binding measured by % Cy5+(FIG. 19A). Additionally, combining OKT8 and TRX2 increased the amount of particle binding (FIG. 19B) while providing no enhancement of transfection (FIGS. 18A and 18B).

This data indicates that the epitope that the Fab binds on the target protein may be important in determining its ability to mediate efficient particle uptake, transfection and translation and that efficient binding to the target does not guarantee efficient transfection.

TABLE 11
Target post-insertion LNP Fab densities

| Fab clone | Fab density 1 (g/mol) | Fab density 2 (g/mol) |
|---|---|---|
| SP34-mlam | 12 | 17 |
| Hu291 | 6 | 12 |
| OKT8 | 3 | 6 |
| TRX2 | 3 | 6 |
| OKT8/TRX2 | 3 | 6 |
| F5 | 17 | — |

### Example 15—In Vitro Protein Expression—CD3, CD8, CD4 and CD8/CD4 Targeted

This example shows targeting human CD3 T cells with either anti-CD3, anti-CD8 anti-CD4 or anti-CD8 and anti-CD4 Fabs post-inserted into Cy5/GFP mRNA LNPs at various Fab densities and their effect on particle binding, transfection, viability, CD69 upregulation and IFNγ secretion.

LNPs were prepared using the mixing process described in Example 6, the buffer exchange process described in Example 21. Using methods similar to Example 12, hSP34, TRX2, and Ibalizumab Fab-lipid conjugates and a non-T cell specific anti-HER2 lipid-conjugate were post-inserted at various densities (specified in FIGS. 20A and 20B into LNPs containing Lipid 8 and Cy5/GFP mRNA. Transfections were performed with human CD3 T cells at approximately 2.5 μg/mL mRNA for approximately 24 hr and stained for CD69 (Biolegend, 310930) and CD4 (Biolegend, 344648) to differentiate CD8 from CD4 cells by FACS analysis.

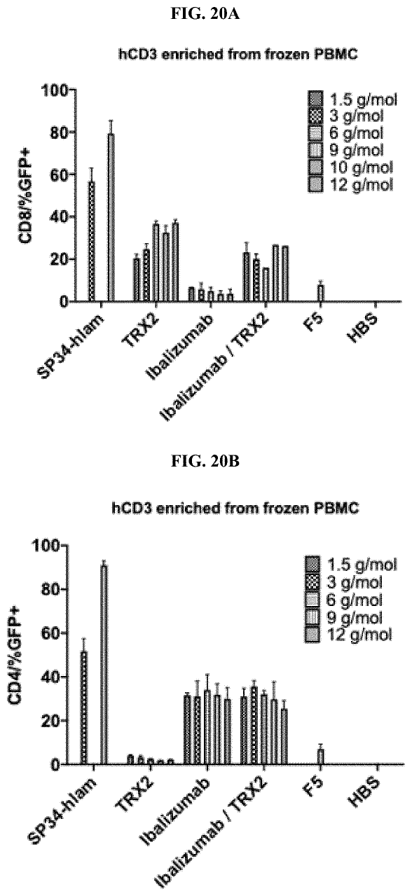

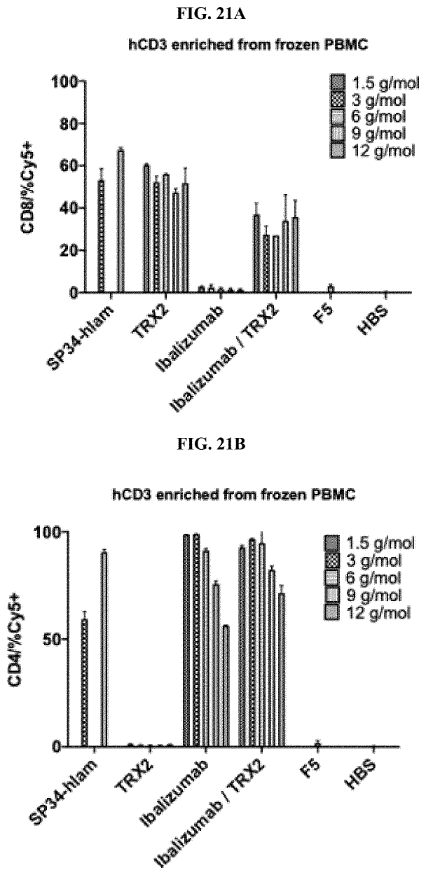

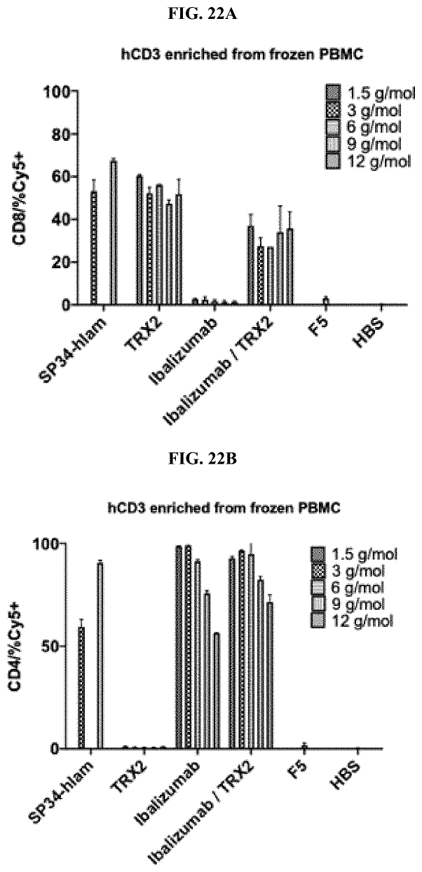

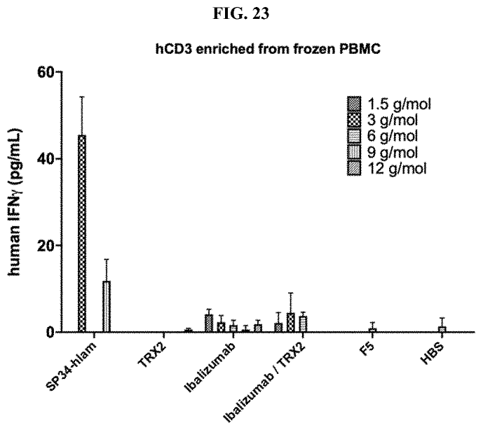

Consistent with previous results, hSP34 and TRX2 mediated specific LNP binding and transfection to CD3 and CD8 cells respectively (FIGS. 20A and 20B and FIGS. 21A 21B). A CD4 targeting Fab based on the V_{H} and V_{L} sequences of Ibalizumab mediated high binding and transfection of CD4 T cells while displaying minimal off-target binding and transfection of CD8 T cells. When TRX2 and Ibalizumab Fabs were post inserted into the same LNPs, high levels of binding and transfection were observed in both CD4 and CD8 cells using a broad range of Fab densities. While hSP34 drive high levels of CD69 upregulation (FIGS. 22A and 22B), TRX2 alone, Ibalizumab-Fab alone and TRX2 combined with Ibalizumab-Fab mediated much lower levels of CD69. In addition, SP34 drove higher levels of IFNγ (FIG. 23) than TRX2, Ibalizumab-Fab or the combination thereof.

This study shows that both CD4 and CD8 T cells can be efficiently transfected with CD3-targeted and CD8/CD4-targeted LNPs, CD8 cells can be specifically (avoidance of CD4 transfection) transfected with CD8-targeted LNPs and CD4 cells can be specifically (avoidance of CD8 transfection) transfected with CD4-targeted LNPs using a broad range of Fab densities in all cases. Additionally, using anti-CD8 Fab, anti-CD4 Fab or both anti-CD8 and anti-CD4 Fab can mediate efficient transfection while avoiding high CD69 upregulation and IFNγ secretion.

### Example 16—In Vitro Experimental Protocol for Whole Blood Transfection

This Example describes the method used to transfect immune cells in whole blood using Fab targeted mRNA LNPs.

Venous blood from healthy volunteers was anti-coagulated in heparin tubes (BD Biosciences #367526) and seeded at 50 μL in a 96-well round-bottom plate. Transfection of whole blood was carried out simply by adding nanoparticles containing 5 μg/mL mRNA to the cells and co-culturing at 37° C. until the time of analysis. To assess transfection efficiency, cells were analyzed 24-hours post-transfection by flow cytometry. LNPs used (with and without post-inserted targets) at 2.5 μg/mL:RDM073.23. Cells obtained from human blood were analyzed by flow cytometry. Prior to the analysis of whole blood transfection efficiency, red blood cells were lysed twice with VersaLyse Lysing Solution (Beckman Coulter #A09777) for 10 minutes at room temperature. Primary antibodies applied in the flow cytometry analysis of whole blood included the following: CD4-FITC (1:200) (BD Biosiences #555346), CD19-BUV395 (1:400) (BD Biosiences #563551), CD56-BUV737 (1:400) (BD Biosiences #741842). Fixable Viability Dye eFluor780 (eBiosciences #65-0865-14) was used to assess viability for all samples. For flow analysis, 1×10⁵ cells were Fc-blocked (BD Biosciences #564219) for 5 minutes on ice, followed by labeling dead cells with fixable viability dye eFluor780 and surface staining for 30 minutes on ice with specific antibodies.

Compensation for each fluorochrome was performed in the multicolor flow panels using positive and negative compensation beads. Fluorescence minus one (FMO) samples and unstained controls were included to determine the level of background fluorescence and to set the gates for the negative cell populations versus the positive cell populations.

All samples were acquired on a BD LSRFortessa X-20 (BD Biosciences) running FACSDIVA software (Becton Dickinson). All data collected were analyzed using FlowJo 10.7.1 software and GraphPad Prism version 9.0.

### Example 17—In Vitro-Cell Specific Protein Expression (Mcherry) in Human Whole Blood

This example describes specifically targeting human T cells in whole blood with anti-CD3, anti-CD8, anti-CD2, anti-CD5, anti-CD7 Fabs or combinations thereof post-inserted into mCherry mRNA LNPs at various Fab densities and their effect on transfection and CD69 upregulation secretion.

LNPs were prepared using the vortex mixing process described in Example 2 using the component ratios described in Table 12 below. Conjugate from a process described in Example 4 was post-inserted after particle formation). Particle properties were characterized using methods described in Example 3 and are described below in Table 13.

TABLE 12

| | nmol lipid per 100 μg mRNA | | | | |
|---|---|---|---|---|---|
| | | | | DMG- | |
| Batch | Lipid 8 | Cholesterol | DSPC | PEG2000 | Conjugate |
| 73.23 | 1500 | 1200 | 300 | 75 | 0.1-0.6* |

*depending on specific conjugate. TRX2 used in 0.6 nmol per 100 μg mRNA.

TABLE 13

| Batch | Size (nm) | PDI | EE (%) | Zeta at pH 7.4 (mV) |
|---|---|---|---|---|
| 73.23 | 123.6 | 0.217 | 93.2 | N.D. |

Using the methods described in Example 16, the transfection efficiency with the classic ionosphere formulation with Fab clones hSP34 (anti-CD3), TRX2 (anti-CD8), He3 (anti-CD5), anti-CD2 (TS2, 9.6 or 9-1), anti-CD7 (TH-69) or mutOKT8, post-inserted respectively (densities described in Table 14), and combinations of these, directly in human whole blood. LNPs with Lipid 8 were transfected in WB with 2.5 μg/mL mCherry mRNA for 24 hours.

TABLE 14

| Conjugate 1 | Conjugate 2 | Density (g/mol) | Target 1 | Target 2 |
|---|---|---|---|---|
| hSP34 | — | 9.00 | CD3 | — |
| TRX2 | — | 9.00 | CD8 | — |
| TRX2 | hSP34 | 9.00 | CD8 | CD3 |
| He3 | — | 3.00 | CD5 | — |
| He3 | TRX2 | 3.00 | CD5 | CD8 |
| TS2/18.1 | — | 1.50 | CD2 | — |
| 9.6 | — | 1.50 | CD2 | — |
| TS2/18.1 | TRX2 | 1.50 | CD2 | CD8 |
| 9.6 | TRX2 | 1.50 | CD2 | CD8 |
| TH-69 | — | 3.00 | CD7 | — |
| TH-69 | TRX2 | 3.00 | CD7 | CD8 |
| 9-1 | TS1/18.1 | 1.50 | CD2 | CD2 |
| 9-1 | 9.6 | 1.50 | CD2 | CD2 |
| He3 | TH-69 | 3.00 | CD5 | CD7 |
| mutOKT8 | — | 9.00 | NT | — |
| DSPE-PEG | — | 9.00* | NT | — |
| TH-69 | hSP34 | 3.00 | CD7 | CD3 |
| TS2/18.1 | He3 | 1.50 | CD2 | CD5 |

*DSPE-PEG amount added to match the amount added from 9 g/mol of a ~48 kD Fab

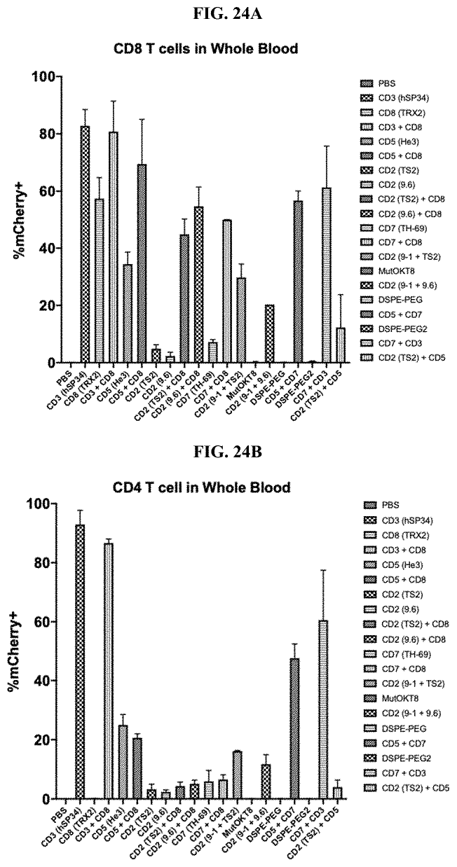

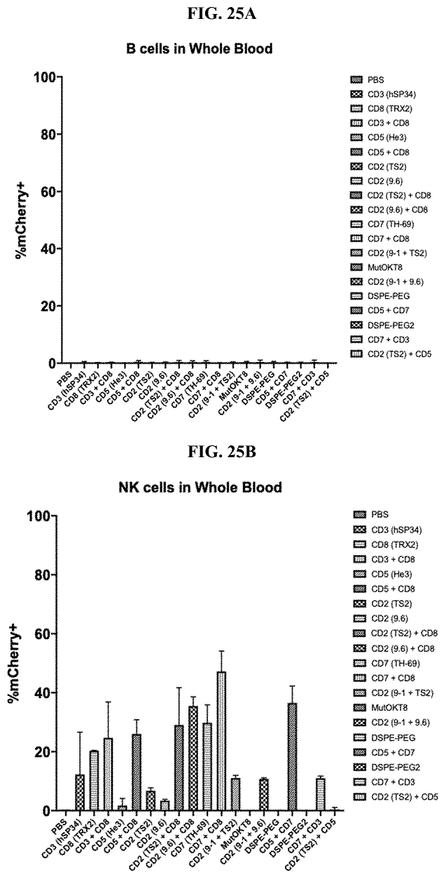

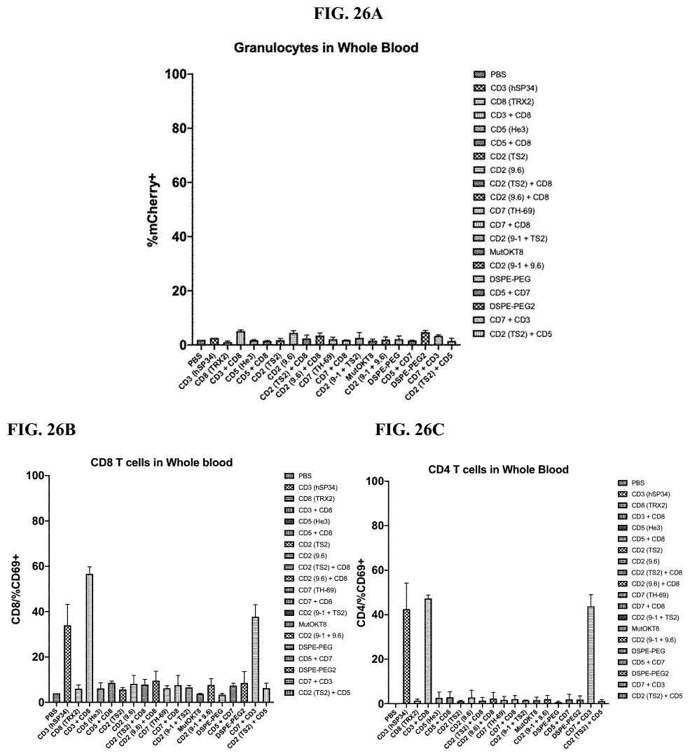

All of the targeting Fabs enable observable transfection relative to background with varying degrees of efficiency depending on the target and clone. The most efficient transfection of both CD8 and CD4 cells was observed using hSP34, He3 and combinations of hSP34/He3, He3/TH-69, hSP34/TRX2, He3/TRX2, hSP34/TH-69, TH-69/TRX2, 9.6/9-1, TS2/9-1 (FIGS. 24A and 24B). TRX2 efficiently transfects CD8 T-cells without transfection observed in CD4 T-cells (FIGS. 24A and 24B). Additive effects were observed in terms of transfection efficiency for combinations with TRX2/He3, 9.6/9-1, TS2/9-1 and He3/TH-69 (FIGS. 24A and 24B) indicating synergistic effects can be mediated by targeting two different targets or two different epitopes on the same target. Transfection was also observed in NK cells with CD3, CD8, CD2 and CD7 targeting in addition to combinations thereof (FIG. 25B). Generally, no off-target transfection is observed in B-cells (FIG. 25A) and Granulocytes (FIG. 26A). High CD69 upregulation was only observed in CD4 and CD8 cells with CD3-targeting or CD3 targeting in combination with other targets such as CD8 or CD7 (FIGS. 26B and 26C). Additionally, non-targeted LNPs post-inserted with similar DPSE-PEG relative to Fab targeted formulations and LNPs post-inserted with mutOKT8 Fab did not exhibit transfection of any of the immune cell types indicating specific transfection is mediated by Fab targeting (FIGS. 24A and 24B).

This study shows that in whole blood, CD4 and CD8 T cells can be efficiently transfected with CD3-targeted, CD5-targeted, CD7-targeted or CD2-targeted LNPs as well as targeting combinations thereof, CD8 cells can be specifically (avoidance of CD4 transfection) transfected with CD8-targeted LNPs, transfection can be skewed towards CD8 cells versus CD4 cells using CD8-targeting in combination with CD5, CD7 or CD2 targeting.

Using Fabs targeting different targets or Fab clones that bind the same target but known to target different epitopes (e.g., anti-CD2 clones 9.6 and 9-1) in combination can lead to synergistic increases in transfection efficiency. NK cells were transfected with CD8, CD7 or CD2 targeting Fabs or combinations thereof consistent with known surface expression of these markers on human NK cells or NK cell subsets. While LNPs with anti-CD3, anti-CD8, anti-CD5, anti-CD7 or anti-CD2 Fabs or combinations thereof can mediate efficient transfection of T cells and NK cells (for some Fabs), minimal transfection was observed in B cells or Granulocytes indicating high specific uptake and transfection enabled by Fab targeting given non-targeted Fab (mutOKT8) or nontargeted LNPs did not transfect T cells or NK cells. Additionally, using anti-CD8, anti-CD5, anti-CD7 or anti-CD2 Fabs or combinations thereof can mediate efficient transfection without driving high CD69 expression.

### Example 18—In-Vivo Reprogramming of Immune Cells with Lnp Expressing Mcherry

This example describes the time course of reprogramming of immune cells in humanized mice treated with LNP expressing mCherry.

### Mice Strains and Humanization

The NCG mouse (NOD-Prkdc^{em26Cd52} I12rg^{em26Cd22}/NjuCrl) mouse model was purchased from Charles River Laboratories. 4 weeks old male mice were engrafted with 10 million PBMC of qualified donor (by Charles river) in sterile PBS by tail vein injection and were shipped to Tidal facility. Individual body weight was monitored twice a week and blood samples were collected at appropriate interval to evaluate human immune cells engraftment.

### Evaluation of Human T-Cell Engraftment in the Immunodeficient Mice

50 μl blood was collected by tail vein bleed from each mouse. Red blood cells were lysed using Versalyse, RBC lysis solution following protocol as instructed by manufacturer (Beckman Coulter A09777). Cells were stained with hCD45 & hCD3 to determine the engraftment of human T-cells. After 30 days of PBMC injection, mice had anywhere from 30-60% huCD45+. These humanized mice were evaluated for reprogramming of immune cells by LNPs expressing mCherry.

### Reprogramming of Immune Cells

At time zero, 9 mice were injected with mCherry expressing LNPs prepared using cationic Lipid 8 and the mixing process described in Example 6, the buffer exchange process described in Example 21, and targeted with hSP34-lipid using the process described in Example 5 (Lot#201109APG-NF70-409), by i.v. at 3 mg/kg or 6 mice were injected with appropriate buffer. At each time point, 24, 48 and 96 h, 3 mice treated with LNPs or 2 mice treated with buffer were sacrificed. Terminal blood and tissues collection was performed to determine mCherry expression in different organs and immune cells as below.

### Tissue and Blood Sample Collection

At above specified timepoints, mice were anesthetized with CO₂ before sample collection. For blood collection, the chest was opened to expose the heart. Up to 300 μl blood was drawn from the left ventricle and dispensed into a K3EDTA mini collect tube (Greiner Bio-One). Then a new syringe was used to draw remaining blood from the heart as much as possible. All the immune organs; spleen, bone marrow, thymus and all the lymph nodes (linginual, axillary, submandibual and mesentry) were isolated along with liver. Immune cells were isolated from spleen, thymus and lymph nodes via smearing and shredding it through syringe and cell suspension was filtered through 70 μM cell strainer and was washed with PBS. A piece of liver tissue was gently grinded with tissue homogenizer and the homogenized tissue was incubated with digestive solution (10 ml HBSS supplemented with 0.05% of type IV collagenase (Sigma C5138-5G) 0.02% BSA (Sigma A2153-100G), 0.001% DNASE I, Grade II (Sigma 10104159001) and 1 mM calcium chloride (Sigma C7902-500G) for 30 min at 37° C. After 30 mins the digestion was terminated with 10 ml of ice cold solution of HBSS.

### Immunophenotyping Analysis

Immune cells from blood and all the above organs were processed with Versalyse, RBC lysis buffer as per manufacturing instructions. Immune cells were stained with live/dead fixable dye and surface markers with standard flow analysis protocol as shown in below panel. Attune, Thermo flow cytometer was used to determine positive population.

TABLE 15
Panel 1

| Antigen | Fluorophore | Clone | Company | Catalog# |
|---|---|---|---|---|
| Alive Dead dye | Zombie Aqua | NA | BioLegend | 423102 |
| Anti-human CD45 | FITC | 2D1 | BioLegend | 368508 |
| Anti-human CD3 | PerCP/Cyanine5.5 | UCTH1 | BioLegend | 300430 |
| Anti-human CD8 | APC-Fire750 | SK1 | BioLegend | 344746 |
| Anti-human CD4 | BV711 | SK3 | BioLegend | 344648 |
| Anti-human CD69 | BV421 | FN50 | BioLegend | 310930 |
| Anti-human CD137 | BV421 | 4B4-1 | BioLegend | 309820 |
| Anti-human279 | APC | NAT05 | BioLegend | 367406 |
| PD-1 | | | | |
| Anti-human CD366 | APC | F38-2E2 | BioLegend | 345012 |
| mCherry | mCherry | NA | NA | NA |

TABLE 16
Panel 2

| Antigen | Fluorophore | Clone | Company | Catalog# |
|---|---|---|---|---|
| Alive Dead dye | Zombie Aqua | NA | BioLegend | 423102 |
| Anti-human | FITC | 2D1 | BioLegend | 368508 |
| Anti-human/mouse | BV785 | M1/70 | BioLegend | 101243 |
| CD11b | | | | |
| Anti-mouse F4/80 | APC | BM8 | BioLegend | 123116 |
| Anti-human CD19 | BV711 | SJ25C1 | BioLegend | 563036 |
| mCherry | mCherry | NA | | NA |

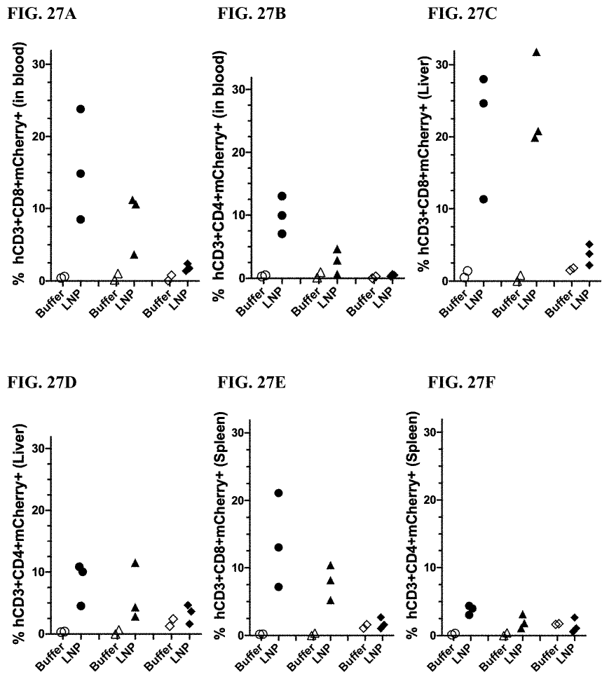

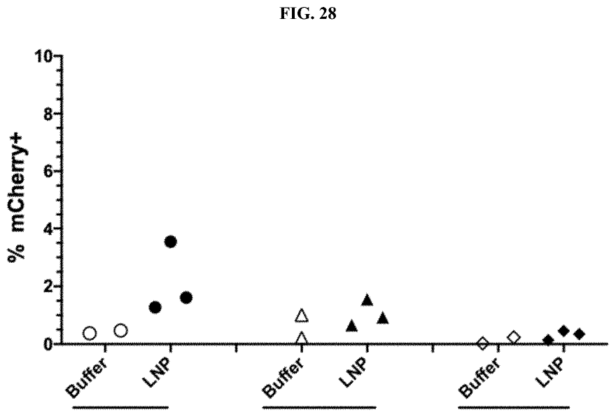

huNCG-PBMC mice treated with CD3 targeted LNP expressing mCherry at 3 mg/kg showed mcherry in T cells of blood, liver and spleen. CD8+ T cells (FIGS. 27A, 27C and 27E) showed highest mCherry expression, with up to ˜30% of CD8+ T cells in blood, liver and spleen. CD4+ T cells (FIGS. 27B, 27D and 27F) showed up to ˜15% mCherry expression in blood, liver and spleen. No reprogramming was seen in other organs analyzed. The expression of mCherry is restricted to CD3+ cells. Minimal or no mCherry expression was observed in liver myeloid, macrophages or Kupffer cells (FIG. 28).
Overall, CD3 targeted mCherry LNP specifically reprogrammed T cells with minimal or no expression in myeloid population.

### Example 19—In-Vivo Reprogramming of Immune Cells with Lnp Expressing Mcherry or Cd20 Car with Either Cd3 and or Cd8 Targeting Antibody

This Example describes in vivo reprogramming with LNP expressing either mCherry or CD20 CAR and compares CD3 vs CD8 targeting or combination of both.

### Mice Strains and Humanization

NSG female mice were purchased from Jackson lab. At 8 weeks of age mice were i.v. injected with 20 million PBMC (in house isolated leukopak, donor 555046, Precision for Medicine).

### Evaluation of Human T-cell Engraftment in the Immunodeficient Mice

50 μl blood was collected by tail vein bleed from each mouse. Red blood cells were lysed using Versalyse, RBC lysis solution following protocol as instructed by the manufacturer (Beckman Coulter A09777). Cells were stained with hCD45 & hCD3 to determine the engraftment of human T-cells. After 30 days of PBMC injection, mice had anywhere from 60-80% huCD45+. These humanized mice were evaluated for reprogramming of immune cells as below.

Reprogramming of Immune Cells with mCherry & CD20 CAR with CD3 and/or CD8 Targeting Antibody

At time zero, mice (n=5) were i.v. injected at the dose of 3 mg/kg with either i) Buffer or LNP expressing; ii) TTR-023 mRNA targeted with 17 g/mol of hSP34; iii) TTR-023 mRNA targeted with 9 g/mol of hSP34; iv) TTR-023 mRNA targeted with 9 g/mol of TRX2; v) TTR-023 mRNA targeted with 9 g/mol of TRX2+9 g/mol of hSP34; vi) mCherry mRNA (n=3 mice) targeted with 17 g/mol of hSP34. After 24 h, 50 ml blood was collected and processed as mentioned in example 18. At 96 h, 2nd dose of either LNPs (as above) or buffer was injected in mice at 3 mg/kg. After the 2nd dose, terminal blood and organs were collected and processed as described in example 18 at 40 h time point.

LNPs were prepared using cationic Lipid 8 and the mixing process described in Example 6, the buffer exchange process described below in Example 21, and targeted with hSP34-lipid or TRX2-lipid using the process described in Example 5. The table below summarizes the formulations and lot numbers used.

TABLE 17

| | Lot Number for 1st | Lot Number for 2nd |
|---|---|---|
| Test Article Description | Dose Material | Dose Material |
| TTR-023 LNPs inserted with 17 g/mol hSP34 | 210128APG-NT317 | 210201APG-NT317 |
| TTR-023 LNPs inserted with 9 g/mol hSP34 | 210128APG-NT309 | 210201APG-NT309 |
| TTR-023 LNPs inserted with 9 g/mol TRX2 | 210128APG-NT809 | 210201APG-NT809 |
| TTR-023 LNPs inserted with 9 g/mol hSP34 | 210128APG-NT38 | 210201APG-NT38 |
| and 9 g/mol TRX2 | | |
| mCherry LNPs inserted with 17 g/mol hSP34 | 210128APG-NM317 | 210201APG-NM317 |
| 5.3 wt % sucrose in HBS | 210128APG-S1 | 210128APG-S1 |

### Immunophenotyping Analysis

Similar immunophenotyping analysis was done as described in example 18 with panels listed below. CD20 CAR expression was evaluated by detecting M1 tag expressed by CD20 CAR with primary M1 antibody followed by secondary antibody.

TABLE 18
Panel 1

| Antigen | Fluorophore | Clone | Company | Catalog# |
|---|---|---|---|---|
| Alive Dead dye | Zombie Aqua | NA | BioLegend | 423102 |
| Anti-human CD45 | FITC | 2D1 | BioLegend | 368508 |
| Anti-human CD3 | PerCP/ | UCTH1 | BioLegend | 300430 |
| | Cyanine5.5 | | | |
| Anti-human CD8 | APC-Fire750 | SK1 | BioLegend | 344746 |
| Anti-human CD4 | BV711 | SK3 | BioLegend | 344648 |
| Anti-human CD69 | BV421 | FN50 | BioLegend | 310930 |
| Anti-human CD137 | BV421 | 4B4-1 | BioLegend | 309820 |
| M1 tag | NA | NA | Sigma | M1-F3040 |
| Secondary antibody | APC | NA | Southern | 1090-11S |
| to M1 | | | biotech | |
| mCherry | mCherry | NA | NA | NA |

TABLE 19
Panel 2

| Antigen | Fluorophore | Clone | Company | Catalog# |
|---|---|---|---|---|
| Alive Dead dye | Zombie Aqua | NA | BioLegend | 423102 |
| Anti-human | FITC | 2D1 | BioLegend | 368508 |
| Anti-human/mouse | BV786 | M1/70 | BioLegend | 101243 |
| CD11b | | | | |
| Anti-mouse F4/80 | BV421 | BM8 | BioLegend | 123132 |
| Anti-human CD19 | BV711 | SJ25C1 | BioLegend | 563036 |
| mCherry | mCherry | NA | NA | NA |
| M1 tag | NA | NA | Sigma | M1-F3040 |
| Secondary antibody | APC | NA | Southern biotech | 1090-1 IS |
| to M1 | | | | |

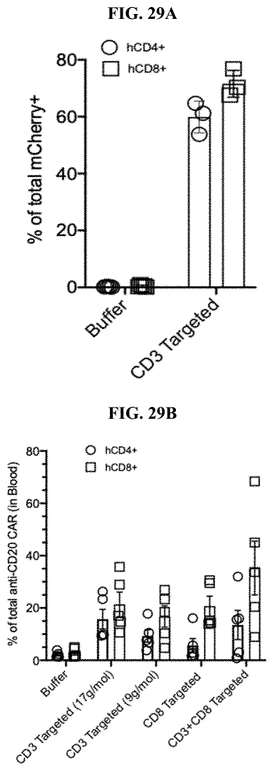

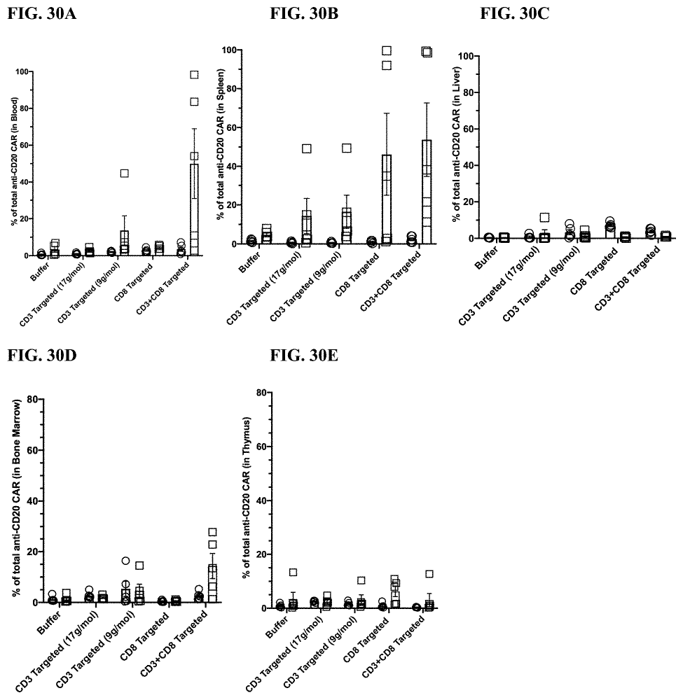

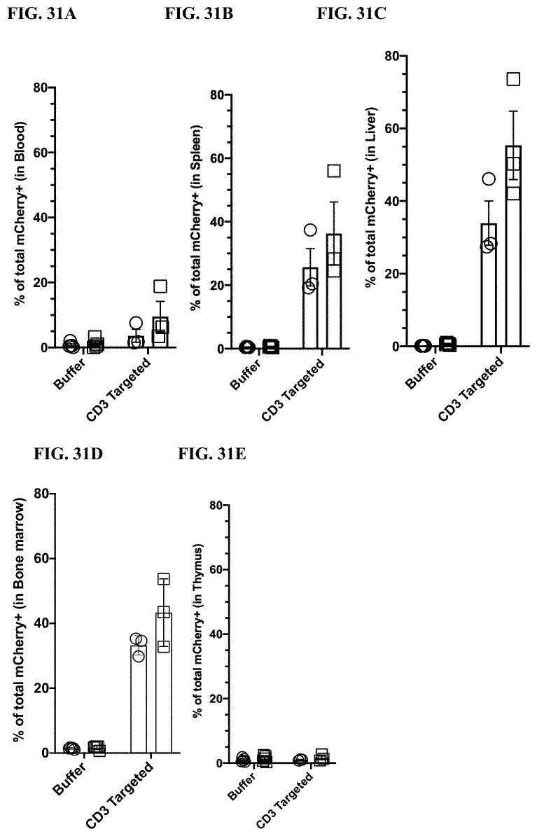

After 24 hr of first dose at 3 mg/kg, >60% T cells are reprogrammed with mCherry mRNA using anti-CD3 targeting (FIG. 29A). Both CD3 and CD8 targeting showed >20% of T cells reprogrammed with CD20 CAR mRNA, whereas combination of both showed ˜30% T cells reprogrammed with CD20 CAR mRNA (FIG. 29B). After 40 hr of 2^{nd} dose at 3 mg/kg of anti-CD20 CAR expressing LNP >30% of T cells are reprogrammed with anti-CD20 CAR using CD3 targeting; Spleen>Blood>Liver>Bone Marrow>Thymus (FIG. 30A-E). No significant increase in reprogramming of T cells is observed with increasing density of anti-CD3 targeting (FIG. 30A-E). After 40 hr of 2^{nd} dose at 3 mg/kg of anti-CD20 CAR expressing LNP targeted with CD8 showed maximum reprogramming in spleen (>50%) as compared to other tissue (FIG. 30A-E). As expected, CD8-targeted selectively reprograms CD8+ T cells over CD4+ cells. Combination of CD3 and CD8 targeting shows the most robust reprogramming in multiple tissues; Blood=Spleen>BoneMarrow>Thymus>Liver (FIG. 30A-E). After 40 h of 2^{nd} dose >60% of T cells are reprogrammed with mCherry mRNA using anti-CD3 targeting; Spleen>Liver>Bone Marrow>Blood (FIG. 31A-E). No reprogramming is observed in thymus or lymph node at this time point with mCherry expressing LNPs.
Overall, there is difference in distribution of CD3 or CD8 targeted reprogrammed cells that appears to also mRNA cargo dependent. Without wishing to be bound by theory, it could also be due to different kinetics of redistribution of already reprogrammed T cells in periphery and other organs. CD8 targeting shows specificity for reprogramming of CD8 T cells in blood and all the organs tested. No reprogramming was observed in myeloid cells in blood or organs.

### Example 20: Pharmacokinetics Study in Mice with LNPs

This example describes the pharmacokinetics of LNPs in female BALC/c mice.

LNPs were prepared using the vortex mixing process described in Example 2 using the component ratios described in Table 20 below. Particle properties were characterized using methods described in Example 3 and are described below in Table 21.

TABLE 20

| | nmol lipid per 100 μg mRNA | | | | |
|---|---|---|---|---|---|
| | | | | DMG- | |
| Batch | Lipid 8 | Cholesterol | DSPC | PEG2000 | Dil-C18(3)-DS |
| 83.1 | 1500 | 1200 | 300 | 75 | 9 |

TABLE 21

| Batch | Size (nm) | PDI | EE (%) |
|---|---|---|---|
| 83.1 | 157.4 | 0.165 | 79.6* |

*Encapsulation efficiency possibly affected by DiI-C18(3)-DS fluorescence interfering with mRNA quantification

8 female BALB/c mice were purchased from Janvier Labs (Le Genest-Saint-Isle, France) and acclimated for one week. Food was provided ad libitum.

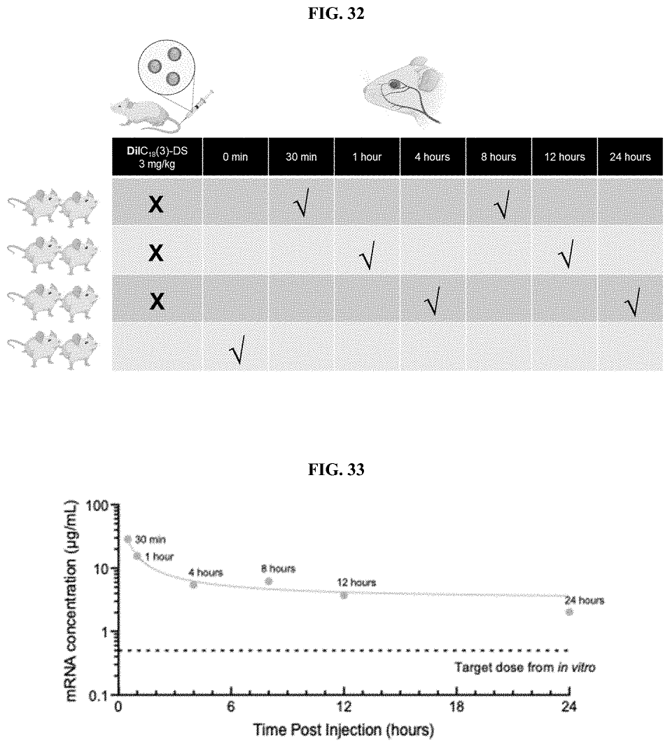

Mice were injected intravenously through the tail vein with a single dose of 3 mg/kg LNPs formulated with 1,1′-Dioctadecyl-3,3,3′,3′-Tetramethylindocarbocyanine-5,5′-Disulfonic Acid (DiI-C18(3)-DS), and mCherry mRNA. Blood samples were obtained from the facial vein and sample collection occurred at times ranging from 30 minutes to 24 hours (n=2 for each time point). Samples were centrifuged at 10,000g×10 minutes, and serum was stored at 4° C. until the time of analysis. The pharmacokinetics of LNPs of the formulation described in Table 20 were determined in Balb/c mice following collection of blood at the timepoints outlined in FIG. 32 and are shown in FIG. 33, where the LNPs are cleared only slowly from the circulation over 24 h.

The fluorescence quantification was carried out using a fluorescence microplate reader (Spark multimode reader, Tecan). Readings were from the top with excitation/emission wavelengths at 555/570 nm. Quantification of nanoparticles in circulation was performed through interpolation using a standard curve.

Using an average mouse weight of 25 g and approximate blood volume of 2 mL to calculate a theoretical initial LNP concentration of 37.5 μg/mL in serum, ˜80% of the injected dose was detected in plasma after 30 min, ˜40% after 1 hour and −10% after 8 hours (FIG. 33).

This study shows that the current formulation can maintain mRNA levels in the circulation above a concentration of 0.5 μg/mL for more than 24 hours when dosed at an mRNA dose of 3 mg/kg.

### Example 21—Preparation of LNPS by Microfluidic In-Line Mixing and Tangential Flow Filtration Using Exemplary Ionizable Lipids

This example describes preparation of LNPs using scalable unit operations, namely in-line microfluidic mixing followed by tangential flow filtration (TFF) for ethanol removal and buffer exchange.

Using the mixing process in Example 6, multiple LNP batches were pooled together, totaling 60 mL at an RNA concentration of 300 μg/mL. Ethanol removal and buffer exchange was subsequently performed using tangential flow filtration (TFF).

Following mixing, ethanol removal and buffer exchange were performed on the resulting LNP suspension using a hollow fiber TFF module (Repligen, US P/N D02-E100-05-N). Briefly, the TFF module was rinsed with DI water and pumped dry before use. LNPs were then added to the reservoir, and the exchange buffer (25 mM pH 7.4 HEPES buffer with 150 mM NaCl) was used as the diafiltration buffer. The TFF module was primed, and diafiltrations (DVs) were then initiated by ramping up the peristaltic pump to target flow rate and adjusting Retentate valve until target transmembrane pressure (TMP) is reached. A flow rate of 212 mL/min and a TMP of 3.5 psi were the target operating parameters for the system once diafiltration was initiated. Throughout the diafiltration process, the TMP was kept constant by adjusting the retentate valve. Permeate flow rate was monitored and did not decrease significantly over time. Six diafiltrations were performed, with samples set aside at the end of each diafiltration to later track the buffer exchange process. Final ethanol content was <0.1%, as measured by refractive index measurements on DV samples, and pH measurements confirmed the buffer exchange into the exchange buffer. Upon the completion of six diafiltrations, the pump was stopped, and a concentration of the resulting LNP suspension was subsequently performed.

The concentration of the LNP suspension was performed using the same TFF module that was used during the buffer exchange process. TMP and flow rate, after pump ramp up, from buffer exchange process were maintained and the suspension was allowed to concentrate by stopping the addition of diafiltration buffer. The resulting LNP suspension was collected and filtered with a 0.2 μm syringe filter. The suspension was sampled for analytical purposes and then stored at 4° C. until further use.

Using the LNP characterization process in Example 3, LNP batch was characterized to determine the average hydrodynamic diameter and mRNA content (total and dye-accessible); set forth in Table 22 below. As seen in Table 22, the microfluidic mixing process with ethanol removal and buffer exchange by TFF results in sub-100 nm particles exhibiting narrow polydispersity and good mRNA encapsulation (<20% dye accessible RNA).

TABLE 22

| | | | | | Dye- | |
|---|---|---|---|---|---|---|
| | | DLS Z- | | Total | Accessible | Dye- |
| | Process | Avg. | | mRNA | mRNA | Accessible |
| Sample ID/ | Point/ | Diameter | DLS | Content | Content | mRNA |
| Lot Number | Description | (nm) | PDI | (μg/mL) | (μg/mL) | (%) |
| 210107MTM-NR20 | LNPs after 6 DVs | 64 | 0.08 | 282 | 11 | 4% |
| | in HBS | | | | | |
| 210107MTM-NR30 | LNPs after | 64 | 0.10 | 1111 | 43 | 4% |
| | Concentration | | | | | |
| | (pre-filter) | | | | | |
| 210107MTM-NR40 | LNPs after | 63 | 0.06 | 1099 | 30 | 3% |
| | Concentration | | | | | |
| | and 0.2 um | | | | | |
| | filtration | | | | | |

### Example 22: Effect of PEG in Whole Blood Transfection of mRNA LNPs

This example describes specifically targeting human T cells in whole blood with anti-CD3 Fab post-inserted into mCherry mRNA LNPs with or without DiR labeling and with varying levels of PEG incorporated during particle formation to determine the effect of PEG on LNP binding (DiR signal) and transfection efficiency (mCherry).

SP34-Fab lipid conjugate from the process described in Example 4 is a mixture of 3 PEG-lipid variants (DSPE-PEG2k-Fab, DSPE-PEG2k-maleimide(quenched), DSPE-PEG2k-OCH3) therefore the effect of an additional PEG (DMG-PEG200) was explored. LNPs were prepared using the vortex mixing process described in Example 2 using the component ratios described in Table 23 below and conjugate was post-inserted after particle formation. Particle properties were characterized using methods described in Example 3 and are described below in Table 24.

TABLE 23

| | nmol lipid per 100 μg mCherry mRNA | | | | | |
|---|---|---|---|---|---|---|
| | | | | DMG- | | |
| Batch | Lipid 8 | Cholesterol | DSPC | PEG2000 | DiR | Conjugate |
| RDM085.8 | 1500 | 1200 | 300 | 75 | 4.5 | 1.0 |
| RDM0138 | 1575 | 1200 | 300 | — | 4.5 | 1.0 |
| RDM073.19 | 1500 | 1200 | 300 | 75 | — | 1.0 |
| RDM149 | 1575 | 1200 | 300 | — | — | 1.0 |

TABLE 24

| Batch | Size (nm) | PDI | EE (%) |
|---|---|---|---|
| RDM085.8 | 103.2 | 0.113 | 96.3 |
| RDM0138 | 113.8 | 0.096 | 87.4 |
| RDM073.19 | 119.4 | 0.129 | 83.4 |
| RDM149 | 170.5 | 0.083 | 92.3 |

Venous blood from healthy volunteers was anti-coagulated in Hirudin tubes (Sarsted #04.1959.001) and seeded at 50 μl in a 96-well round-bottom plate. Transfection of whole blood was carried out by adding LNPs containing 2.5 μg/mL mRNA to the cells and co-culturing at 37° C. until the time of analysis. To assess binding of DiR labeled LNPs, cells were analyzed 2-hours post-LNP addition and for transfection efficiency, cells were analyzed after 24-hours of incubation by flow cytometry.

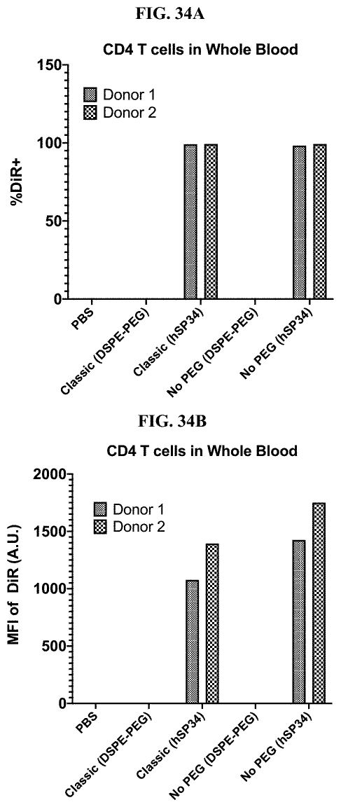

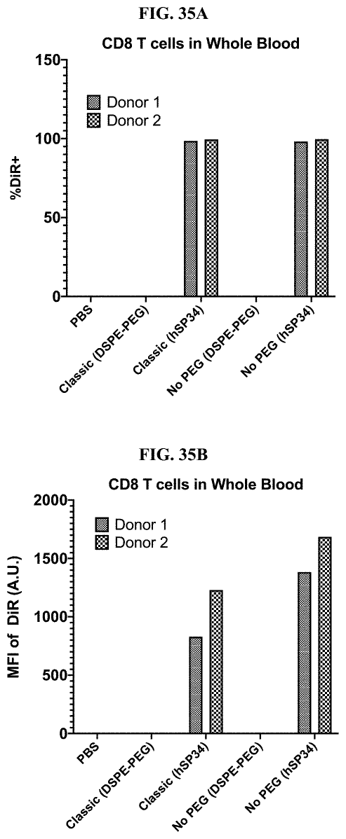

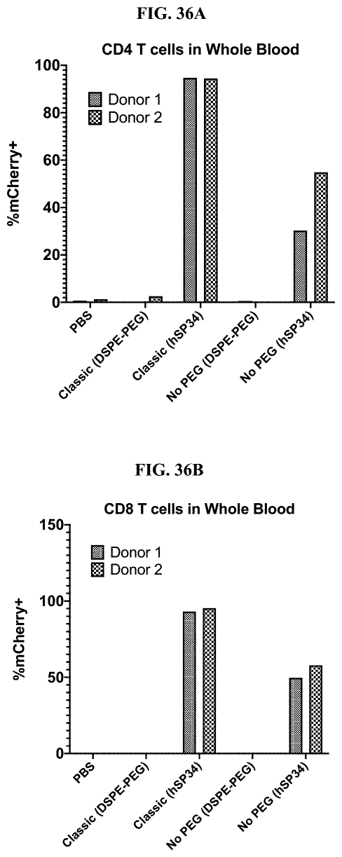

For non-targeted LNPs, only DSPE-PEG2k was post-inserted to match SP34 targeted LNPs, labeled DSPE-PEG in FIGS. 34A-36B. No detectable binding (FIGS. 34A-35B) or transfection (FIGS. 36A and 36B) was observed for non-targeted LNPs indicating SP34 mediated highly specific transfection via CD3 targeting. For SP34-Fab lipid conjugate post-inserted particles, LNPs lacking DMG-PEG200 during particle formation exhibited higher binding by DiR signal for CD4 (FIGS. 34A and 34B) and CD8 (FIGS. 35A and 34B) T cells but lower transfection efficiency (FIGS. 36A and 36B) than LNPs that contained DMG-PEG200 during particle formation.

This study indicates introduction of a 4th PEG-lipid variant into the particle in addition to the 3 PEG-lipid variants that get added as part of the post-insertion process can have a dramatic effect on particle uptake and transfection efficiency, in this case, an approximate 2-3 fold difference was observed in transfection efficiency.

### Example 23—Lipid 8 and Lipid 5 LNP Properties, In Vitro Cell Viability and Protein Expression in Primary Human T-Cells

This example describes the relative in vitro toxicity of CD3 targeted LNPs derived from Lipid 8 and Lipid 5 in primary human T-cell transfection of GFP-mRNA. Nanoparticles are first produced using a mixing process followed by buffer exchange. Particles thus produced were subsequently tested in vitro in human CD3+ T cells to assess T-cell viability at three LNP doses, LNP association with cells, and expression of a reporter gene.

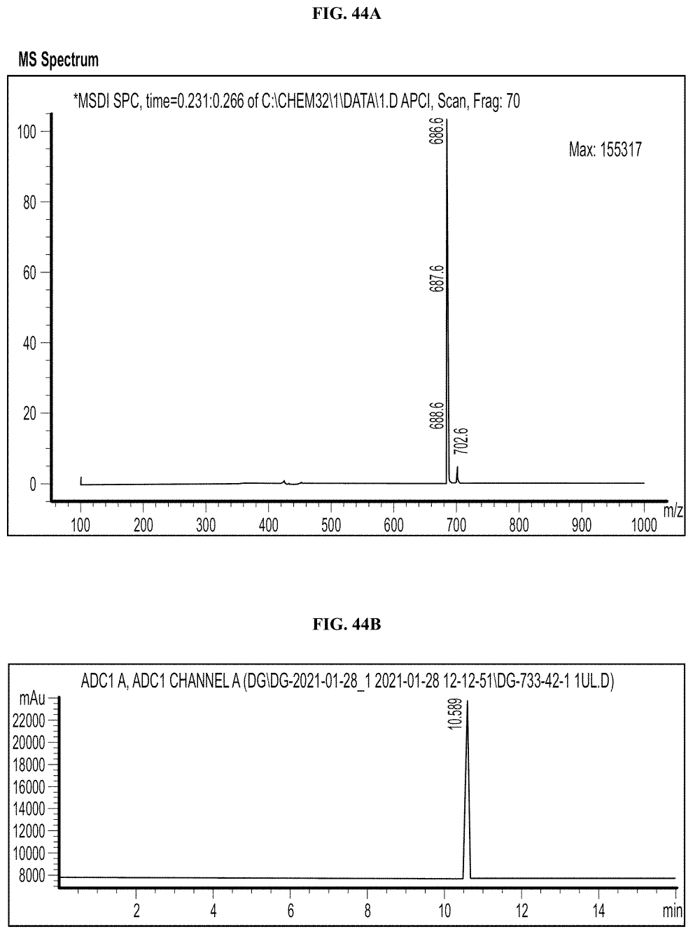

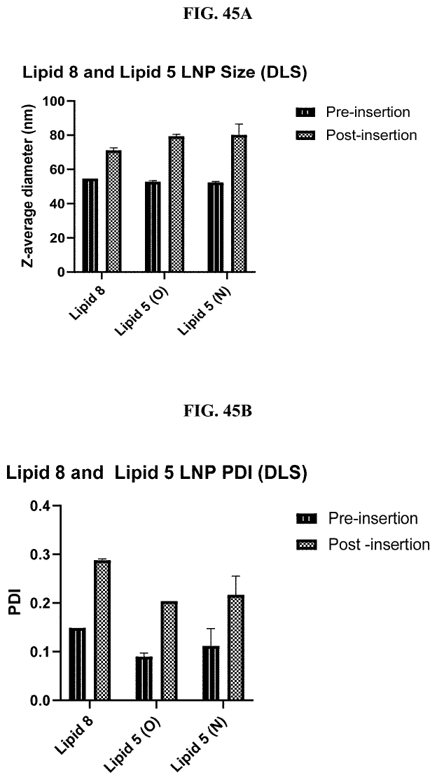

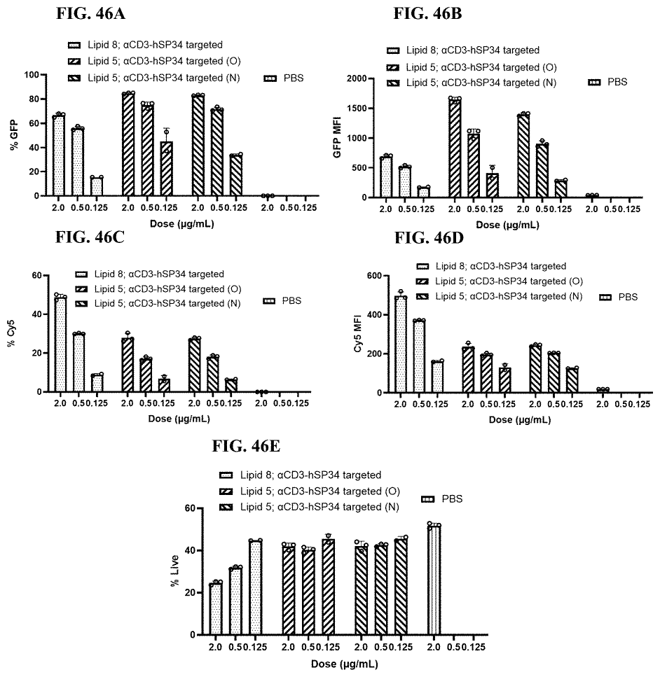

Lipid 8 and Lipid 5 LNPs encapsulating a 90-10 (w/w) mixture of GFP-mRNA and Cyanine-5 dye labelled mRNA (TriLink Biotechnologies Inc.) were prepared using the mixing process described in Example 6, the buffer exchange process described in Example 21. Lipid 5 LNPs were produced using Lipid 5 stock solutions that had been stored frozen at −20 C for either 2 weeks or 1 day (Lipid 5 (0) and Lipid 5 (N), respectively). Both formulations resulted in particles exhibiting hydrodynamic diameters in the sub-100 nm range and moderate polydispersities, as well as good mRNA encapsulation and recovery (Table 25, <25% dye accessible mRNA and >80% encapsulated mRNA was recovered using the Triton-deformulation procedure described in Example 3). As seen in FIGS. 45A and 45B, Lipid 5 LNP exhibited a larger change in hydrodynamic diameter (relative to Lipid 8 LNP) upon insertion of an anti-CD3 hSP34-PEG2k-DSPE conjugate using the insertion procedure described in Example 4. The resulting targeted LNPs were evaluated in primary human T-cells using the in vitro transfection protocol described in example 8. As seen in FIGS. 46A-46E, the PBS control arm exhibited about 50% T-cell viability while doses of 0.125, 0.5 and 2 ug mRNA/mL per well of Lipid 8 LNPs exhibited a dose dependent toxicity towards T-cells with T-cell viability dropping from about 45% live at 0.125 ug mRNA/mL per well to about 25% live at 2 ug mRNA/mL per well. In contrast, Lipid 5 LNPs (both sample “0” and “N”) were consistently better tolerated by T-cells with 40-45% T-cell viability observed at all three dose levels. Lower toxicities observed with Lipid 5 LNPs may be attributed to more rapid degradation and clearance of Lipid 5 from T-cells driven by hydrolytic and/or enzymatic degradation of labile ester bonds in the Lipid 5 molecule.

Dose dependent expression of GFP protein was observed with both ionizable lipids (5 and 8), however, as illustrated by both % GFP+ and GFP MFI values (FIGS. 46A and B), Lipid 5 LNPs resulted in greater overall protein expression at all three mRNA dose levels suggesting improved cytosolic availability of the mRNA payload with Lipid 5 LNPs.

TABLE 25
Lipid 8 and Lipid 5 LNP mRNA content

| | Theoretical | Measured total | Ribogreen Dye |
|---|---|---|---|
| | total | (Triton method) | Accessible |
| Ionizable | mRNA | mRNA | mRNA |
| Lipid | (ug/mL) | (ug/mL) | (ug/mL) |
| Lipid 8 | 45 | 40 | 4 |
| Lipid 5 (O) | 45 | 38 | 7 |
| Lipid 5 (N) | 45 | 36 | 7 |

Overall, these data show that CD3-targeted LNPs formed with Lipid 5 showed both lower cellular toxicity and higher transfection activity in human T-cells compared to LNPs prepared with Lipid 8.

### Example 24—Standard Procedure for In-Vivo Reprogramming of Immune Cells with DiI LNP Expressing GFP

The following standard procedure for in-vivo reprogramming of immune cells with DiI LNP expressing GFP was used in the experiments in Example 29.

### Mice Strains and Humanization

The NSG (NOD.Cg-Prkdcscid Il2rgtmlWjl/SzJ) mouse model was purchased from Jax Laboratories. 6-8 weeks old male mice were engrafted with 10 million PBMC of qualified donor in sterile PBS by tail vein injection. Individual body weight was monitored twice a week and blood samples were collected at appropriate interval to evaluate human immune cells engraftment.

### Evaluation of Human T-Cell Engraftment in the Immunodeficient Mice

50 μl blood was collected by tail vein bleed from each mouse. Red blood cells were lysed using Versalyse, RBC lysis solution following protocol as instructed by manufacturer (Beckman Coulter A09777). Cells were stained with hCD45 & hCD3 to determine the engraftment of human T-cells. After 15 days of PBMC injection, mice had anywhere from 30-60% huCD45+. These humanized mice were evaluated for reprogramming of immune cells by LNPs expressing DiI dye and GFP.

### Reprogramming of Immune Cells

At time zero, mice (n=4 per group) were injected with GFP expressing DiI LNPs (by i. v. at 0.3 mg/kg or 0.1 mg/kg with appropriate buffer. At each time point, 24 or 48h depending on the example mice treated with either LNPs or buffer were sacrificed. Terminal blood and tissues collection was performed to determine DiI and GFP expression in different organs and immune cells as below.

Tissue and blood sample collection.

At above specified timepoints, mice were anesthetized with CO₂ before sample collection. For blood collection, the chest was opened to expose the heart. Up to 300 μl blood was drawn from the left ventricle and dispensed into a K3EDTA mini collect tube (Greiner Bio-One). Then a new syringe was used to draw remaining blood from the heart as much as possible. All the immune organs; spleen, bone marrow was isolated along with liver and lung. Immune cells were isolated from spleen, via smearing and shredding it through syringe and cell suspension was filtered through 70 μM cell strainer and was washed with PBS. Bone marrow was flushed with needle to collect all the immune cells. A piece of liver and lung tissue was gently grinded with tissue homogenizer and the homogenized and cells were isolated using militenyi liver dissociation kit, (Miltenyi Biotec, Catalog#130-105-807) and lung dissociation kit (Miltenyi Biotec, Catalog#130-0950927) and instruction were followed according to the manufacturing instruction.

### Immunophenotyping Analysis

Immune cells from blood and all the above organs were processed with Versalyse, RBC lysis buffer as per manufacturing instructions. Immune cells were stained with live/dead fixable dye and surface markers with standard flow analysis protocol as shown in below panel. BD symphony flow cytometer was used to determine positive population.

Panel

| Antigen | Fluorophore | Clone | Company | Catalog# |
|---|---|---|---|---|
| DiI | APC | NA | NA | NA |
| GFP | mRNA | NA | NA | NA |
| Anti-CD45 | BUV395 | HI30 | BD Biosciences | 563792 |
| Anti-CD3 | BUV805 | UCHT1 | BD Biosciences | 612895 |
| Anti-CD4 | BV711 | SK3 | BioLegend | 344648 |
| Anti-CD8 | BV421 | RPA-TB | BD Biosciences | 562428 |
| Anti-CD45 | BB700 | 30-F11 | BD Biosciences | 566439 |
| Anti-CD11b | BV785 | M1/70 | BioLegend | 101243 |
| Anti-F4/80 | PE Dazzle | BM8 | BioLegend | 123146 |
| Anti-CD31 | BUV737 | MEC 13.3 | BD Biosciences | 612802 |
| TruStain | NA | NA | BioLegend | 462103 |
| Monocyte Blocker | | | | |
| Arc Amine | NA | NA | NA | 01-3333-42 |
| Comp beads | | | | |
| UltraComp eBeads | NA | NA | Invitrogen | 01-3333-42 |
| LIVE/ | NA | NA | Invitrogen | L34974 |
| DEAD Far | | | | |
| Red Stain | | | | |
| TruStain Fc X | NA | NA | Invitrogen | 422302 |

### Example 25—Alternative Ethanol Removal and Buffer Exchange Process

In addition to the ethanol removal and buffer exchange process described in Example 6, an alternative process can be used to produce LNPs of the present disclosure. Particularly, following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with exchange buffer (25 mM pH 7.4 HEPES buffer with 150 mM NaCl). The LNP suspension (1.5 mL) was then loaded into the device and centrifuged at 500 rcf until the volume was reduced by half (0.75 mL). The suspension was then diluted with exchange buffer (0.75 mL) to bring the suspension back to the original volume. This process of two-fold concentration and two-fold dilution was repeated five additional times for a total of six discontinuous diafiltration steps. The retentate containing the LNPs in the exchange buffer was recovered from the centrifugal ultrafiltration device and stored at 4° C. until further use.

### Example 26—Lipid 8 and Lipid 5 LNP Properties, and In Vitro Cell Viability and Protein Expression in Primary Human T-Cells

This example compares the properties of LNPs prepared using Lipid 5 and Lipid 8 and the GFP protein expression in primary human T-cells. Both LNP formulations (Table 26) were prepared using the microfluidic mixing process as described in Example 6 and using a discontinuous diafiltration process for ethanol removal as described in Example 25. The LNPs were formulated using eGFP encoding mRNA (TriLink Biotechnologies, California, US) and labeled with 0.01 mol % DiIC18(5)-DS (Invitrogen, Massachusetts, US) using the lipid ratios shown in Table 26 below. The LNPs were then inserted with a targeting conjugate using the specified conditions to provide the final targeted LNP formulations. The LNPs were characterized as described in Example 3. The characteristics of the LNPs are shown in Table 27.

TABLE 26
LNP formulation composition and Antibody conjugate insertion conditions

| | | | Lipid- | | Conjugate | |
|---|---|---|---|---|---|---|
| | | | PEG | | Insertion | |
| Ionizable | Formulation | Lipid- | Content | Targeting | Density | Insertion |
| Lipid | No. | PEG | (mol %) | Conjugate | (g/mol) | Condition |
| Lipid 8 | EXP210021 | DMG- | 2.5 | hSP34 | 9 | 60° C. for 1 h in |
| | 79-N00H60T | PEG | | | | pH 7.4 HBS |
| Lipid 5 | EXP210021 | DMG- | 2.5 | hSP34 | 9 | 37° C. for 4 h in |
| | 79-N02H374T | PEG | | | | pH 7.4 HBS |

TABLE 27
LNP Size, Charge (Dynamic Light Scattering) and mRNA encapsulation (Ribogreen assay)

| | | | Pre-Insertion | Pre-Insertion | Pre-Insertion |
|---|---|---|---|---|---|
| | | Post-Insertion | Zeta Potential at | Dye- | mRNA |
| | | PLS Z-Avg. | pH 5.5 | Accessible | content |
| | | Diameter (nm)/ | (mV)/pH 7.4 | mRNA | (ug/mL)*/% |
| Ionizable Lipid | Formulation No. | PDI | (mV) | (%) | encapsulation |
| Lipid 8 | EXP21002179-N00H60T | 75/0.24 | +21.9/+3.5 | 12 | |
| Lipid 5 | EXP21002179-N02H374T | 81/0.24 | +23.3/+3.0 | 14 | |

*mRNA content determined using the Triton-deformulation procedure described in Example 3

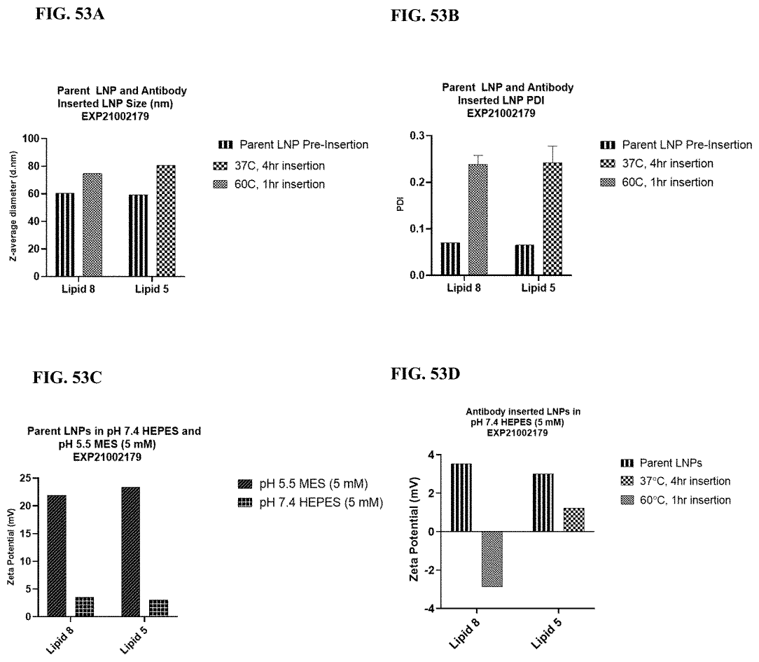

Both Lipid 5 and Lipid 8 formulations resulted in particles exhibiting hydrodynamic diameters in the sub-100 nm range (Table 27 and FIG. 53A) with narrow polydispersity (<0.1) prior to antibody conjugate insertion and moderately higher polydispersities (<0.3) after antibody conjugate insertion. Additionally, low levels of dye accessible mRNA (<15%) and high RNA encapsulation efficiencies (>80% mRNA was recovered in final formulation relative to the total RNA used in LNP batch preparation) were observed in both formulations. These improvements to LNP size distribution are attributed to the use of discontinuous diafiltration for ethanol removal (described in Example 25) relative to ethanol removal by buffer exchange using a size exclusion column (described in Example 6). As seen in FIGS. 53B and 53C, Lipid 5 and Lipid 8 LNPs exhibited positive zeta potential at pH 5.5, and either near neutral or slightly negative charge at pH 7.4 both prior to and after antibody insertion (FIG. 53D), suggesting a change in LNP ionization state as expected.

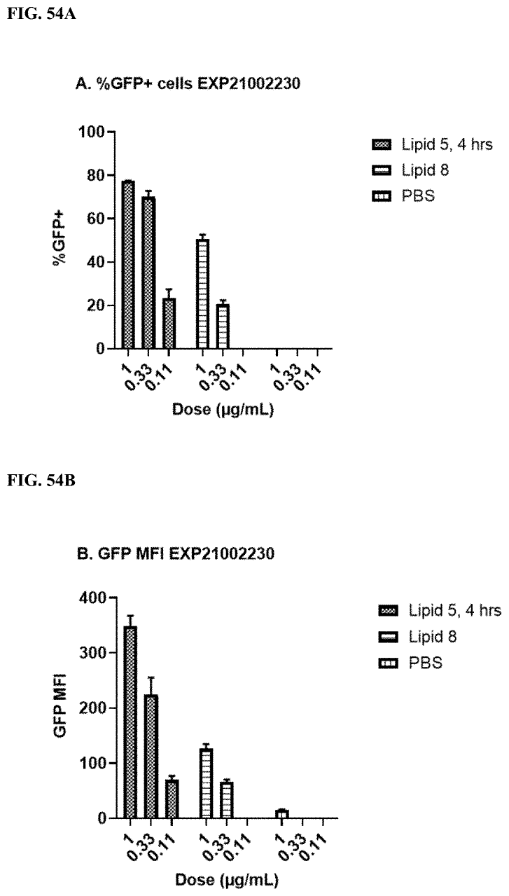

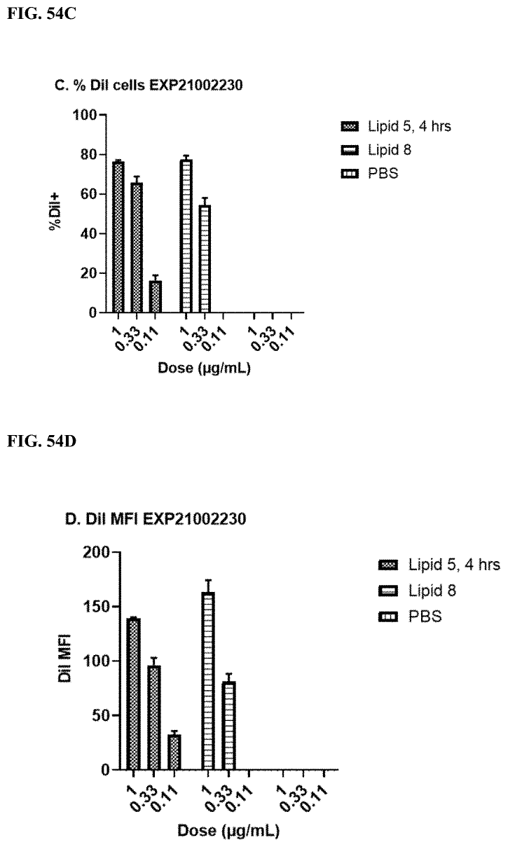

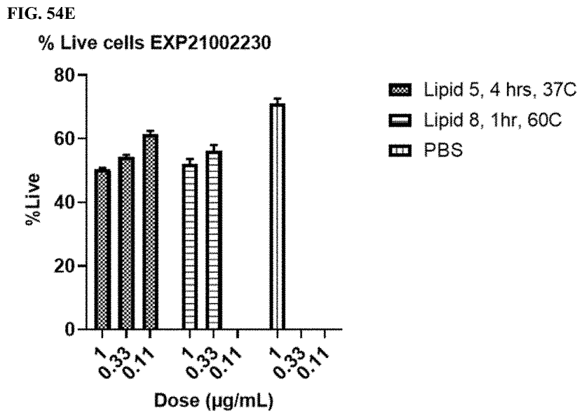

The resulting targeted LNPs were evaluated in primary human T-cells using the in vitro transfection protocol described in Example 8. Dose dependent expression of GFP protein was observed with both ionizable lipids (5 and 8) as illustrated by similar % GFP+ and GFP MFI values (FIGS. 54A and 54B). However, a two-fold higher mean fluorescence intensity (GFP MFI) was observed with the Lipid 5 LNP (at both 0.5 ug/mL and 1.0 ug/mL dose/well) suggesting more efficient cytosolic release of the mRNA payload (and thus greater GFP protein expression) with the Lipid 5 formulation relative to the Lipid 8 formulation. As illustrated by the DiI+ and DiI MFI values, both formulations were equally associated with cells suggesting the conjugate insertion process is not dependent on the ionizable lipid chemistry (FIGS. 54C and 54D). As seen in FIG. 54E, both formulations were well tolerated by T-cells at and below 1.0 μg/mL dose (minimal drop in cell viability was observed relative to the PBS control).

### Example 27—Lipid 5, Lipid 8 and DLn-MC3-DMA LNP Properties and In Vitro GFP Protein Expression in Primary Human T-Cells

This example compares the properties of LNPs prepared using Lipid 5, Lipid 8 and DLn-MC3-DMA LNP properties and in vitro GFP protein expression in primary human T-cells. All LNP formulations (Table 28) were prepared using the microfluidic mixing process (described in Example 6) and using a discontinuous diafiltration process for ethanol removal (described in Example 25). The LNPs were formulated using eGFP encoding mRNA (TriLink Biotechnologies, California, US) and labeled with 0.01 mol % DiIC18(5)-DS (Invitrogen, Massachusetts, US) using the lipid ratios shown in Table 28 below. The LNPs were then inserted with a targeting conjugate using the specified conditions to provide the final targeted LNP formulations. The LNPs were characterized as described in Example 3.

TABLE 28
LNP Formulation composition and antibody insertion conditions

| | | | | Targeting | |
|---|---|---|---|---|---|
| | | | | Conjugate/ | Antibody |
| | | | Lipid-PEG | Insertion | conjugate |
| | | | Content | density | insertion |
| Ionizable Lipid | Formulation No. | Lipid-PEG | (mol %) | (g/mol) | condition |
| DLin-MC3-DMA | EXP210034 | DPG-PEG | 1.5 | hSP34/9 | 60° C. for 0.5 h |
| | 71-N1H3 | | | | in pH 7.4 HBS |
| Lipid 8 | EXP210034 | DPG-PEG | 1.5 | hSP34/9 | 60° C. for 0.5 h |
| | 71-N2H3 | | | | in pH 7.4 HBS |
| Lipid 5 | EXP210034 | DPG-PEG | 1.5 | hSP34/9 | 37° C. for 4 h |
| | 71-N3H3 | | | | inpH 7.4 HBS |

TABLE 29
LNP Size, Charge (Dynamic Light Scattering) and mRNA encapsulation (Ribogreen assay)

| | | | | Pre- | Pre- |
|---|---|---|---|---|---|
| | | Post- | Pre- | Insertion | insertion |
| | | Insertion | Insertion | Dye- | total |
| | | DLS Z-Avg. | Zeta Potential | Accessible | mRNA |
| | | Diameter | at pH 5.5(mV)/ | mRNA | content |
| Ionizable Lipid | Formulation No. | (nm)/PDI | pH 7.4(mV) | (%) | (ug/mL) |
| DLin-MC3-DMA | EXP21003471-N1H3 | 107/0.20 | 16.4/−0.1 | 9.1 | |
| Lipid 8 | EXP21003471-N2H3 | 103/0.19 | 16.2/1.6 | 10 | |
| Lipid 5 | EXP21003471-N3H3 | 92/0.11 | 20.1/4.4 | 6.4 | |

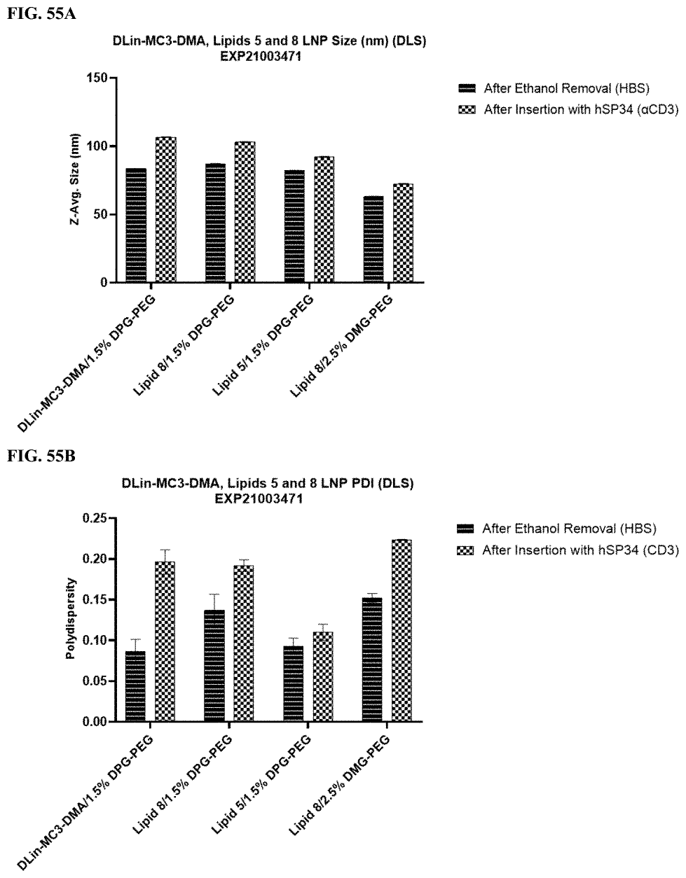

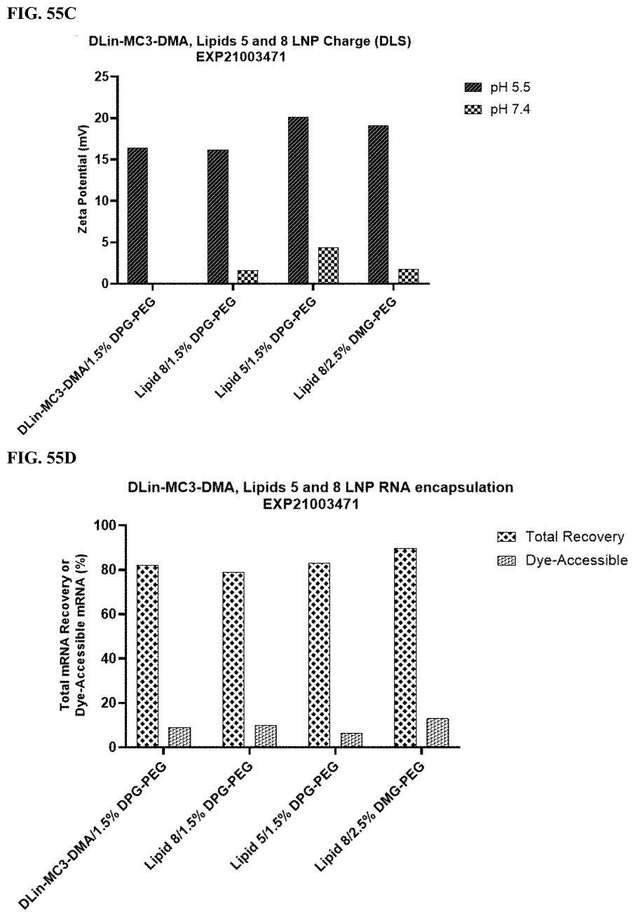

DLn-MC3-DMA, Lipid 5 and Lipid 8 were formulated using 1.5 mole % DPG-PEG. As seen in Table 29 and FIGS. 55A to 55B, all LNPs display sub-100 nm hydrodynamic diameter (DLS) prior to antibody insertion and roughly 100 nm or smaller post antibody conjugate insertion. Lipid 5 LNP polydispersity remains narrow (<0.15) post antibody conjugate insertion, while DLn-MC3-DMA and Lipid 8 exhibit a slightly larger change polydispersity (—0.2 after insertion. Additionally, low levels of dye accessible mRNA (<15%) and high RNA encapsulation efficiencies (>80% mRNA in parent LNP samples) were observed in all formulations (Table 29 and FIG. 55D). As shown in FIG. 55C, all three formulations exhibited positive zeta potential at pH 5.5, and either near neutral or slightly negative charge at pH 7.4 prior to antibody insertion, suggesting a change in LNP ionization state as expected. The resulting targeted LNPs were evaluated in primary human T-cells using the in vitro transfection protocol described in example 8.

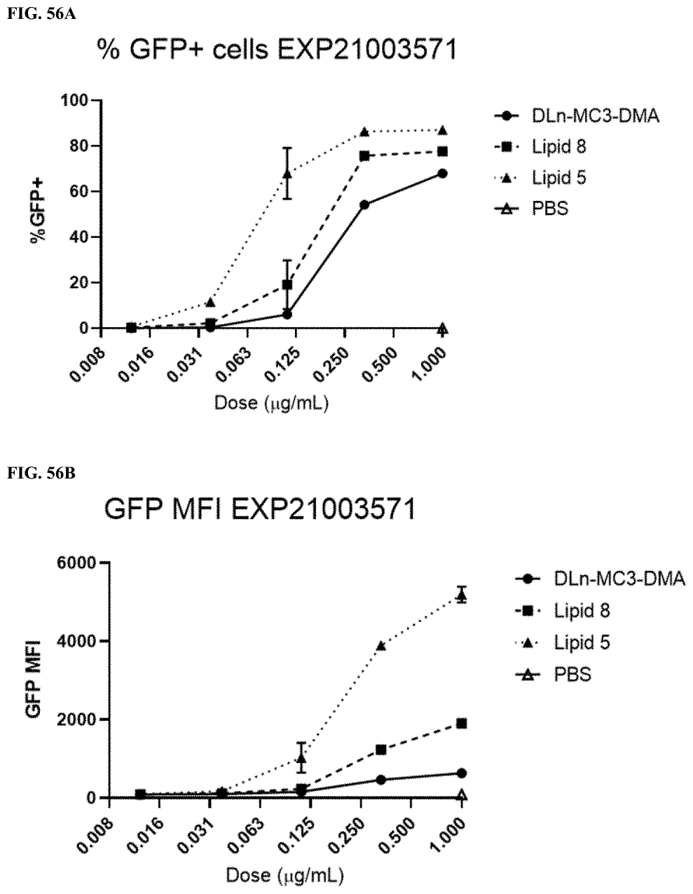

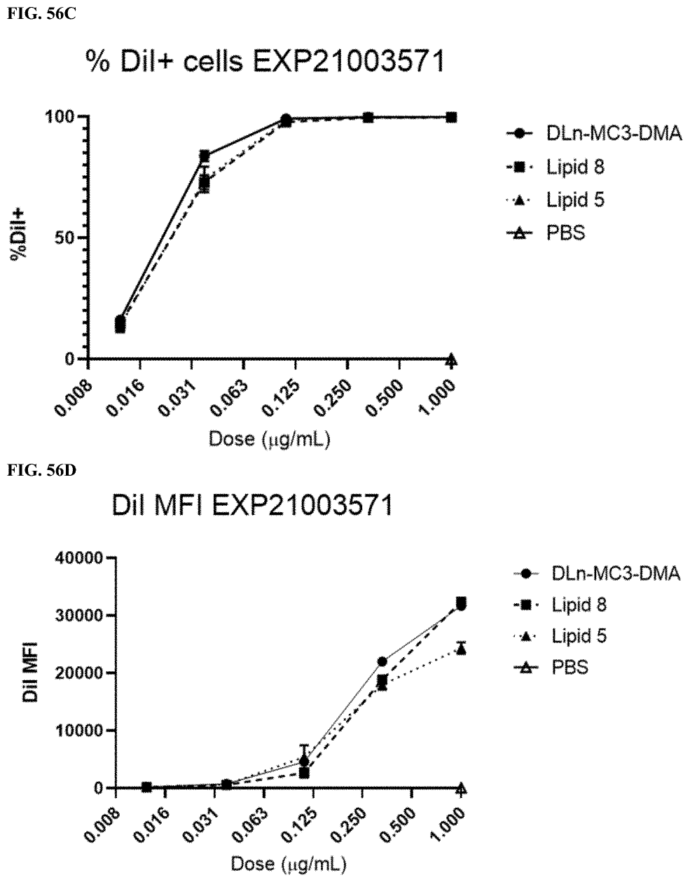

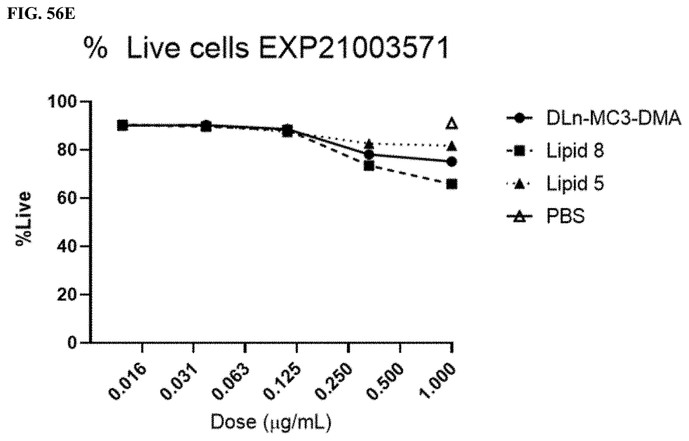

As seen in FIG. 56E, all formulations were well tolerated by T-cells below 0.125 μg/mL dose (similar to the PBS control). However, the two ester based lipids, DLn-MC3-DMA and Lipid 5 were better tolerated at higher doses, with Lipid 5 being the least toxic at 1 ug/mL dose. As illustrated by the DiI+ and DiI MFI values (FIGS. 56C and 56D), all formulations show similar levels of cell association at most dose levels tested suggesting that the conjugate insertion process is not dependent on the ionizable lipid chemistry. In all cases, dose dependent expression of GFP protein was observed (FIGS. 56A and 56B). However, at all doses tested Lipid 5 outperformed both Lipid 8 and DLn-MC3-DMA, showing >2 fold higher mean fluorescence intensity (GFP MFI) at 0.5 ug/mL and 1.0 ug/mL dose/well relative to Lipid 8 and >5 fold relative to DLn-MC3-DMA, suggesting more efficient cytosolic release of the mRNA payload (and thus greater GFP protein expression) with the Lipid 5 formulation relative to both Lipid 8 and DLn-MC3-DMA formulations.

### Example 28—Lipid 5 LNP Formulation Stability After Freeze Thaw Stress

This example illustrates the stability of Lipid 5 LNP formulations after one freeze thaw cycle. Lipid 5 LNP formulation compositions shown in Table 30 were prepared using the microfluidic mixing process (described in Example 6) and using a discontinuous diafiltration process for ethanol removal (described in Example 25). The LNPs were formulated using eGFP encoding mRNA (TriLink Biotechnologies, California, US) and labeled with 0.06 mol % DiIC18(5)-DS (Invitrogen, Massachusetts, US). The LNPs were then inserted with a targeting conjugate using the specified conditions to provide the final targeted LNP formulation. The LNPs were characterized for size by DLS and mRNA content as described in Example 3.

Following the preparation of the targeted LNP formulation, the formulation was split into two portions. One portion was exchanged into 40 mM pH 7.4 HEPES buffer, and the other portion was exchanged into 30 mM pH 7.4 Tris buffer. The buffer exchange was performed using a discontinuous diafiltration method where the LNP formulation sample was transferred into a centrifugal ultrafiltration device with 100,000 kDa MWCO regenerated cellulose membrane (Amicon Ultra-4, MilliporeSigma, Massachusetts, US), then diluted 10-fold with the exchange buffer, and concentrated back to the original volume by centrifuging at 500 rcf. This dilution and concentration step was repeated one additional time. The exchanged LNP samples were then divided into separate aliquots that were mixed with concentrated sodium chloride and sucrose solutions to provide the final freeze-thaw sample formulations. Samples of each freeze-thaw formulation were then stored at 4° C., frozen at −80° C., or flash frozen in liquid nitrogen. The samples were later thawed at room temperature and tested for size by DLS and in vitro T cell transfection.

TABLE 30
LNP Formulation composition and antibody insertion conditions

| | | | Lipid- | Targeting | Insertion |
|---|---|---|---|---|---|
| | | | PEG | Conjugate/ | |
| | Ionizable | | Content | Insertion | |
| Formulation No. | Lipid | Lipid-PEG | (mol %) | density (g/mol) | Condition |
| EXP21001639-N14 | Lipid 5 | DMG-PEG | 2.5 | hSP34/9 | 37° C. for 14 h in |
| | | | | | pH 7.4 HBS |

TABLE 31
Lipid 5 LN P Size and Poly dispersity (1 DLS) prior to insertion

| | Post-Insertion | Post- | Pre- |
|---|---|---|---|
| | DLS Z- | Insertion | Insertion Dye- |
| | Avg. Diameter | DLS | Accessible ) |
| Formulation No. | (nm) | PDI | mRNA (% |
| EXP2100 1639-N14 | 106 | 0.19 | 24 |

TABLE 32
LNP formulation composition and list of storage condition/freezing
methods for Lipid 5 LNPs

| | | | Sucrose | | |
|---|---|---|---|---|---|
| Lipid 5 LNP, | Buffer | Buffer Conc. | Conc. | NaCl Conc. | |
| Formulation No. | Species | (mM) | (wt %) | (mM) | Storage Condition |
| EXP21001639-N24 | pH 7.4 HEPES | 20 | 9.6 | 0 | 4° C. |
| EXP21001639-N28 | pH 7.4 HEPES | 20 | 9.6 | 0 | −80° C. |
| EXP21001639-N2N | pH 7.4 HEPES | 20 | 9.6 | 0 | LN2* |
| EXP21001639-N34 | pH 7.4 HEPES | 20 | 9.6 | 25 | 4° C. |
| EXP21001639-N38 | pH 7.4 HEPES | 20 | 9.6 | 25 | −80° C. |
| EXP21001639-N3N | pH 7.4 HEPES | 20 | 9.6 | 25 | LN2 |
| EXP21001639-N44 | pH 7.4 HEPES | 20 | 9.6 | 50 | 4° C. |
| EXP21001639-N48 | pH 7.4 HEPES | 20 | 9.6 | 50 | −80° C. |
| EXP21001639-N4N | pH 7.4 HEPES | 20 | 9.6 | 50 | LN2 |
| EXP21001639-N54 | pH 7.4 HEPES | 20 | 18.6 | 0 | 4° C. |
| EXP21001639-N58 | pH 7.4 HEPES | 20 | 18.6 | 0 | −80° C. |
| EXP21001639-N5N | pH 7.4 HEPES | 20 | 18.6 | 0 | LN2 |
| EXP21001639-N84 | pH 7.4 Tris | 15 | 9.6 | 0 | 4° C. |
| EXP21001639-N88 | pH 7.4 Tris | 15 | 9.6 | 0 | −80° C. |
| EXP21001639-N8N | pH 7.4 Tris | 15 | 9.6 | 0 | LN2 |
| EXP21001639-N94 | pH 7.4 Tris | 15 | 18.6 | 0 | 4° C. |
| EXP21001639-N98 | pH 7.4 Tris | 15 | 18.6 | 0 | −80° C. |
| EXP21001639-N9N | pH 7.4 Tris | 15 | 18.6 | 0 | LN2 |

*LN2: Liquid Nitrogen

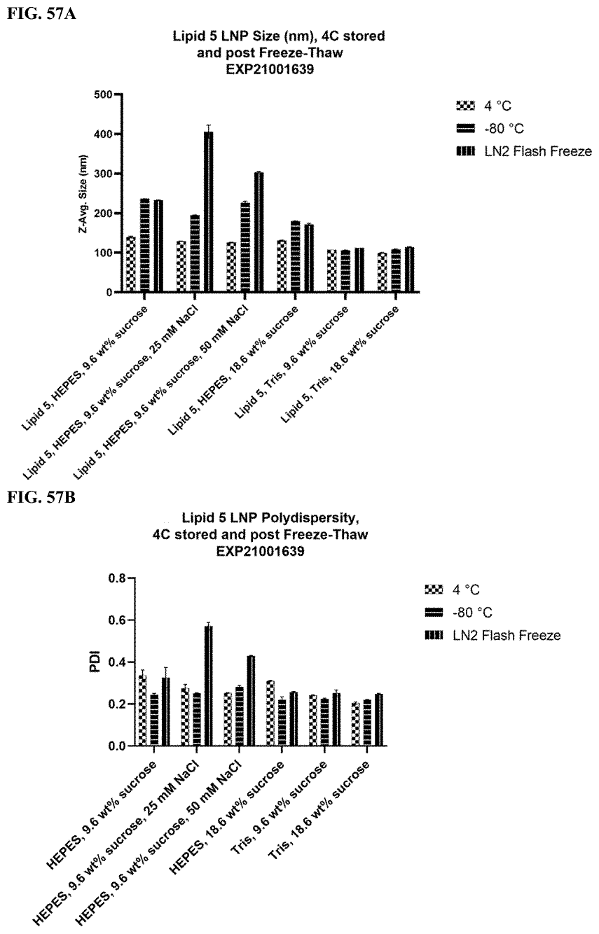

As shown in FIGS. 57A and 57B, the freezing method used (flash frozen in Liquid Nitrogen versus storage in −80 C freezer) did not impact the post freeze-thaw LNP size distributions with both hydrodynamic radius and polydispersity trending similarly between the two methods. However, the cryoprotectant and buffer composition significantly impacted post freeze-thaw LNP size characteristics. Frozen storage in HEPES buffer with no added salt or with 25 and 50 mM NaCl as well as with either 9.6 wt. % or 18 wt. % sucrose resulted in significant increases in LNP polydispersity. In contrast, storage in TRIS buffer and either 9.6 wt. % or 18 wt. % sucrose preserved the LNP size and polydispersity effectively relative to the 4 C stored LNP that were not subjected to freeze-thaw stress. All formulations were evaluated in primary human T-cells using the in vitro transfection protocol described in Example 8.

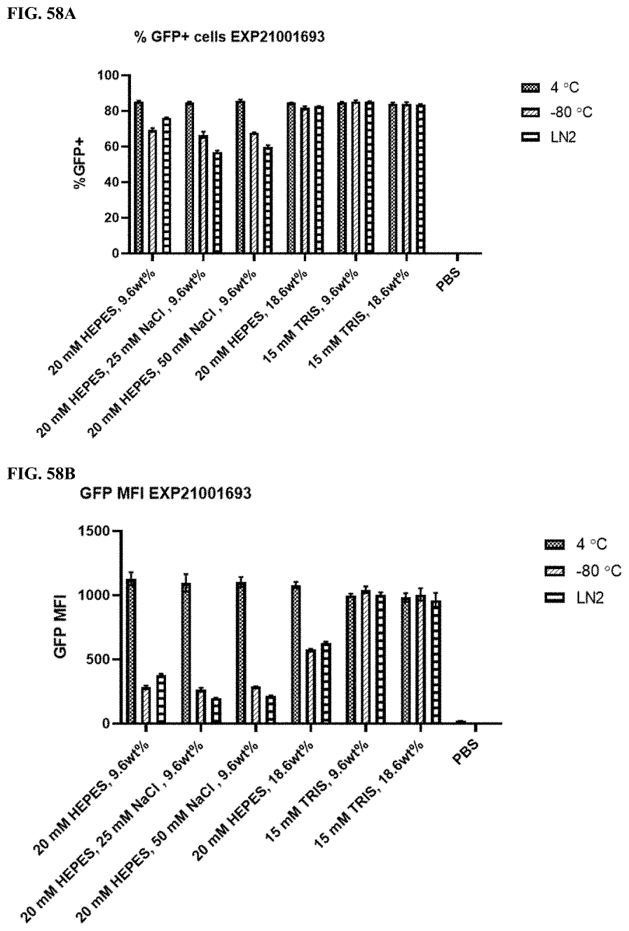

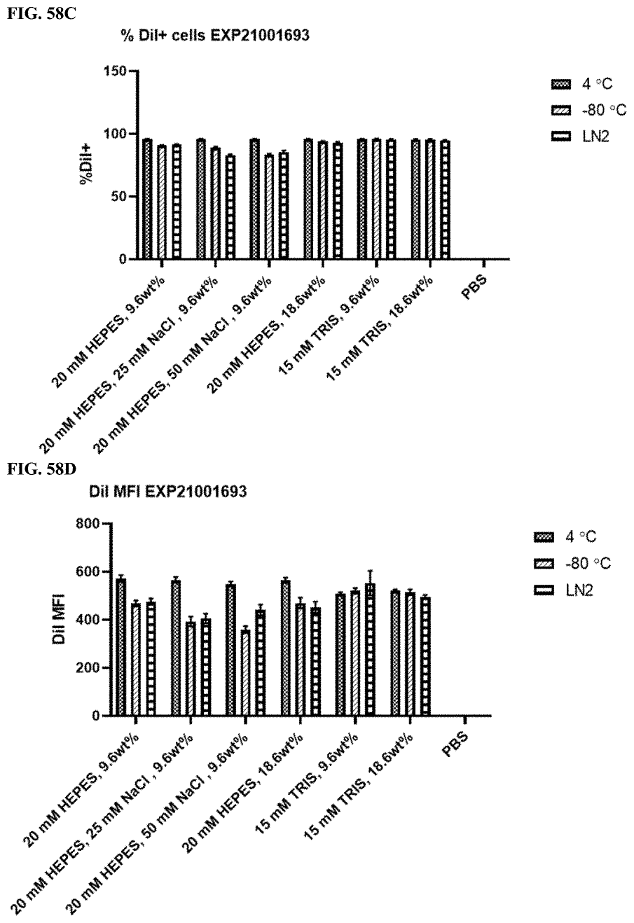

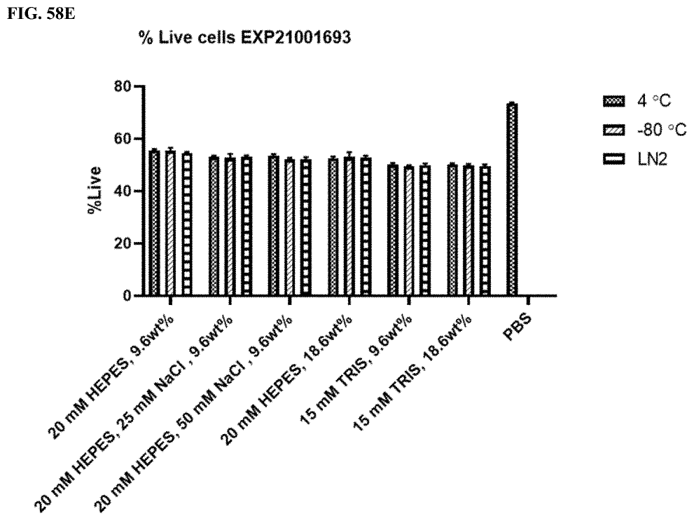

As seen in FIGS. 58A and 58B, all LNPs stored in HEPES buffer lost the ability to transfect T-cells after the freeze-thaw cycle, while formulations stored in TRIS buffer retained the ability to transfect T-cells post freeze-thaw activity. As seen in FIGS. 58C and 58D, cell association (as measured by the DiI %+ cells and DiI MFI values) of the HEPES buffer compositions was slightly diminished after the freeze thaw cycle while that of the TRIS buffer formulations was maintained. Lower cell association levels and changes in LNP size properties after freeze-thaw stress in the HEPES buffer formulations is likely responsible for the loss of activity observed. Notably, storage in TRIS buffer maintains both LNP properties and their ability to transfect T-cells after frozen storage and freeze-thaw stress.

### Example 29—Impact of PEG-lipid Anchor and PEG % on In Vivo Reprogramming

The aim of this study was to identify the optimum PEG-lipid and mol % for in vivo reprogramming of immune cells. We had hypothesized that an intermediate anchor length would be optimum for engagement of T-cells/NK cells or other immune cells in the blood since short chained anchors like PEG-DMPE or PEG-DMG (both C14) would be lost too quickly, while PEG-lipids with longer acyl chains, like PEG-DSPE and PEG-DSG (both C18), would be too stable and result in a decrease in transfection efficiency.

### Part A. Anti-CD3 Lipid 8 LNPs

In this study, we used LNPs targeted to CD3 (hsp34 Fab′-PEG-DSPE conjugate) and incorporating the Lipid 8 to test LNPs prepared with DMG-PEG (C14), DPG-PEG (C16), DPPE-PEG (16), or DSG-PEG (C18) at either 1.5 or 2.5 mol % of the total lipid.

The LNP formulations in the table below were prepared using the microfluidic mixing method described in Example 6 and discontinuous diafiltration method described in Example 25. The LNPs were formulated using eGFP encoding mRNA (TriLink Biotechnologies, California, US) and labeled with 0.06 mol % DiIC18(5)-DS (Invitrogen, Massachusetts, US). The LNPs were then inserted with a targeting conjugate using the specified conditions to provide the targeted LNP formulations. The final targeted LNPs formulations were prepared by mixing the LNP suspensions with a concentrated sucrose solution to provide the final LNP formulations with 5.3 wt % sucrose. The LNPs were characterized as described in Example 3.

TABLE 33
Formulation TABLE

| | | | Lipid- | | Conjugate | |
|---|---|---|---|---|---|---|
| | | | PEG | | Insertion | |
| Formulation | Ionizable | Lipid- | Content | Targeting | Density | Insertion |
| No. | Lipid | PEG | (mol %) | Conjugate | (g/mol) | Condition |
| EXP21001532-N1T | Lipid 8 | DMG-PEG | 2.5 | hSP34 | 9 | 60° C. for 1 h in pH |
| | | | | | | 7.4 HBS |
| EXP21001532-N2T | Lipid 8 | DPG-PEG | 2.5 | hSP34 | 9 | 60° C. for 1 h in pH |
| | | | | | | 7.4 HBS |
| EXP21001532-N3T | Lipid 8 | DSG-PEG | 2.5 | hSP34 | 9 | 60° C. for 1 h in pH |
| | | | | | | 7.4 HBS |
| EXP21001532-N4T | Lipid 8 | DPPE-PEG | 1.5 | hSP34 | 9 | 60° C. for 1 h in pH |
| | | | | | | 7.4 HBS |
| EXP21001532-N5T | Lipid 8 | DPPE-PEG | 2.5 | hSP34 | 9 | 60° C. for 1 h in pH |
| | | | | | | 7.4 HBS |
| EXP21001532-N6T | Lipid 8 | DSPE-PEG | 1.5 | hSP34 | 9 | 60° C. for 1 h in pH |
| | | | | | | 7.4 HBS |
| EXP21001532-N7T | Lipid 8 | DSPE-PEG | 2.5 | hSP34 | 9 | 60° C. for 1 h in pH |
| | | | | | | 7.4 HBS |

TABLE 34
Formulation Analysis Results

| | Post- | | Pre-Insertion | Pre-Insertion | Pre-Insertion |
|---|---|---|---|---|---|
| | Insertion | | Zeta | Zeta | Dye- |
| | DLS Z-Avg. | Post- | Potential at | Potential at | Accessible |
| | Diameter | Insertion | pH 5.5 | pH 7.4 | mRNA |
| Formulation No. | (nm) | DLS PDI | (mV) | (mV) | (%) |
| EXP21001532-N1T | 76 | 0.18 | 21.3 | 1.8 | 13 |
| EXP21001532-N2T | 86 | 0.17 | 17.8 | 1.0 | 8.7 |
| EXP21001532-N3T | 96 | 0.18 | 17.1 | −3.4 | 10 |
| EXP21001532-N4T | 119 | 0.23 | 20.1 | −5.9 | 12 |
| EXP21001532-N5T | 73 | 0.15 | 17.2 | −7.0 | 11 |
| EXP21001532-N6T | 108 | 0.17 | 17.9 | −4.9 | 10 |
| EXP21001532-N7T | 77 | 0.18 | 18.6 | −6.6 | 10 |

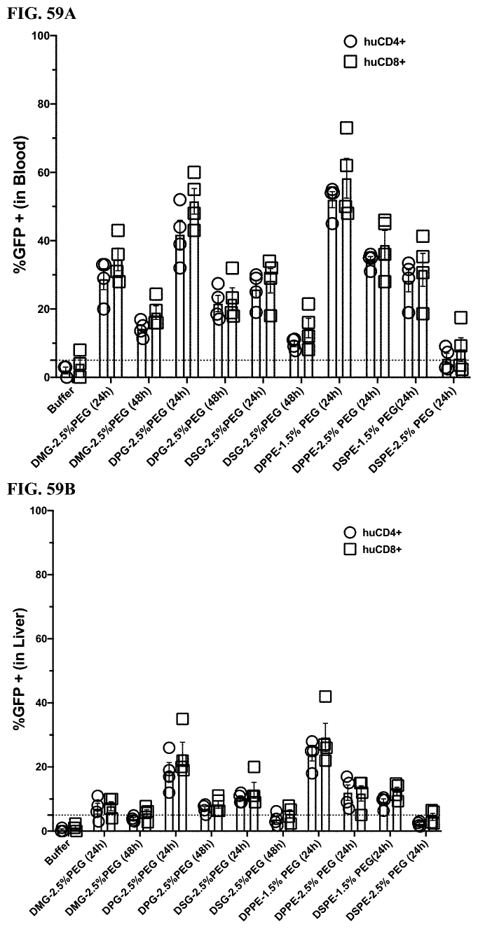

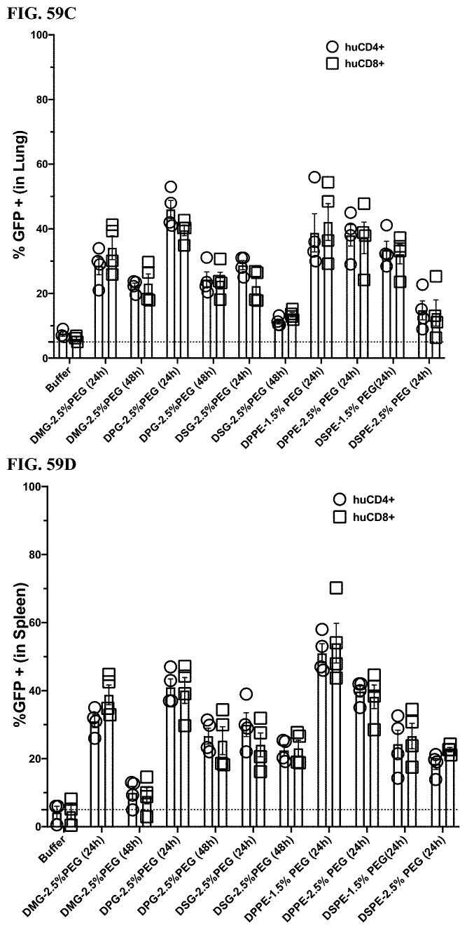

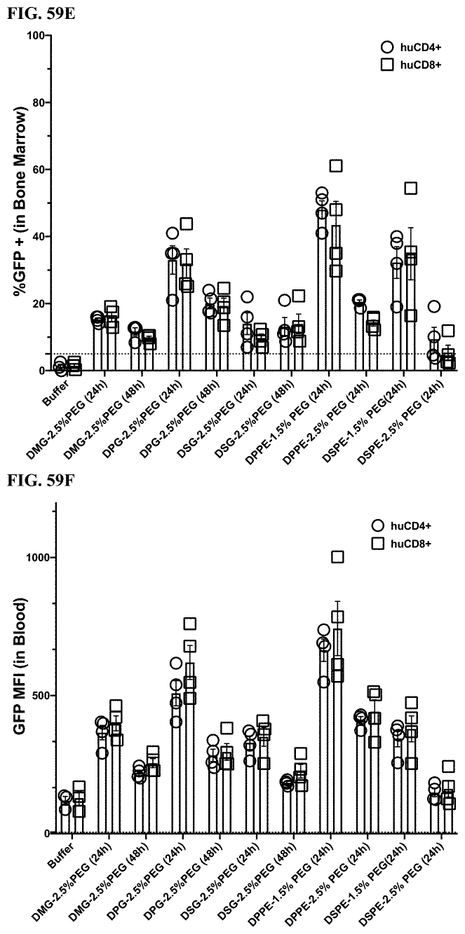

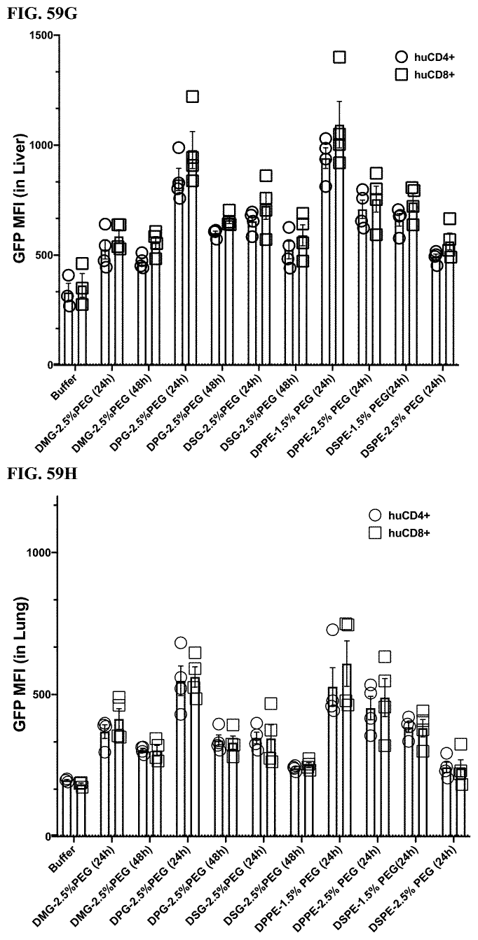

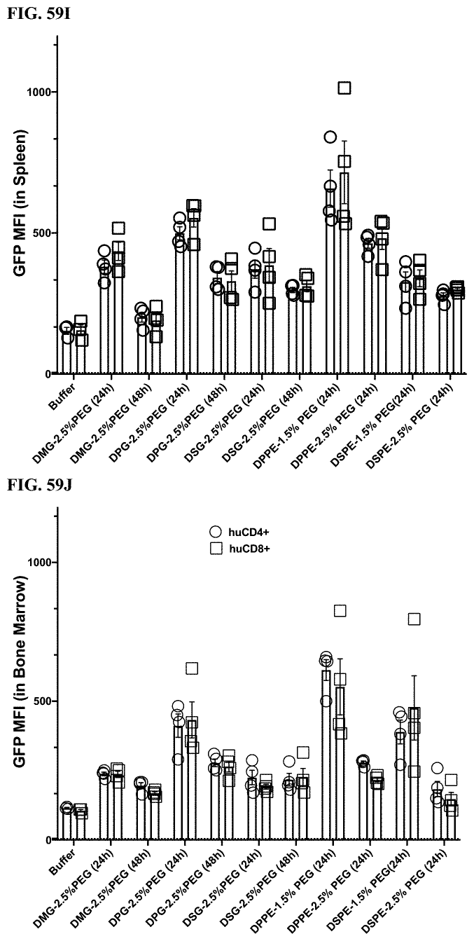

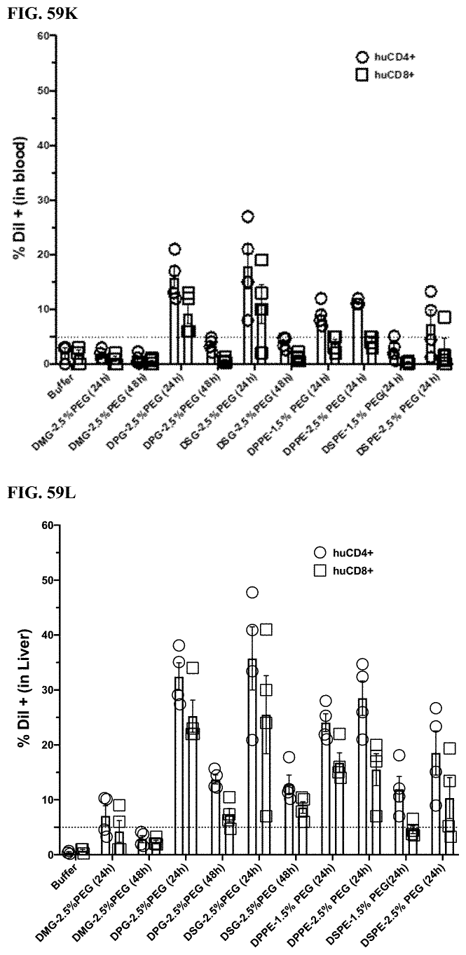

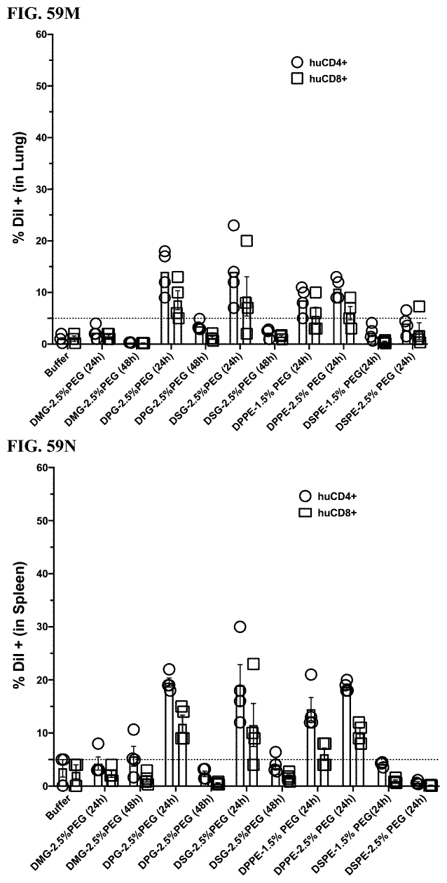

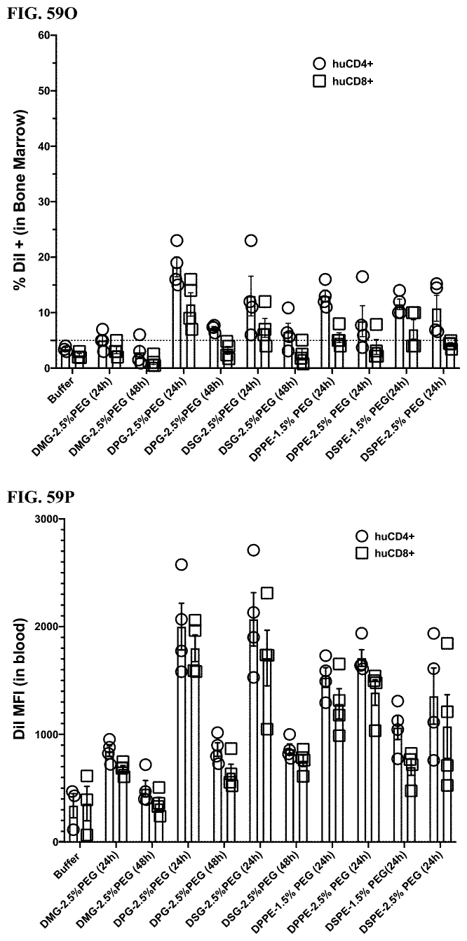

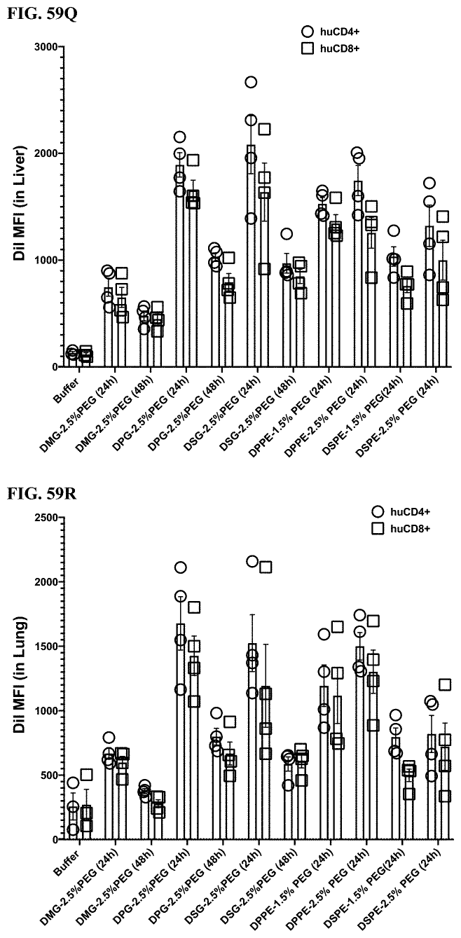

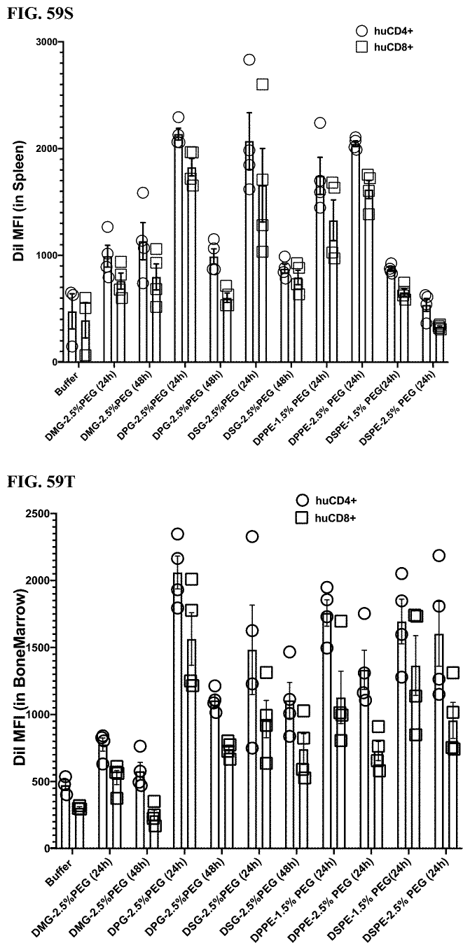

Results of in vivo reprogramming of immune cells with CD3-targeted DiI/GFP LNP at the dose of 0.3 mg/kg after 24 or 48 h with either DMG, DPG or DSG-PEG 2.5% or after 24 h with either DPPE or DSPE 1.5 or 2.5% are show in FIGS. 59A to 59T.

All formulations with anti-CD3 hsp34 clone targeted Lipid 8 LNPs showed peak GFP expression at 24 h, with maximum expression in blood>lung>spleen>bone marrow >liver. Irrespective of diacyl glycerol or phosphoethanolamine backbone, DPG-PEG or DPPE-PEG, i.e., C16 anchor length shows maximum reprogramming with 1.5% PEG-lipid, compared to PEG-lipids of other acyl chain lengths (C14 or C18). GFP, MFI showed similar trend as GFP % positive T cells. The percent positive DiI or DiI MFI also showed a similar trend as that of GFP positive T cells, indicating that with CD3 targeted Lipid 8 LNPs the binding efficiency of LNPs correlates with their reprogramming ability.

### Part B. Anti-CD3 or Anti-CD8 Lipid 5 LNPs

In this study, we used LNPs targeted to CD3 (hsp34 Fab′-PEG-DSPE conjugate) and LNPs targeted to CD8 (TRX2 Fab′-PEG-DSPE conjugate, or V2-PEG-DSPE Nb conjugate) incorporating the Lipid 5 to test LNPs prepared with DMG-PEG (C14) or DPG-PEG (C16) at 1.5 mol % of the total lipid.

The LNP formulations in the table below were prepared using the microfluidic mixing method described in Example 6 and discontinuous diafiltration method described in Example 25. The LNPs were formulated using unmodified eGFP encoding mRNA (TriLink Biotechnologies, California, US; Catalog #L-7601) and labeled with 0.06 mol % DiIC18(5)-DS (Invitrogen, Massachusetts, US). The LNPs were then inserted with a targeting conjugate using the specified conditions to provide the targeted LNP formulations. The final targeted LNPs formulations were prepared by mixing the LNP suspensions with a concentrated sucrose solution to provide the final LNP formulations with 9.6 wt % sucrose. The LNPs were characterized as described in Example 3.

TABLE 35
LNP Formulations

| | | | Lipid- | | Conjugate | |
|---|---|---|---|---|---|---|
| | | | PEG | | Insertion | |
| Formulation | Ionizable | Lipid- | Content | Targeting | Density | Insertion |
| No. | Lipid | PEG | (mol %) | Conjugate | (g/mol) | Condition |
| EXP21002551- | Lipid 5 | DMG-PEG | 1.5 | TRX2 | 9 | 37° C. for 4 h in pH |
| N6H-TRX2S | | | | | | 7.4 HBS |
| EXP21002551- | Lipid 5 | DMG-PEG | 2.5 | TRX2 | 9 | 37° C. for 4 h in pH |
| N7H-TRX2S | | | | | | 7.4 HBS |
| EXP21002414- | Lipid 5 | DPG-PEG | 1.5 | TRX2 | 9 | 37° C. for 4 h in pH |
| N2H-TRX2S | | | | | | 7.4 HBS |
| EXP21002551- | Lipid 5 | DPG-PEG | 2.5 | TRX2 | 9 | 37° C. for 4 h in pH |
| N5H-TRX2S | | | | | | 7.4 HBS |
| EXP21002551- | Lipid 5 | DMG-PEG | 1.5 | hSP34 | 9 | 37° C. for 4 h in pH |
| N6H-SP34S | | | | | | 7.4 HBS |
| EXP21002551- | Lipid 5 | DMG-PEG | 2.5 | hSP34 | 9 | 37° C. for 4 h in pH |
| N7H-SP34S | | | | | | 7.4 HBS |
| EXP21002414- | Lipid 5 | DPG-PEG | 1.5 | hSP34 | 9 | 37° C. for 4 h in pH |
| N2H-SP34S | | | | | | 7.4 HBS |
| EXP21002551- | Lipid 5 | DPG-PEG | 2.5 | hSP34 | 9 | 37° C. for 4 h in pH |
| N5H-SP34S | | | | | | 7.4 HBS |
| EXP21002551- | Lipid 5 | DMG-PEG | 1.5 | V2 | 5.86 | 37° C. for 4 h in pH |
| N6H-NbV2 | | | | | | 7.4 HBS |
| EXP21002551- | Lipid 5 | DMG-PEG | 2.5 | V2 | 5.86 | 37° C. for 4 h in pH |
| N7H-Nb V2 | | | | | | 7.4 HBS |
| EXP21002414- | Lipid 5 | DPG-PEG | 1.5 | V2 | 5.86 | 37° C. for 4 h in pH |
| N2H-Nb V2 | | | | | | 7.4 HBS |
| EXP21002551- | Lipid 5 | DPG-PEG | 2.5 | V2 | 5.86 | 37° C. for 4 h in pH |
| N5H-Nb V2 | | | | | | 7.4 HBS |

TABLE 36
Formulation Analysis Results

| | | | Pre-Insertion | Pre-Insertion | |
|---|---|---|---|---|---|
| | Post-Insertion | | Zeta | Zeta | Pre-Insertion |
| | DLS Z-Avg. | Post- | Potential at | Potential at | Dye-Accessible |
| | Diameter | Insertion | pH 5.5 | pH 7.4 | mRNA |
| Formulation No. | (nm) | DLS PDI | (mV) | (mV) | (%) |
| EXP21002551- | 83 | 0.06 | 23.7 | 5.2 | 14 |
| N6H-TRX2S | | | | | |
| EXP21002551- | 70 | 0.15 | 21.9 | 4.0 | 30 |
| N7H-TRX2S | | | | | |
| EXP21002414- | 90 | 0.12 | 27.9 | 7.3 | 6.9 |
| N2H-TRX2S | | | | | |
| EXP21002551- | 76 | 0.19 | 22.9 | 5.5 | 17 |
| N5H-TRX2S | | | | | |
| EXP21002551- | 96 | 0.16 | 23.7 | 5.2 | 14 |
| N6H-SP34S | | | | | |
| EXP21002551- | 138 | 0.26 | 21.9 | 4.0 | 30 |
| N7H-SP34S | | | | | |
| EXP21002414- | 104 | 0.14 | 27.9 | 7.3 | 6.9 |
| N2H-SP34S | | | | | |
| EXP21002551- | 79 | 0.17 | 22.9 | 5.5 | 17 |
| N5H-SP34S | | | | | |
| EXP2100255 1- | 85 | 0.10 | 23.7 | 5.2 | 14 |
| N6H-Nb V2 | | | | | |
| EXP2100255 1- | 69 | 0.11 | 21.9 | 4.0 | 30 |
| N7H-Nb V2 | | | | | |
| EXP21002414- | 90 | 0.12 | 27.9 | 7.3 | 6.9 |
| N2H-Nb V2 | | | | | |
| EXP21002551- | 82 | 0.21 | 22.9 | 5.5 | 17 |
| N5H-Nb V2 | | | | | |

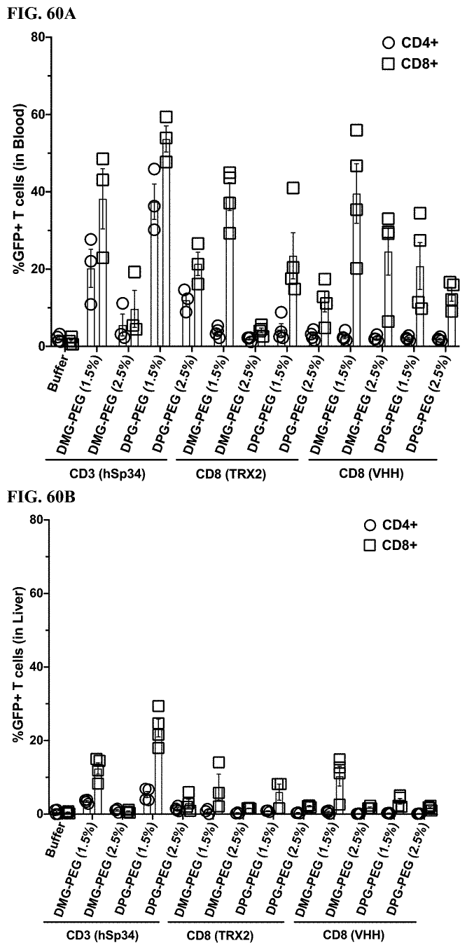

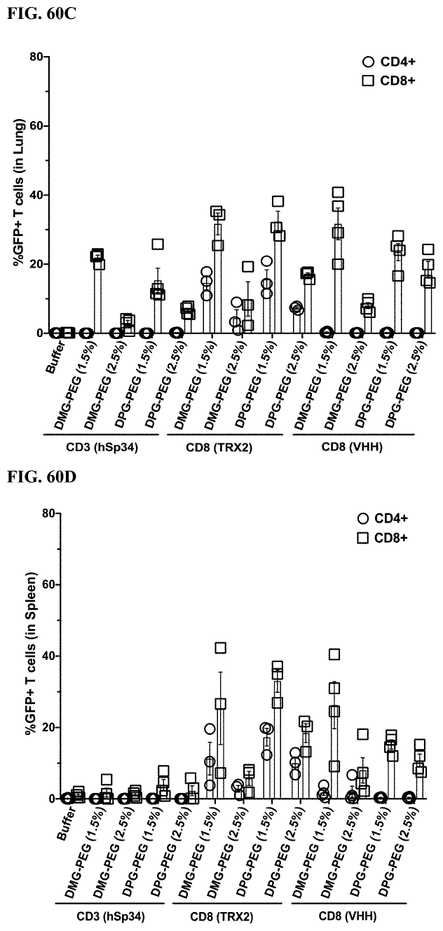

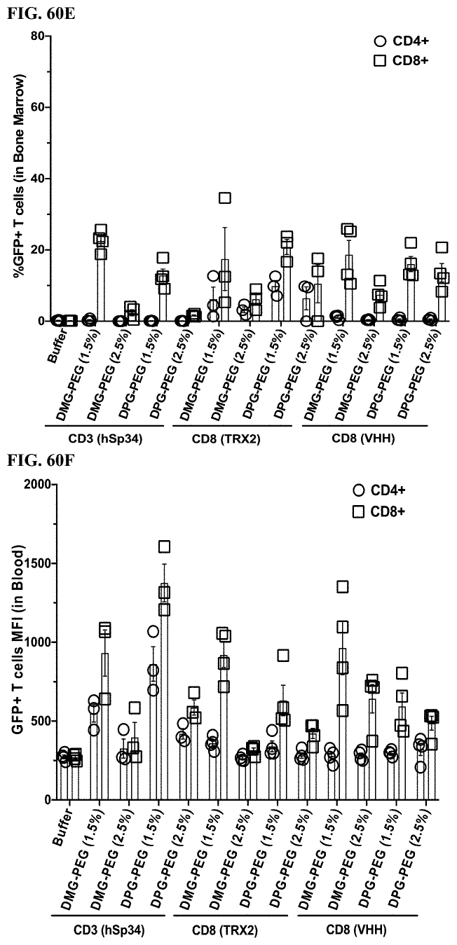

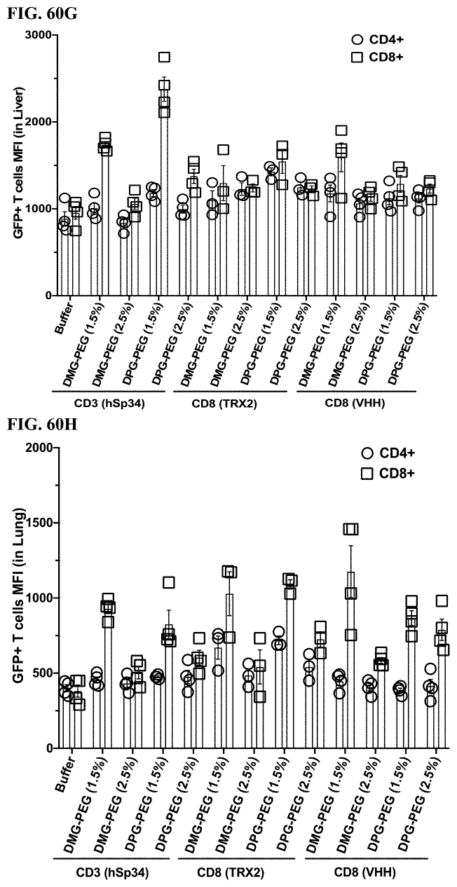

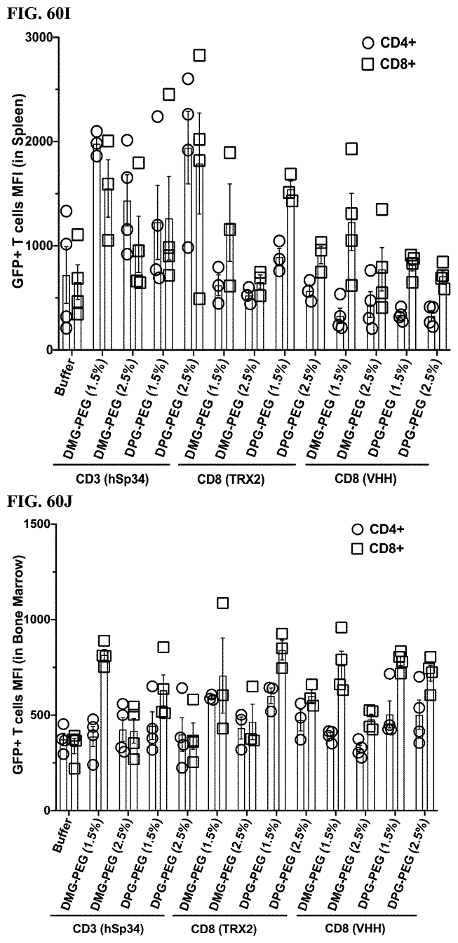

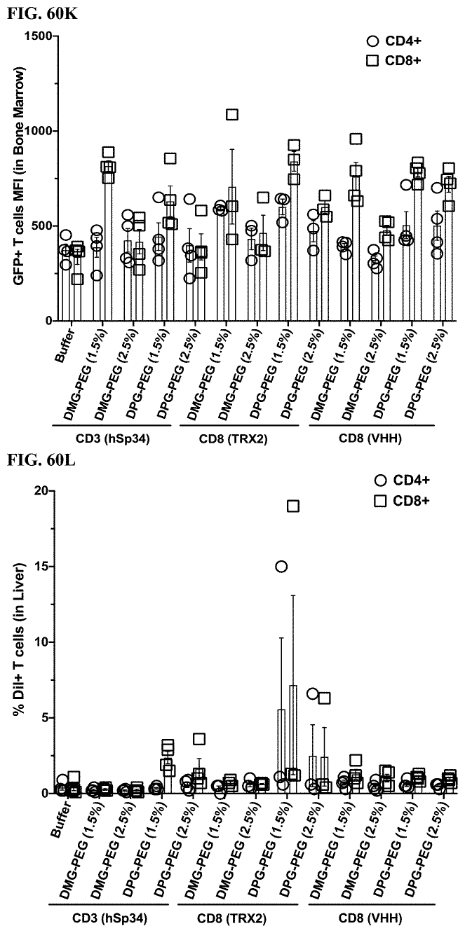

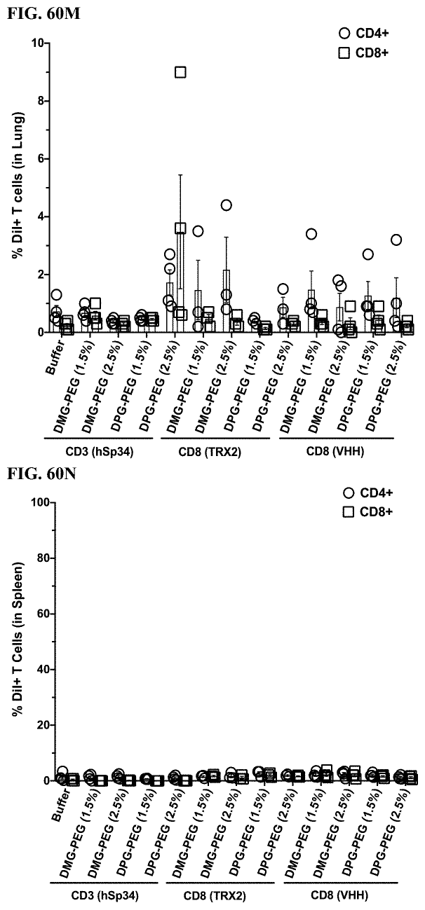

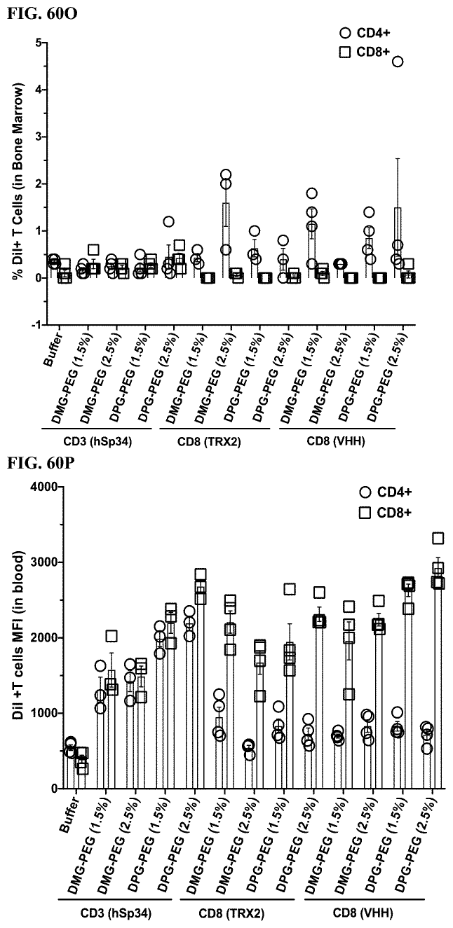

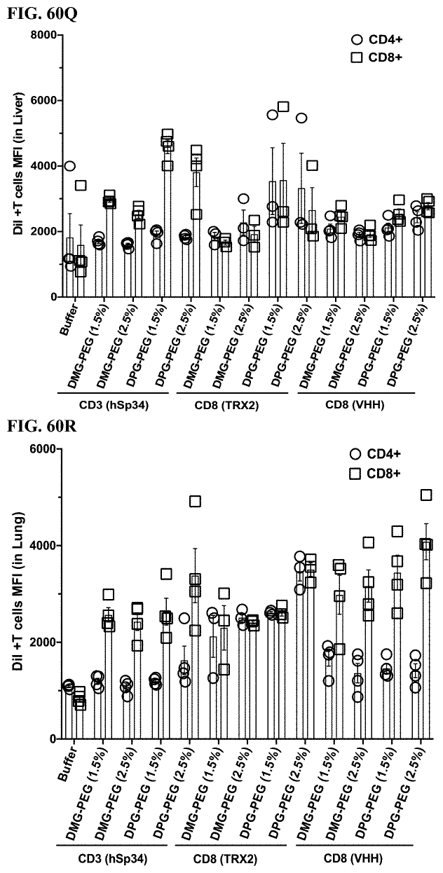

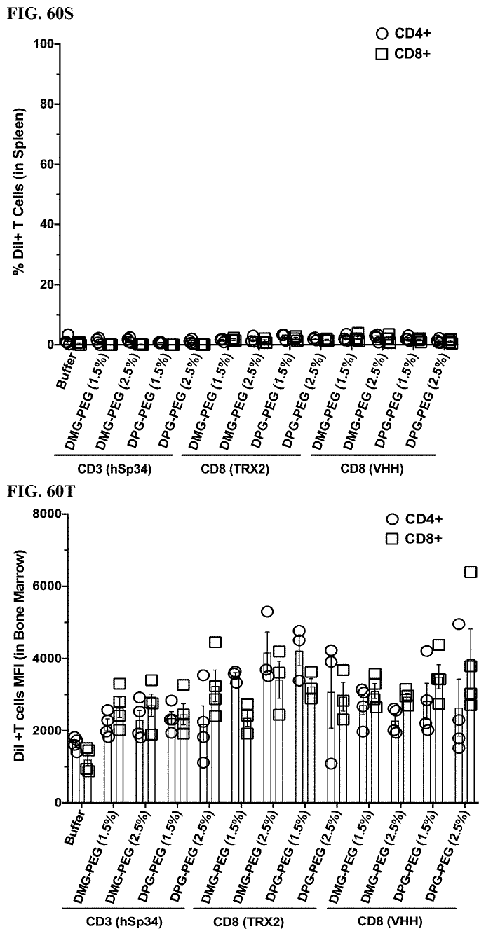

Results of in vivo reprogramming of immune cells with CD3- or CD8-targeted DiI/GFP LNP at 0.3 mg/kg of Lipid 5 with either DMG-PEG or DPG-PEG (1.5 or 2.5%) after 24 h are show in FIGS. 60A to 60T.

Lipid 5 CD3 targeted LNPs showed reprogramming of both CD4 and CD8 T cells, whereas CD8 targeted antibody TRX2 or CD8 Nanobody is specific for reprogramming CD8 T cells as expected. Similarly, CD3 targeted LNPs binds to both CD4 and CD8 T cells, and CD8 targeted LNPs with either antibody or Nanobody only binds to CD8 T cells. CD3 targeted Lipid 5 LNPs showed similar GFP expression with both DMG or DPG (i.e., C14, or C16 lipid anchor length) with 1.5% PEG, whereas in blood CD8 antibody or Nanobody targeted LNPs showed maximum GFP expression with DMG-PEG, which was 2-fold more compared to DPG-PEG at 24h. In other tissues (e.g., lung, spleen and bone marrow), CD8 targeting LNPs with either antibody or Nanobody showed similar GFP expression with both DMG-PEG and DPG-PEG-1.5%. GFP MFI showed similar trend as that of GFP expression. % DiI positive T cells are only observed in blood but not in other compartments, and DiI MFI is also maximum in blood compartment.

### Part C. Anti-CD8, Anti-CD11a, and Anti-CD4 Lipid 5 LNPs

In this study, we used LNPs targeted to CD8 (Nanobody-PEG-DSPE conjugate), LNPs targeted to CD11a (hMHM24 Fab-PEG-DSPE conjugate), and LNPs targeted to CD4 (Nanobody-PEG-DSPE conjugate or Ibalizumab-PEG-DSPE conjugate) incorporating Lipid 5 to test LNPs prepared with DPG-PEG (C16) at 1.5 mol % of the total lipid.

The LNP formulations in the table below were prepared using the microfluidic mixing method described in Example 6 and discontinuous diafiltration method described in Example 25. The LNPs were formulated using eGFP encoding mRNA (TriLink Biotechnologies, California, US) and labeled with 0.06 mol % DiIC18(5)-DS (Invitrogen, Massachusetts, US). The LNPs were then inserted with a targeting conjugate using the specified conditions to provide the targeted LNP formulations. The LNPs were characterized as described in Example 3.

TABLE 37
LNP Formulations

| | | | Lipid- | | Conjugate | |
|---|---|---|---|---|---|---|
| | | | PEG | | Insertion | |
| | Ionizable | Lipid- | Content | Targeting | Density | Insertion |
| Formulation No. | Lipid | PEG | (mol %) | Conjugate | (g/mol) | Condition |
| EXP21003027-N201 | Lipid 5 | DPG-PEG | 1.5 | hSP34 | 9 | 37° C. for 4 h in pH |
| | | | | | | 7.4 HBS |
| EXP21003027-N105 | Lipid 5 | DMG-PEG | 1.5 | BDSn | 5.3 | 37° C. for 4 h in pH |
| | | | | (Nanobody) | | 7.4 HBS |
| EXP21003027-N206 | Lipid 5 | DPG-PEG | 1.5 | BDSn | 5.3 | 37° C. for 4 h in pH |
| | | | | (Nanobody) | | 7.4 HBS |
| EXP21003027-N107 | Lipid 5 | DMG-PEG | 1.5 | hMHM24 | 6.12 | 37° C. for 4 h in pH |
| | | | | Fab | | 7.4 HBS |
| EXP21003027-N208 | Lipid 5 | DPG-PEG | 1.5 | hMHM24 | 6.12 | 37° C. for 4 h in pH |
| | | | | Fab | | 7.4 HBS |
| EXP21003027-N109 | Lipid 5 | DMG-PEG | 1.5 | T023200008 | 1.86 | 37° C. for 4 h in pH |
| | | | | (Nanobody) | | 7.4 HBS |
| EXP21003027-N210 | Lipid 5 | DPG-PEG | 1.5 | T023200008 | 1.86 | 37° C. for 4 h in pH |
| | | | | (Nanobody) | | 7.4 HBS |
| EXP21003027-N111 | Lipid 5 | DMG-PEG | 1.5 | Ibalizumab | 6.12 | 37° C. for 4 h in pH |
| | | | | Fab | | 7.4 HBS |
| EXP21003027-N212 | Lipid 5 | DPG-PEG | 1.5 | Ibalizumab | 6.12 | 37° C. for 4 h in pH |
| | | | | Fab | | 7.4 HBS |

TABLE 38
Formulation Analysis Results

| | | | Pre- | Pre- | |
|---|---|---|---|---|---|
| | Post- | | Insertion | Insertion | Pre- |
| | Insertion | | Zeta | Zeta | Insertion |
| | DLS Z-Avg. | Post- | Potential at | Potential at | Dye- |
| Formulation | Diameter | Insertion | pH 5.5 | pH 7.4 | Accessible |
| No. | (nm) | DLS PDI | (mV) | (mV) | mRNA (%) |
| EXP21003027-N201 | 94 | 0.13 | Not measured | Not measured | 8.4 |
| EXP21003027-N105 | 85 | 0.07 | 21.5 | 5.5 | 11 |
| EXP21003027-N206 | 89 | 0.07 | Not measured | Not measured | 8.4 |
| EXP21003027-N107 | 81 | 0.10 | 21.5 | 5.5 | 11 |
| EXP21003027-N208 | 86 | 0.08 | Not measured | Not measured | 8.4 |
| EXP21003027-N109 | 83 | 0.10 | 21.5 | 5.5 | 11 |
| EXP21003027-N210 | 87 | 0.06 | Not measured | Not measured | 8.4 |
| EXP21003027-N111 | 83 | 0.10 | 21.5 | 5.5 | 11 |
| EXP21003027-N212 | 88 | 0.06 | Not measured | Not measured | 8.4 |

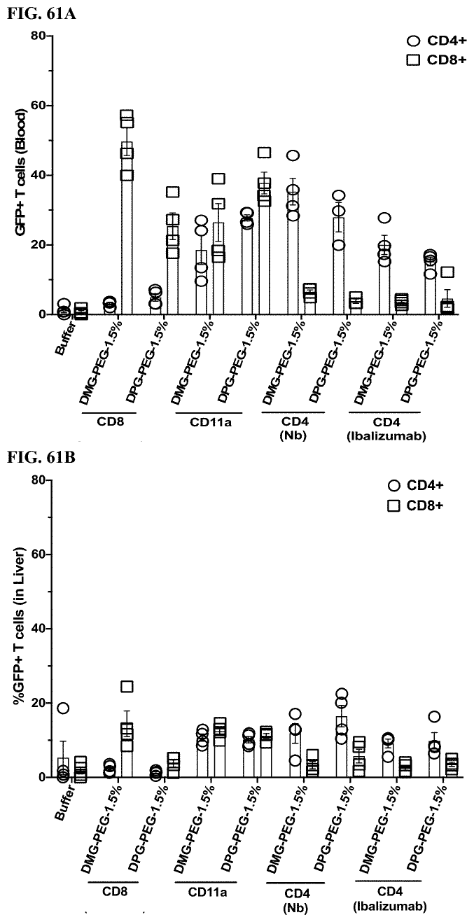

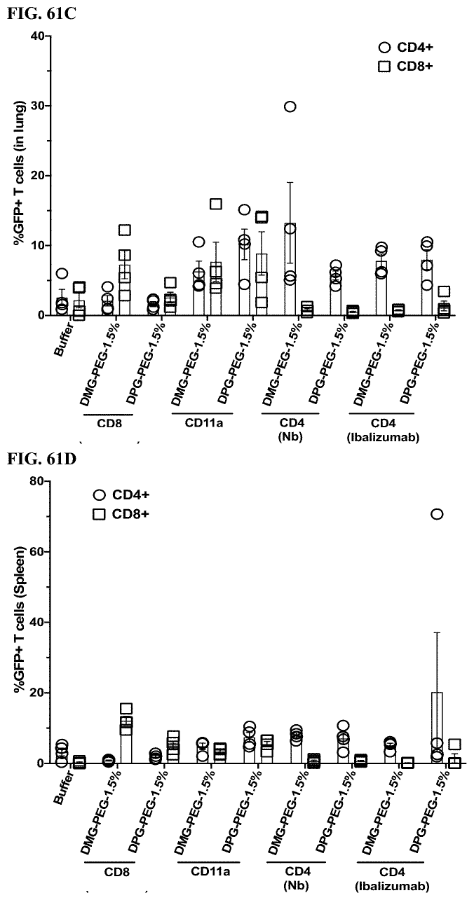

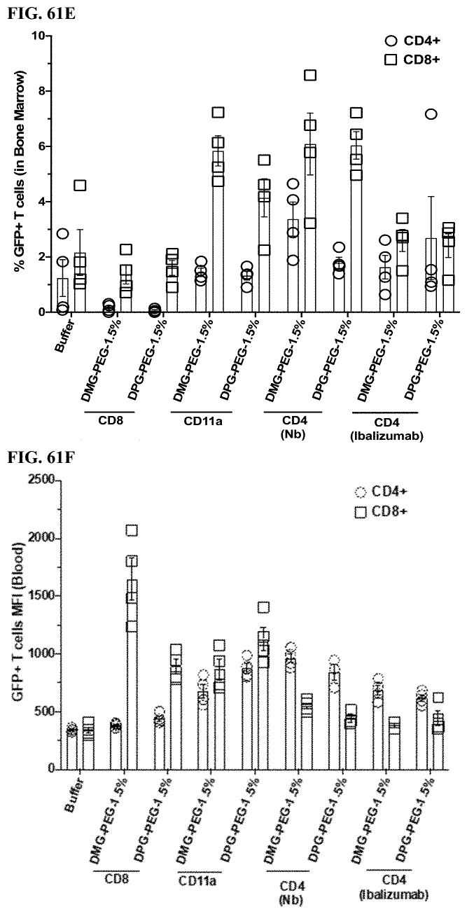

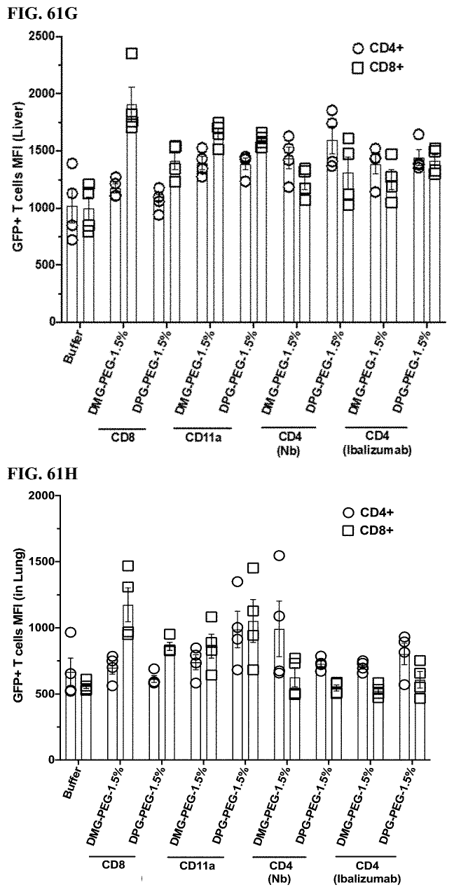

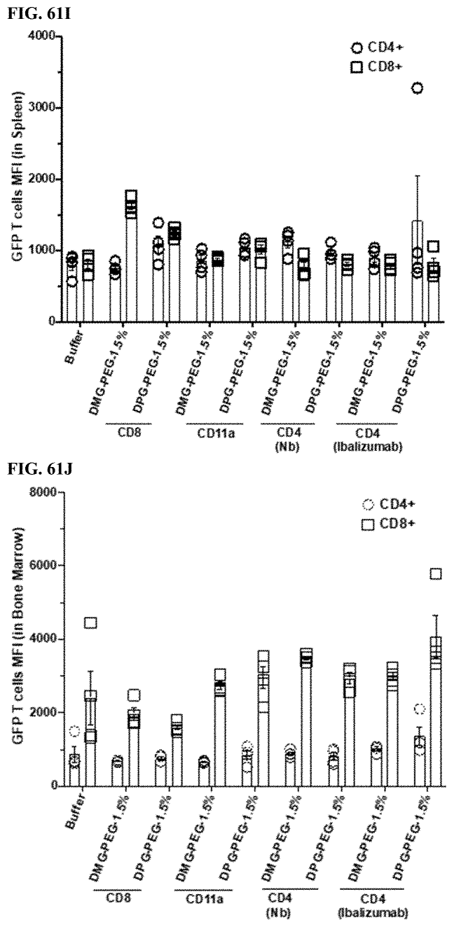

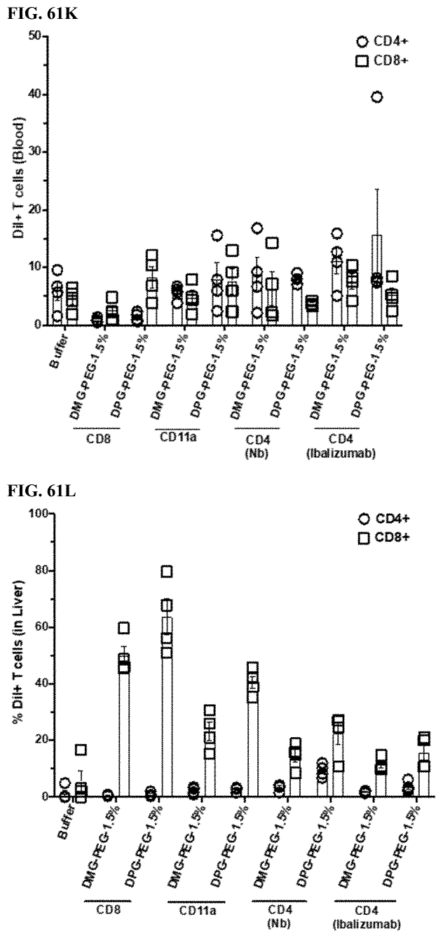

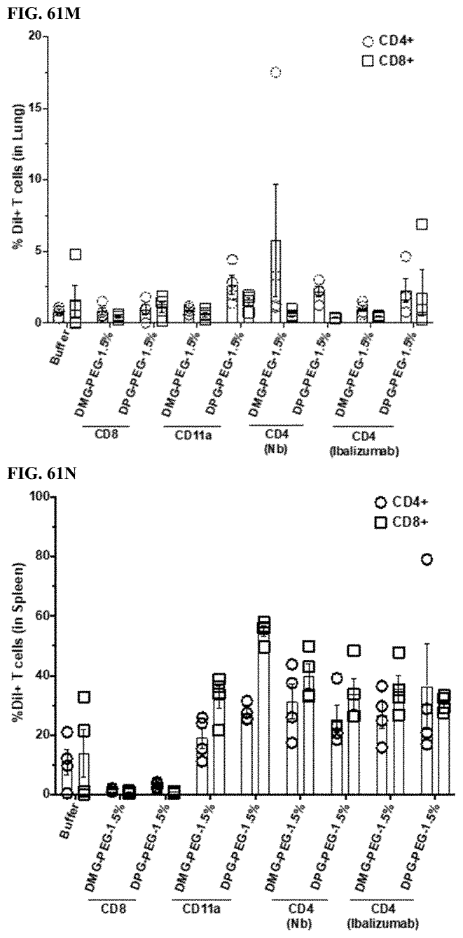

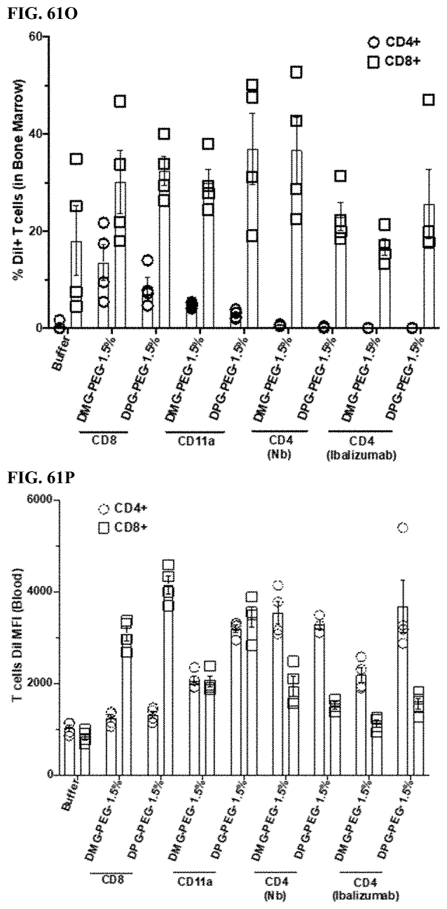

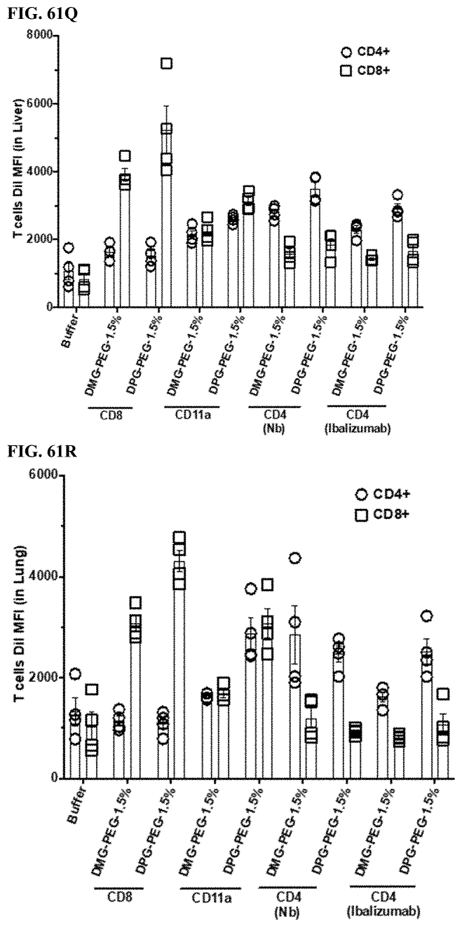

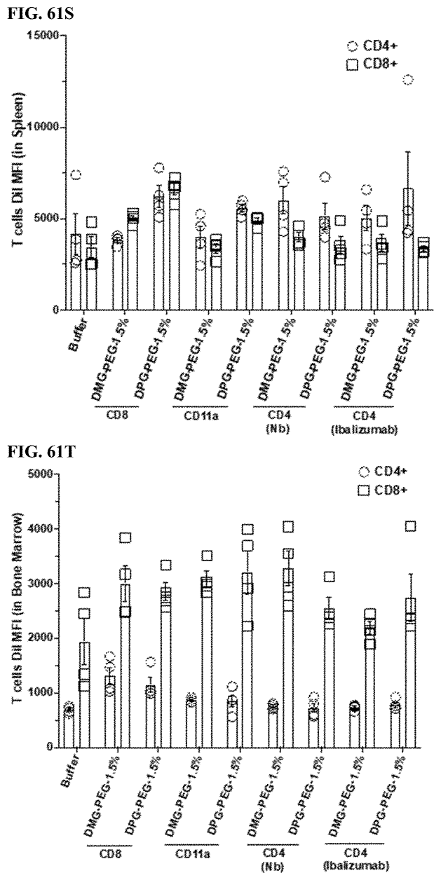

Results of in vivo reprogramming of immune cells with above LNPs at 0.3 mg/kg of Lipid 5 with either DMG-PEG or DPG-PEG (1.5 mol %) after 24 h are show in FIGS. 61A to 61T.

Lipid 5 LNP with CD8 Nanobody targeted LNPs showed GFP expression specifically in only CD8 T cells and not CD4 T cells. Similarly, CD4 targeting with either Nanobody or antibody specifically showed GFP expression in only CD4 T cells and CD11a targeting showed both CD4 and CD8 T cells expressing GFP. CD8 Nanobody LNP showed maximum GFP expression with DMG-PEG-1.5% as compared to DPG-PEG while CD11a Fab and both CD4 Nanobody and Fab antibody showed similar GFP expression with both DMG-PEG and DPG-PEG (1.5 mol %). GFP MFI showed similar trend as that of GFP % T cells. % DiI positive T cells and MFI were maximum in blood, liver and lung with different CD8, CD11a or CD4 targeted antibody or Nanobody.

### Part D. Anti-CD7 Lipid 5 LNPs

In this study, we used LNP targeted to CD7 (V1-PEG-DSPE conjugate) incorporating the Lipid 5 to test LNPs prepared with DMG-PEG (C14) or DPG-PEG (C16) at either 2.5 or 1.5 mol % of the total lipid.

The LNP formulations were prepared using the microfluidic mixing method described in Example 6 and discontinuous diafiltration method described in Example 25. The LNPs were formulated using eGFP encoding mRNA (TriLink Biotechnologies, California, US) and labeled with 0.06 mol % DiIC18(5)-DS (Invitrogen, Massachusetts, US). The LNPs were then inserted with a targeting conjugate using the specified conditions to provide the targeted LNP formulations. The LNPs were characterized as described in Example 3.

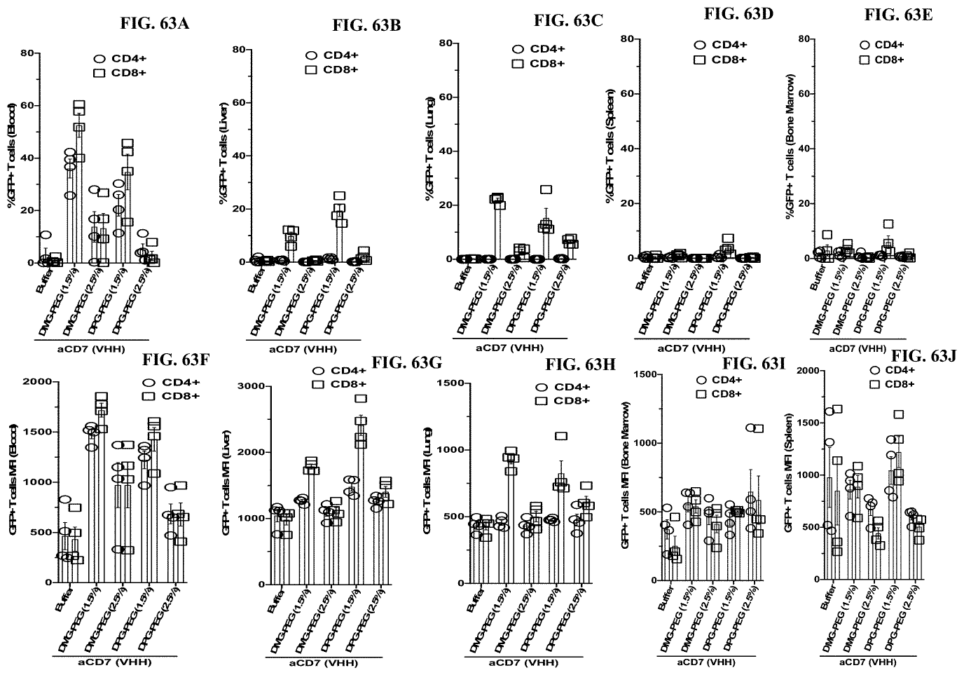

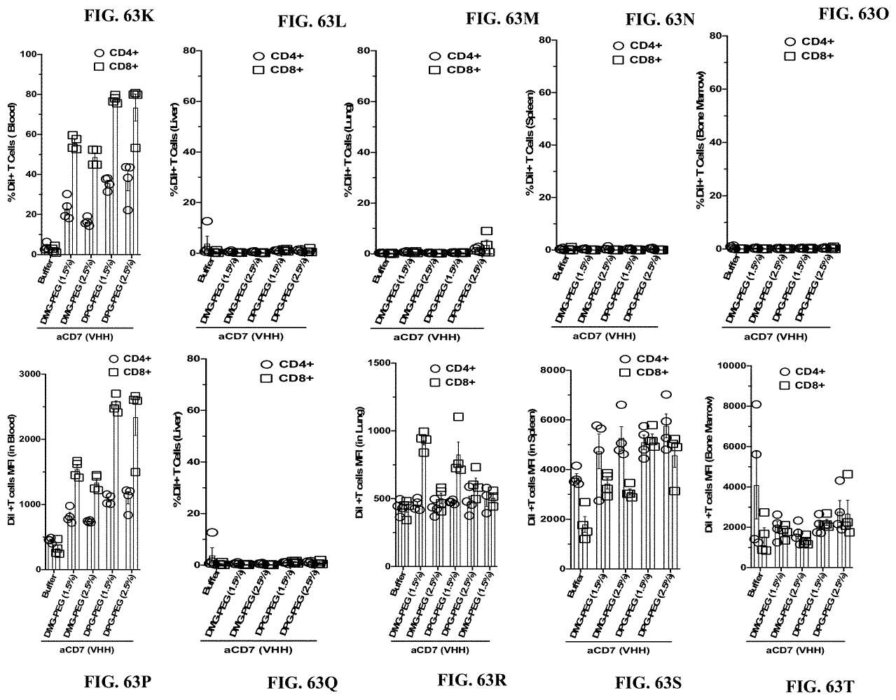

Results of in vivo reprogramming of immune cells with the LNPs at 0.3 mg/kg of Lipid 5 with either DMG-PEG or DPG-PEG (1.5%) after 24 h are show in FIGS. 63A to 63T.

Lipid 5, CD7 Nanobody targeted LNPs showed maximum GFP expressing T cells with DMG-PEG (50%) as compared to DPG-PEG (35%), both with 1.5 mol % PEG-lipid. Other tissues liver and lung showed equal 20% GFP expressing T cells with both DMG-PEG and DPG-PEG-1.5%. GFP MFI showed similar trend. DiI positive T cells and DiI MFI showed maximum binding only in blood where most GFP expression is observed.

### Example 30—Lipid 5, Lipid 8, DLn-MC3-DMA LNPs In Vivo Reprogramming Comparison

In this example, LNPs utilizing Lipid 5, Lipid 8, or DLn-MC3-DMA (0.1 mg/kg dose) targeted to CD3 (SP34) or CD8 (V2 (Nanobody)) were tested for their ability to reprogram immune cells in vivo.

The LNP formulations in the table below were prepared using the microfluidic mixing method described in Example 6 and discontinuous diafiltration method described in Example 25. The LNPs were formulated using eGFP encoding mRNA (TriLink Biotechnologies, California, US) and labeled with 0.06 mol % DiIC18(5)-DS (Invitrogen, Massachusetts, US). The LNPs were then inserted with a targeting conjugate using the specified conditions to provide the final targeted LNP formulations. The LNPs were characterized as described in Example 3.

TABLE 39
LNP Formulations

| | | | Lipid- | | Conjugate | |
|---|---|---|---|---|---|---|
| | | | PEG | | Insertion | |
| | Ionizable | Lipid- | Content | Targeting | Density | Insertion |
| Formulation No. | Lipid | PEG | (mol %) | Conjugate | (g/mol) | Condition |
| EXP21003471-N101 | DLin-MC3-DMA | DPG-PEG | 1.5 | hSP34 | 9 | 60° C. for 0.5 h in |
| | | | | | | pH 7.4 HBS |
| EXP21003471-N202 | Lipid 8 | DPG-PEG | 1.5 | hSP34 | 9 | 60° C. for 0.5 h in |
| | | | | | | pH 7.4 HBS |
| EXP21003471-N303 | Lipid 5 | DPG-PEG | 1.5 | hSP34 | 9 | 37° C. for 4 h in |
| | | | | | | pH 7.4 HBS |
| EXP21003471-N111 | DLin-MC3-DMA | DPG-PEG | 1.5 | V2 | 5.86 | 60° C. for 0.5 h in |
| | | | | (Nanobody) | | pH 7.4 HBS |

TABLE 40
Formulation Analysis Results

| | | | Pre- | Pre- | Pre- |
|---|---|---|---|---|---|
| | Post- | | Insertion | Insertion | Insertion |
| | Insertion | | Zeta | Zeta | Dye- |
| | DLS Z-Avg. | Post- | Potential at | Potential at | Accessible |
| Formulation | Diameter | Insertion | pH 5.5 | pH 7.4 | mRNA |
| No. | (nm) | DLS PDI | (mV) | (mV) | (%) |
| EXP21003471- | 107 | 0.20 | 16.4 | −0.1 | 9.1 |
| N101 | | | | | |
| EXP21003471- | 103 | 0.19 | 16.2 | 1.6 | 10 |
| N202 | | | | | |
| EXP21003471- | 92 | 0.11 | 20.1 | 4.4 | 6.4 |
| N303 | | | | | |
| EXP21003471- | 92 | 0.16 | 16.4 | −0.1 | 9.1 |
| N111 | | | | | |

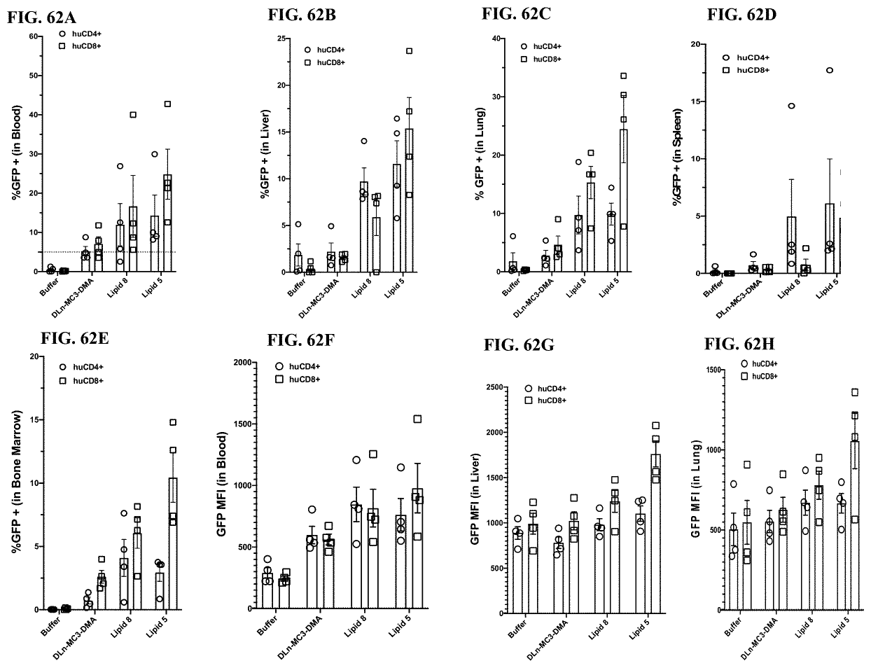

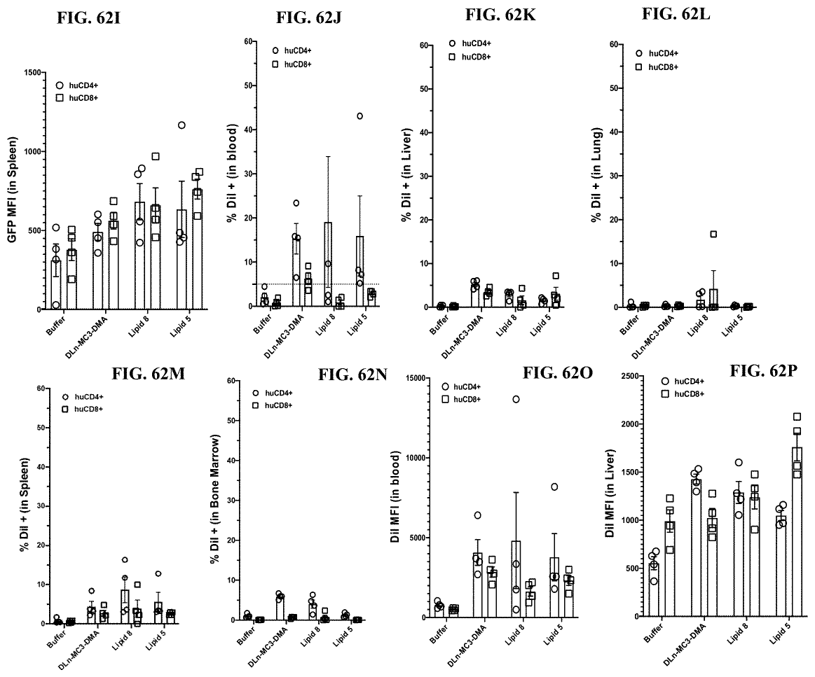

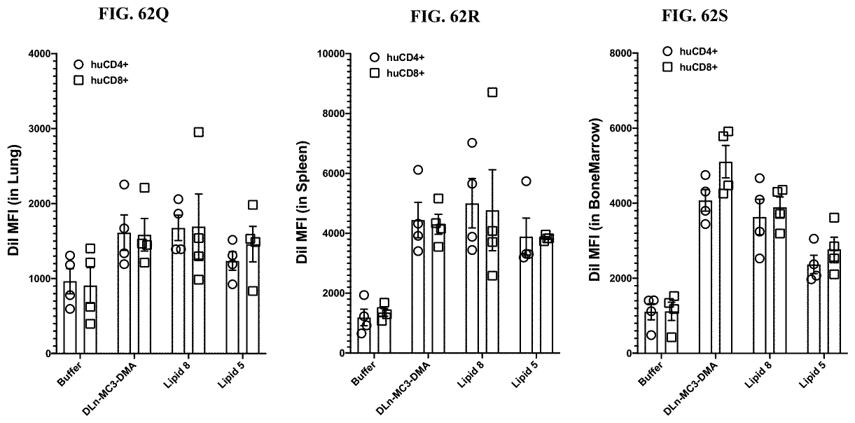

Results of in vivo reprogramming of immune cells with above LNPs at 0.1 mg/kg with DPG-PEG (1.5%) after 24 h are show in FIGS. 62A to 62S.

Comparison of DLn-MC3-DMA, Lipid 8 and Lipid 5 with DPG-PEG-1.5% and CD3 (hsp34) targeted LNPs at 0.1 mg/kg dose, Lipid 5 is superior for reprogramming T cells and showed maximum reprogramming of T cells with maximum GFP expression in blood and lung about 25% (blood=lung>liver>bone marrow). GFP MFI showed similar trend. All 3 lipids showed similar DiI positive T cells indicating that all lipids may bind equally but Lipid 5 is better at reprogramming T cells.

### Example 31—In Vitro Protein Expression—LNP Transfection of NK and T Cells in Co—Culture

This example describes targeting co-cultured human NK and T cells with anti-CD3, anti-CD7, anti-CD11a, anti-CD18, anti-CD56 (Lorvotuzumab) anti-CD137 (4B4-1) and anti-CD2 (RPA-2.10v1) Fab or Nanobodies post-inserted into GFP mRNA DiI labeled LNPs and their effect on transfection and translation.

Primary human T cells were purified using magnetic-based CD3 negative selection. 20 million purified T cells were activated using anti-CD3/anti-CD28 coated beads for 48 hours in media containing 100 IU/mL IL-2. Following activation, activation beads were removed, and T cells were expanded for an additional 48 hours in media containing 100 IU/mL IL-2. After the expansion period, T cells were concentrated to 1 million cells/mL in preparation for co-transfection with primary human NK cells.

CD3-depleted PBMCs were purified using magnetic-based CD3 positive selection and retaining of the negative fraction. 20 million CD3 depleted PBMCs were added to 1 well of a 6 well GREX plate in media containing 10 ng/mL IL-15 for 7 days. On day 7, each well was split in 2 and cells were cultured further in media containing 10 ng/mL IL-15 for an additional 7 days. On day 14, NK cells were concentrated to 1 million cells/mL in preparation for co-transfection with primary human T cells.

LNPs were prepared using the mixing process described in Example 6, the buffer exchange process described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of Nb-conjugated differed in using 1:1:4 Nb:DSPE-SKPEG-maileimide:DSPE-2KPEG-OCH3 and a 50 kD UF membrane (Millipore Corp, Billerica, Mass. USA) for separation of Nb-conjugate from free Nb. Using methods similar to Example 12, conjugated Fabs and conjugated Nb were post-inserted at various densities (Table 41) into LNPs containing Lipid 8 and GFP mRNA with DiI dye. into the LNPs containing Lipid 8, GFP mRNA, and DiI dye.

TABLE 41
Fab or Nb density post-inserted onto the
surface of the LNP

| Targeting moiety | |
|---|---|
| αCD11a HzMHM24 bDS Fab | 3, 5, 9 g/mol |
| αCD18 h1B4 bDS Fab | 3, 5, 9 g/mol |
| αCD7 V1 | 3, 5, 9 g/mol |
| αCD56 A1 Fab bDS | 3, 9, 19 g/mol |
| αCD56 A2 Fab bDS | 3, 9, 19 g/mol |
| αCD56 A3 Fab bDS | 3, 9, 19 g/mol |
| αCD56 Lorvotuzumab Fab bDS | 3, 6, 9 g/mol |
| αCD137 4B4-1 Fab bDS | 3, 9, 18 g/mol |
| αCD2 9.6 Fab bDS | 0.75, 1.5, 3 g/mol |
| αCD2 TS2/18.1 Fab bDS | 0.75, 1.5, 3 g/mol |
| αCD2 RPA-2.10v1 Fab bDS | 0.75, 1.5, 3 g/mol |
| αCD2 Lo-CD2b Fab bDS | 0.75, 1.5, 3 g/mol |
| αCD2 35.1 Fab bDS | 0.75, 1.5, 3 g/mol |
| αCD2 OKT11 Fab bDS | 0.75, 1.5, 3 g/mol |
| Dead Fab mutOKT8 | 9 g/mol |
| αCD8 TRX2 NoDS (interchain | 9 g/mol |
| disulfide knockout) | |
| HEPES Buffered Saline | No LNP |

Anti-CD56 A1 Fab sequence
A1 bDS HC (SEQ ID NO: 26):
QVQLQQSGPGLVKPSQTLSLTCAISGDSVSSNSAAWNWIRQSPSNWIRQSP
SGLEWLGRTYYRSKWYNDYAVSVKSRITINPDTSKNQFSLQLNSVTPEDTA
VYYCARENIAAWTWAFDIWGQGTMVTVSSASTKGPSVFPLAPSSKSTSGGT
AALGCLVKDYFPEPVTVSWNSGALTSGVHTCPAVLQSSGLYSLSSVVTVPS
SSLGTQTYICNVNHKPSNTKVDKKVEPKSSDKTHTCGGHHHHHH
A1 bDS LC (SEQ ID NO: 27):
EIVMTQSPGTLSLSPGERATLSCRASQSVSSSYLAWYQQKPGLAPRLLIYD
TSLRATDIPDRFSGSGSGTAFTLTISRLEPEDFAVYYCQQYGSSPTFGQGT
KVEIKRTVAAPSVFIFPPSDEQLKSGTASVVCLLNNFYPREAKVQWKVDNA
LQSGNSQESVTEQDSKDSTYSLCSTLTLSKADYEKHKVYACEVTHQGLSSP
VTKSFNRGES
Anti-CD56 A2 Fab sequence
A2 bDS HC (SEQ ID NO: 28):
EVQLVQSGAEVKKPGSSVKVSCKASGGTFTGYYMHWVRQAPGQGLEWMGWI
NPNSGGTNYAQKFQGRVTMTRDTSISTAYMELSRLRSDDTAVYYCARDLSS
GYSGYFDYWGQGTLVTVSSASTKGPSVFPLAPSSKSTSGGTAALGCLVKDY
FPEPVTVSWNSGALTSGVHTCPAVLQSSGLYSLSSVVTVPSSSLGTQTYIC
NVNHKPSNTKVDKKVEPKSSDKTHTCGGHHHHHH
A2 bDS LC (SEQ ID NO: 29):
DVVMTQSPLSLPVTPGEPASISCRSSQSLLHSNGYNYLNWYLQKPGQSPQL
LIYLGSNRASGVPDRFSGSGSGTDFTLKISRVEGEDVGDYYCMQALQSPFT
FGQGTKLEIKRTVAAPSVFIFPPSDEQLKSGTASVVCLLNNFYPREAKVQW
KVDNALQSGNSQESVTEQDSKDSTYSLCSTLTLSKADYEKHKVYACEVTHQ
GLSSPVTKSFNRGES
Anti-CD56 A3 Fab sequence
A3 bDS HC (SEQ ID NO: 30):
EVQLVQSGAEVKKPGSSVKVSCKASGGTFTGYYMHWVRQAPGQGLEWMGWI
NPNSGGTNYAQKFQGRVTMTRDTSISTAYMELSRLRSDDTAVYYCARDLSS
GYSGYFDYWGQGTLVTVSSASTKGPSVFPLAPSSKSTSGGTAALGCLVKDY
FPEPVTVSWNSGALTSGVHTCPAVLQSSGLYSLSSVVTVPSSSLGTQTYIC
NVNHKPSNTKVDKKVEPKSSDKTHTCGGHHHHHH
A3 bDS LC (SEQ ID NO: 31):
DVVMTQSPLSLPVTPGEPASISCRSSQSLLHSNGYNFLDWYLQKPGQSPQL
LIYLGSNRASGVPDRFSGSGSGTDFTLKISRVEADDVGVYYCMQSLQTPWT
FGHGTKVEIKRTVAAPSVFIFPPSDEQLKSGTASVVCLLNNFYPREAKVQW
KVDNALQSGNSQESVTEQDSKDSTYSLCSTLTLSKADYEKHKVYACEVTHQ
GLSSPVTKSFNRGES
Anti-CD56 Lorvotuzumab Fab sequence
Lorvotuzumab bDS HC (SEQ ID NO: 32):
QVQLVESGGG VVQPGRSLRL SCAASGFTFS SFGMHWVRQA
PGKGLEWVAYISSGSFTIYY ADSVKGRFTI SRDNSKNTLY
LQMNSLRAED TAVYYCARMR KGYAMDYWGQ
GTLVTVSSASTKGPSVFPLAPSSKSTSGGTAALGCLVKDYFPEPVTVSWNS
GALTSGVHTCPAVLQSSGLYSLSSVVTVPSSSLGTQTYICNVNHKPSNTKV
DKKVEPKSSDKTHTCHHHHHH
Lorvotuzumab bDS LC (SEQ ID NO: 33):
DVVMTQSPLSLPVTLGQPASISCRSSQIIIHSDGNTYLEWFQQRPGQSPRR
LIYKVSNRFSGVPDRFSGSGSGTDFTLKISRVEAEDVGVYYCFQGSHVPHT
FGQGTKVEIKRTVAAPSVFIFPPSDEQLKSGTASVVCLLNNFYPREAKVQW
KVDNALQSGNSQESVTEQDSKDSTYSLCSTLTLSKADYEKHKVYACEVTHQ
GLSSPVTKSFNRGES
Anti-CD2 RPA-2.10v1 Fab sequence
RPA-2.10v1 bDS HC (SEQ ID NO: 34):
EVKLVESGGGLVKPGGSLKLSCAASGFTFSSYDMSWVRQTPEKRLEWVASI
SGGGFLYYLDSVKGRFTISRDNARNILYLHMTSLRSEDTAMYYCARSSYGE
IMDYWGQGTSVTVSSASTKGPSVFPLAPSSKSTSGGTAALGCLVKDYFPEP
VTVSWNSGALTSGVHTCPAVLQSSGLYSLSSVVTVPSSSLGTQTYICNVNH
KPSNTKVDKKVEPKSSDKTHTCHHHHHH
RPA-2.10V1 bDS LC (SEQ ID NO: 35):
DILLTQSPAILSVSPGERVSFSCRASQRIGTSIHWYQQRTTGSPRLLIKYA
SESISGIPSRFSGSGSGTDFTLSINSVESEDVADYYCQQSHGWPFTFGGGT
KLEIERTVAAPSVFIFPPSDEQLKSGTASVVCLLNNFYPREAKVQWKVDNA
LQSGNSQESVTEQDSKDSTYSLCSTLTLSKADYEKHKVYACEVTHQGLSSP
VTKSFNRGES
Anti-CD137 4B4-1 Fab sequence
4B4-1 bDS HC (SEQ ID NO: 36):
QVQLQQPGAELVKPGASVKLSCKASGYTFSSYWMHWVKQRPGQVLEWIGEI
NPGNGHTNYNEKFKSKATLTVDKSSSTAYMQLSSLTSEDSAVYYCARSFTT
ARGFAYWGQGTLVTVSASTKGPSVFPLAPSSKSTSGGTAALGCLVKDYFPE
PVTVSWNSGALTSGVHTCPAVLQSSGLYSLSSVVTVPSSSLGTQTYICNVN
HKPSNTKVDKKVEPKSSDKTHTCHHHHHH
4B4-1 bDS LC (SEQ ID NO: 37):
DIVMTQSPATQSVTPGDRVSLSCRASQTISDYLHWYQQKSHESPRLLIKYA
SQSISGIPSRFSGSGSGSDFTLSINSVEPEDVGVYYCQDGHSFPPTFGGGT
KLEIKRTVAAPSVFIFPPSDEQLKSGTASVVCLLNNFYPREAKVQWKVDNA
LQSGNSQESVTEQDSKDSTYSLCSTLTLSKADYEKHKVYACEVTHQGLSSP
VTKSFNRGES

On the day of LNP transfection, 50 thousand T cells and 50 thousand NK cells were added to each well of a 96 well culture plate in a total volume of 1004 containing 100 IU/mL IL-2. 104 of each test LNP was added to each well to facilitate simultaneous transfection of primary human T cells and NK cells at 2.5 ug/mL mRNA.

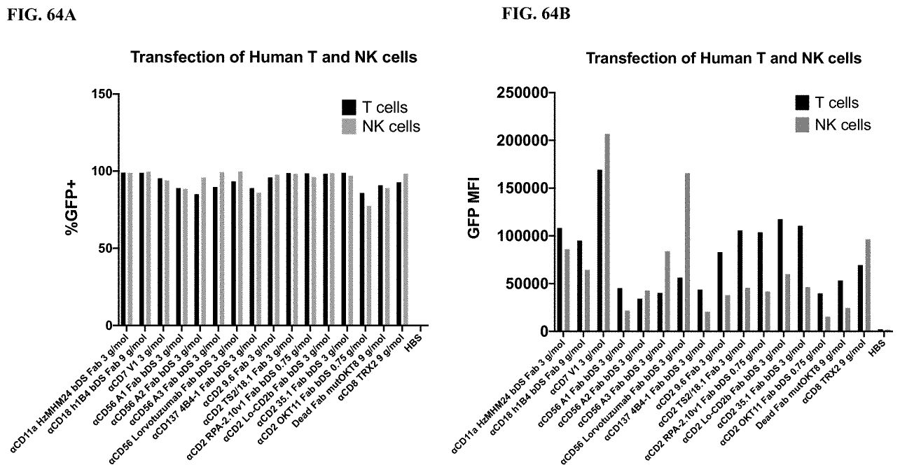

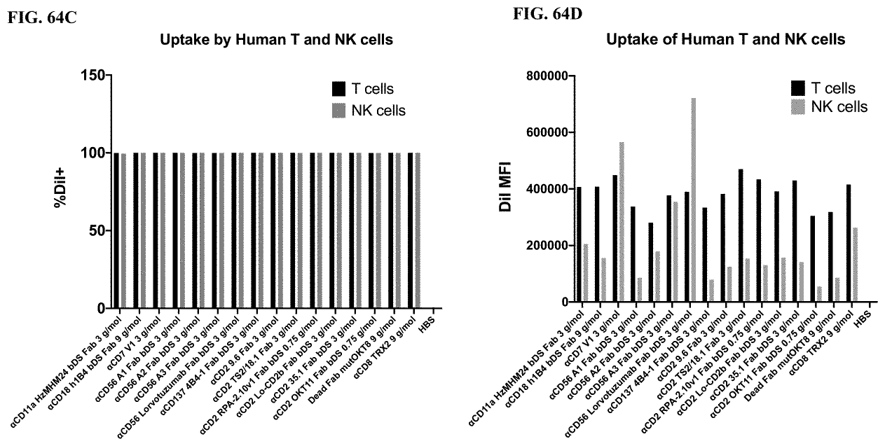

24 hours after LNP transfection, cell culture media was aspirated from T cell and NK cell co-cultures after centrifugation. T cells and NK cells were resuspended with anti-CD3 and anti-CD56 fluorescently labeled antibodies to facilitate analysis of LNP transfection independently in each cell type within the co-culture for 20 minutes at room temperature. Following incubation, cells were concentrated by centrifugation and resuspended in 1× PBS for analysis by flow cytometry. Following acquisition by flow cytometry, T cells and NK cells were analyzed independently using FlowJo (flow cytometry analysis software). In either CD3+ cells (T cells) or CD56+ cells (NK cells), the frequency of GFP positive events relative to GFP negative events was calculated (FIG. 64A). Additionally, the overall fluorescence of GFP was quantified by assessment of mean fluorescence intensity (MFI, FIG. 64B). Similar frequency and MFI analysis were performed for the DiI dye (FIG. 64C, FIG. 64D). Together, these metrics enabled quantification of LNP transfection efficiency of all targeted LNPs tested in primary human T cell and NK cell co-cultures.

In contrast to experiments performed with unstimulated T cells or whole blood, both ex vivo expanded NK and T cells show high % DiI and % GFP of LNPs post-inserted with the non-target specific mutOKT8 Fab. Despite this difference in % frequency, when comparing formulations by MFI, there was clear separation between mutOKT8 non-targeted LNPs and many of the surface antigen targeted LNPs. Consistent with previous studies, T cells were transfected by anti-CD7, anti-CD8, anti-CD2, anti-CD11a and anti-CD18 targeted LNPs while minimal to no transfection of T cells was observed for anti-CD137 or anti-CD56 targeted LNPs. Similarly, NK cells could also be transfected by anti-CD7, anti-CD8, anti-CD2, anti-CD11a and anti-CD18 targeted LNPs. But, in contrast to T cells, the CD56 targeted LNPs with Lorvotuzumab or the A3 clone only show highly specific transfection of NK cells.

This data indicates that Fabs or nanobodies are capable of enabling transfection/translation for both NK cells and T cells using anti-CD7, anti-CD8, anti-CD2, anti-CD11a or anti-CD18 targeted LNPs, while using anti-CD56 targeted LNPs is capable of translations/translation for NK cells with high specificity over other immune cells.

### Example 32—Peg-lipid Conjugation and In Vitro Protein Expression—CD3 Targeting Fabs with and without Natural Inter-Chain Disulfide

This example describes the conjugation, purity of either anti-CD3 Fabs with and without the natural interchain disulfide as well as their T cell transfection post-inserted into Cy5/GFP mRNA LNPs.

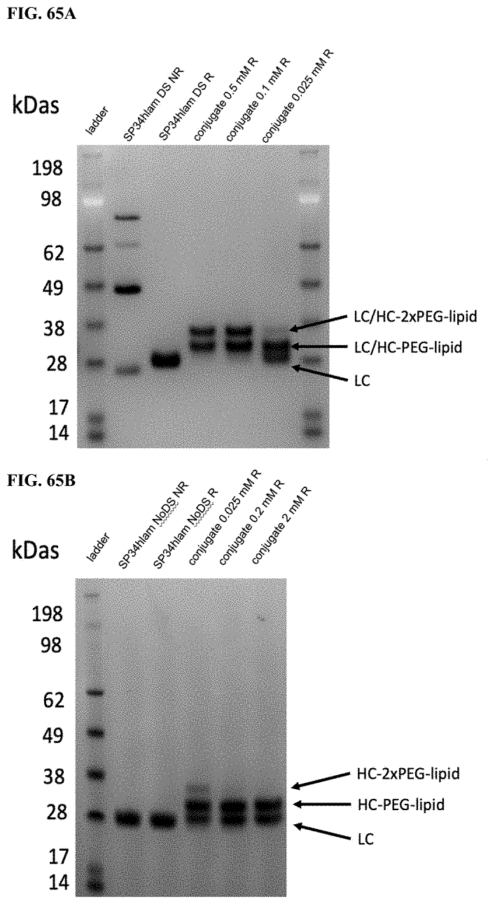

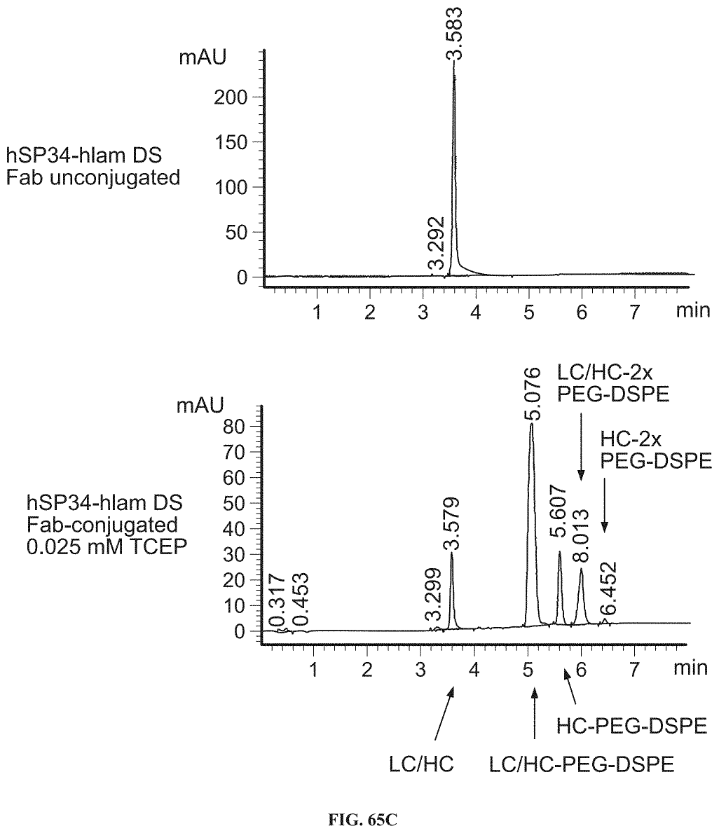

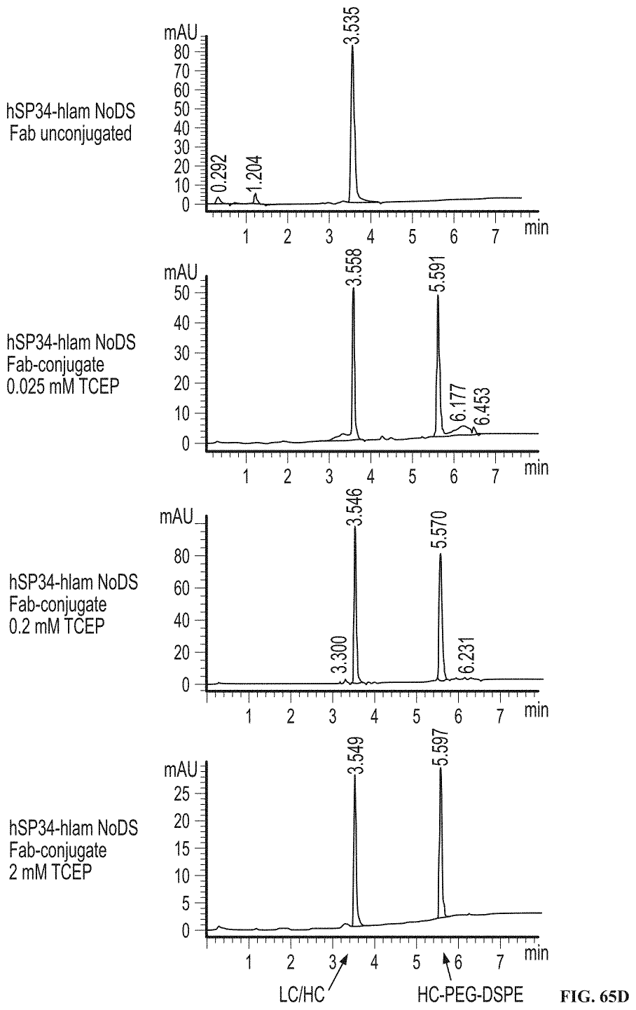

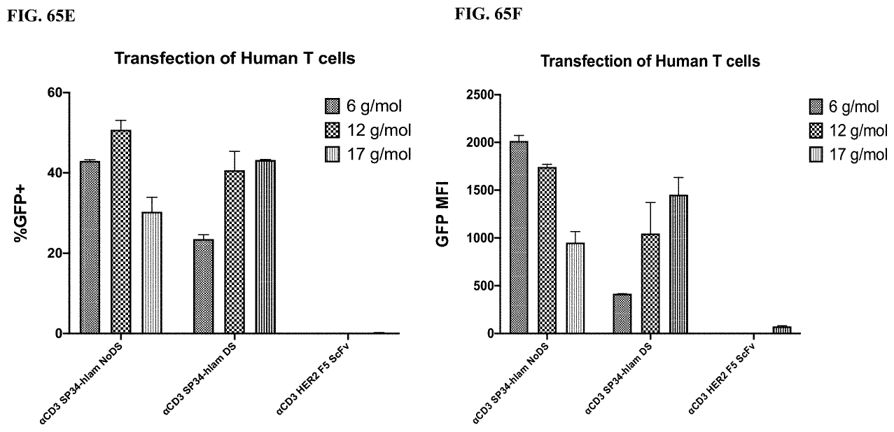

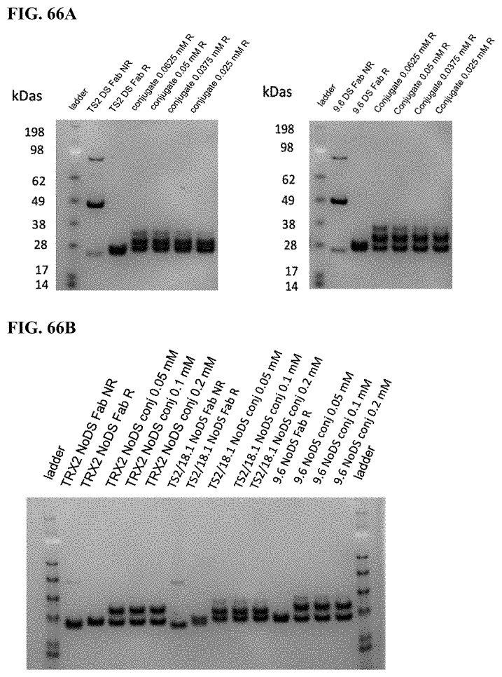

LNPs were prepared using the mixing process described in Example 6, the buffer exchange process described in Example 21. Conjugates were generated using a method similar to that of Example 4 except 0.025, 0.1, 0.5 mM TCEP was used for hSP34 DS, 0.025, 0.2, 2 mM TCEP was used for hSP34-hlam NoDS (interchain disulfide knockout) for reduction prior to conjugation and the conjugation reactions were performed at 37 C for 2 hr. SDS-PAGE (FIGS. 65A, 65B) was performed using the manufacturers recommended conditions with 1 ug of protein (Thermo, 4-12% Bis-Tris MiniGel). RP-HPLC (FIGS. 65C, 65D) was performed using an Agilent 300 SB-C8 at 0.5 mL/min with a column temperature of 60° C., Mobile Phase A: Water with 0.1% TFA, Mobile Phase B: Acetonitrile with 0.1% TFA, Gradient % B: 0 min 5%, 1 min 5%, 6.5 min 95%, 8 min 95% injecting 10 ul with a target of 1-25 ug of protein. Using methods similar to Example 12, ant-CD3 hSP34 (with and without natural interchain disulfide, DS (with interchain disulfide) vs. NoDS (without interchain disulfide), see sequences below) PEG-lipid conjugated Fabs were post-inserted at various Fab densities (6, 12, 17 g Fab/mol total lipid) into LNPs containing Lipid 8 and Cy5/GFP mRNA. Transfections were performed with human CD3 T cells at approximately 2.5 μg/mL mRNA for approximately 24 hr. Levels of transfection of CD3 T cells was measured by flow cytometry.

anti-HuCD3 hSP34-hlam Fabs NoDS (without interchain
disulfide) and DS (with interchain disulfide)
hSP34-hlam NoDS HC (SEQ ID NO: 38):
EVQLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRHG
NFGNSYISYWAYWGQGTLVTVSSASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSSDKTHTC
hSP34-hlam NoDS LC (SEQ ID NO: 39):
QTVVTQEPSLTVSPGGTVTLTCGSSTGAVTSGNYPNWVQQKPGQAPRGLIG
GTKFLAPGTPARFSGSLLGGKAALTLSGVQPEDEAEYYCVLWYSNRWVFGG
GTKLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGST
VEKTVAPAESS
hSP34-hlam DS HC (SEQ ID NO: 40):
EVQLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRHG
NFGNSYISYWAYWGQGTLVTVSSASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSCDKTHTC
hSP34-hlam DS LC (SEQ ID NO: 41):
QTVVTQEPSLTVSPGGTVTLTCGSSTGAVTSGNYPNWVQQKPGQAPRGLIG
GTKFLAPGTPARFSGSLLGGKAALTLSGVQPEDEAEYYCVLWYSNRWVFGG
GTKLTVLSQPKAAPSVTLFPPSSEELQANKATLVCLVSDFYPGAVTVAWKA
DGSPVKVGVETTKPSKQSNNKYAASSYLSLTPEQWKSHRSYSCRVTHEGST
VEKTVAPAECS

The SP34 DS Fab runs around 49 kD on a non-reduced gel (FIG. 65A) while the SP34 NoDS Fab LC and HC run similar to each other at slightly less than 28 kD on a non-reduced gel (FIG. 65B) confirming that the interchain disulfide has been knocked out by mutating the corresponding cysteines in the heavy and light chain to serine. SP34 DS Fab that was reduced at varying levels of TCEP exhibited high levels of LC/HC single and double PEG-lipid conjugation as shown in both the gel and RP-HPLC chromatogram (FIG. 65C) where the condition with the highest purity was 0.025 mM TCEP during reduction while 0.1 and 0.5 mM TCEP had intractable amounts of double conjugate. In contrast, the SP34 NoDS Fab shows high purity at the full range of TCEP evaluated up to 2 mM TCEP highlighting that removal of the interchain disulfide has a dramatic effect on the ability to generate highly pure (single lipid) conjugated Fab. For T cell transfection studies, 0.025 mM TCEP generated SP34 DS Fab was selected given it was the purest of the reaction conditions and had similar recoveries to the other conditions after UF purification (TABLE 42) and 0.2 mM TCEP generated SP34 NoDS Fab was selected given it had both high purity and gave the best recovery after UF purification (TABLE 43).

TABLE 42
Relationship between TCEP concentration during
reduction and final recovery of SP34-hlam
DS conjugate after UF purification

| | % Recovery |
|---|---|
| TCEP mM | Post UF |
| 0.5 | 20.5 |
| 0.1 | 27.6 |
| 0.025 | 24.3 |

TABLE 43
Relationship between TCEP concentration
during reduction and final recovery of
SP34-hlam NoDS conjugate after UF purification

| | % Revovery Post |
|---|---|
| TCEP mM | UF |
| 2 | 14.0 |
| 0.2 | 55.5 |
| 0.025 | 25.9 |

The SP34 NoDS Fab mediated higher % transfection (FIG. 65E) and higher GFP expression levels (FIG. 65F) than the SP34 DS Fab at the lowest, 6 g/mol, and middle, 12 g/mol, Fab densities indicating that the potency of this conjugate is higher than that of SP34 DS Fab which is consistent with its correspondingly higher purity (1 PEG-Lipid per Fab, no LC-PEG-lipid).

This data indicates that knocking out the natural interchain disulfide enables highly efficient, site-specific conjugation towards the c-terminal cysteine with a single PEG-lipid. This broadens the range of reducing agent that can be employed to obtain high purity conjugate (1 PEG-Lipid per Fab) with high process recovery and avoids conjugation of 2 or more PEG-Lipids per Fab which can reduce the final transfection efficiency of targeted LNPs for immune cells.

### Example 33—Fab-peg-lipid Conjugation and Purity—CD2 and CD8 Targeting FABs with and without Natural Inter-Chain Disulfide

This example describes the conjugation and purity of anti-CD2 and anti-CD8 Fabs with and without their natural interchain disulfide (FIG. 47).

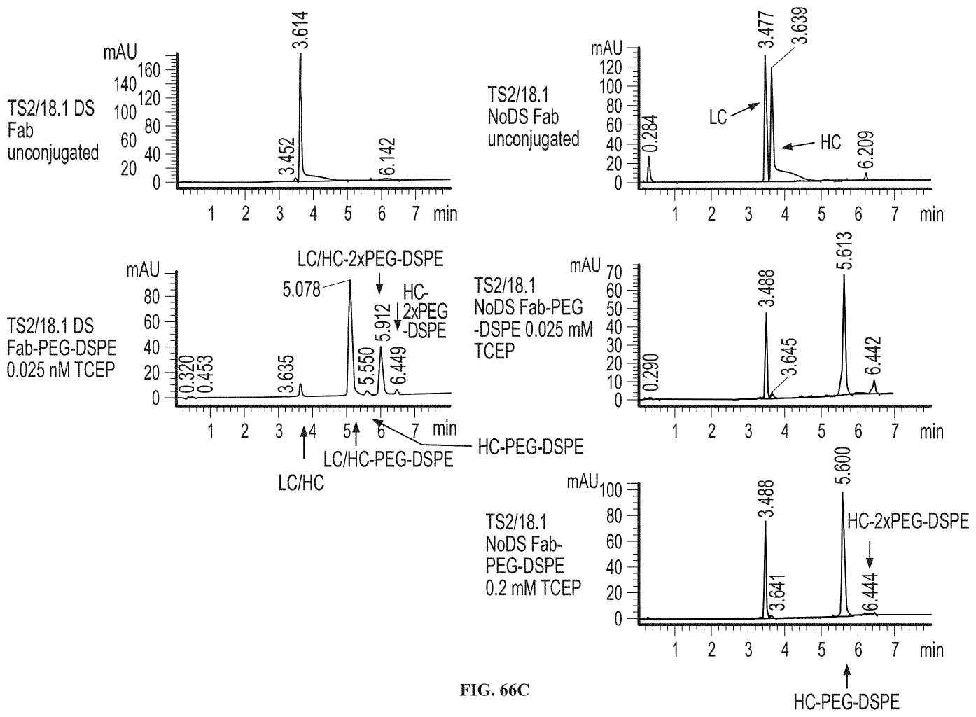

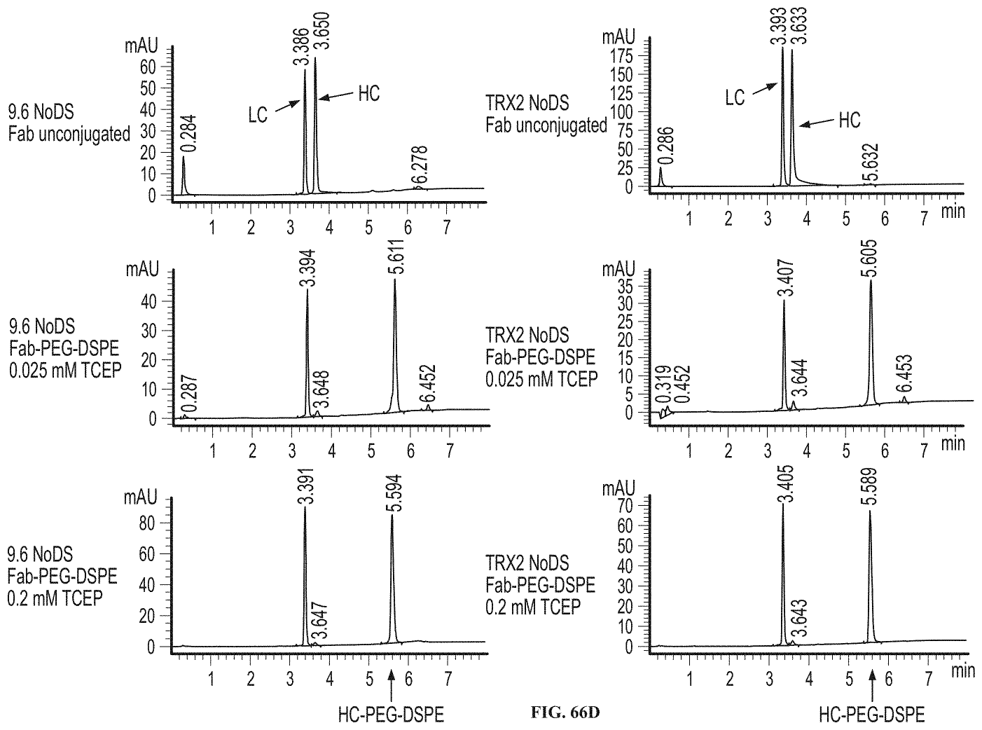

Conjugates were generated using a method similar to that of Example 4 except 0.025, 0.0375, 0.05, 0.0625 mM TCEP was used for anti-CD2 TS2/18.1 and 9.6 DS Fabs, 0.05, 0.1, 0.2 mM TCEP was used for anti-CD2 TS2/18.1 and 9.6 NoDS Fabs (see sequences below), 0.025, 0.05, 0.1, 0.2 mM TCEP was used for anti-CD8 TRX2 NoDS Fab for reduction prior to conjugation and the conjugation reactions were performed at 37 C for 2 hr. SDS-PAGE (FIGS. 66A and 66B) was performed using the manufacturers recommended conditions with 1 ug of protein (Thermo, 4-12% Bis-Tris MiniGel). RP-HPLC (FIGS. 66C and 66D) was performed using an Agilent 300 SB-C8 at 0.5 mL/min with a column temperature of 60° C., Mobile Phase A: Water with 0.1% TFA, Mobile Phase B: Acetonitrile with 0.1% TFA, Gradient % B: 0 min 5%, 1 min 5%, 6.5 min 95%, 8 min 95% injecting 10 ul with a target of 1-25 ug of protein.

Anti-CD2 TS2/18.1 DS Fab
TS2/18.1 DS HC (SEQ ID NO: 42):
EVQLVESGGGLVMPGGSLKLSCAASGFAFSSYDMSWVRQTPEKRLEWVAYI
SGGGFTYYPDTVKGRFTLSRDNAKNTLYLQMSSLKSEDTAMYYCARQGANW
ELVYWGQGTLVTVSAASTKGPSVFPLAPSSKSTSGGTAALGCLVKDYFPEP
VTVSWNSGALTSGVHTFPAVLQSSGLYSLSSVVTVPSSSLGTQTYICNVNH
KPSNTKVDKKVEPKSCDKTHTC
TS2/18.1 DS LC (SEQ ID NO: 43):
DIVMTQSPATLSVTPGDRVFLSCRASQSISDFLHWYQQKSHESPRLLIKYA
SQSISGIPSRFSGSGSGSDFTLSINSVEPEDVGVYFCQNGHNFPPTFGGGT
KLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC
Anti-CD2 9.6 DS Fab
9.6 DS HC (SEQ ID NO: 44):
QVQLQQPGAELVRPGSSVKLSCKASGYTFTRYWIHWVKQRPIQGLEWIGNI
DPSDSETHYNQKFKDKATLTVDKSSGTAYMQLSSLTSEDSAVYYCATEDLY
YAMEYWGQGTSVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSCDKTHTC
9.6 DS LC (SEQ ID NO: 45):
NIMMTQSPSSLAVSAGEKVTMTCKSSQSVLYSSNQKNYLAWYQQKPGQSPK
LLIYWASTRESGVPDRFTGSGSGTDFTLTISSVQPEDLAVYYCHQYLSSHT
FGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGEC

The TS2/18.1 and 9.6 DS Fabs run around 49 kD on a non-reduced gel (FIG. 26-1) while the TS2/18.1, 9.6 and TRX2 NoDS Fabs LC and HC run similar to each other at slightly less than 28 kD on a non-reduced gel (FIG. 66B) confirming that the interchain disulfide has been knocked out by mutating the corresponding the cysteines in the heavy and light chain to serine. The TS2/18.1 and 9.6 DS Fabs reduced at varying levels of TCEP prior to conjugation exhibited high levels of LC/HC single and double PEG-lipid conjugation as shown by both the SDS-PAGE (FIG. 66A) and RP-HPLC chromatograms (TS2/18.1 only, FIG. 66C) where the condition with the highest purity was 0.025 mM TCEP during reduction and higher TCEP levels increased the amount of LC conjugate and double conjugate (2 PEG-lipid per Fab). In contrast, the TS2/18.1, 9.6 and TRX2 NoDS Fabs shows high purity at the full range of TCEP evaluated up to 0.2 mM TCEP highlighting that removal of the interchain disulfide has a dramatic effect on the ability to generate highly pure (1 PEG-lipid per Fab) conjugate.

This data across a number immune cell targeting Fabs indicates that knocking out the natural interchain disulfide is a generalizable approach to enable highly efficient, site-specific conjugation towards the c-terminal cysteine on the heavy chain while avoiding conjugation to the light chain and conjugating more than one PEG-lipid per Fab.

### Example 34—In Vitro Protein Expression—CD3 and TCR Targeting Comparison and SP34 Fab with and without Buried Disulfide

This example describes targeting human CD3 T cells with either anti-CD3 or anti-TCR Fabs and an anti-CD3 Fab with and without a buried interchain disulfide (FIG. 47) post-inserted into Cy5/GFP mRNA LNPs and their effect on transfection and IFNγ secretion.

LNPs were prepared using the mixing process described in Example 6, the buffer exchange process described in Example 21. Conjugates were generated using a method similar to that of Example 4 except 0.1 mM TCEP was used for reduction prior to conjugation and the conjugation reaction was performed at 37 C for 2 hr. Using methods similar to Example 12, anti-CD3 hSP34 (with and without buried disulfide, bDS vs. NoDS), TR66 (Bortoletto et al Optimizing anti-CD3 affinity for effective T cell targeting against tumor cells, Eu. J of Immun. 2002; Frank et al Combining T cell specific activation and in vivo gene delivery through CD3-targeted lentiviral vectors, Blood Adv 2020), anti-CD3 TRX4, anti-CD3 humanized UCHT1 (HzUCHT1), and anti-CD3 Teplizumab PEG-lipid conjugated Fabs were post-inserted into LNPs containing Lipid 8 and Cy5/GFP mRNA. Transfections were performed with human CD3 T cells at approximately 2.5 μg/mL mRNA for approximately 24 hr. Levels of transfection of both CD8 and CD4 cells was measured by flow cytometry using an anti-CD4 antibody (clone SK3) to distinguish the two cell types. IFNγ in the supernatants was measured using the manufacturers recommended procedure (R&D Systems, DY285B).

Anti-CD3 hSP34-hlam bDS Fab sequence
hSP34-hlam bDS HC (SEQ ID NO: 46):
EVQLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRHG
NFGNSYISYWAYWGQGTLVTVSSASTKGPSVFPLAPSSKSTSGGTAALGCL
VKDYFPEPVTVSWNSGALTSGVHTCPAVLQSSGLYSLSSVVTVPSSSLGTQ
TYICNVNHKPSNTKVDKKVEPKSSDKTHTCHHHHHH
hSP34-hlam bDS LC (SEQ ID NO: 47):
QTVVTQEPSLTVSPGGTVTLTCGSSTGAVTSGNYPNWVQQKPGQAPRGLIG
GTKFLAPGTPARFSGSLLGGKAALTLSGVQPEDEAEYYCVLWYSNRWVFGG
GTKLTVLSQPKAAPSVTLFPPSSEELQANKATLVCLVSDFYPGAVTVAWKA
DGSPVKVGVETTKPSKQSNNKYAACSYLSLTPEQWKSHRSYSCRVTHEGST
VEKTVAPAESS
Anti-CD3 TR66 bDS Fab sequence
TR66 bDS HC (SEQ ID NO: 48):
QVQLQQSGAELARPGASVKMSCKTSGYTFTRYTMHWVKQRPGQGLEWIGYI
NPSRGYTNYNQKFKDKATLTTDKSSSTAYMQLSSLTSEDSAVYYCARYYDD
NYSLDYWGQGTTLTVSSASTKGPSVFPLAPSSKSTSGGTAALGCLVKDYFP
EPVTVSWNSGALTSGVHTCPAVLQSSGLYSLSSVVTVPSSSLGTQTYICNV
NHKPSNTKVDKKVEPKSSDKTHTCHHHHHH
TR66 bDS LC (SEQ ID NO: 49):
QIVLTQSPSSLSASLGEKVTMTCRASSSVSYMNWYQQKPGTSPKRWIYDTS
KVASGVPDRFSGSGSGTSYSLTISSMEAEDAATYYCQQWSSNPLTFGAGTK
LELKRTVAAPSVFIFPPSDEQLKSGTASVVCLLNNFYPREAKVQWKVDNAL
QSGNSQESVTEQDSKDSTYSLCSTLTLSKADYEKHKVYACEVTHQGLSSPV
TKSFNRGES
Anti-CD3 TRX4 bDS Fab sequence
TRX4 bDS HC (SEQ ID NO: 50):
EVQLLESGGGLVQPGGSLRLSCAASGFTFSSFPMAWVRQAPGKGLEWVSTI
STSGGRTYYRDSVKGRFTISRDNSKNTLYLQMNSLRAEDTAVYYCAKFRQY
SGGFDYWGQGTLVTVSSASTKGPSVFPLAPSSKSTSGGTAALGCLVKDYFP
EPVTVSWNSGALTSGVHTCPAVLQSSGLYSLSSVVTVPSSSLGTQTYICNV
NHKPSNTKVDKKVEPKSSDKTHTCHHHHHH
TRX4 bDS LC (SEQ ID NO: 51):
DIQLTQPNSVSTSLGSTVKLSCTLSSGNIENNYVHWYQLYEGRSPTTMIYD
DDKRPDGVPDRFSGSIDRSSNSAFLTIHNVAIEDEAIYFCHSYVSSFNVFG
GGTKLTVLGQPKANPTVTLFPPSSEELQANKATLVCLISDFYPGAVTVAWK
ADGSPVKAGVETTKPSKQSNNKYAACSYLSLTPEQWKSHRSYSCQVTHEGS
TVEKTVAPTESS
Anti-CD3 HzUCHT1 bDS Fab sequence
HzUCHT1(Y59T) bDS HC (SEQ ID NO: 52):
EVQLVESGGGLVQPGGSLRLSCAASGYSFTGYTMNWVRQAPGKGLEWVALI
NPTKGVSTYNQKFKDRFTISVDKSKNTAYLQMNSLRAEDTAVYYCARSGYY
GDSDWYFDVWGQGTLVTVSSASTKGPSVFPLAPSSKSTSGGTAALGCLVKD
YFPEPVTVSWNSGALTSGVHTCPAVLQSSGLYSLSSVVTVPSSSLGTQTYI
CNVNHKPSNTKVDKKVEPKSSDKTHTCHHHHHH
HzUCHT1 bDS LC (SEQ ID NO: 53):
DIQMTQSPSSLSASVGDRVTITCRASQDIRNYLNWYQQKPGKAPKLLIYYT
SRLESGVPSRFSGSGSGTDYTLTISSLQPEDFATYYCQQGNTLPWTFGQGT
KVEIKRTVAAPSVFIFPPSDEQLKSGTASVVCLLNNFYPREAKVQWKVDNA
LQSGNSQESVTEQDSKDSTYSLCSTLTLSKADYEKHKVYACEVTHQGLSSP
VTKSFNRGES
Anti-CD3 Teplizumab bDS Fab sequence
Teplizumab bDS HC (SEQ ID NO: 54):
QVQLVQSGGGVVQPGRSLRLSCKASGYTFTRYTMHWVRQAPGKGLEWIGYI
NPSRGYTNYNQKVKDRFTISRDNSKNTAFLQMDSLRPEDTGVYFCARYYDD
HYCLDYWGQGTPVTVSSASTKGPSVFPLAPSSKSTSGGTAALGCLVKDYFP
EPVTVSWNSGALTSGVHTCPAVLQSSGLYSLSSVVTVPSSSLGTQTYICNV
NHKPSNTKVDKKVEPKSSDKTHTCHHHHHH
Teplizumab bDS LC (SEQ ID NO: 55):
DIQMTQSPSSLSASVGDRVTITCSASSSVSYMNWYQQTPGKAPKRWIYDTS
KLASGVPSRFSGSGSGTDYTFTISSLQPEDIATYYCQQWSSNPFTFGQGTK
LQITRTVAAPSVFIFPPSDEQLKSGTASVVCLLNNFYPREAKVQWKVDNAL
QSGNSQESVTEQDSKDSTYS

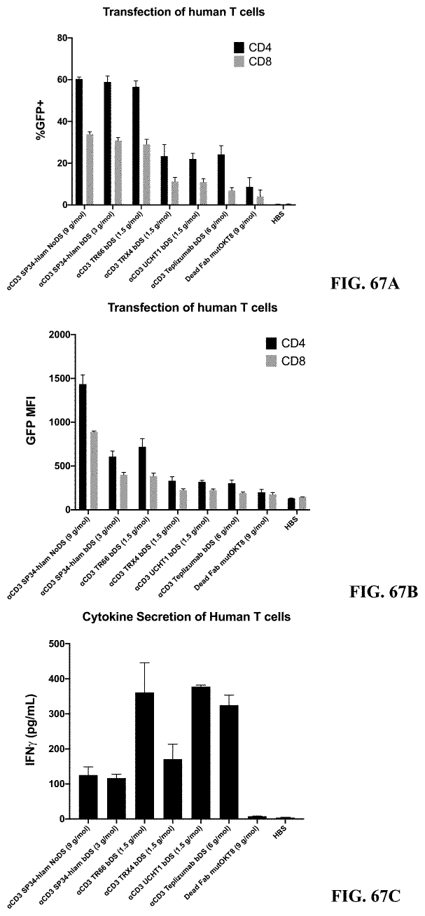

The SP34 NoDS Fab mediated higher % transfection (FIG. 67A) and higher GFP expression levels (FIG. 67B) quantified by mean fluorescence intensity, MFI) than the other Fab constructs. While the SP34 bDS and TR66 bDS Fabs mediated similar levels of % transfection by GFP the expression levels were lower than that of the SP34 NoDS Fab quantified by mean fluorescence intensity. For SP34 the NoDS and bDS Fab formats had similar levels of IFNγ secretion (FIG. 67C). Additionally, while TR66, TRX4, HzUCHT1 and Teplizumab had higher levels of IFNγ secretion than SP34, they exhibited lower levels of GFP expression (FIG. 67B).

This data indicates that across multiple CD3 targeting Fabs, whether they have kappa or lambda light chains, many are capable of mediating high transfection/translation in either the NoDS or bDS exemplifying CD3 as a robust T cell target for mediating CD8 and CD4 T cell transfection and translation. For the SP34 clone, the NoDS format is preferred over the bDS format with regards to T cell transfection/translation efficiency. Additionally, this data suggests that T cell activation does not guarantee efficient transfection and translation.

### Example 35—In Vitro Protein Expression—CD8 Targeted Fab with and without Buried Disulfide and Other CD2 Targeted Fab Clones

This example describes targeting human CD8 T cells with anti-CD8 Fab in a NoDS or bDS format or anti-CD2 Fabs post-inserted into Cy5/GFP mRNA LNPs and their effect on transfection and translation.

LNPs were prepared using the mixing process described in Example 6, the buffer exchange process described in Example 21. Fab-lipid conjugates generated from methods described in Example 4. Using methods similar to Example 12, ant-CD3 hSP34, anti-CD8 TRX2 and anti-CD2 clones Lo-CD2b (ATCC, PTA-802), 35.1 (ATCC, HB-222) and OKT11 (ATCC, CRL-8027) PEG-lipid conjugated Fabs were post-inserted into LNPs containing Lipid 8 and Cy5/GFP mRNA. Transfections were performed with human CD3 T cells at approximately 2.5 μg/mL mRNA for approximately 24 hr. Levels of transfection of CD8 cells was measured by flow cytometry.

Anti-CD8 TRX2 bDS Fab sequence
TRX2 bDS HC (SEQ ID NO: 56):
QVQLVESGGGVVQPGRSLRLSCAASGFTFSDFGMNWVRQAPGKGLEWVALI
YYDGSNKFYADSVKGRFTISRDNSKNTLYLQMNSLRAEDTAVYYCAKPHYD
GYYHFFDSWGQGTLVTVSSASTKGPSVFPLAPSSKSTSGGTAALGCLVKDY
FPEPVTVSWNSGALTSGVHTCPAVLQSSGLYSLSSVVTVPSSSLGTQTYIC
NVNHKPSNTKVDKKVEPKSSDKTHTC
TRX2 bDS LC (SEQ ID NO: 57):
DIQMTQSPSSLSASVGDRVTITCKGSQDINNYLAWYQQKPGKAPKLLIYNT
DILHTGVPSRFSGSGSGTDFTFTISSLQPEDIATYYCYQYNNGYTFGQGTK
VEIKRTVAAPSVFIFPPSDEQLKSGTASVVCLLNNFYPREAKVQWKVDNAL
QSGNSQESVTEQDSKDSTYSLCSTLTLSKADYEKHKVYACEVTHQGLSSPV
TKSFNRGES
Anti-CD2 Lo-CD2b bDS Fab sequence
Lo-CD2b bDS HC (SEQ ID NO: 58):
EVQLVESGGGLVQPGASLKLSCVASGFTFSDYWMSWVRQTPGKPMEWIGHI
KYDGSYTNYAPSLKNRFTISRDNAKTTLYLQMSNVRSEDSATYYCAREAPG
AASYWGQGTLVTVSSASTKGPSVFPLAPSSKSTSGGTAALGCLVKDYFPEP
VTVSWNSGALTSGVHTCPAVLQSSGLYSLSSVVTVPSSSLGTQTYICNVNH
KPSNTKVDKKVEPKSSDKTHTC
Lo-CD2b bDS LC (SEQ ID NO: 59):
DVVLTQTPVAQPVTLGDQASISCRSSQSLVHSNGNTYLEWFLQKPGQSPQL
LIYKVSNRFSGVPDRFIGSGSGSDFTLKISRVEPEDWGVYYCFQGTHDPYT
FGAGTKLELKRTVAAPSVFIFPPSDEQLKSGTASVVCLLNNFYPREAKVQW
KVDNALQSGNSQESVTEQDSKDSTYSLCSTLTLSKADYEKHKVYACEVTHQ
GLSSPVTKSFNRGES
Anti-CD2 35.1 bDS Fab sequence
35.1 bDS HC (SEQ ID NO: 60):
EVQLQQSGAELVKPGASVKLSCRTSGFNIKDTYIHWVKQRPEQGLKWIGRI
DPANGNTKYDPKFQDKATVTADTSSNTAYLQLSSLTSEDTAVYYCVTYAYD
GNWYFDVWGAGTAVTVSSASTKGPSVFPLAPSSKSTSGGTAALGCLVKDYF
PEPVTVSWNSGALTSGVHTCPAVLQSSGLYSLSSVVTVPSSSLGTQTYICN
VNHKPSNTKVDKKVEPKSSDKTHTC
35.1 bDS LC (SEQ ID NO: 61):
DIKMTQSPSSMYVSLGERVTITCKASQDINSFLSWFQQKPGKSPKTLIYRA
NRLVDGVPSRFSGSGSGQDYSLTISSLEYEDMEIYYCLQYDEFPYTFGGGT
KLEMKRTVAAPSVFIFPPSDEQLKSGTASVVCLLNNFYPREAKVQWKVDNA
LQSGNSQESVTEQDSKDSTYSLCSTLTLSKADYEKHKVYACEVTHQGLSSP
VTKSFNRGES
Anti-CD2 OKT11 bDS Fab sequence
OKT11 bDS HC (SEQ ID NO: 62):
QVQLQQPGAELVRPGTSVKLSCKASGYTFTSYWMHWIKQRPEQGLEWIGRI
DPYDSETHYNEKFKDKAILSVDKSSSTAYIQLSSLTSDDSAVYYCSRRDAK
YDGYALDYWGQGTSVTVSSASTKGPSVFPLAPSSKSTSGGTAALGCLVKDY
FPEPVTVSWNSGALTSGVHTCPAVLQSSGLYSLSSVVTVPSSSLGTQTYIC
NVNHKPSNTKVDKKVEPKSSDKTHTC
OKT11 bDS LC (SEQ ID NO: 63):
DIVMTQAAPSVPVTPGESVSISCRSSKTLLHSNGNTYLYWFLQRPGQSPQV
LIYRMSNLASGVPNRFSGSGSETTFTLRISRVEAEDVGIYYCMQHLEYPYT
FGGGTKLEIERTVAAPSVFIFPPSDEQLKSGTASVVCLLNNFYPREAKVQW
KVDNALQSGNSQESVTEQDSKDSTYSLCSTLTLSKADYEKHKVYACEVTHQ
GLSSPVTKSFNRGES

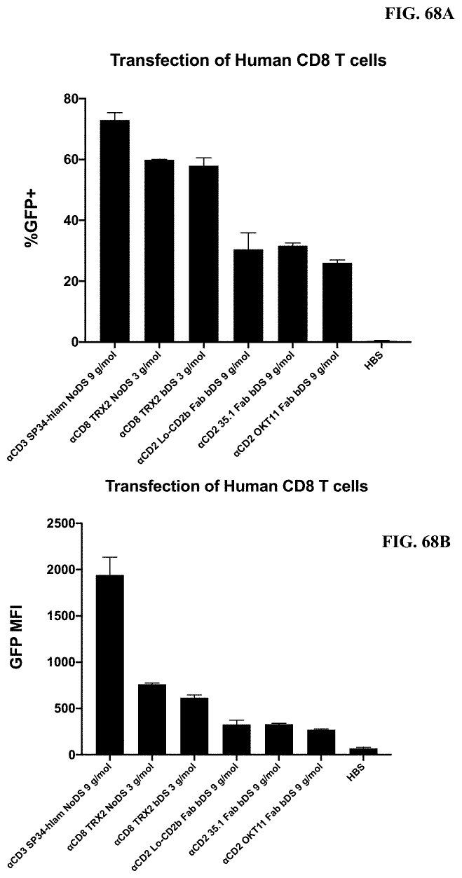

The interchain disulfide knockout (NoDS) anti-CD8 TRX2 Fab had slightly higher % transfection (FIG. 68A) and GFP expression levels (FIG. 68B) than the buried disulfide (bDS) TRX2 Fab however the difference was small. Similar to Example 17, none of the CD2 targeting Fabs explored herein performed as well as anti-CD3 or anti-CD8 Fab.

This data indicates that the TRX2 clone in the NoDS format is preferred however Fabs in the bDS format can mediate efficient T cell transfection/translation. Additionally, CD8 and CD3 are preferred targets over CD2 for the clones evaluated.

### Example 36—In Vitro Protein Expression—Cd8 Targeted Fab with and without Buried Disulfide and Other Cd2 Targeted Fab Clones

This example describes targeting human T cells by co-targeting with anti-CD3 and ant-CD11a or anti-CD3 and anti-CD18 Fabs post-inserted into Cy5/GFP mRNA LNPs and their effect on transfection/translation and IFNγ cytokine secretion.

LNPs were prepared using the mixing process described in Example 6, the buffer exchange process described in Example 21. Fab-lipid conjugates generated from methods described in Example 4. Using methods similar to Example 12, ant-CD3 hSP34, anti-CD11a HzMHM24, anti-CD18 Erlizumab PEG-lipid conjugated Fabs were post-inserted into LNPs containing Lipid 8 and Cy5/GFP mRNA. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK7) to distinguish the two cell types. IFNγ in the supernatants was measured using the manufacturers recommended procedure (R&D Systems, DY285B).

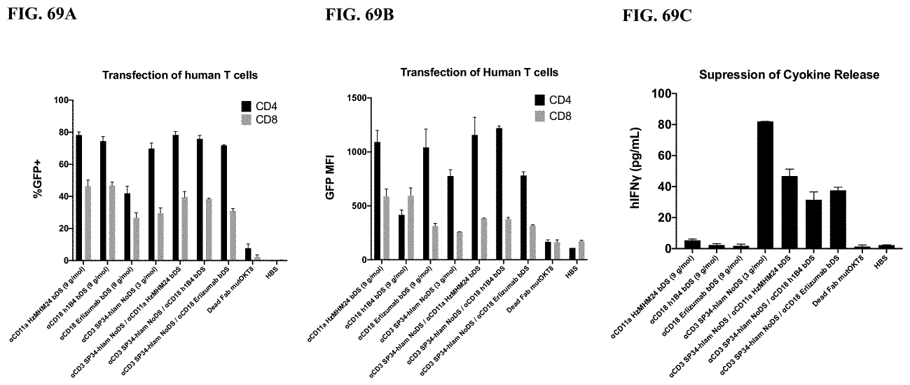

Targeting either CD11a or CD18 alone mediated transfection (FIG. 69A) and GFP expression (FIG. 69B) for both CD8 and CD4 T cells. Importantly, by co-targeting either anti-CD3 clone with any of the anti-CD11a or CD18 clones, the levels of IFNγ secretion was reduced by nearly 50% while the levels of transfection and translation were either similar or higher than CD3 targeting alone (FIG. 69C).

This data indicates that targeting CD11a and CD18 can mediate efficient immune cell transfection/translation and that co-targeting CD3 with either CD11a or CD18 can substantially reduce the cytokine release from T cells without negatively impacting T cell transfection and protein translation. Another anti-CD3 clone in the NoDS Fab format can approach similar levels of transfection/translation of SP34 in the NoDS Fab format.

Anti-CD11a HzMHM24 bDS Fab sequence
HzMHM24 bDS HC (SEQ ID NO: 64):
EVQLVESGGGLVQPGGSLRLSCAASGYSFTGHWMNWVRQAPGKGLEWVGMI
HPSDSETRYNQKFKDRFTISVDKSKNTLYLQMNSLRAEDTAVYYCARGIYF
YGTTYFDYWGQGTLVTVSSASTKGPSVFPLAPSSKSTSGGTAALGCLVKDY
FPEPVTVSWNSGALTSGVHTCPAVLQSSGLYSLSSVVTVPSSSLGTQTYIC
NVNHKPSNTKVDKKVEPKSSDKTHTCHHHHHH
HzMHM24 bDS LC (SEQ ID NO: 65):
DIQMTQSPSSLSASVGDRVTITCRASKTISKYLAWYQQKPGKAPKLLIYSG
STLQSGVPSRFSGSGSGTDFTLTISSLQPEDFATYYCQQHNEYPLTFGQGT
KVEIKRTVAAPSVFIFPPSDEQLKSGTASVVCLLNNFYPREAKVQWKVDNA
LQSGNSQESVTEQDSKDSTYSLCSTLTLSKADYEKHKVYACEVTHQGLSSP
VTKSFNRGES
Anti-CD18 h1B4 bDS Fab sequence
hlB4 bDS HC (SEQ ID NO: 66):
EVQLVESGGDLVQPGRSLRLSCAASGFTFSDYYMSWVRQAPGKGLEWVAAI
DNDGGSISYPDTVKGRFTISRDNAKNSLYLQMNSLRVEDTALYYCARQGRL
RRDYFDYWGQGTLVTVSTASTKGPSVFPLAPSSKSTSGGTAALGCLVKDYF
PEPVTVSWNSGALTSGVHTCPAVLQSSGLYSLSSVVTVPSSSLGTQTYICN
VNHKPSNTKVDKKVEPKSSDKTHTCHHHHHH
h1B4 bDS LC (SEQ ID NO: 67):
DIQMTQSPSSLSASVGDRVTITCRASESVDSYGNSFMHWYQQKPGKAPKLL
IYRASNLESGVPSRFSGSGSGTDFTFTISSLQPEDIATYYCQQSNEDPLTF
GQGTKLEIKRTVAAPSVFIFPPSDEQLKSGTASVVCLLNNFYPREAKVQWK
VDNALQSGNSQESVTEQDSKDSTYSLCSTLTLSKADYEKHKVYACEVTHQG
LSSPVTKSFNRGES
Anti-CD18 Erlizumab bDS Fab sequence
Erlizumab bDS HC (SEQ ID NO: 68):
EVQLVESGGGLVQPGGSLRLSCATSGYTFTEYTMHWMRQAPGKGLEWVAGI
NPKNGGTSHNQRFMDRFTISVDKSTSTAYMQMNSLRAEDTAVYYCARWRGL
NYGFDVRYFDVWGQGTLVTVSSASTKGPSVFPLAPSSKSTSGGTAALGCLV
KDYFPEPVTVSWNSGALTSGVHTCPAVLQSSGLYSLSSVVTVPSSSLGTQT
YICNVNHKPSNTKVDKKVEP
KSSDKTHTCHHHHHH
Erlizumab bDS LC (SEQ ID NO: 69):
DIQMTQSPSSLSASVGDRVTITCRASQDINNYLNWYQQKPGKAPKLLIYYT
STLHSGVPSRFSGSGSGTDYTLTISSLQPEDFATYYCQQGNTLPPTFGQGT
KVEIKRTVAAPSVFIFPPSDEQLKSGTASVVCLLNNFYPREAKVQWKVDNA
LQSGNSQESVTEQDSKDSTYSLCSTLTLSKADYEKHKVYACEVTHQGLSSP
VTKSFNRGES

### Example 37—In Vitro Protein Expression—CO—Targeted LNPS With CD4 and CD8 Fabs or CD4 CD8 Fab-ScFv Bispecific

This example describes targeting human T cells with anti-CD4 or anti-CD8 Fabs, anti-CD4 and anti-CD8 Fabs and a CD4 Fab with a CD8 ScFv off the CD4 Fab light chain (Fab-ScFv) post-inserted into Cy5/GFP mRNA LNPs and their effect on transfection and IFNγ secretion.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4. Using methods similar to Example 12, anti-CD3 hSP34, anti-CD4 Ibalizumab, anti-CD8 TRX2 conjugated Fabs and CD4/CD8 Ibalizumab/TRX2 Fab-ScFv were post-inserted into LNPs containing Lipid 8 and GFP mRNA with DiI dye. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK3) to distinguish the two cell types. IFNγ in the supernatants was measured using the manufacturers recommended procedure (R&D Systems, DY285B).

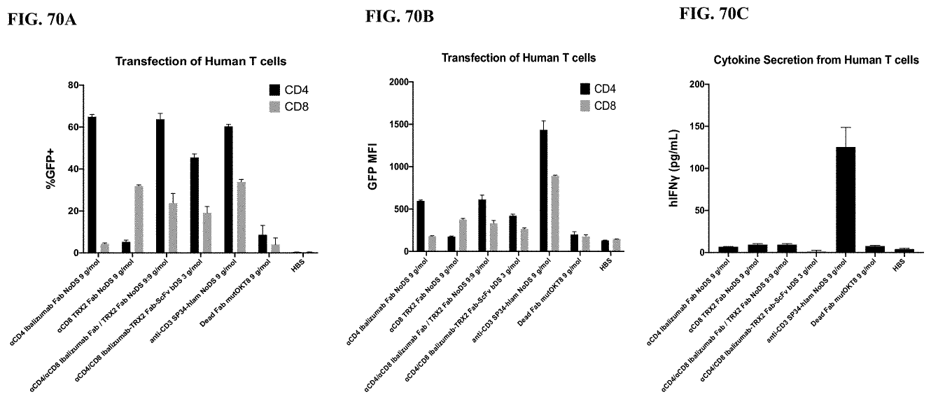

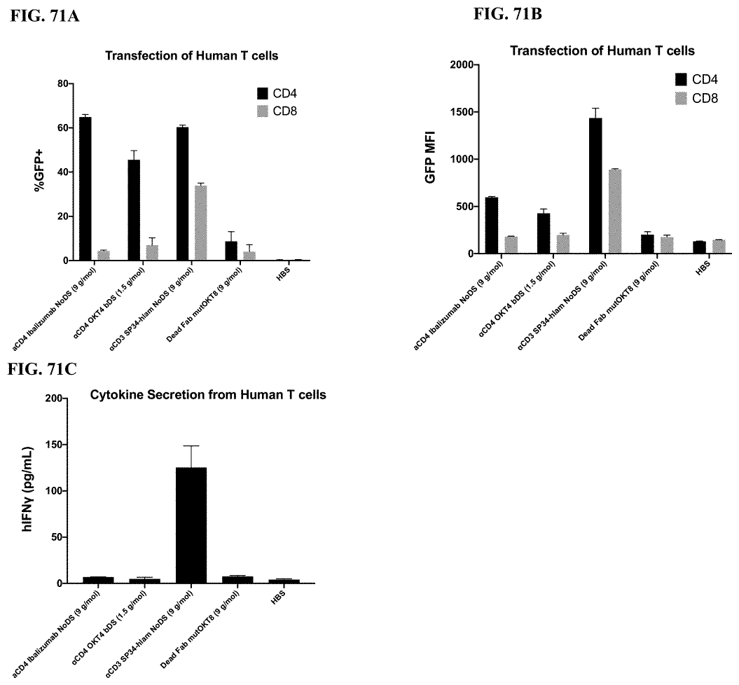

Post-inserting both anti-CD8 and anti-CD4 Fab together shows similar CD8 and CD4 T cell transfection and protein expression relative to the Fabs individually post-inserted (FIGS. 70A and 70B). Compared to post-inserting the CD4 and CD8 Fabs together, the CD4/CD8 Fab-ScFv bispecific shows slightly lower CD4 and CD8 T cell transfection. None of the CD4, CD8 or CD4/CD8 co-targeting conditions mediated substantial IFNγ release in contrast to CD3 targeting with SP34 Fab (FIG. 70C).

This data indicates that a bispecific targeting moiety can be leveraged to have a single protein construct target 2 different immune cell types with minimal loss in targeting function over post-inserting targeting moieties individually into the same LNP.

Anti-CD4/CD8 Ibalizumab/TRX2 bDS Fab-ScFv sequence
Ibalizumab/TRX2 bDS Fab-ScFv HC (SEQ ID NO: 70):
QVQLQQSGPEVVKPGASVKMSCKASGYTFTSYVIHWVRQKPGQGLDWIGYI
NPYNDGTDYDEKFKGKATLTSDTSTSTAYMELSSLRSEDTAVYYCAREKDN
YATGAWFAYWGQGTLVTVSSASTKGPSVFPLAPSSKSTSGGTAALGCLVKD
YFPEPVTVSWNSGALTSGVHTCPAVLQSSGLYSLSSVVTVPSSSLGTQTYI
CNVNHKPSNTKVDKKVEPKSSDKTHTCHHHHHH
Ibalizumab/TRX2 bDS Fab-ScFv LC (SEQ ID NO: 71):
DIVMTQSPDSLAVSLGERVTMNCKSSQSLLYSTNQKNYLAWYQQKPGQSPK
LLIYWASTRESGVPDRFSGSGSGTDFTLTISSVQAEDVAVYYCQQYYSYRT
FGGGTKLEIKRTVAAPSVFIFPPSDEQLKSGTASVVCLLNNFYPREAKVQW
KVDNALQSGNSQESVTEQDSKDSTYSLCSTLTLSKADYEKHKVYACEVTHQ
GLSSPVTKSFNRGESGGGGSGGGGSGGGGSQVQLVESGGGVVQPGRSLRLS
CAASGFTFSDFGMNWVRQAPGKGLEWVALIYYDGSNKFYADSVKGRFTISR
DNSKNTLYLQMNSLRAEDTAVYYCAKPHYDGYYHFFDSWGQGTLVTVSSGG
GGSGGGGSGGGGSGGGGSDIQMTQSPSSLSASVGDRVTITCKGSQDINNYL
AWYQQKPGKAPKLLIYNTDILHTGVPSRFSGSGSGTDFTFTISSLQPEDIA
TYYCYQYNNGYTFGQGTKVEIK

### Example 38—In Vitro Protein Expression—CD4 Targeted Fabs without Natural Interchain Disulfide or with Buried Interchain Disulfide

This example describes targeting human T cells with anti-CD3 or anti-CD4 Fabs post-inserted into Cy5/GFP mRNA LNPs and their effect on transfection and IFNγ secretion.

LNPs were prepared using the mixing process described in Example 6, the buffer exchange process described in Example 21. Fab-lipid conjugates generated from methods described in Example 4. Using methods similar to Example 12, ant-CD3 hSP34, anti-CD4 Ibalizumab, anti-CD4 humanized OKT4 PEG-lipid conjugated Fabs and Nb were post-inserted into LNPs containing Lipid 8 and Cy5/GFP mRNA. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK4) to distinguish the two cell types. IFNγ in the supernatants was measured using the manufacturers recommended procedure (R&D Systems, DY285B).

Amongst the CD4 targeted Fabs, Ibalizumab mediated higher % transfection (FIG. 71A) and GFP expression levels (FIG. 71B) quantified by mean fluorescence intensity, MFI) however it was lower than anti-CD3 SP34 Fab. None of the anti-CD4 Fabs mediated substantial IFNγ secretion levels over non-targeted mutOKT8 Fab while anti-CD3 SP34 Fab exhibited higher levels of IFNγ (FIG. 71C).

This data indicates that anti-CD4 Fabs without the natural interchain disulfide (Ibalizumab, NoDS) or with a buried interchain disulfide (OKT4, bDS) can mediate highly specific LNP transfection and protein translation of CD4+ T cells versus CD8+ T cells and targeting CD4 can avoid T cell activation and IFNγ release.

Anti-CD4 Ibalizumab NoDS Fab sequence
Ibalizumab NoDS LC (SEQ ID NO: 72):
QVQLQQSGPEVVKPGASVKMSCKASGYTFTSYVIHWVRQKPGQGLDWIGYI
NPYNDGTDYDEKFKGKATLTSDTSTSTAYMELSSLRSEDTAVYYCAREKDN
YATGAWFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYI
CNVNHKPSNTKVDKKVEPKSSDKTHTC
Ibalizumab NoDS HC (SEQ ID NO: 73):
DIVMTQSPDSLAVSLGERVTMNCKSSQSLLYSTNQKNYLAWYQQKPGQSPK
LLIYWASTRESGVPDRFSGSGSGTDFTLTISSVQAEDVAVYYCQQYYSYRT
FGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGES
Anti-CD4 OKT4 bDS Fab sequence
OKT4 bDS LC (SEQ ID NO: 74):
EVQLVESGGGLVQPGGSLRLSCAASGFTFSNYAMSWVRQAPGKRLEWVSAI
SDHSTNTYYPDSVKGRFTISRDNAKNTLYLQMNSLRAEDTAVYYCARKYGG
DYDPFDYWGQGTLVTVSSASTKGPSVFPLAPSSKSTSGGTAALGCLVKDYF
PEPVTVSWNSGALTSGVHTCPAVLQSSGLYSLSSVVTVPSSSLGTQTYICN
VNHKPSNTKVDKKVEPKSSDKTHTCHHHHHH
OKT4 bDS HC (SEQ ID NO: 75):
DIQMTQSPSSLSASVGDRVTITCQASQDINNYIAWYQHKPGKGPKLLIHYT
STLQPGIPSRFSGSGSGRDYTLTISSLQPEDFATYYCLQYDNLLFTFGGGT
KVEIKRTVAAPSVFIFPPSDEQLKSGTASVVCLLNNFYPREAKVQWKVDNA
LQSGNSQESVTEQDSKDSTYSLCSTLTLSKADYEKHKVYACEVTHQGLSSP
VTKSFNRGES

### Example 39—In Vitro Protein Expression—Other CD4 Targeted FAB Clones and a CD4 Targeted Nanobody

This example describes targeting human T cells with anti-CD3 or anti-CD4 Fabs post-inserted into GFP mRNA DiI labeled LNPs and their effect on transfection and IFNγ secretion.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of Nb-conjugated differed in using 1:1:4 Nb:DSPE-3.4KPEG-maileimide:DSPE-2KPEG-OCH3 and a 50 kD UF membrane (Millipore Corp, Billerica, Mass. USA) for separation of Nb-conjugate from free Nb. Using methods similar to Example 12, anti-CD3 hSP34, anti-CD4 Ibalizumab, anti-CD4 hBF5 conjugated Fabs and conjugated Nb (derived from llama immunization) were post-inserted into LNPs containing Lipid 8 and GFP mRNA with DiI dye. Transfections were performed with human CD3 T cells at approximately 2.5 μg/mL mRNA for approximately 24 hr. Levels of transfection of both CD8 and CD4 cells was measured by flow cytometry using an anti-CD4 antibody (OKT3) to distinguish the two cell types. IFNγ in the supernatants was measured using the manufacturers recommended procedure (R&D Systems, DY285B).

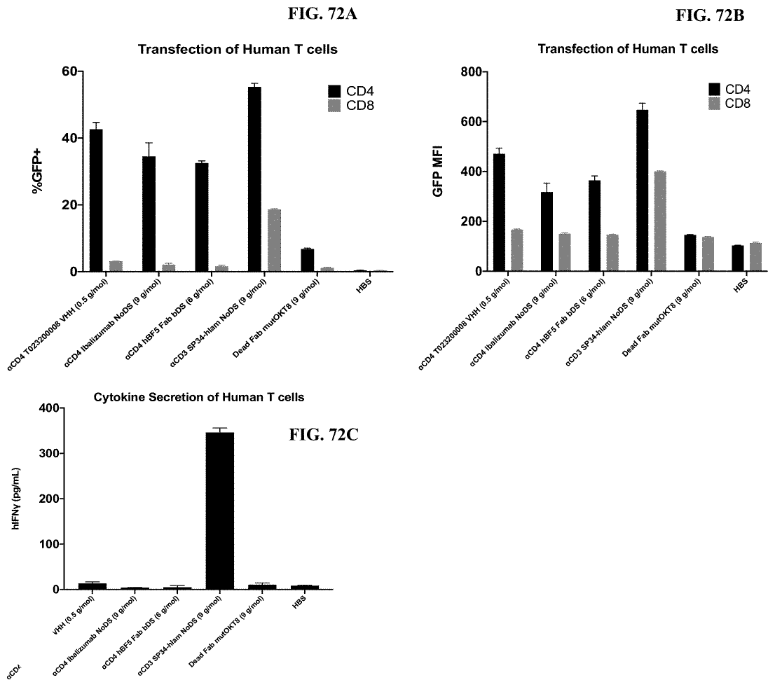

Amongst the CD4 targeted conjugates, the anti-CD4 mediated slightly higher % transfection (FIG. 72A) and GFP expression levels (FIG. 72B) quantified by mean fluorescence intensity, MFI) however it was lower than anti-CD3 SP34 Fab. For the CD4 targeted Fabs and Nb the transfection and translation was only observed in the CD4+ T cell population. None of the anti-CD4 Fabs mediated substantial IFNγ secretion levels over non-targeted mutOKT8 Fab while anti-CD3 SP34 Fab exhibited higher levels of IFNγ.

This data indicates that both Fabs and Nanobodies can mediate highly specific LNP transfection and protein translation by CD4+ T cells versus CD8 T cells and targeting CD4 can avoid T cell activation and IFNγ release.

Anti-CD4 T023200008 Nb sequence
CDR1, CDR2, CDR3 underlined based on IMGT
designation:
(SEQ ID NO: 76)
EVQLVESGGGSVQPGGSLTLSCGTSGRTFNVMGWFRQAPGKEREFVAAVRW
SSTGIYYTQYADSVKSRFTISRDNAKNTVYLEMNSLKPEDTAVYYCAADTY
NSNPARWDGYDFRGQGTLVTVSSGGCGGHHHHHH

### Example 40—In Vitro Protein Expression—CD8 Targeted Nanobody Clone

This example describes targeting human T cells with anti-CD3, anti-CD8 Fab or anti-CD8 Nanobodies post-inserted into GFP mRNA DiI labeled LNPs and their effect on transfection and IFNγ secretion.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of Nb-conjugated differed in using 1:1:4 Nb:DSPE-SKPEG-maileimide:DSPE-2KPEG-OCH3 and a 50 kD UF membrane (Millipore Corp, Billerica, Mass. USA) for separation of Nb-conjugate from free Nb. Using methods similar to Example 12 conjugated Fabs and conjugated Nb (derived from llama or alpaca immunization) were post-inserted into LNPs containing Lipid 8 and GFP mRNA with DiI dye. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK3) to distinguish the two cell types. IFNγ in the supernatants was measured using the manufacturers recommended procedure (R&D Systems, DY285B).

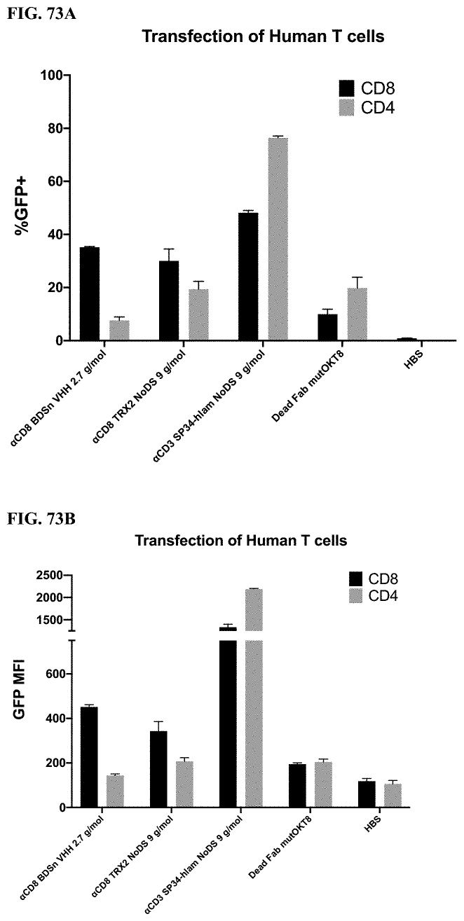

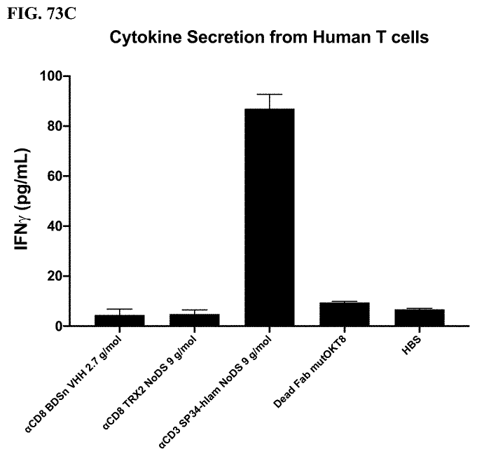

The CD8 targeted Nb conjugate exhibited higher % transfection (FIG. 73A) and GFP expression levels (FIG. 73B) than anti-CD8 TRX2. For the CD8 targeted Nb, transfection and translation was only observed in the CD8 T cell population relative to the mutOKT8 Fab. The anti-CD8 Nb did not mediate substantial IFNγ secretion levels over non-targeted mutOKT8 Fab while anti-CD3 SP34 Fab exhibited higher levels of IFNγ (FIG. 73C).

This data indicates that both Fabs and Nanobodies can mediate highly specific LNP transfection and protein translation by CD8 T cells versus CD4 T cells and targeting CD8 can avoid T cell activation and IFNγ release.

Anti-CD8 BDSn Nb sequence
CDR1, CDR2, CDR3 underlined based on IMGT
designation:
(SEQ ID NO: 77)
EVQLVESGGGLVQAGGSLRLSCAASGSTFSDYGVGWFRQAPGKGREFVADI
DWNGEHTSYADSVKGRFATSRDNAKNTAYLQMNSLKPEDTAVYYCAADALP
YTVRKYNYWGQGTQVTVSSGGCGGHHHHHH

### Example 41—In Vitro Protein Expression—CD3 and Cd7 Targeted Nanobodies with 2K OR 5K Peg

This example describes targeting human T cells with anti-CD3, anti-CD7 Fab or anti-CD8 Nanobodies post-inserted into GFP mRNA DiI labeled LNPs and their effect on transfection and IFNγ secretion.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of Nb-conjugated differed in using 1:1:4 Nb:DSPE-SKPEG-maileimide or DSPE-3.4KPEG-maileimide:DSPE-2KPEG-OCH3 and a 50 kD UF membrane (Millipore Corp, Billerica, Mass. USA) for separation of Nb-conjugate from free Nb. Using methods similar to Example 12 conjugated Fabs and conjugated Nb (E11 and G03), V1 (anti-CD7) were post-inserted into LNPs containing Lipid 8 and GFP mRNA with DiI dye. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK3) to distinguish the two cell types.

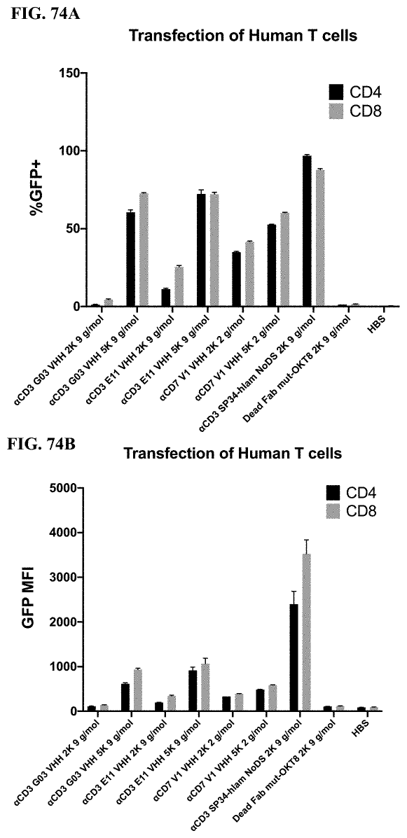

For both anti-CD3 Nb clones and the anti-CD7 Nb the longer, 5K PEG, improved % transfection (FIG. 74A) and GFP expression levels (FIG. 74B) over the 2K PEG. For the anti-CD3 Nbs the difference between the conjugate PEG lengths was more dramatic than for the anti-CD7 Nbs.

This data indicates that Nanobody conjugates can benefit from a PEG length longer than 2K and that different clones can have varying degrees of improvement.

Anti-CD3 T0170117G03-A Nb sequence
(SEQ ID NO: 78)
EVQLVESGGGPVQAGGSLRLSCAASGRTYRGYSMGWFRQAPGKEREFVAAI
VWSGGNTYYEDSVKGRFTISRDNAKNIMYLQMTSLKPEDSATYYCAAKIRP
YIFKIAGQYDYWGQGTLVTVSSAGGGSGGHHHHHHC
Anti-CD3 T0170060E11 Nb sequence
(SEQ ID NO: 79)
EVQLVESGGGLVQPGGSLRLSCAASGDIYKSFDMGWYRQAPGKQRDLVAVI
GSRGNNRGRTNYADSVKGRFTISRDGTGNTVYLLMNKLRPEDTAIYYCNTA
PLVAGRPWGRGTLVTVSSGGGSGGHHHHHHC
Anti-CD7 V1 Nb sequence
(SEQ ID NO: 80)
DVQLQESGGGLVQAGGSLRLSCAVSGYPYSSYCMGWFRQAPGKEREGVAAI
DSDGRTRYADSVKGRFTISQDNAKNTLYLQMNRMKPEDTAMYYCAARFGPM
GCVDLSTLSFGHWGQGTQVTVSITGGGCHHHHHHHH

### Example 42—In vitro protein expression—CD8, CD3, CD28, CD4 and TCR targeted NANOBODIES WITH 2K OR 5K PEG

This example describes targeting human T cells with anti-CD8, anti-CD3, anti-CD4 Fab and anti-CD8, anti-CD3, anti-CD28, anti-CD4 and anti-TCR Nanobodies post-inserted into GFP mRNA DiI labeled LNPs and their effect on transfection and IFNγ secretion.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of Nb-conjugated differed in using 1:1:4 Nb:DSPE-2KPEG-maileimide:DSPE-2KPEG-OCH3 or Nb:DSPE-SKPEG-maileimide:DSPE-2KPEG-OCH3 and a 50 kD UF membrane (Millipore Corp, Billerica, Mass. USA) for separation of Nb-conjugate from unconjugated Nb. Using methods similar to Example 12 conjugated Fabs and conjugated Nb (derived from llama or alpaca immunization) were post-inserted into LNPs containing Lipid 8 and GFP mRNA with DiI dye. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK3) to distinguish the two cell types.

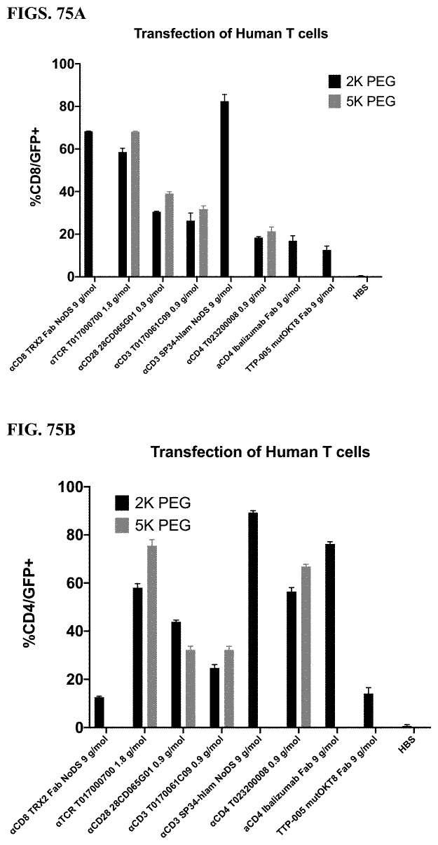

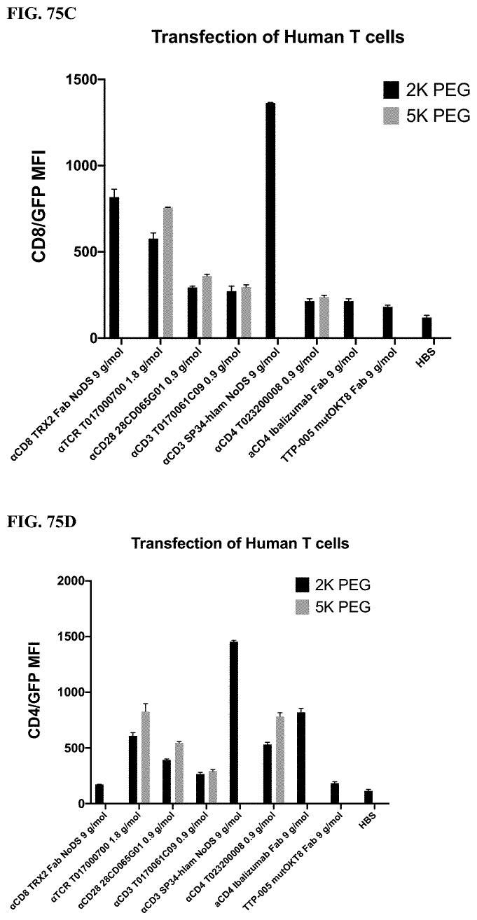

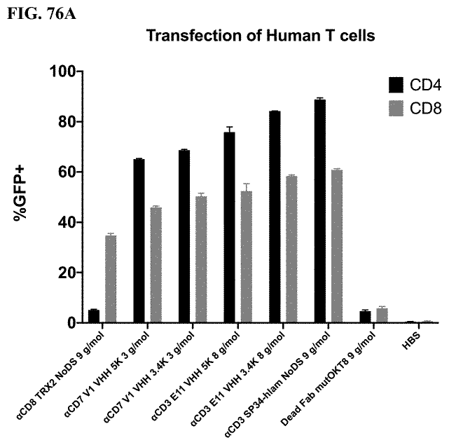

For all of the targets evaluated, nanobodies conjugated with the longer, 5K PEG, generally improved % transfection (FIGS. 75A and 75B) and GFP expression levels (FIGS. 75C and 75D) over the 2K PEG with exception of anti-CD8 clone E05 which showed the reverse relationship. The magnitude of improvement appears to be clone specific.

This data indicates that regardless of the target, the preferred PEG length for a Nanobody PEG-lipid conjugate is greater than 2K.

Anti-TCR T017000700 Nb sequence
CDR1, CDR2, CDR3 underlined based on IMGT
designation:
(SEQ ID NO: 81)
EVQLVESGGGVVQPGGSLRLSCVASGYVHKINFYGWYRQAPGKEREKVAHI
SIGDQTDYADSAKGRFTISRDESKNTVYLQMNSLRPEDTAAYYCRALSRIW
PYDYWGQGTLVTVSSGGCGGHHHHHH
Anti-CD28 28CD065G01 Nb sequence
(SEQ ID NO: 82)
EVQLVESGGGLVQPGGSLRLSCAASGSIFRLHTMEWYRRTPETQREWVATI
TSGGTTNYPDSVKGRFTISRDDTKKTVYLQMNSLKPEDTAVYYCHAVATED
AGFPPSNYWGQGTLVTVSSGGCGGHHHHHH
Anti-CD3 T0170061C09 Nb sequence
(SEQ ID NO: 83)
EVQLVESGGGPVQAGGSLRLSCAASGRTYRGYSMGWFRQAPGREREFVAAI
VWSDGNTYYEDSVKGRFTISRDNAKNTMYLQMTSLKPEDSATYYCAAKIRP
YIFKIAGQYDYWGQGTLVTVSSGGCGGHHHHHH
Anti-CD4 T023200008 Nb sequence
(SEQ ID NO: 76)
EVQLVESGGGSVQPGGSLTLSCGTSGRTFNVMGWFRQAPGKEREFVAAVRW
SSTGIYYTQYADSVKSRFTISRDNAKNTVYLEMNSLKPEDTAVYYCAADTY
NSNPARWDGYDFRGQGTLVTVSSGGCGGHHHHHH

### Example 43—In Vitro Protein Expression—Cd8, Cd7 and Cd3 Targeted Nanobodies with 5K or 3.4K PEG

This example describes targeting human T cells with anti-CD8, anti-CD3 Fab and anti-CD8, anti-CD7 and anti-CD3 Nanobodies post-inserted into GFP mRNA DiI labeled LNPs and their effect on transfection.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of Nb-conjugated differed in using 1:1:4 Nb:DSPE-3.4KPEG-maileimide:DSPE-2KPEG-OCH3 or Nb:DSPE-SKPEG-maileimide:DSPE-2KPEG-OCH3 and a 50 kD UF membrane (Millipore Corp, Billerica, Mass. USA) for separation of Nb-conjugate from unconjugated Nb. Using methods similar to Example 12 conjugated Fabs and conjugated Nb (derived from llama or alpaca immunization) were post-inserted into LNPs containing Lipid 5 and GFP mRNA with DiI dye at temperature of 37 C for 4 hrs. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK3) to distinguish the two cell types.

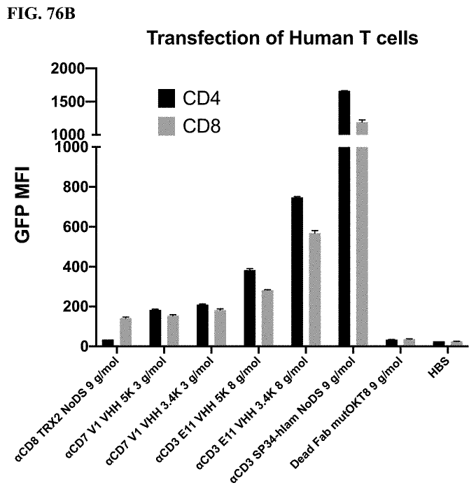

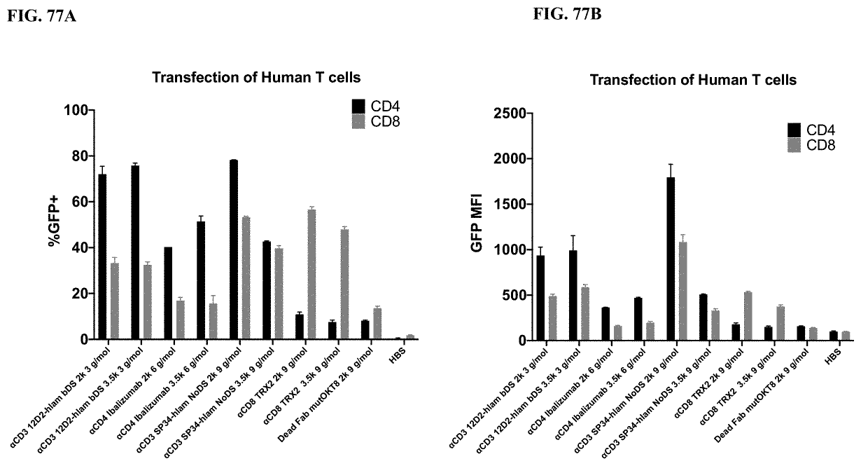

For all of the targets evaluated, Nb conjugated with the shorter, 3.4K PEG was similar if not slightly better than the longer 5K PEG in % transfection (FIG. 76A) and GFP expression levels (FIG. 76B) or in the case of the anti-CD7 V1 Nb 3.4K PEG mediated higher transfection and expression than the 5K PEG.

This data indicates that regardless of the target, the generally preferred PEG length for Nanobody-PEG-lipid conjugates is 3.4K PEG versus the shorter 2K PEG as previously described in EXAMPLE 42 or the longer 5K PEG as described herein.

### Example 44—In Vitro Protein Expression—2K Versus 3.4K Peg Spacer for Fabs

This example describes targeting human T cells with anti-CD3, anti-CD4, anti-CD8, anti-CD28, Fabs post-inserted into GFP mRNA DiI labeled LNPs and their effect on transfection.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4. Using methods similar to Example 12 conjugated Fab (12D2) was post-inserted into LNPs containing Lipid 8 and GFP mRNA with DiI dye. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK3) to distinguish the two cell types.

While most Fab clones did not differ in % transfection (FIG. 77A) and GFP expression levels (FIG. 77B) between the 2 PEG lengths, anti-CD4 Ibalizumab showed an increase in transfection efficiency going from 2K PEG to 3.4K PEG while anti-CD3 SP34 transfection efficiency decreased going from 2K PEG to 3.4K PEG.

This data indicates that generally a 2K PEG spacer is preferred for Fab-PEG-lipid conjugates however some clones can gain benefit from a longer PEG spacer. Additionally, it indicates anti-CD3 clone 12D2 with a buried disulfide with either a 2K or 3.4K PEG can efficiently transfect both CD8 and CD4 T cell subsets.

Anti-CD3 12D2 bDS Fab sequence
12D2 bDS HC (SEQ ID NO: 84):
EVKLVESGGGLVQPGRSLRLSCAASGFNFYAYWMGWVRQAPGKGLEWIGEI
KKDGTTINYTPSLKDRFTISRDNAQNTLYLQMTKLGSEDTALYYCAREERD
GYFDYWGQGVMVTVSSASTKGPSVFPLAPSSKSTSGGTAALGCLVKDYFPE
PVTVSWNSGALTSGVHTCPAVLQSSGLYSLSSVVTVPSSSLGTQTYICNVN
HKPSNTKVDKKVEPKSSDKTHTCGGHHHHHH
12D2 bDS LC (SEQ ID NO: 85):
QFVLTQPNSVSTNLGSTVKLSCKRSTGNIGSNYVNWYQQHEGRSPTTMIYR
DDKRPDGVPDRFSGSIDRSSNSALLTINNVQTEDEADYFCQSYSSGIVFGG
GTKLTVLSQPKAAPSVTLFPPSSEELQANKATLVCLVSDFYPGAVTVAWKA
DGSPVKVGVETTKPSKQSNNKYAACSYLSLTPEQWKSHRSYSCRVTHEGST
VEKTVAPAESS

### Example 45—In Vitro Protein Expression—CD8 Targeted Nanobody mRNA Titration

This example describes targeting human T cells with anti-CD3, anti-CD8 Fab or anti-CD8 Nanobodies post-inserted into GFP mRNA DiI labeled LNPs and their effect on transfection and protein expression.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of Nb-conjugated differed in using 1:1:4 Nb:DSPE-3.4KPEG-maileimide:DSPE-2KPEG-OCH3 and a 50 kD UF membrane (Millipore Corp, Billerica, Mass. USA) for separation of Nb-conjugate from free Nb. Using methods similar to Example 12 conjugated Fabs and conjugated Nb (derived from alpaca immunization) were post-inserted into LNPs containing Lipid 8 and GFP mRNA with DiI dye. Transfections were performed with human CD3 T cells at approximately 2.5, 0.5 and 0.1 μg/mL mRNA for approximately 24 hr. Levels of transfection of both CD8 and CD4 cells was measured by flow cytometry using an anti-CD4 antibody (SK3) to distinguish the two cell types.

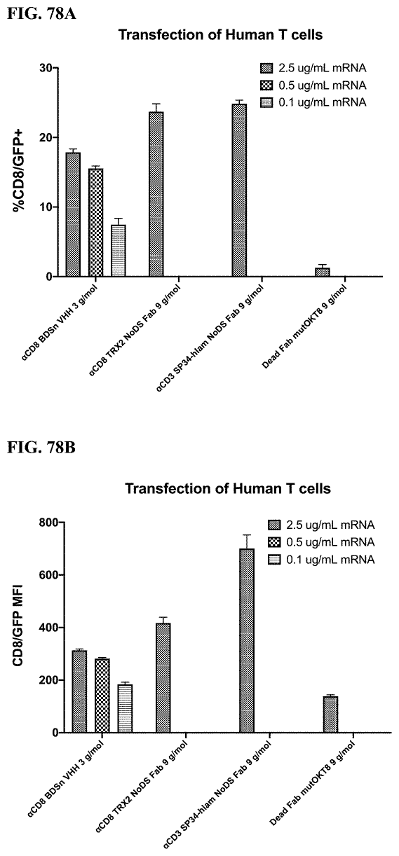

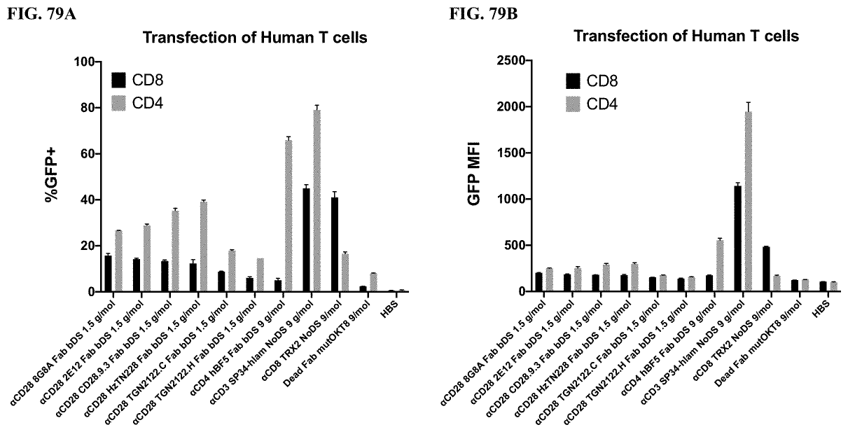

The CD8 targeted Nb conjugate exhibited % transfection (FIG. 78A) and GFP expression levels (FIG. 78B) greater than the mutOKT8 negative control down to 0.1 ug/mL mRNA.

This data indicates that Nanobodies conjugated with 3.4K PEG-lipid can mediate highly potent T cell transfection with low levels of mRNA concentration in solution.

### Example 46—In Vitro Protein Expression—CD28 Targeted Fab Clones

This example describes targeting human T cells with anti-CD28, anti-CD8, anti-CD4, anti-CD3 Fabs post-inserted into GFP mRNA LNPs (doped with DiI dye) and their effect on transfection and IFNγ secretion.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from similar methods described in Example 4. Using methods similar to Example 12, anti-CD28 8G8A, anti-CD28 2E12, anti-CD28 CD28.9.3, anti-CD28 HzTN228, anti-CD28 TGN2122.C/H.

PEG-lipid conjugated Fabs were post-inserted into LNPs containing Lipid 8 and GFP mRNA and doped with DiI dye. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK3) to distinguish the two cell types. IFNγ in the supernatants was measured using the manufacturers recommended procedure (R&D Systems, DY285B).

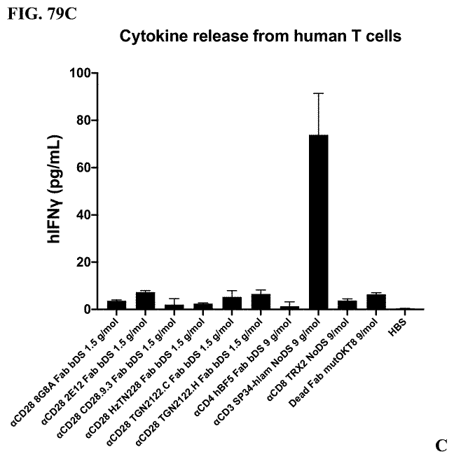

While most of the CD28 targeting Fabs show CD8 and CD4 T cell GFP transfection (FIG. 79A) and expression levels (FIG. 79B) greater than that of the mutOKT8 post-inserted particles, none of the clones evaluated surpass single T cell subset targeting with anti-CD4 hBF5, anti-CD8 TRX2 or targeting both subsets with anti-CD3 SP34. Other than SP34, none of the clones evaluated elicited substantial IFNγ secretion over mutOKT8 LNPs (FIG. 79C).

This data indicates that despite being able to transfect both CD4 and CD8 T cell subsets, there is not an advantage in transfection/translation efficiency by targeting CD28 versus targeting CD4, CD8 or CD3 for the clones evaluated.

Anti-CD28 8G8A Fab sequence
8G8A bDS HC (SEQ ID NO: 86):
EVQLQQSGPELVKPGASVKMSCKASGYTFTSYVIQWVKQKPGQGLEWIGSI
NPYNDYTKYNEKFKGKATLTSDKSSITAYMEFSLTSEDSALYCARWGDGNY
WGRGTLTVSSASTKGPSVFPLAPSSKSTSGGTAALGCLVKDYFPEPVTVSW
NSGALTSGVHTCPAVLQSSGLYSLSSVVTVPSSSLGTQTYICNVNHKPSNT
KVDKKVEPKSSDKTHTCGGHHHHHH
8G8A bDS LC (SEQ ID NO: 87):
DIEMTQSPAIMSASLGERVTMTCTASSSVSSSYFHWYQKPGSSPKLCIYST
SNLASGVPPRFSGSGSTSYSLTISMEAEDAATYFCHQYHRSPTFGGGTKLE
TKRTVAAPSVFIFPPSDEQLKSGTASVVCLLNNFYPREAKVQWKVDNALQS
GNSQESVTEQDSKDSTYSLCSTLTLSKADYEKHKVYACEVTHQGLSSPVTK
SFNRGES
Anti-CD28 2E12 Fab sequence
2E12 bDS HC (SEQ ID NO: 88):
QVQLKESGPGLVAPSQSLSITCTVSGFSLTGYGVNWVRQPPGKGLEWLGMI
WGDGSTDYNSALKSRLSITKDNSKSQVFLKMNSLQTDDTARYYCARDGYSN
FHYYVMDYWGQGTSVTVSSASTKGPSVFPLAPSSKSTSGGTAALGCLVKDY
FPEPVTVSWNSGALTSGVHTCPAVLQSSGLYSLSSVVTVPSSSLGTQTYIC
NVNHKPSNTKVDKKVEPKSSDKTHTCGGHHHHHH
2E12 bDS LC (SEQ ID NO: 89):
DIVLTQSPASLAVSLGQRATISCRASESVEYYVTSLMQWYQQKPGQPPKLL
ISAASNVESGVPARFSGSGSGTDFSLNIHPVEEDDIAMYFCQQSRKVPWTF
GGGTKLEIKRRTVAAPSVFIFPPSDEQLKSGTASVVCLLNNFYPREAKVQW
KVDNALQSGNSQESVTEQDSKDSTYSLCSTLTLSKADYEKHKVYACEVTHQ
GLSSPVTKSFNRGES
Anti-CD28 CD28.9.3 Fab sequence
CD28.9.3 bDS HC (SEQ ID NO: 90):
QVKLQQSGPGLVTPSQSLSITCTVSGFSLSDYGVHWVRQSPGQGLEWLGVI
WAGGGTNYNSALMSRKSISKDNSKSQVFLKMNSLQADDTAVYYCARDKGYS
YYYSMDYWGQGTTVTVSSASTKGPSVFPLAPSSKSTSGGTAALGCLVKDYF
PEPVTVSWNSGALTSGVHTCPAVLQSSGLYSLSSVVTVPSSSLGTQTYICN
VNHKPSNTKVDKKVEPKSSDKTHTCGGHHHHHH
CD28.9.3 bDS LC (SEQ ID NO: 91):
DIVLTQSPAS LAVSLGQRAT ISCRASESVEYYVTSLMQWY QQKPGQPP
KLLIFAASNVES GVPARFSGSG SGTNFSLNIHPVDEDDVAMY FCQQSR
KVPYTFGGGTKLEIKRTVAAPSVFIFPPSDEQLKSGTASVVCLLNNFYPRE
AKVQWKVDNALQSGNSQESVTEQDSKDSTYSLCSTLTLSKADYEKHKVYAC
EVTHQGLSSPVTKSFNRGES
Anti-CD28 HzTN228 Fab sequence
HzTN228 bDS HC (SEQ ID NO: 92):
QVQLQESGPGLVKPSETLSLTCAVSGFSLTSYGVHWIRQPGKGLEWLGVIW
PGTNFNSALMSRLTISEDTSKNQVSLKLSSVTAADTAVYCARDRAYGNYLY
AMDYWGQGTLVTVSSASTKGPSVFPLAPSSKSTSGGTAALGCLVKDYFPEP
VTVSWNSGALTSGVHTCPAVLQSSGLYSLSSVVTVPSSSLGTQTYICNVNH
KPSNTKVDKKVEPKSSDKTHTCGGHHHHHH
HzTN228 bDS LC (SEQ ID NO: 93):
DIQMTQSPSLSASVGDRVTITCRASESVEYVTSLMQWYQKPGKAPKLLIYA
ASNVDSGVPSRFSGSGTDFTLTISLQPEDIATYCQSRKVPFTFGGGTKVEI
KRTVAAPSVFIFPPSDEQLKSGTASVVCLLNNFYPREAKVQWKVDNALQSG
NSQESVTEQDSKDSTYSLCSTLTLSKADYEKHKVYACEVTHQGLSSPVTKS
FNRGES
Anti-CD28 TGN2122.C Fab sequence
TGN2122.C bDS HC (SEQ ID NO: 94):
QVQLVQSGAEVKKPGASVKVSCKASGYTFTDYKIHWVRQAPGQGLEWIGYI
YPYSGSSDYNQKFKSRATLTVDNSISTAYMELSRLRSDDTAVYYCARGGDA
MDYWGQGTLVTVSSASTKGPSVFPLAPSSKSTSGGTAALGCLVKDYFPEPV
TVSWNSGALTSGVHTCPAVLQSSGLYSLSSVVTVPSSSLGTQTYICNVNHK
PSNTKVDKKVEPKSSDKTHTCGGHHHHHH
TGN2122.C bDS LC (SEQ ID NO: 95):
DIQMTQSPSSLSASVGDRVTITCGASENIYGALNWYQRKPGKAPKLLIYGA
TNLADGVPSRFSGSGSGRDYTLTISSLQPEDFATYFCQNILGTWTFGGGTK
VEIKRTVAAPSVFIFPPSDEQLKSGTASVVCLLNNFYPREAKVQWKVDNAL
QSGNSQESVTEQDSKDSTYSLCSTLTLSKADYEKHKVYACEVTHQGLSSPV
TKSFNRGES
Anti-CD28 TGN2122.H Fab sequence
TGN2122.H bDS HC (SEQ ID NO: 96):
EVQLVESGGGLVQPGGSLRLSCAASGFTFNIYYMSWVRQAPGKGLELVAAI
NPDGGNTYYPDTVKGRFTISRDNAKNSLYLQMNSLRAEDTAVYYCARYGGP
GFDSWGQGTLVTVSSASTKGPSVFPLAPSSKSTSGGTAALGCLVKDYFPEP
VTVSWNSGALTSGVHTCPAVLQSSGLYSLSSVVTVPSSSLGTQTYICNVNH
KPSNTKVDKKVEPKSSDKTHTCGGHHHHHH
TGN2122.H bDS LC (SEQ ID NO: 97):
ENVLTQSPATLSLSPGERATLSCSASSSVSYMHWYQQKPGQAPRLWIYDTS
KLASGIPARFSGSGSRNDYTLTISSLEPEDFAVYYCFPGSGFPFMYTFGGG
TKVEIKRTVAAPSVFIFPPSDEQLKSGTASVVCLLNNFYPREAKVQWKVDN
ALQSGNSQESVTEQDSKDSTYSLCSTLTLSKADYEKHKVYACEVTHQGLSS
PVTKSFNRGES

### Example 47—In Vitro Protein Expression—CD3, CD4, CD7, CD8, CD11a, CD18, CD28, and TCR Targeted Fabs and Nanobodies

This example describes targeting human T cells with anti-CD3, anti-CD4, anti-CD7, anti-CD8, anti-CD11a, anti-CD18, anti-CD28 and anti-TCR Fabs and Nbs post-inserted into GFP mRNA DiI labeled LNPs and their effect on transfection and IFNγ secretion.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of Nb-conjugated differed in using 1:1:4 Nb:DSPE-3.4KPEG-maileimide:DSPE-2KPEG-OCH3 and a 50 kD UF membrane (Millipore Corp, Billerica, Mass. USA) for separation of Nb-conjugate from free Nb. Using methods similar to Example 12 conjugated Fabs and conjugated Nbs were post-inserted into LNPs containing Lipid 8 and GFP mRNA with DiI dye. Transfections were performed with human CD3 T cells at approximately 2.5 μg/mL, 0.5, ug/mL and 0.1 ug/mL mRNA for approximately 24 hr. Levels of transfection of both CD8 and CD4 cells was measured by flow cytometry using an anti-CD4 antibody (SK3) distinguish the two cell types.

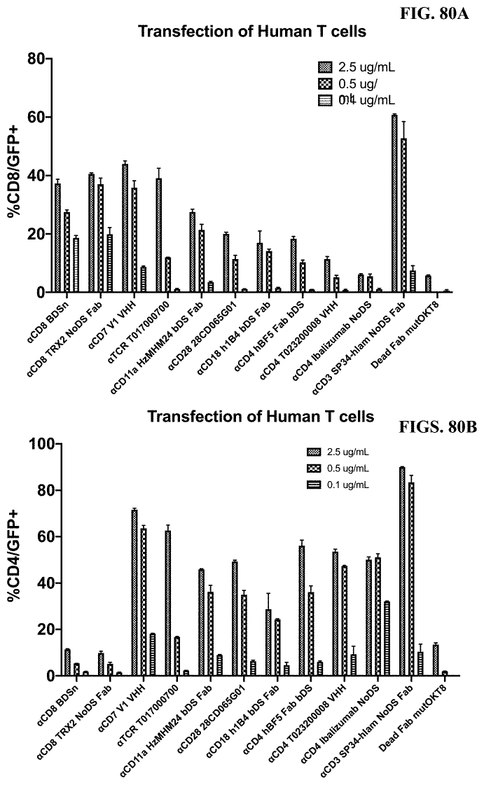

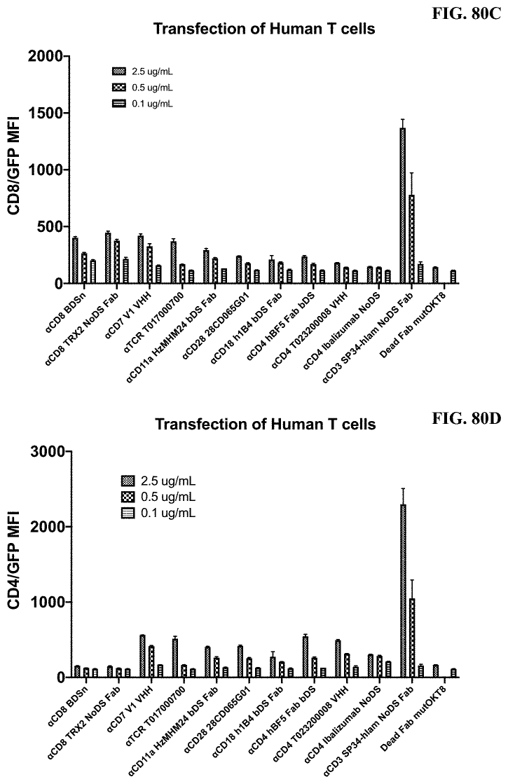

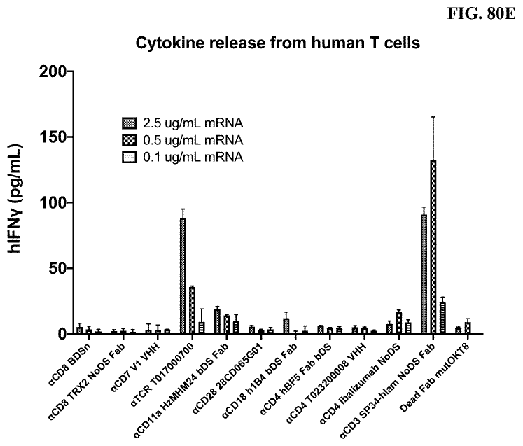

All of the clones evaluated mediated some level of transfection (FIG. 80A, 80B) and GFP expression levels (FIG. 80C, 80D) relative to the mutOKT8 LNP control. For targeting both CD8 and CD4 T cell subset simultaneously anti-CD3 and anti-CD7 stand out as having the highest transfection/translation between both cell subsets at both the highest and second highest mRNA dose. The anti-TCR clone has high transfection/translation efficiency at the highest dose however falls off at the 2nd highest dose. For specific T cell subset targeting, targeting anti-CD8 or anti-CD4 provides the highest specificity for their corresponding subsets regardless of the use of a Fab or Nb. Targeting either CD3 or TCR elicited T cell activation and secretion of IFNγ while the other targeting clones did not elicit levels substantially over the mutOKT8 LNP (FIG. 80E).

This data indicates that targeting CD3 or CD7 with a Fab or Nb is preferred to enable high transfection of both CD4 and CD8 T cell subsets. For targeting CD4 or CD8 T cell subsets individually, use of subset specific anti-CD4 or anti-CD8 Fab or Nb is preferred to enable high transfection of its corresponding T cell subset. Targeting CD3 or TCR can elicit IFNγ secretion while targeting CD4, CD7, CD8, CD11a, anti-CD18 or anti-CD28 can avoid IFNγ secretion.

### Example 48—In Vitro Protein Expression—CD7 and Cd8 Co—Targeted LNPs

This example describes targeting human T cells with anti-CD7 anti-CD8 Nbs post-inserted alone or together into GFP mRNA DiI labeled LNPs and their effect on transfection and IFNγ secretion.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of Nb-conjugated differed in using 1:1:4 Nb:DSPE-SKPEG-maileimide:DSPE-2KPEG-OCH3 and a 50 kD UF membrane (Millipore Corp, Billerica, Mass. USA) for separation of Nb-conjugate from free Nb. Using methods similar to Example 12 conjugated Fabs and conjugated Nb were post-inserted into LNPs containing Lipid 8 and GFP mRNA with DiI dye. Transfections were performed with human CD3 T cells at approximately 2.5 μg/mL mRNA for approximately 24 hr. Levels of transfection of both CD8 and CD4 cells was measured by flow cytometry using an anti-CD4 antibody (SK3) to distinguish the two cell types. IFNγ in the supernatants was measured using the manufacturers recommended procedure (R&D Systems, DY285B).

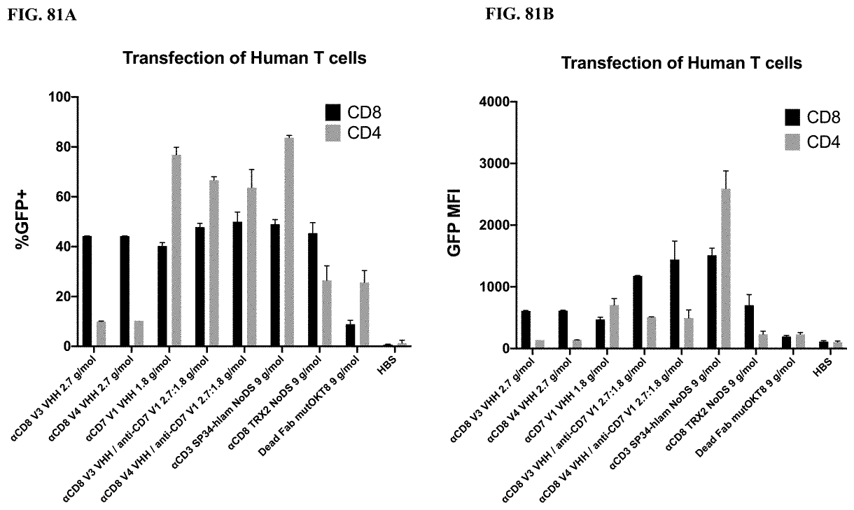

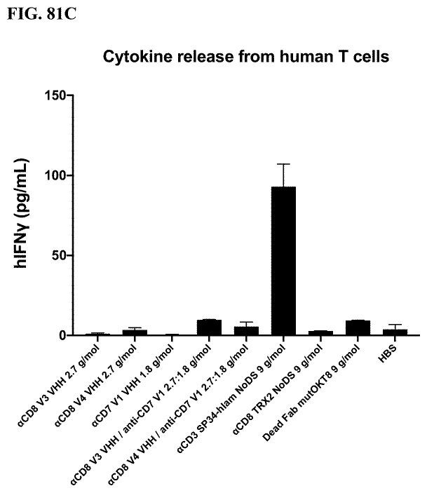

For both anti-CD8 Nb clones (V3 and V4) combined with the anti-CD7 V1 Nb the % transfection (FIG. 81A) and GFP expression levels (FIG. 81B) in CD8 T cell subset were higher than targeting CD8 alone or CD7 alone with Nb or CD8 targeting with the TRX2 NoDS Fab. The CD7/CD8 targeting combination is approaching similar levels of GFP expression to the anti-CD3 SP34 NoDS Fab for CD8 T cells while maintaining similar if not lower levels of Transfection in CD4 T cells. Despite CD8/CD7 co-targeting achieving similar levels of transfection/translation to that of anti-CD3 Fab in the CD8 T cell population, there was not substantial amounts of IFNγ secreted by the T cells relative to the non-specific mutOKT8 control LNPs (FIG. 81C).

This data indicates that co-targeting CD7 and CD8 can mediate highly efficient transfection in the CD8 T cell population while avoiding substantial amounts of IFNγ secretion.

### Example 49—In Vitro Protein Expression—CD7 and CD8 Bispecific Targeted LNPs and CD8 Targeted SCFV

This example describes targeting human T cells with anti-CD8 TRX2 Fab NoDS or anti-CD8 TRX2 ScFv, anti-CD7 or anti-CD8 Nbs post-inserted alone or together and bispecific designs described in FIG. 47 including anti-CD7/anti-CD8 2×VHH (V1/V2), anti-CD8/anti-CD7 2×VHH (V2/V1) or anti-CD7/anti-CD8 VHH-CH1/VHH-Vk bDS post-inserted into GFP mRNA DiI labeled LNPs and their effect on transfection/translation and IFNγ secretion.

LNPs were prepared using the microfluidic mixing process described in Example 6 and discontinuous diafiltration method described in Example 25. The LNPs were formulated using eGFP encoding mRNA (TriLink Biotechnologies, California, US), Lipid 8 as the ionizable lipid, and labeled with 0.01 mol % DiIC18(5)-DS (Invitrogen, Massachusetts, US). Fab-lipid conjugates generated from methods described in Example 4 while generation of ScFv or Nb conjugation differed in using 1:1:4 Nb:DSPE-3.4KPEG-maileimide:DSPE-2KPEG-OCH3 and a 50 kD UF membrane (Millipore Corp, Billerica, Mass. USA) for separation of ScFv or Nb-conjugate from free protein. Using methods similar to Example 12 conjugated Fabs, conjugated ScFv and conjugated Nb were post-inserted into LNPs containing Lipid 8 and GFP mRNA with DiI dye. Transfections were performed with human CD3 T cells at approximately 2.5 ng/mL mRNA for approximately 24 hr. Levels of transfection of both CD8 and CD4 cells was measured by flow cytometry using an anti-CD4 antibody (SK3) to distinguish the two cell types. IFNγ in the supernatants was measured using the manufacturers recommended procedure (R&D Systems, DY285B).

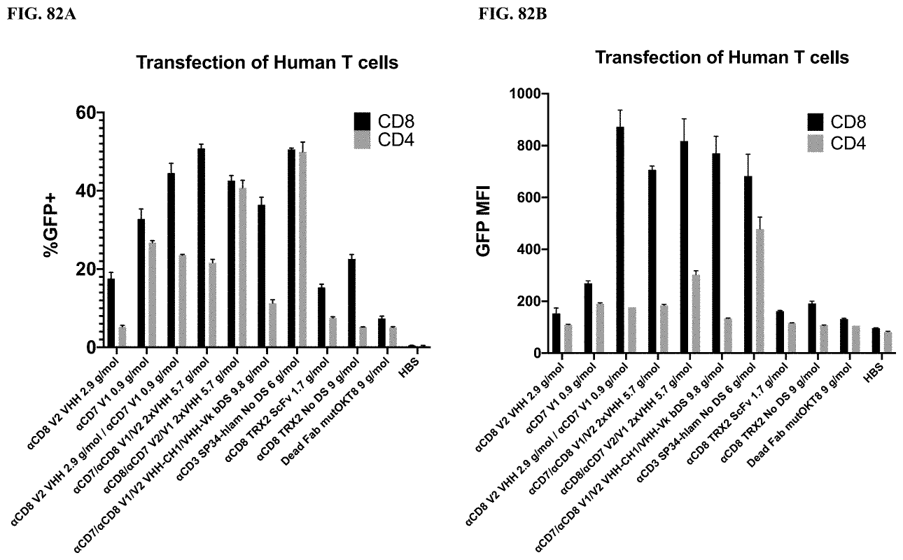

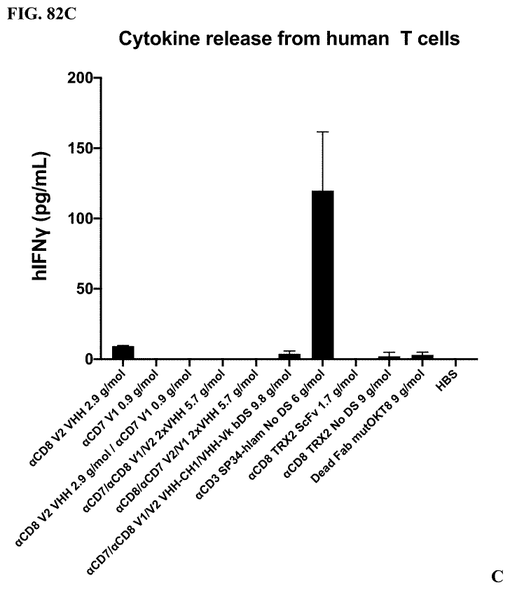

The TRX2 ScFv mediated slightly lower % transfection (FIG. 82A) and GFP expression levels (FIG. 82B) in the CD8 T cell subset relative to the anti-CD8 TRX2 NoDS Fab however the signal was greater than the non-targeted mutOKT8 Fab LNP. The LNPs co-targeting CD8 and CD7 with either anti-CD8 and anti-CD7 Nb post-inserted together or post-inserted bispecifics including anti-CD⁷/anti-CD8 2×VHH, anti-CD8/anti-CD7 2×VHH and anti-CD⁷/anti-CD8 V_{HH}-CH1/V_{HH}-Vk bDS all exhibited high levels of CD8 T cell % transfection and higher levels of GFP expression than the anti-CD3 SP34-hlam NoDS Fab indicating a synergistic effect of combining CD8 and CD7 targeting with Nbs similar to the observation in EXAMPLE 17 co-targeting CD8 (clone TRX2) and CD7 (clone TH-69) with Fabs. Despite CD7/CD8 co-targeting achieving similar or better transfection/translation to that of anti-CD3 SP34 NoDS Fab in the CD8 T cell subset there was not substantial amounts of IFNγ secreted by the T cells relative to the non-specific mutOKT8 control LNPs and in contrast to SP34 (FIG. 82C).

This data indicates an ScFv alone is capable is mediating similar transfection efficiency to that of Fab. Additionally, it indicates that a synergistic effect on transfection/translation can be achieved when targeting both CD7 and CD8 on the same LNP whether the targeting moieties are post inserted together as individual proteins or post inserted as dual-targeting bi-specifics.

Anti-CD8 TRX2 ScFv sequence (SEQ ID NO: 98):
QVQLVESGGGVVQPGRSLRLSCAASGFTFSDFGMNWVRQAPGKGLEWVALI
YYDGSNKFYADSVKGRFTISRDNSKNTLYLQMNSLRAEDTAVYYCAKPHYD
GYYHFFDSWGQGTLVTVSSGGGGSGGGGSGGGGSGGGGSDIQMTQSPSSLS
ASVGDRVTITCKGSQDINNYLAWYQQKPGKAPKLLIYNTDILHTGVPSRFS
GSGSGTDFTFTISSLQPEDIATYYCYQYNNGYTFGQGTKVEIKGGGSGGCG
GHHHHHH
V1 VHH-CH1 bDS HC (SEQ ID NO: 99):
DVQLQESGGGLVQAGGSLRLSCAVSGYPYSSYCMGWFRQAPGKEREGVAAI
DSDGRTRYADSVKGRFTISQDNAKNTLYLQMNRMKPEDTAMYYCAARFGPM
GCVDLSTLSFGHWGQGTQVTVSITASTKGPSVFPLAPSSKSTSGGTAALGC
LVKDYFPEPVTVSWNSGALTSGVHTCPAVLQSSGLYSLSSVVTVPSSSLGT
QTYICNVNHKPSNTKVDKKVEPKSSDKTHTCGGHHHHHH

### Example 50—Lipid 2, Lipid 6, Lipid 12 and Lipid 13 LNP Properties and In Vitro GFP Protein Expression in Primary Human T-Cells

This example compares the properties of LNPs prepared using Lipid 2, Lipid 6, Lipid 12 and Lipid 13 and in vitro GFP protein expression in primary human T-cells. LNP formulations were prepared using the microfluidic mixing process (described in Example 6) and using a discontinuous diafiltration process for ethanol removal (described in Example 25). The LNPs were formulated using eGFP encoding mRNA (TriLink Biotechnologies, California, US) and labeled with 0.01 mol % DiIC18(5)-DS (Invitrogen, Massachusetts, US) using the lipid ratios shown in the Formulation Table 44 below. The LNPs were then inserted with a targeting conjugate using the specified conditions to provide the final targeted LNP formulations. The LNPs were characterized as described in Example 3.

TABLE 44
LNP Formulation composition and antibody insertion conditions

| | | | | Targeting | |
|---|---|---|---|---|---|
| | | | Lipid- | Conjugate/ | Antibody |
| | | | PEG | Insertion | conjugate |
| | | Lipid- | Content | density | insertion |
| Ionizable Lipid | Formulation No. | PEG | (mol %) | (g/mol) | condition |
| Lipid 2 | EXP21003810- | DPG- | 1.5 | hSP34/9 | 37° C. for 4 h in |
| | N1M3 | PEG | | | pH 6.5 MBS |
| Lipid 6 | EXP21003810- | DPG- | 1.5 | hSP34/9 | 37° C. for 4 h in |
| | N2M3 | PEG | | | pH 6.5 MBS |
| Lipid 12 | EXP21003810- | DPG- | 1.5 | hSP34/9 | 37° C. for 4 h in |
| | N3M3 | PEG | | | pH 6.5 MBS |
| Lipid 13 | EXP21003810- | DPG- | 1.5 | hSP34/9 | 37° C. for 4 h in |
| | N4M3 | PEG | | | pH 6.5 MBS |

TABLE 45
LNP size, charge (Dynamic Light Scattering) and mRNA
encapsulation (Ribogreen assay)

| | | | | | Pre- |
|---|---|---|---|---|---|
| | | Pre- | Pre-Insertion | Pre- | insertion |
| | | Insertion | Zeta Potential | Insertion | total |
| | | DLS Z-Avg. | at pH 5.5 | Dye- | mRNA |
| Ionizable | | Diameter | (mV)/pH 7.4 | Accessible | content |
| Lipid | Formulation No. | (nm)/PDI | (mV) | mRNA (%) | (ug/mL) |
| Lipid 2 | EXP21003810-N1H | 62.3/0.08 | 26.2/11.4 | 9.1 | 59.4 |
| Lipid 6 | EXP21003810-N2H | 82.0/0.07 | 25.5/2.27 | 9.2 | 54.6 |
| Lipid 12 | EXP21003810-N3H | 75.9/0.02 | 12.9/−1.06 | 10.4 | 79.2 |
| Lipid 13 | EXP21003810-N4H | 76.3/0.09 | 12.2/−5.20 | 16.5 | 77.4 |

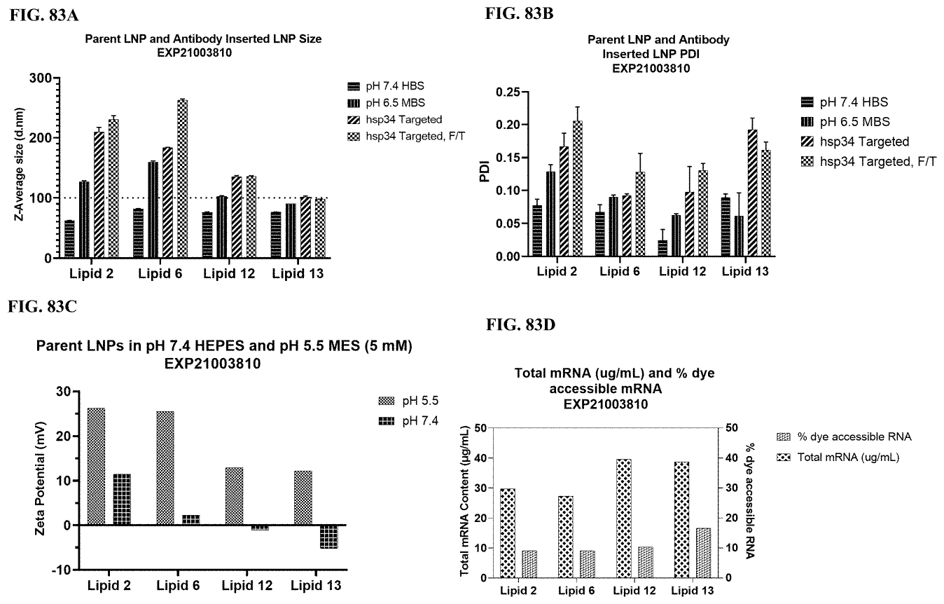

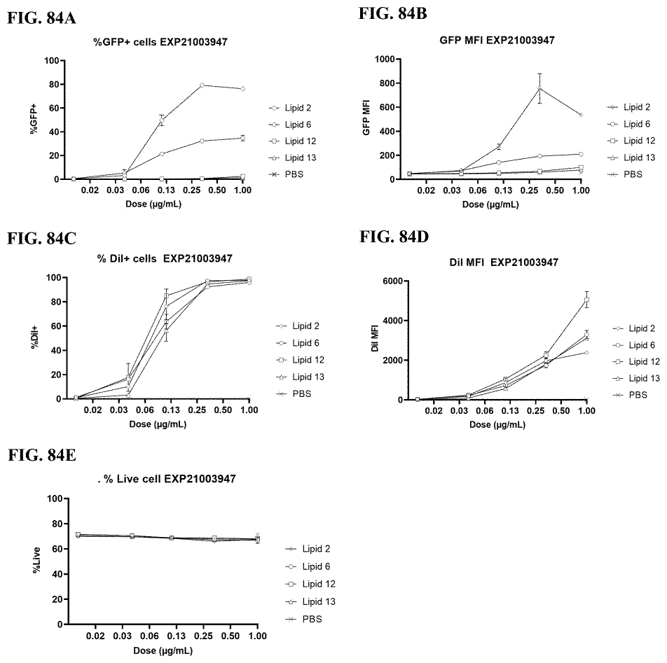

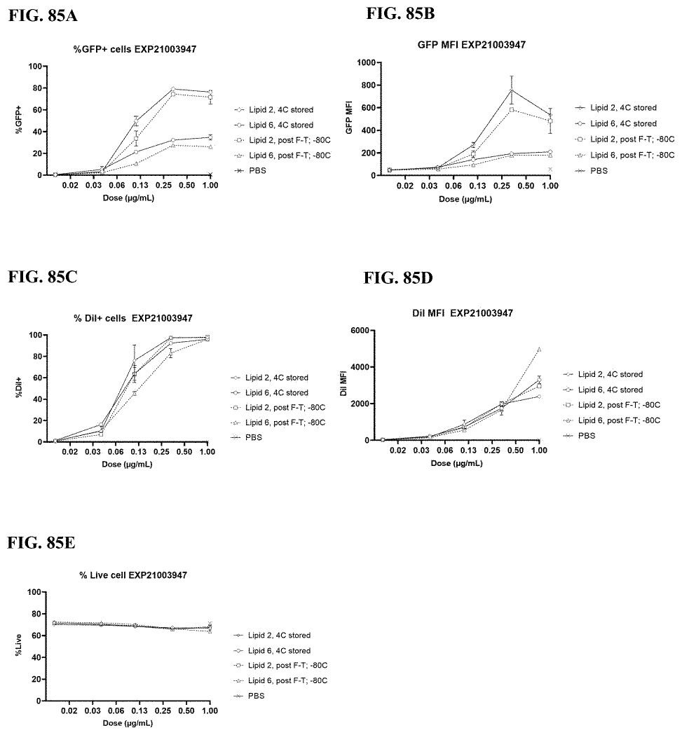

Lipid 2, Lipid 6, Lipid 12 and Lipid 13 were formulated using 1.5 mole % DPG-PEG, as seen in Table 44 and Table 45, all LNPs display sub-100 nm hydrodynamic diameter (DLS) in pH 7.4 HEPES buffer. Buffer exchange into pH 6.5 MES and antibody insertion resulted in size and polydispersity increase in all four lipid compositions. However, Lipid 2 and Lipid 6 LNPs showed significantly greater size distribution changes compared to Lipid 12 and Lipid 13 LNPs (FIG. 83A and FIG. 83B). As seen in FIG. 83C, under both physiological and acidic pH conditions (pH 7.4 and pH 5.5), Lipid 2 and Lipid 6 showed a greater positive surface charge relative to Lipid 12 and 13 indicating a significant shift in the LNP apparent pKa (in Lipid 12 and 13 LNPs) to lower values resulting from the mono- and di-hydroxyethyl substitution of the ionizable amine head groups, respectively. Additionally, in all four LNP compositions, low levels of dye accessible mRNA (<20%) and good RNA encapsulation efficiencies (>80% mRNA in parent LNP samples) were observed (Table 45 and FIG. 83D). The resulting targeted LNPs were evaluated in primary human T-cells using the in vitro transfection protocol described in example 8. As seen in FIG. 84E, all formulations were well tolerated by T-cells at all LNP doses tested (T-cell viability remained similar to the PBS control). As illustrated by the DiI+ and DiI MFI values (FIGS. 84C and 84D), all formulations show similar levels of cell association at most dose levels tested suggesting that the conjugate insertion process is not dependent on the ionizable lipid chemistry. Lipids 2 and Lipid 6 LNPs exhibited dose dependent expression of GFP protein (FIGS. 84A and 84B). However, at all doses tested Lipid 12 and Lipid 13 LNPs performed poorly potentially due to non-optimal LNP surface charge properties and diminished cytosolic access in T-cells. Additionally, Lipid 2 and Lipid 6 LNPs retained function after being subjected to freeze-thaw stress as illustrated in FIG. 85. Both compositions showed minor changes in particle size distributions after frozen storage at −80 C relative to particles stored at 4 C as seen in FIG. 83A and FIG. 83B. Furthermore, both compositions retained the ability to bind and transfect primary human T-cells post freeze-thaw with similar levels of % DiI+ and DiI MFI values as well as similar levels protein expression (% GFP+ cells and GFP MFI values) observed after refrigerated (4 C) and frozen (−80 C) storage conditions.

### Example 51—DiI T Cell Transfection Experiments

CD3+ T cells were isolated from frozen peripheral blood mononuclear cells using an EasySep Human T Cell Isolation Kit on a RoboSep automated cell isolation system from STEMCELL. T cells were plated into a round bottom 96-well plate in RPMI cell culture media supplemented with glutamax, 10% fetal bovine serum, pen-strep, and 40 ng/mL IL-2. 100 μL of cell suspension was seeded per well at a density of 1M T cells/mL (100K T cells/well). Cells were allowed to rest for two hours in a 37° C. incubator, and then were transfected by gently adding 10 μL of a 22 μg/mL (by mRNA) nanoparticle suspension, resulting in a final mRNA concentration of 2 μg/mL (unless otherwise noted). Cells were gently mixed with a pipette and then incubated for 24 hours in a 37° C. incubator. After incubation the cells were diluted with FACS buffer (BD 554657) and analyzed using a BD Fortessa flow cytometer. Data were analyzed using FlowJo software from BD biosciences.

### Example 52—CD4 and CD69 Staining

After 24 hours, cells were transferred to a 96-well conical bottom polypropylene plate and centrifuged at 350×g for 5 minutes. Supernatants were removed and transferred to a fresh conical bottom polypropylene plate for further analysis. Cells were washed by adding 200 μL FACS buffer (BD 554657), centrifuging at 350×g for 5 min, and then aspirating the supernatant from each well. BV421 anti-human CD69 (BioLegend 310930 clone FN50) and BV711 anti-human CD4 (BioLegend 344648 clone SK3) antibodies were diluted 100× by adding 100 μL of each antibody to 10 mL FACS buffer. 100 μL of the diluted antibody solution was added to each well and the plate was incubated at room temperature for 20 minutes. The plates were then washed by centrifuging at 350×g for 5 min, removing the supernatant, re-suspending in 200 μL FACS buffer, centrifuging at 350×g for 5 min and aspirating the supernatant from each well. Following the wash, cells were resuspended in 100 μL of 1.6% formaldehyde and stored at 4° C. until FACS analysis. FACS analysis was performed using a BD Fortessa equipped with a High Throughput Sampler.

### Example 53—Human IFN-γ ELISA

IFN-γ was assayed using an R&D Duoset IL-2 ELISA kit, PN DY285B. Briefly: an Immulon 2HB 96-well plate (Thermo X1506319) was coated by adding 100 μL of a 2 μg/mL solution of the R&D IL-2 capture antibody to each well and then incubating the plate overnight at 4° C. The plate was washed three times with wash buffer (0.05 TWEEN-20 in pH 7.4 TRIS buffered saline, Thermo 28360), blocked with reagent diluent (0.1% BSA in wash buffer) for one hour at room temperature, and then washed an additional three times with wash buffer. Supernatants were diluted three-fold in reagent diluent and then 100 μl of diluted supernatant was added to each well. IFN-γ standards were prepared on the same plate by serial dilution. Plates were incubated for two hours at room temperature, washed three times with wash buffer. 1004 of detection antibody diluted in reagent diluent was added, incubated for 2 hours at room temperature, and then the plate was washed three times with wash buffer. 1004 of Streptavidin-HRP was added, incubated for 20 minutes at room temperature, and then the plate was washed three times with wash buffer. 1004 of substrate solution (Thermo N301) was added, incubated for 20 minutes at room temperature and then the reaction was quenched by adding 1004 of stop solution (Invitrogen SS04). Optical density at 450 nm was read on a Spectramax M5 plate reader. IFN-γ concentration was quantified relative to a standard curve based on contemporaneously analyzed IFN-γ standards.

### Enumerated Embodiments

1. A compound represented by Formula I:

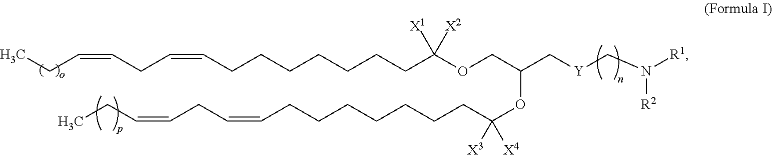

or a salt thereof, wherein:
R¹ and R² are independently C₁₋₃alkyl, or R¹ and R² are taken together with the nitrogen atom to form an optionally substituted piperidinyl or morpholinyl;
Y is selected from the group consisting of —O—, —OC(O)—, —OC(S)—, and —CH₂—;
X¹, X², X³, and X⁴ are hydrogen or X¹ and X² or X³ and X⁴ are taken together to form an oxo;
n is 0 or 3;
o and p are independently an integer selected from 2-6;
wherein the compound is not a compound selected from the group consisting of

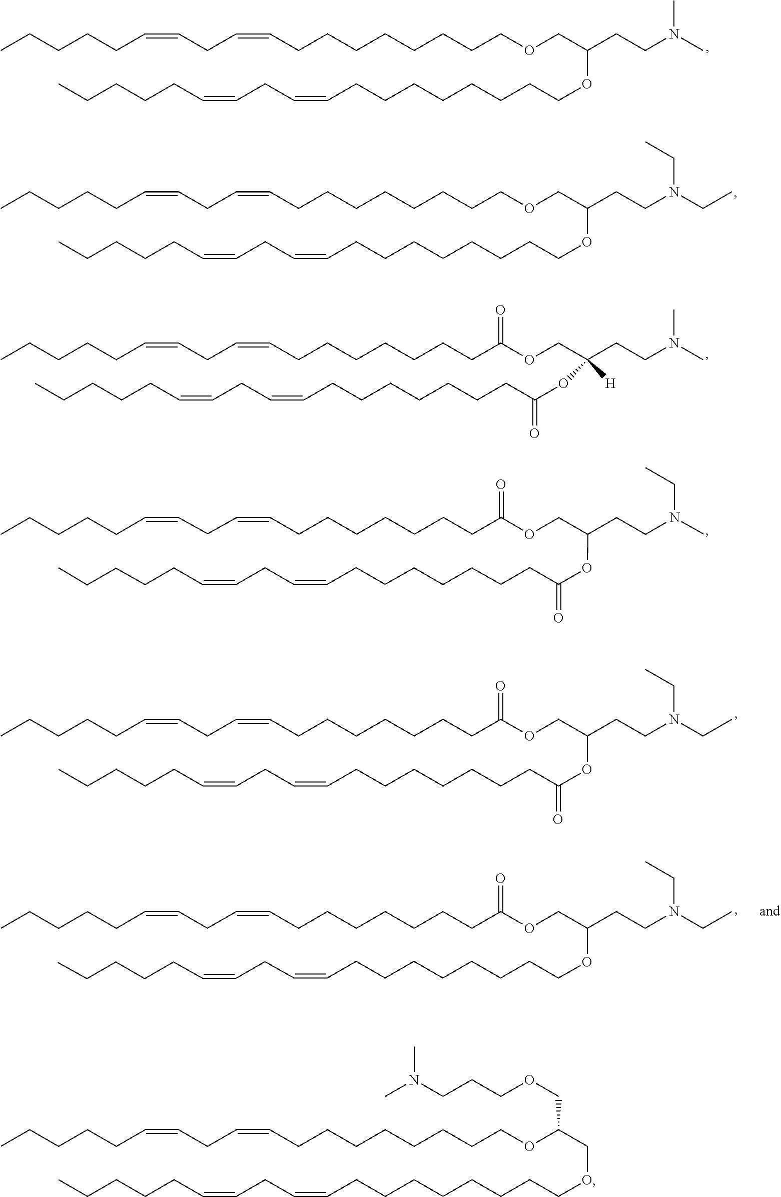

or a salt thereof.
2. The compound of embodiment 1, wherein o and p are 2.
3. The compound of embodiment 1, wherein o and p are 4.
4. The compound of embodiment 1, wherein o and p are 6.
5. The compound of any one of embodiments 1-4, wherein X1 and X2 are taken together to form an oxo and X3 and X4 are taken together to form an oxo.
6. The compound of any one of embodiments 1-4, wherein X1, X2, X3, and X4 are hydrogen.
7. The compound of any one of embodiments 1-6, wherein Y is selected from the group consisting of —O—, —OC(O)—, and —CH₂—.
8. The compound of embodiment 7, wherein Y is —O—.
9. The compound of embodiment 7, wherein Y is —OC(O)—.
10. The compound of embodiment 7, wherein Y is —CH₂—.
11. The compound of any one of embodiments 1-10, wherein R1 and R2 are independently C₁₋₃alkyl.
12. The compound of embodiment 11, wherein R1 and R2 are —CH3.
13. The compound of embodiment 11, wherein R1 and R2 are —CH2CH3.
14. The compound of any one of embodiments 1-13, wherein n is 0.
15. The compound of any one of embodiments 1-13, wherein n is 3.
16. A compound represented by Formula II:

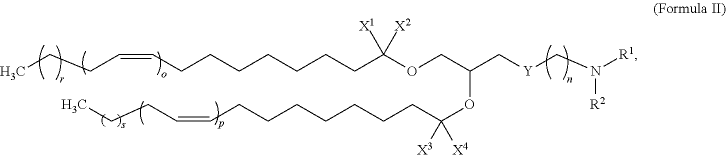

or a salt thereof, wherein:
R¹ and R² are independently C₁₋₃alkyl, or R¹ and R² are taken together with the nitrogen atom to form an optionally substituted piperidinyl or morpholinyl;
Y is selected from the group consisting of —O—, —OC(O)—, —OC(S)—, and —CH₂—;
X¹, X², X³, and X⁴ are hydrogen or X¹ and X² or X³ and X⁴ are taken together to form an oxo; n is 0-4;
o is 1 and r is an integer selected from 3-8 or o is 2 and r is an integer selected from 1-8,
p is 1 and s is an integer selected from 3-8 or p is 2 and s is an integer selected from 1-8,
wherein,
when o and p are both 1, r and s are independently 4, 5, 7, or 8, and
when o and p are both 2, r and s are independently 1, 2, 4, or 5.
17. The compound of embodiment 16, wherein X1 and X² are taken together to form an oxo and X³ and X⁴ are taken together to form an oxo.
18. The compound of embodiment 16 or 17, wherein X¹, X², X³, and X⁴ are hydrogen.
19. The compound of any one of embodiments 16-18, wherein Y is selected from the group consisting of —O—, —OC(O)—, and —CH₂—.
20. The compound of embodiment 19, wherein Y is —O—.
21. The compound of embodiment 19, wherein Y is —OC(O)—.
22. The compound of embodiment 19, wherein Y is —CH₂—.
23. The compound of any one of embodiments 16-22, wherein R¹ and R² are independently C₁₋₃alkyl.
24. The compound of embodiment 23, wherein R¹ and R² are —CH3.
25. The compound of embodiment 23, wherein R¹ and R² are —CH2CH3.
26. The compound of any one of embodiments 16-25, wherein n is 0.
27. The compound of any one of embodiments 16-25, wherein n is 3.
28. A compound selected from the group consisting of:

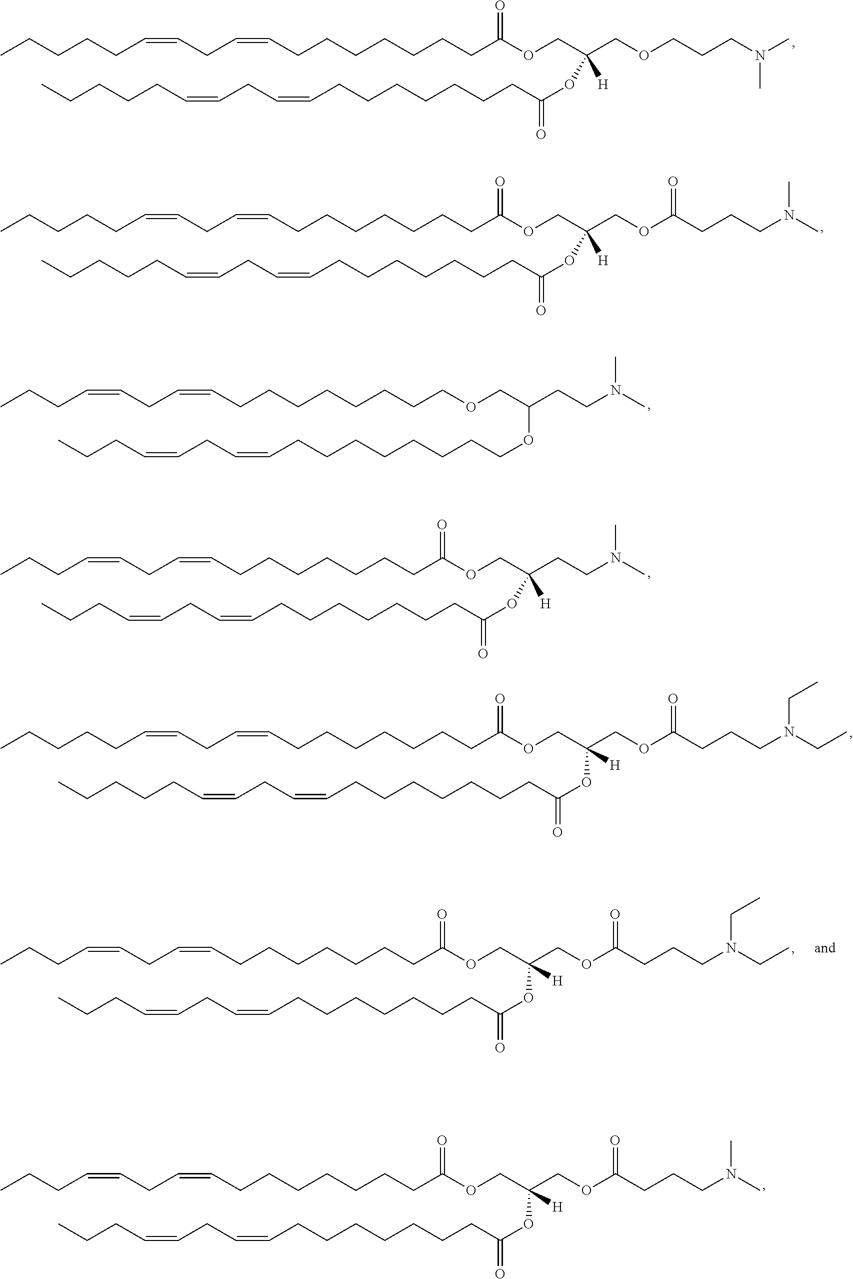

or a salt thereof.
29. The compound of embodiment 16, wherein the compound is a compound of Formula III:

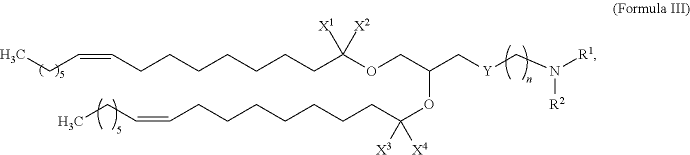

or a salt thereof, wherein:
R¹ and R² are independently C₁₋₃alkyl, or R¹ and R² are taken together with the nitrogen atom to form an optionally substituted piperidinyl;
Y is selected from the group consisting of —O—, —OC(O)—, —OC(S)—, and —CH₂—;
X¹, X², X³, and X⁴ are hydrogen or X¹ and X² or X³ and X⁴ are taken together to form an oxo; and
n is an integer selected from 0-4.
30. The compound of embodiment 29, wherein R¹ and R² are independently C₁₋₃alkyl.
31. The compound of embodiments 29 or 30, wherein R¹ and R² are —CH3.
32. The compound of any one of embodiments 29-31, wherein Y is —O—.
33. The compound of any one of embodiments 29-32, wherein X¹ and X² are taken together to form an oxo and X³ and X⁴ are taken together to form an oxo.
34. The compound of any one of embodiments 29-33, wherein n is 3.
35. A compound of formula:

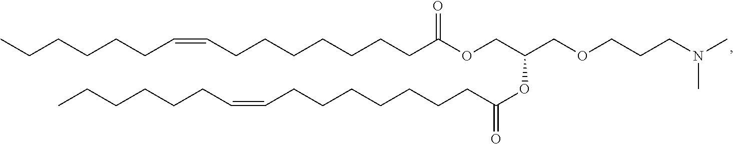 [SMILES: CCCCCC/C=C\CCCCCCCC(=O)OC[C@H](COCCCN(C)C)OC(=O)CCCCCCC/C=C\CCCCCC]

or a salt thereof.
36. A lipid nanoparticle (LNP) comprising a lipid blend comprising the compound of any one of embodiments 1-35 or a lipid of Table 1.
37. The LNP of embodiment 36, wherein the lipid blend further comprises one or more of a sterol, a neutral phospholipid, a PEG-lipid, and a lipid-immune cell targeting group conjugate.
38. The LNP of embodiment 35 or 36, wherein the compound is present in the lipid blend in a range of 30-70-60 mole percent.
39. The LNP of any one of embodiments 36-38, wherein the sterol (e.g., cholesterol) is present in the lipid blend in a range of 20-70 mole percent.
40. The LNP of any one of embodiments 36-39, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC).
41. The LNP of any one of embodiments 36-40, wherein the neutral phospholipid is present in the lipid blend in a range of 1-10 mole percent.
42. The LNP of any one of embodiments 36-41, wherein the PEG-lipid is selected from the group consisting of PEG-distearoylglycerol (PEG-DSG), PEG-dipalmitoylglycerol (PEG-DAG, e.g., PEG-DMG, PEG-DPG, and PEG-DSG), PEG-dimyristoyl-glycerol (PEG-DMG), PEG-distearoyl-phosphatidylethanolamine (PEG-DSPE), PEG-dipalmitoyl-phosphatidylethanolamine (PEG-DPPE) and PEG-dimyrstoyl-phosphatidylethanolamine (PEG-DMPE).
43. The LNP of any one of embodiments 36-42, wherein the PEG-lipid is present in the lipid blend in a range of 1-10 mole percent.
44. The LNP of any one of embodiments 36-43, wherein the lipid-immune cell targeting group conjugate is present in the lipid blend in a range of 0.1-0.3 mole percent or 0.002-0.2 mole percent.
45. The LNP of embodiment 44, wherein the targeting group is a T cell targeting group.
46. The LNP of embodiment 45, wherein the T cell targeting group is an antibody or antigen binding fragment thereof that binds a T cell antigen.
47. The LNP of embodiment 46, wherein the T cell antigen is selected from the group consisting of CD2, CD3, CD4, CD5, CD7, CD8, CD28, CD137, and T-cell receptor (TCR) (3 (e.g., CD3 or CD8).
48. The LNP of any one of embodiments 36-47, wherein the T cell-targeting group is covalently coupled to a lipid via a polyethylene glycol (PEG) containing linker.
49. The LNP of embodiment 48, wherein the lipid is distearoyl-phosphatidylethanolamine (DSPE), dipalmitoyl-phosphatidylethanolamine (DPPE), dimyrstoyl-phosphatidylethanolamine (DMPE), distearoyl-glycero-phosphoglycerol (DSPG), distearoyl-glycerol (DSG), dimyristoyl-glycerol (DMG), or ceramide.
50. The LNP of embodiment 48 or 49, wherein the PEG is selected from the group consisting of PEG 2000, PEG 1000, PEG 3000, PEG 3450, PEG 4000, or PEG 5000.
51. The LNP of any one of embodiments 36-50, wherein the lipid blend further comprises free PEG-distearoyl-phosphatidylethanolamine (PEG-DSPE), PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-1succinyl[methoxy(polyethylene glycol)] (PEG-ceramide), and DSPE-PEG-cysteine, or a derivative thereof 52. The LNP of embodiment 51, wherein the derivative of the PEG-lipid has a hydroxyl or a carboxylic acid end group at the PEG terminus.
53. The LNP of any one of embodiments 36-52, wherein the LNP has a mean diameter in the range of 50-200 nm.
54. The LNP of embodiment 53, wherein the LNP has a mean diameter of about 100 nm.
55. The LNP of any one of embodiments 36-54, wherein the LNP has a polydispersity index in a range from 0.05 to 1.
56. The LNP of any one of embodiments 36-55, wherein the LNP has a zeta potential of from about −10 mV to about +30 mV at pH 5.
57. The LNP of any one of embodiments 36-55, wherein the LNP has a zeta potential of from about −30 mV to about +5 mV at pH 7.4.
58. The LNP of any one of embodiments 36-57, further comprising a nucleic acid disposed therein.
59. The LNP of embodiment 58, wherein the nucleic acid is DNA or RNA.
60. A lipid nanoparticle (LNP) comprising a lipid blend comprising a lipid-T-cell-targeting group conjugate and optionally a lipid set forth in Table 1.
61. The LNP of embodiment 60, wherein the T-cell targeting group is an antibody that binds a T cell antigen.
62. The LNP of embodiment 61, wherein the T cell antigen is selected from the group consisting of CD2, CD3, CD4, CD5, CD7, CD8, CD28, CD137, and T-cell receptor (TCR) (3.
63. The LNP of embodiment 62, wherein the T cell antigen is CD2, CD3, CD7, or CD8.
64. The LNP of any one of embodiments 60-63, wherein the T-cell-targeting group is covalently coupled to the lipid via a polyethylene glycol (PEG) containing linker.
65. The LNP of embodiment 64, wherein the lipid is distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.
66. The LNP of embodiment 64 or 65, wherein the PEG is PEG 2000, PEG 1000, PEG 3000, PEG 3450, PEG 4000, or PEG 5000.
67. The LNP of any one of embodiments 60-66, wherein the lipid-T-cell targeting group conjugate is present in the lipid blend in a range of 0.002-0.2 mole percent.
68. The LNP of any one of embodiments 60-67, wherein the lipid bend further comprises one or more of a cationic lipid, sterol, a neutral phospholipid, and a PEG-lipid.
69. The LNP of embodiment 68, wherein the ionizable cationic lipid is a compound of any one of embodiments 1-35, or a lipid set forth in Table 1.
70. The LNP of embodiment 68 or 69, wherein the ionizable cationic lipid is present in the lipid blend in a range of 40-60 mole percent.
71. The LNP of any one of embodiments 68-70, wherein the sterol (e.g., cholesterol) is present in the lipid blend in a range of 30-50 mole percent.
72. The LNP of any one of embodiments 68-71, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), sphingomyelin.
73. The LNP of any one of embodiments 68-72, wherein the neutral phospholipid is present in the lipid blend in a range of 1-10 mole percent.
74. The LNP of any one of embodiments 68-73, wherein the PEG-lipid is selected from the group consisting of PEG-distearoylglycerol (PEG-DSG), PEG-diacylglycerol (PEG-DAG, e.g., PEG-DMG, PEG-DPG, and PEG-DSG), PEG-dimyristoyl-glycerol (PEG-DMG), PEG-distearoyl-phosphatidylethanolamine (PEG-DSPE) and PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG), 1,2-Dipalmitoyl-rac-glycero-3-methylpolyoxyethylene (PEG-DPG), 1,2-Dioleoyl-rac-glycerol, methoxypolyethylene Glycol (DOG-PEG) and N-palmitoyl-sphingosine-1-1succinyl[methoxy(polyethylene glycol)] (PEG-ceramide).
75. The LNP of any one of embodiments 68-74, wherein the PEG-lipid is present in the lipid blend in a range of 2-4 mole percent.

76. The LNP of any one of embodiments 68-75, wherein the lipid blend further comprises free PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl[methoxy(polyethylene glycol)] (PEG-ceramide), and DSPE-PEG-cysteine, or a derivative thereof.

77. The LNP of any one of embodiments 60-75, wherein the LNP has a mean diameter in the range of 50-200 nm.
78. The LNP of embodiment 77, where the LNP has a mean diameter of about 100 nm.
79. The LNP of any one of embodiments 60-78, wherein the LNP has a polydispersity index in a range from 0.05 to 1.
80. The LNP of any one of embodiments 60-79, wherein the LNP has a zeta potential of from about −10 mV to about +30 mV at pH 5.
81. The LNP of any one of embodiments 60-80, further comprising a nucleic acid disposed therein.
82. The LNP of embodiment 81, wherein the nucleic acid is DNA or RNA (e.g., an mRNA, tRNA, or siRNA).
83. The LNP of embodiment 81 or 82, wherein the number of the nucleotides in the nucleic acid is from about 400 to about 6000.
84. A method of delivering a nucleic acid to an immune cell (e.g., a T-cell), the method comprising exposing the immune cell to an LNP of any one of embodiments 36-83 containing a nucleic acid under conditions that permit the nucleic acid to enter the immune cell.
85. A method of delivering a nucleic acid to an immune cell (e.g., a T-cell) in a subject in need thereof, the method comprising administering to the subject a composition comprising the LNP of any one of embodiments 36-83 containing a nucleic acid thereby to deliver the nucleic acid to the immune cell.
86. A method of targeting the delivering of a nucleic acid (e.g., mRNA) to an immune cell (e.g., a T-cell) in a subject, the method comprising administering to the subject an LNP of any one of embodiments 36-83 containing the nucleic acid so as to facilitate targeted delivery of the nucleic acid to the immune cell.

### INCORPORATION BY REFERENCE

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, scientific articles, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A lipid nanoparticle (LNP) comprising a lipid blend for targeted delivery of a nucleic acid into an immune cell, the lipid blend comprising:
(a) a lipid-immune cell targeting group conjugate comprising the compound of Formula IV: [Lipid]-[optional linker]-[immune cell targeting group], and
(b) an ionizable cationic lipid comprising 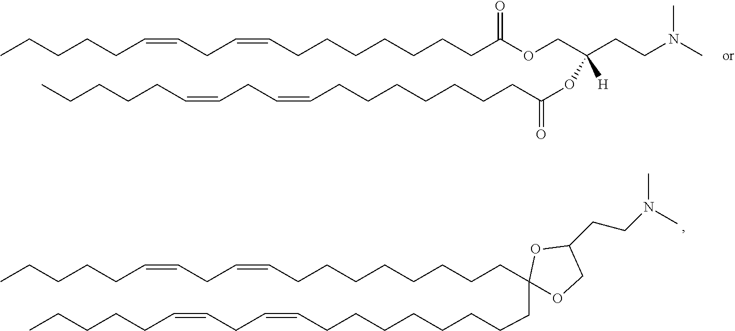
wherein the LNP further comprises a nucleic acid disposed therein.

2-46. (canceled)

47. A method of targeting the delivery of a nucleic acid to an immune cell of a subject, comprising contacting the immune cell with a lipid nanoparticle (LNP), wherein the LNP comprises:
(a) An ionizable cationic lipid,
(b) A conjugate comprising the compound of the following formula:
[Lipid]-[optional linker]-[immune cell targeting group];
(c) A sterol or other structural lipid;
(d) A neutral phospholipid
(e) A free Polyethylene glycol (PEG) lipid, and
(f) the nucleic acid,
wherein the LNP provides at least one of the following benefits:
(i) increased specificity of targeted delivery to the immune cell compared to a reference LNP;
(ii) increased half-life of the nucleic acid or a polypeptide encoded by the nucleic acid in the immune cell compared to a reference LNP;
(iii) increased transfection rate compared to a reference LNP; and
(iv) a low level of dye accessible mRNA (<15%) and high RNA encapsulation efficiencies, wherein at least 80% mRNA was recovered in final formulation relative to the total RNA used in LNP batch preparation.

48. A method of expressing a polypeptide of interest in a targeted immune cell of a subject, comprising contacting the immune cell with a lipid nanoparticle (LNP), wherein the LNP comprises:
(a) An ionizable cationic lipid;
(b) A conjugate comprising the following structure:
[Lipid]-[optional linker]-[immune cell targeting group];
(c) A sterol or other structural lipid;
(d) A neutral phospholipid
(e) A free Polyethylene glycol (PEG) lipid, and
(f) a nucleic acid encoding the polypeptide.

49. The method of claim 48, wherein the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP; and
(v) a low level of dye accessible mRNA (<15%) and high RNA encapsulation efficiencies, wherein at least 80% mRNA was recovered in final formulation relative to the total RNA used in LNP batch preparation.

50. A method of modulating cellular function of a target immune cell of a subject, comprising administering to the subject a lipid nanoparticle (LNP), wherein the LNP comprises:
(a) An ionizable cationic lipid,
(b) A conjugate comprising the following structure:
[Lipid]-[optional linker]-[immune cell targeting group];
(c) A sterol or other structural lipid;
(d) A neutral phospholipid;
(e) A free Polyethylene glycol (PEG) lipid, and
(f) a nucleic acid encoding a polypeptide for modulating the cellular function of the immune cell.

51. The method of claim 50, wherein the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP; and
(v) the LNP can be administered at a lower dose compared to a reference LNP to reach the same biologic effect in the immune cell; and
(vi) a low level of dye accessible mRNA (<15%) and high RNA encapsulation efficiencies, wherein at least 80% mRNA was recovered in final formulation relative to the total RNA used in LNP batch preparation.

52. The method of claim 50, wherein the modulation of cell function comprises reprogramming the immune cells to initiate an immune response.

53. The method of claim 50, wherein the modulation of cell function comprises modulating antigen specificity of the immune cell.

54. A method of treating, ameliorating, or preventing a symptom of a disorder or disease in a subject in need thereof, comprising administering to the subject a lipid nanoparticle (LNP) for delivering a nucleic acid into an immune cell of the subject, wherein the LNP comprises:
(a) An ionizable cationic lipid,
(b) A conjugate comprising the following structure:
[Lipid]-[optional linker]-[immune cell targeting group];
(c) A sterol or other structural lipid;
(d) A neutral phospholipid;
(e) A free Polyethylene glycol (PEG) lipid, and
(f) the nucleic acid,
Wherein the nucleic acid modulates the immune response of the immune cell, therefore to treat or ameliorate the symptom.

55. The method of claim 50, wherein the LNP provides at least one of the following benefits:
(i) increased specificity of delivery of the nucleic acid into the immune cell compared to a reference LNP;
(ii) increased half-life of the nucleic acid or a polypeptide encoded by the nucleic acid in the immune cell compared to a reference LNP;
(iii) increased transfection rate compared to a reference LNP;
(v) the LNP can be administered at a lower dose compared to a reference LNP to reach the same treatment efficacy; and
(vi) a low level of dye accessible mRNA (<15%) and high RNA encapsulation efficiencies, wherein at least 80% mRNA was recovered in final formulation relative to the total RNA used in LNP batch preparation.

56. The method of claim 54, wherein the disorder is an immune disorder, an inflammatory disorder, or cancer.

57. The method of claim 54, wherein the nucleic acid encodes an antigen for use in a therapeutic or prophylactic vaccine for treating or preventing an infection by a pathogen.

58. The method of claim 47, wherein the ionizable cationic lipid is

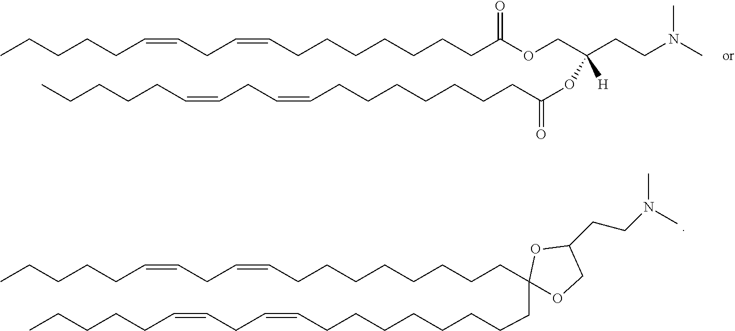

59. The method of claim 47, wherein the immune cell targeting group comprises an antibody that binds a T cell antigen.

60. The method of claim 59, wherein the T cell antigen is CD3, CD8, or both CD3 and CD8.

61. (canceled)

62. The method of claim 59, wherein the antibody is a human or humanized antibody.

63. The method of claim 47, wherein the immune cell targeting group is covalently coupled to a lipid in the lipid blend via a polyethylene glycol (PEG) containing linker.

64. The method of claim 63, wherein the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.

65. The method of claim 63, wherein the PEG is PEG 2000.

66. The method of claim 47,
wherein the lipid-immune cell targeting group conjugate is present in the lipid blend in a range of 0.002-0.2 mole percent.

67. The method of claim 47, wherein the ionizable cationic lipid is present in the lipid blend in a range of 40-60 mole percent.

68. The method of claim 47, wherein the sterol is cholesterol.

69. The method of claim 47, wherein the sterol is present in the lipid blend in a range of 30-50 mole percent.

70. The method of claim 47, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), sphingomyelin (SM).

71. The method of claim 47, wherein the neutral phospholipid is present in the lipid blend in a range of 1-10 mole percent.

72. The method of claim 47, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols.

73. The method of claim 47, wherein the free PEG-lipid comprises a diacylphosphatidylethanolamines comprising Dipalmitoyl (C16) chain or Distearoyl (C18) chain.

74. The method of claim 47, wherein the free PEG-lipid is present in the lipid blend in a range of 2-4 mole percent.

75. The method of claim 47, wherein the free PEG-lipid comprises the same or a different lipid as the lipid in the lipid-immune cell targeting group conjugate.

76. The method of claim 47, wherein the LNP has a mean diameter in the range of 50-200 nm.

77. The method of claim 76, where the LNP has a mean diameter of about 100 nm.

78. The method of claim 47, wherein the LNP has a polydispersity index in a range from 0.05 to 1.

79. The method of claim 47, wherein the LNP has a zeta potential of from about −10 mV to about +30 mV at pH 5.

80. The method of claim 47, wherein the nucleic acid is DNA or RNA.

81. The method of claim 80, wherein the RNA is an mRNA, tRNA, siRNA, or microRNA.

82. The method of claim 81, wherein the mRNA encodes a receptor, a growth factor, a hormone, a cytokine, an antibody, an antigen, an enzyme, or a vaccine.

83. The method of claim 81, wherein the mRNA encodes a polypeptide capable of regulating immune response in the immune cell.

84. The method of claim 81, wherein the mRNA encodes a polypeptide capable of reprogramming the immune cell.

85. The method of claim 81, wherein the mRNA encodes a synthetic T cell receptor (synTCR) or a Chimeric Antigen Receptor (CAR).

86. The method of claim 47, wherein the immune cell targeting group comprises an antibody, and the antibody is a Fab or an immunoglobulin single variable domain.

87. The method of claim 47, wherein the immune cell targeting group comprises an antibody fragment selected from the group consisting of a Fab, F(ab′)2, Fab′-SH, Fv, and scFv fragment.

88. The method of claim 86, wherein the immune cell targeting group comprises a Fab that comprises one or more interchain disulfide bonds.

89. The method of claim 88, wherein the Fab comprises a heavy chain fragment that comprises F174C and C233S substitutions, and a light chain fragment that comprises S176C and C214S substitutions, numbering according to Kabat.

90. The method of claim 86, wherein the immune cell targeting group comprises a Fab that comprises a cysteine at the C-terminus of the heavy or light chain fragment.

91. The method of claim 86, wherein the Fab further comprises one or more amino acids between the heavy chain fragment of the Fab and the C-terminal cysteine.

92. The method of claim 87, wherein the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker.

93. The method of claim 86, wherein the immune cell targeting group comprises an immunoglobulin single variable domain.

94. The method of claim 93, wherein the immunoglobulin single variable domain comprises a cysteine at the C-terminus.

95. The method of claim 94, wherein the immunoglobulin single variable domain comprises a VHH domain and further comprises a spacer comprising one or more amino acids between the VHH domain and the C-terminal cysteine.

96. The method of claim 86, wherein the immune cell targeting group comprises two or more VHH domains.

97. The method of claim 96, wherein the two or more VHH domains are linked by an amino acid linker.

98. The method of claim 96, wherein the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds.

99. The method of claim 86, wherein the immune cell targeting group comprises a VHH domain linked to an antibody CH1 domain, and wherein the antibody CH1 domain is linked to an antibody light chain constant domain by one or more disulfide bonds.

100. The method of claim 99, wherein the antibody CH1 domain comprises F174C and C233S substitutions, and the light chain constant domain comprises S176C and C214S substitutions, numbering according to Kabat.

101. The method of claim 47, wherein the immune cell targeting group comprises a Fab that comprises:
(a) a heavy chain fragment comprising the amino acid sequence of SEQ ID NO: 1 and a light chain fragment comprising the amino acid sequence of SEQ ID NO:2 or 3;
(b) a heavy chain fragment comprising the amino acid sequence of SEQ ID NO: 6 and a light chain fragment comprising the amino acid sequence of SEQ ID NO: 7.

102. The method of claim 47, wherein no more than 5% non-immune cells are transfected by the LNP.

103. The method of claim 47, wherein half-life of the nucleic acid delivered by the LNP or a polypeptide encoded by the nucleic acid delivered by the LNP is at least 10% longer than half-life of nucleic acid delivered by a reference LNP or a polypeptide encoded by the nucleic acid delivered by the reference LNP.

104. The method of claim 47, wherein at least 10% immune cells are transfected by the LNP.

105. The method of claim 47, wherein expression level of the nucleic acid delivered by the LNP is at least 10% higher than expression level of nucleic acid delivered by a reference LNP.

106-170. (canceled)

171. An immunoglobulin single variable domain (ISVD) that binds to human CD8, wherein the ISVD comprises three complementatity determining domains CDR₁, CDR₂, and CDR₃, wherein
(a) the CDR₁ comprises GSTFSDYG (SEQ ID NO: 100),
(b) the CDR₂ comprises IDWNGEHT (SEQ ID NO: 101), and
(c) the CDR₃ comprises AADALPYTVRKYNY (SEQ ID NO: 102).

172-173. (canceled)

174. A polypeptide comprising GSTFSDYG (SEQ ID NO: 100), IDWNGEHT (SEQ ID NO: 101), and AADALPYTVRKYNY (SEQ ID NO: 102).

175. A polypeptide comprising the ISVD of claim 171.

176-179. (canceled)

180. A composition comprising the ISVD of claim 171.

181. A pharmaceutical composition comprising the ISVD of claim 171, and a pharmaceutically acceptable carrier.

182. A method of treating a disease or disorder related to CD8 in a subject, comprising administering the pharmaceutical composition of claim 181 to the subject.

183. (canceled)

184. The method of claim 54, wherein the immune cell targeting group comprises an antibody that binds a Natural Killer (NK) cell antigen.

185. The method of claim 184, wherein the NK cell antigen is CD7, CD8, or CD56.

186. The method of claim 72, wherein the free PEG-lipid is PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-di stearoylglycerol (PEG-D SG), PEG-diacylglycerol (PEG-DAG, e.g., PEG-DMG, PEG-DPG, and PEG-DSG), PEG-ceramide, PEG-di stearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.
